SEQUENCE LISTING (48 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 363 aa, SARS-CoV-2
VTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1268 aa, SARS-CoV
MPMGSLQPLATLYLLGMLVASVLASGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 4 - protein, 363 aa, SARS-CoV
VTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 5 - protein, 1270 aa, SARS-CoV-2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 363 aa, SARS-CoV-2
VTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1353 aa, MERS-CoV
MIHSVFLLMFLLTPTESYVDVGPDSAKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSAIIRKIYPAFMLGSSVGNFSYGKMGRFFNHTLVLLPDGCGTLLRAFYCILEPRSGNYCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLFGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVLQLVNANQYSPCVSIVPSTVWEDGDYYRKQLSPLEGGGWLVASGSTVAMTEQLQMGFGITVQYGTDTNSVCPKLEFANDTKIVSQLGNCVEYSLYGVSGRGVFQNCTAVGVPQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFESVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDSANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLSFIAGLVALALCVFFILCCTGCGTNCMGKLKCNRCCDRYEEYDLEPHKVHVH

SEQ ID NO: 8 - protein, 369 aa, MERS-CoV
ITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDSANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLSFIAGLVALALCVFFILCCTGCGTNCMGKLKCNRCCDRYEEYDLEPHKVHVH

SEQ ID NO: 9 - protein, 1173 aa, HCoV-229E
MFVLLVAYALLHIAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKLVTSGLGTVDADYKKCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 10 - protein, 392 aa, HCoV-229E
LQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 11 - protein, 1356 aa, HCoV-NL63
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 12 - protein, 394 aa, HCoV-NL63
LQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 13 - protein, 1351 aa, HCoV-HKU1
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRRKRRGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDNLEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMYVKWPWYIWLLIVILFIIFLMILFFICCCTGCGSACFSKCHNCCDEYGGHNDFVIKASHDD

SEQ ID NO: 14 - protein, 360 aa, HCoV-HKU1
VTMDVLNKNQKLIANAFNKALLSIQNGFTATNSALAKIQSVVNANAQALNSLLQQLFNKFGAISSSLQEILSRLDNLEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMYVKWPWYIWLLIVILFIIFLMILFFICCCTGCGSACFSKCHNCCDEYGGHNDFVIKASHDD

SEQ ID NO: 15 - protein, 1353 aa, HCoV-OC43
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAEDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYIINRTFMIRSCYSGRVSAAFHANSSEPALLFRNIKCNYVFNNSLTRQLQPINYFDSYLGCVVNAYNSTAISVQTCDLTVGSGYCVDYSKNRRSRGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 16 - protein, 359 aa, HCoV-OC43
VTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 17 - protein, 856 aa, HIV
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 18 - protein, 345 aa, HIV
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 19 - protein, 676 aa, Ebola
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 20 - protein, 175 aa, Ebola
EAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 21 - protein, 577 aa, Influenza
MPMGSLQPLATLYLLGMLVASCLGTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 22 - protein, 574 aa, Influenza
MPMGSLQPLATLYLLGMLVASCLGQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 23 - protein, 595 aa, Influenza
MPMGSLQPLATLYLLGMLVASCLGTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDSETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 24 - protein, 597 aa, Influenza
MPMGSLQPLATLYLLGMLVASCLGTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 25 - protein, 221 aa, Influenza
GIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 26 - DNA, 5428 bp, Synthetic Polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 27 - protein, 297 aa, SARS-CoV-2
DKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTDYKDDDDK

SEQ ID NO: 28 - protein, 23 aa, Synthetic Polypeptide
MFVFLVLLPLVSSQCVNLTTRTQ

SEQ ID NO: 29 - protein, 30 aa, Synthetic Polypeptide
MRVKEKYQHLWRWGWRWGTMLLGMLMICSA

SEQ ID NO: 30 - protein, 32 aa, Synthetic Polypeptide
MGVTGILQLPRDRFKRTSFFLWVIILFQRTFS

SEQ ID NO: 31 - protein, 24 aa, Synthetic Polypeptide
MPMGSLQPLATLYLLGMLVASCLG

SEQ ID NO: 32 - protein, 25 aa, Synthetic Polypeptide
MELPILKTNAIITILAAVTLCFASS

SEQ ID NO: 33 - protein, 574 aa, RSV
MELPILKTNAIITILAAVTLCFASSQNITEEFYQSTCSAVSKGYLSALRTGWYTSVITIELSNIKENKCNGTDAKVKLIKQELDKYKNAVTELQLLMQSTPAANSRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 34 - protein, 268 aa, RSV
GVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 35 - DNA, 21 bp, Synthetic Polynucleotide
gttcaaggacggcatctactt

SEQ ID NO: 36 - DNA, 21 bp, Synthetic Polynucleotide
acgctctgggacttgttattc

SEQ ID NO: 37 - DNA, 20 bp, Synthetic Polynucleotide
gacaaagtgcaccctgaaga

SEQ ID NO: 38 - DNA, 20 bp, Synthetic Polynucleotide
gggcacaggttggtgatatt

SEQ ID NO: 39 - DNA, 21 bp, Synthetic Polynucleotide
gaacgcctctctgaactcttt

SEQ ID NO: 40 - DNA, 21 bp, Synthetic Polynucleotide
gtcctcggtgatgttgtatgt

SEQ ID NO: 41 - DNA, 21 bp, Synthetic Polynucleotide
gattagagccgccgagattag

SEQ ID NO: 42 - DNA, 21 bp, Synthetic Polynucleotide
ggactgagggaaagacatgag

SEQ ID NO: 43 - protein, 386 aa, SARS-CoV-2
MFVFLVLLPLVSSQCVNLTTRTQVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 312 aa, SARS-CoV-2
MFVFLVLLPLVSSQCVNLTTRTQDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 45 - protein, 375 aa, HIV
MRVKEKYQHLWRWGWRWGTMLLGMLMICSAAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 46 - protein, 207 aa, Ebola
MGVTGILQLPRDRFKRTSFFLWVIILFQRTFSEAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 47 - protein, 323 aa, RSV
MELPILKTNAIITILAAVTLCFASSQNITEEFYQSTCSAVSKGYLSALRTGSGGSGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 48 - protein, 245 aa, Influenza
MPMGSLQPLATLYLLGMLVASCLGGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

(19) United States
(12) Patent Application Publication
(10) Pub. No.: US 2024/0307486 A1
(43) Pub. Date: Sep. 19, 2024

(54) METHODS AND COMPOSITIONS FOR HIGH-POTENCY POLYPEPTIDE-BASED PROTEIN INHIBITION

(71) Applicant: Baylor College of Medicine, Houston, TX (US)

(72) Inventors: Qinghua Wang, Houston, TX (US); Jianpeng Ma, Houston, TX (US)

(21) Appl. No.: 18/282,730

(22) PCT Filed: Mar. 29, 2022
(86) PCT No.: PCT/US2022/071424

§ 371 Date: Sep. 18, 2023

Related U.S. Application Data
(60) Provisional application No. 63/168,107, filed on Mar. 30, 2021.

(51) Int. Cl. A61K 38/16; A61K 45/06; A61P 31/14

(52) U.S. Cl. CPC A61K 38/162; A61K 45/06; A61P 31/14

(57) ABSTRACT

Aspects of the present disclosure relate compositions comprising polypeptides or polynucleotides encoding such polypeptides that interact with target proteins such as viral spike proteins, and to methods of their use for treatment and prevention of disease, such as viral infections and/or post-viral infection syndromes.

### CROSS-REFERENCE TO RELATED APPLICATIONS

This application claims priority to U.S. Provisional Application Ser. No. 63/168,107, filed Mar. 30, 2021, which is incorporated by reference herein in its entirety.

### BACKGROUND

### Technical Field

Aspects of this disclosure relate, generally, to at least the fields of protein biology, molecular biology, virology, and medicine.

### Background

Proteins are major players in carrying out the vast spectrum of biological functions in a given organism. For example, infections by viruses, such as coronaviruses, HIV, Ebola, RSV, and influenza viruses, are mediated by binding of the virus spike proteins to receptors on the surface membranes of host cells. Viral infections cause severe life-threatening conditions like severe acute respiratory syndrome, Middle East respiratory syndrome, COVID-19, or respiratory infections, and other symptoms that can persist and result in post-viral infection syndromes. Unfortunately, proteins have not become a major therapeutic tool in modern medicine. In fact, few therapeutic options for treatment of these infections have proven effective in robust clinical trials.

Thus, there exists a need for compositions and methods for protein or polypeptide-based therapeutics, especially for treatment and prevention of viral infections and/or post-viral infection syndromes.

### SUMMARY

Aspects of the present disclosure are directed to polypeptides that target a protein, including but not limited to a viral spike protein, in vivo, compositions comprising such polypeptides, and methods of use for treatment and prevention of a disease or condition in a subject, including but not limited to a coronavirus (e.g., MERS-CoV, SARS-CoV, SARS-CoV-2, HCoV-229E, HCoV-NL63, HCoV-OC43, HCoV-HKU1) infection, a human immunodeficiency virus (HIV) infection, an Ebola infection, an RSV infection, or an influenza infection. In some embodiments, the polypeptide has an amino acid sequence corresponding to a sequence of the target protein, and the amino acid sequence has at least 10% sequence identity with the corresponding sequence of the target protein. In some embodiments, the polypeptide has an amino acid sequence corresponding to an oligomerization domain of a target protein, and the amino acid sequence has at least 10% sequence identity with the oligomerization domain of the target protein. In some embodiments, the polypeptide comprises an amino acid sequence having at least 10-80% identity with SEQ ID NOS:2, 4, 6, 8, 10, 12, 14, 16, 18, 20, 25, or 34. In some embodiments, the polypeptide has an amino acid sequence corresponding to an oligomerization domain of a coronavirus spike protein, and the amino acid sequence has at least 10-80% identity with SEQ ID NOS:2, 4, 6, 8, 10, 12, 14, or 16. In some embodiments, the polypeptide has an amino acid sequence corresponding to a sequence of an HIV spike protein, and the amino acid sequence has at least 10-80% identity with SEQ ID NO:18. In some embodiments, the polypeptide has an amino acid sequence corresponding to a sequence of an Ebola virus glycoprotein, and the amino acid sequence has at least 10-80% identity with SEQ ID NO:20. In some embodiments, the polypeptide has an amino acid sequence corresponding to a sequence of an influenza virus HA spike protein, and the amino acid sequence has at least 10-80% identity with SEQ ID NO:25. In some embodiments, the polypeptide has an amino acid sequence corresponding to a sequence of an RSV glycoprotein, and the amino acid sequence has at least 10-80% identity with SEQ ID NO:34.

Embodiments of the disclosure include polypeptides; viral spike proteins; protein-interacting polypeptides; viral spike protein-interacting polypeptides; coronavirus spike protein-interacting polypeptides; MERS-CoV spike protein-interacting polypeptides; SARS-CoV spike protein-interacting polypeptides; SARS-CoV-2 spike protein-interacting polypeptides; HCoV-229E spike protein-interacting polypeptides; HCoV-NL63 spike protein-interacting polypeptides; HCoV-HKU1 spike protein-interacting polypeptides; HCoV-OC43 spike protein-interacting polypeptides; HIV spike protein-interacting polypeptides; Ebola virus glycoprotein-interacting polypeptides; influenza virus HA spike protein-interacting polypeptides; RSV glycoprotein-interacting polypeptides;
vectors encoding polypeptides; vectors encoding protein-interacting polypeptides; vectors encoding viral spike protein-interacting polypeptides; vectors encoding coronavirus spike protein-interacting polypeptides; vectors encoding MERS-CoV spike protein-interacting polypeptides; vectors encoding SARS-CoV spike protein-interacting polypeptides; vectors encoding SARS-CoV-2 spike protein-interacting polypeptides; vectors encoding HCoV-229E spike protein-interacting polypeptides; vectors encoding HCoV-NL63 spike protein-interacting polypeptides; vectors encoding HCoV-HKU1 spike protein-interacting polypeptides; vectors encoding HCoV-OC43 spike protein-interacting polypeptides; vectors encoding HIV spike protein-interacting polypeptides; vectors encoding Ebola virus glycoprotein-interacting polypeptides; vectors encoding influenza virus HA spike protein-interacting polypeptides; vectors encoding RSV glycoprotein-interacting polypeptides;
methods for regulation of a target protein or biological function thereof in vivo in a subject; methods for regulation of viral spike proteins or biological function thereof in vivo in a subject; methods for regulation of a coronavirus spike protein or biological function thereof in vivo in a subject; methods for regulation of a MERS-CoV spike protein or biological function thereof in vivo in a subject; methods for regulation of a SARS-CoV spike protein or biological function thereof in vivo in a subject; methods for regulation of a SARS-CoV-2 spike protein or biological function thereof in vivo in a subject; methods for regulation of a HCoV-229E spike protein or biological function thereof in vivo in a subject; methods for regulation of a HCoV-NL63 spike protein or biological function thereof in vivo in a subject; methods for regulation of a HCoV-HKU1 spike protein or biological function thereof in vivo in a subject; methods for regulation of a HCoV-OC43 spike protein or biological function thereof in vivo in a subject; methods for regulation of an HIV spike protein or biological function thereof in vivo in a subject; methods for regulation of an Ebola virus glycoprotein or biological function thereof in vivo in a subject; methods for regulation of a influenza virus HA spike protein or biological function thereof in vivo in a subject; methods for regulation of an RSV glycoprotein or biological function thereof in vivo in a subject;
methods for treatment of a disease; methods for treatment of a viral infection and/or a post-viral infection syndrome; methods for treatment of a coronavirus infection and/or post-coronavirus infection syndrome; methods for treatment of a MERS-CoV infection; methods for treatment of a SARS-CoV infection; methods for treatment of a SARS-CoV-2 infection; methods for treatment of a HCoV-229E infection; methods for treatment of a HCoV-NL63 infection; methods for treatment of a HCoV-OC43 infection; methods for treatment of a HCoV-HKU1 infection; methods for treatment of an HIV infection; methods for treatment of an Ebola infection; methods for treatment of an influenza infection; methods for treatment of an RSV infection;
methods for prevention of a disease; methods for prevention of a viral infection and/or a post-viral infection syndrome; methods for prevention of a coronavirus infection and/or post-coronavirus infection syndrome; methods for the prevention of a MERS-CoV infection; methods for prevention of a SARS-CoV infection; methods for prevention of a SARS-CoV-2 infection; methods for prevention of a HCoV-229E infection; methods for prevention of a HCoV-NL63 infection; methods for prevention of a HCoV-OC43 infection; methods for prevention of a HCoV-HKU1 infection; methods for prevention of an HIV infection; methods for prevention of an Ebola infection; methods for prevention of an influenza infection; methods for prevention of an RSV infection;
methods for reducing severity of a disease; methods for reducing severity of a viral infection and/or post-viral infection syndrome; methods for reducing severity of a coronavirus infection and/or post-coronavirus infection syndrome; methods for reducing severity of a MERS-CoV infection; methods for reducing severity of a SARS-CoV infection; methods for reducing severity of a SARS-CoV-2 infection; methods for reducing severity of a HCoV-229E infection; methods for reducing severity of a HCoV-NL63 infection; methods for reducing severity of a HCoV-OC43 infection; methods for reducing severity of a HCoV-HKU1 infection; methods for reducing severity of an HIV infection; methods for reducing severity of an Ebola infection; methods for reducing severity of an influenza infection; methods for reducing severity of an RSV infection;
methods for delaying the onset of a disease; methods for delaying the onset of a viral infection and/or post-viral infection syndrome; methods for delaying the onset of a coronavirus infection and/or post-coronavirus infection syndrome; methods for delaying the onset of a MERS-CoV infection; methods for delaying the onset of a SARS-CoV infection; methods for delaying the onset of a SARS-CoV-2 infection; methods for delaying the onset of a HCoV-229E infection; methods for delaying the onset of a HCoV-NL63 infection; methods for delaying the onset of a HCoV-OC43 infection; methods for delaying the onset of a HCoV-HKU1 infection; methods for delaying the onset of an HIV infection; methods for delaying the onset of an Ebola infection; methods for delaying the onset of an influenza infection; methods for delaying the onset of an RSV infection;
methods for inhibiting the development of a disease; methods for inhibiting entry of a virus into a cell; methods for inhibiting entry of a coronavirus into a cell; methods for inhibiting entry of a MERS-CoV virus into a cell; methods for inhibiting entry of a SARS-CoV virus into a cell; methods for inhibiting entry of a SARS-CoV-2 virus into a cell; methods for inhibiting entry of a HCoV-229E virus into a cell; methods for inhibiting entry of a HCoV-NL63 virus into a cell; methods for inhibiting entry of a HCoV-OC43 virus into a cell; methods for inhibiting entry of a HCoV-HKU1 virus into a cell; methods for inhibiting entry of an HIV virus into a cell; methods for inhibiting entry of an Ebola virus into a cell; methods for inhibiting entry of an influenza virus into a cell; methods for inhibiting entry of an RSV into a cell;
methods for regulating an interaction between one or more proteins; methods for regulating an interaction between a virus spike protein and host cell membrane; methods for regulating an interaction between a coronavirus spike protein and host cell membrane; methods for regulating an interaction between a MERS-CoV virus spike protein and host cell membrane; methods for regulating an interaction between a SARS-CoV virus spike protein and host cell membrane; methods for regulating an interaction between a SARS-CoV-2 spike protein and a host cell membrane; methods for regulating an interaction between a HCoV-229E spike protein and a host cell membrane; methods for regulating an interaction between a HCoV-NL63 spike protein and a host cell membrane; methods for regulating an interaction between a HCoV-OC43 spike protein and a host cell membrane; methods for regulating an interaction between a HCoV-HKU1 spike protein and a host cell membrane; methods for regulating an interaction between an HIV spike protein and a host cell membrane; methods for regulating an interaction between an Ebola virus glycoprotein and a host cell membrane; methods for regulating an interaction between an influenza virus spike protein and a host cell membrane; methods for regulating an interaction between an RSV glycoprotein and a host cell membrane;
methods of regulating the formation and translocation of a protein complex formed by one or more protein species; methods of regulating the formation and translocation of a virus spike protein to cell surfaces of the subject and/or to viral envelopes; methods of regulating the formation and translocation of a coronavirus spike protein to cell surfaces of the subject and/or to viral envelopes; methods of regulating the formation and translocation of a MERS-CoV spike protein to cell surfaces of the subject and/or to viral envelopes; methods of regulating the formation and translocation of a SARS-CoV spike protein to cell surfaces of the subject and/or to viral envelopes; methods of regulating the formation and translocation of a SARS-CoV-2 spike protein to cell surfaces of the subject and/or to viral envelopes; methods of regulating the formation and translocation of a HCoV-229E spike protein to cell surfaces of the subject and/or to viral envelopes; methods of regulating the formation and translocation of a HCoV-NL63 spike protein to cell surfaces of the subject and/or to viral envelopes; methods of regulating the formation and translocation of a HCoV-OC43 spike protein to cell surfaces of the subject and/or to viral envelopes; methods of regulating the formation and translocation of a HCoV-HKU1 spike protein to cell surfaces of the subject and/or to viral envelopes; methods of regulating the formation and translocation of an HIV spike protein to cell surfaces of the subject and/or to viral envelopes; methods of regulating the formation and translocation of an Ebola virus glycoprotein to cell surfaces of the subject and/or to viral envelopes; methods of regulating the formation and translocation of an influenza virus spike protein to cell surfaces of the subject and/or to viral envelopes; methods of regulating the formation and translocation of an RSV glycoprotein to cell surfaces of the subject and/or to viral envelopes;
methods of reducing the amount of a protein complex formed by one or more protein species in a cell and/or on cell surfaces of the subject; methods of reducing the amount of a virus spike protein on cell surfaces of the subject and/or on viral envelopes; methods of reducing the amount of a coronavirus spike protein on cell surfaces of the subject and/or on viral envelopes; methods of reducing the amount of a MERS-CoV spike protein on cell surfaces of the subject and/or on viral envelopes; methods of reducing the amount of a SARS-CoV spike protein on cell surfaces of the subject and/or on viral envelopes; methods of reducing the amount of a SARS-CoV-2 spike protein on cell surfaces of the subject and/or on viral envelopes; methods of reducing the amount of a HCoV-229E spike protein on cell surfaces of the subject and/or on viral envelopes; methods of reducing the amount of a HCoV-NL63 spike protein on cell surfaces of the subject and/or on viral envelopes; methods of reducing the amount of a HCoV-OC43 spike protein on cell surfaces of the subject and/or on viral envelopes; methods of reducing the amount of a HCoV-HKU1 spike protein on cell surfaces of the subject and/or on viral envelopes; methods of reducing the amount of an HIV spike protein on cell surfaces of the subject and/or on viral envelopes; methods of reducing the amount of an Ebola virus glycoprotein on cell surfaces of the subject and/or on viral envelopes; methods of reducing the amount of an influenza virus spike protein on cell surfaces of the subject and/or on viral envelopes; methods of reducing the amount of an RSV glycoprotein on cell surfaces of the subject and/or on viral envelopes;
methods of promoting proteasomal degradation of a non-native protein complex formed by one or more protein species by a cell; methods of promoting proteasomal degradation of a virus spike protein by a cell; methods of promoting proteasomal degradation of a coronavirus spike protein by a cell; methods of promoting proteasomal degradation of a MERS-CoV spike protein by a cell; methods of promoting proteasomal degradation of a SARS-CoV spike protein by a cell; methods of promoting proteasomal degradation of a SARS-CoV-2 spike protein by a cell; methods of promoting proteasomal degradation of a HCoV-229E spike protein by a cell; methods of promoting proteasomal degradation of a HCoV-NL63 spike protein by a cell; methods of promoting proteasomal degradation of a HCoV-OC43 spike protein by a cell; methods of promoting proteasomal degradation of a HCoV-HKU1 spike protein by a cell; methods of promoting proteasomal degradation of an HIV spike protein by a cell; methods of promoting proteasomal degradation of an Ebola virus glycoprotein by a cell; methods of promoting proteasomal degradation of an influenza virus spike protein by a cell; methods of promoting proteasomal degradation of an RSV glycoprotein by a cell;
nucleic acids; vectors; cells; pharmaceutical compositions; and kits. Because the SARS-CoV-2 virus causes COVID-19, any embodiment discussed in the context of SARS-CoV-2 can be implemented with respect to COVID-19.

Methods of the disclosure can include 1; 2; 3; 4; 5; 6; or more of the following steps: administering a polypeptide to a subject; administering a nucleic acid to a subject; administering a vector to a subject; administering an anti-viral agent to a subject;
diagnosing a subject as having a disease; diagnosing a subject as having a viral infection and/or post-viral infection syndrome; diagnosing a subject as having a coronavirus infection and/or post-coronavirus infection syndrome; diagnosing a subject as having a MERS-CoV infection; diagnosing a subject as having a SARS-CoV infection; diagnosing a subject as having a SARS-CoV-2 infection; diagnosing a subject as having a HCoV-229E infection; diagnosing a subject as having a HCoV-NL63 infection; diagnosing a subject as having a HCoV-OC43 infection; diagnosing a subject as having a HCoV-HKU1 infection; diagnosing a subject as having an HIV infection; diagnosing a subject as having an Ebola infection; diagnosing a subject as having an influenza infection; diagnosing a subject as having an RSV infection;
diagnosing a subject as having symptoms of a disease; diagnosing a subject as having symptoms of a viral infection and/or post-viral infection syndrome; diagnosing a subject as having symptoms of a coronavirus infection and/or post-coronavirus infection syndrome; diagnosing a subject as having symptoms of a MERS-CoV infection; diagnosing a subject as having symptoms of a SARS-CoV infection; diagnosing a subject as having symptoms of a SARS-CoV-2 infection; diagnosing a subject as having symptoms of a HCoV-229E infection; diagnosing a subject as having symptoms of a HCoV-NL63 infection; diagnosing a subject as having symptoms of a HCoV-OC43 infection; diagnosing a subject as having symptoms of a HCoV-HKU1 infection; diagnosing a subject as having symptoms of an HIV infection; diagnosing a subject as having symptoms of an Ebola infection; diagnosing a subject as having symptoms of an influenza infection; diagnosing a subject as having symptoms of an RSV infection;
diagnosing a subject as being at risk of having a disease; diagnosing a subject as being at risk of having a viral infection and/or post-viral infection syndrome; diagnosing a subject as being at risk of having a coronavirus infection and/or post-coronavirus infection syndrome; diagnosing a subject as being at risk of having a MERS-CoV infection; diagnosing a subject as being at risk of having a SARS-CoV infection; diagnosing a subject as being at risk of having a SARS-CoV-2 infection; diagnosing a subject as being at risk of having a HCoV-229E infection; diagnosing a subject as being at risk of having a HCoV-NL63 infection; diagnosing a subject as being at risk of having a HCoV-OC43 infection; diagnosing a subject as being at risk of having a HCoV-HKU1 infection; diagnosing a subject as being at risk of having an HIV infection; diagnosing a subject as being at risk of having an Ebola infection; diagnosing a subject as being at risk of having an influenza infection; diagnosing a subject as being at risk of having an RSV infection;
providing a second therapy for a disease to a subject; obtaining a sample from a subject; detecting a virus in a sample; detecting a coronavirus in a sample; detecting a MERS-CoV virus in a sample; detecting a SARS-CoV virus in a sample; detecting a SARS-CoV-2 virus in a sample; detecting HCoV-229E virus in a sample; detecting HCoV-NL63 virus in a sample; detecting HCoV-OC43 virus in a sample; detecting HCoV-HKU1 virus in a sample; detecting HIV virus in a sample; detecting Ebola virus in a sample; detecting influenza virus in a sample; detecting RSV in a sample;
providing a second therapy for a viral infection and/or post-viral infection syndrome to a subject; providing a second therapy for a coronavirus infection and/or post-coronavirus infection syndrome to a subject; providing a second therapy for a MERS-CoV infection to a subject; providing a second therapy for a SARS-CoV infection to a subject; providing a second therapy for a SARS-CoV-2 infection to a subject; providing a second therapy for a HCoV-229E infection to a subject; providing a second therapy for a HCoV-NL63 infection to a subject; providing a second therapy for a HCoV-OC43 infection to a subject; providing a second therapy for a HCoV-HKU1 infection to a subject; providing a second therapy for an HIV infection to a subject; providing a second therapy for an Ebola infection to a subject; providing a second therapy for an influenza infection to a subject; providing a second therapy for an RSV infection to a subject; and providing three or more types of anti-viral therapy to a subject. Certain embodiments of the disclosure may exclude one or more of the preceding elements and/or steps.

Compositions of the disclosure can include at least 1, 2, 3, 4, 5, or more of the following components: polypeptides, proteins, nucleic acids, vectors, therapeutic agents, anti-viral agents, pharmaceutically acceptable carriers, and excipients. One or more of these components may be specifically excluded from certain embodiments.

Disclosed herein, in some aspects, is a method for regulating a target protein or biological function thereof in vivo in a subject comprising contacting the target protein or a portion thereof and/or a native interacting partner of the target protein or a portion thereof with an effective amount of a polypeptide having an amino acid sequence corresponding to a sequence of the target protein, wherein the length of the polypeptide is greater than 30 amino acids, and wherein: the polypeptide and the target protein form a non-native protein complex upon contact of the target protein with the polypeptide, thereby regulating the target protein or biological function thereof in vivo; the polypeptide and the native interacting partner of the target protein form a non-native protein complex upon contact of the native interacting partner of the target protein with the polypeptide, thereby regulating the target protein or biological function thereof in vivo; and/or the polypeptide, the target protein, and the native interacting partner of the target protein form a non-native protein complex upon contact of the target protein and the native interacting partner of the target protein with the polypeptide, thereby regulating the target protein or biological function thereof in vivo.

In some embodiments, the polypeptide having an amino acid sequence corresponding to a sequence of the target protein has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with the sequence of the target protein. In some embodiments, the polypeptide having an amino acid sequence corresponding to a sequence of the target protein has at least 10% sequence identity with the sequence of the target protein. In some embodiments, the polypeptide having an amino acid sequence corresponding to a sequence of the target protein has at least 20% identity with the sequence of the target protein. In some embodiments, the polypeptide having an amino acid sequence corresponding to a sequence of the target protein has at least 30% identity with the sequence of the target protein. In some embodiments, the polypeptide having an amino acid sequence corresponding to a sequence of the target protein has at least 40% identity with the sequence of the target protein. In some embodiments, the polypeptide having an amino acid sequence corresponding to a sequence of the target protein has at least 50% identity with the sequence of the target protein. In some embodiments, the polypeptide having an amino acid sequence corresponding to a sequence of the target protein has at least 60% identity with the sequence of the target protein. In some embodiments, the polypeptide having an amino acid sequence corresponding to a sequence of the target protein has at least 70% identity with the sequence of the target protein. In some embodiments, the polypeptide having an amino acid sequence corresponding to a sequence of the target protein has at least 80% identity with the sequence of the target protein. In some embodiments, the polypeptide having an amino acid sequence corresponding to a sequence of the target protein has at least 85% identity with the sequence of the target protein. In some embodiments, the polypeptide having an amino acid sequence corresponding to a sequence of the target protein has at least 90% identity with the sequence of the target protein. In some embodiments, the polypeptide having an amino acid sequence corresponding to a sequence of the target protein has at least 95% identity with the sequence of the target protein. In some embodiments, the polypeptide having an amino acid sequence corresponding to a sequence of the target protein comprises the corresponding sequence of the target protein.

Disclosed herein, in some aspects, is a method for regulating a target protein or biological function thereof in vivo in a subject comprising contacting the target protein or a portion thereof and/or a native interacting partner of the target protein or a portion thereof with an effective amount of a polypeptide having an amino acid sequence corresponding to an oligomerization domain of the target protein, wherein the length of the polypeptide is greater than 30 amino acids, and wherein: the polypeptide oligomerizes with the oligomerization domain of the target protein to form a non-native protein complex, thereby regulating the target protein or biological function thereof in vivo; the polypeptide oligomerizes with the oligomerization domain of the native interacting partner of the target protein to form a non-native protein complex, thereby regulating the target protein or biological function thereof in vivo; and/or the polypeptide oligomerizes with the oligomerization domain of the target protein and the native interacting partner of the target protein to form a non-native protein complex, thereby regulating the target protein or biological function thereof in vivo.

In some embodiments, the polypeptide having an amino acid sequence corresponding to an oligomerization domain of the target protein has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with the oligomerization domain of the target protein. In some embodiments, the polypeptide having an amino acid sequence corresponding to an oligomerization domain of the target protein has at least 10% sequence identity with the oligomerization domain of the target protein. In some embodiments, the polypeptide having an amino acid sequence corresponding to an oligomerization domain of the target protein has at least 20% identity with the oligomerization domain of the target protein. In some embodiments, the polypeptide having an amino acid sequence corresponding to an oligomerization domain of the target protein has at least 30% identity with the oligomerization domain of the target protein. In some embodiments, the polypeptide having an amino acid sequence corresponding to an oligomerization domain of the target protein has at least 40% identity with the oligomerization domain of the target protein. In some embodiments, the polypeptide having an amino acid sequence corresponding to an oligomerization domain of the target protein has at least 50% identity with the oligomerization domain of the target protein. In some embodiments, the polypeptide having an amino acid sequence corresponding to an oligomerization domain of the target protein has at least 60% identity with the oligomerization domain of the target protein. In some embodiments, the polypeptide having an amino acid sequence corresponding to an oligomerization domain of the target protein has at least 70% identity with the oligomerization domain of the target protein. In some embodiments, the polypeptide having an amino acid sequence corresponding to an oligomerization domain of the target protein has at least 80% identity with the oligomerization domain of the target protein. In some embodiments, the polypeptide having an amino acid sequence corresponding to an oligomerization domain of the target protein has at least 85% identity with the oligomerization domain of the target protein. In some embodiments, the polypeptide having an amino acid sequence corresponding to an oligomerization domain of the target protein has at least 90% identity with the oligomerization domain of the target protein. In some embodiments, the polypeptide having an amino acid sequence corresponding to an oligomerization domain of the target protein has at least 95% identity with the oligomerization domain of the target protein. In some embodiments, the polypeptide having an amino acid sequence corresponding to an oligomerization domain of the target protein comprises the oligomerization domain of the target protein.

In some embodiments, the target protein is a viral protein. In some embodiments, the target protein is a viral glycoprotein. In some embodiments, the target protein is a viral spike protein. In some embodiments, regulating the target protein or biological function thereof in vivo treats or prevents a disease or condition in the subject. In some embodiments, the disease or condition is a viral infection.

In some embodiments, the non-native protein complex is subjected to proteasomal degradation. In some embodiments, formation of the non-native protein complex inhibits the target protein or biological function thereof in vivo by inhibiting homo-oligomerization of the target protein. In some embodiments, formation of the non-native protein complex inhibits the target protein or biological function thereof in vivo by inhibiting hetero-oligomerization of the target protein. In some embodiments, formation of the non-native protein complex inhibits the target protein or biological function thereof in vivo by inhibiting homo-oligomerization and hetero-oligomerization of the target protein.

Disclosed herein, in some aspects, is:

a method for regulating a coronavirus spike protein or biological function thereof in vivo in a subject comprising contacting the coronavirus spike protein or a portion thereof with an effective amount of a polypeptide comprising an amino acid sequence having at least 10% identity with SEQ ID NOS:2, 4, 6, 8, 10, 12, 14, or 16;

a method for regulating a coronavirus spike protein or biological function thereof in vivo in a subject comprising contacting the coronavirus spike protein or a portion thereof with an effective amount of a polypeptide having an amino acid sequence corresponding to an oligomerization domain of the coronavirus spike protein and having at least 10% identity with SEQ ID NOS:2, 4, 6, 8, 10, 12, 14, or 16, wherein the polypeptide oligomerizes with the oligomerization domain of the coronavirus spike protein to form a non-native protein complex, thereby regulating the coronavirus spike protein or biological function thereof in vivo;

a method for treating or preventing a coronavirus infection in a subject comprising administering to the subject a therapeutically effective amount of a composition comprising a polypeptide comprising an amino acid sequence having at least 10% identity with SEQ ID NOS:2, 4, 6, 8, 10, 12, 14, or 16; and

a method for treating or preventing a coronavirus infection in a subject comprising administering to the subject a therapeutically effective amount of a composition comprising a polypeptide having an amino acid sequence corresponding to an oligomerization domain of a coronavirus spike protein and having at least 10% identity with SEQ ID NOS:2, 4, 6, 8, 10, 12, 14, or 16, wherein the polypeptide oligomerizes with the oligomerization domain of the coronavirus spike protein to form a non-native protein complex.

In some embodiments, the non-native protein complex is subjected to proteasomal degradation. In some embodiments, regulating the coronavirus spike protein or biological function thereof in vivo treats or prevents a coronavirus infection in the subject. In some embodiments, regulating the coronavirus spike protein or biological function thereof in vivo comprises inhibition of formation and translocation of the coronavirus spike protein to cell surfaces of the subject and/or to viral envelopes. In some embodiments, regulating the coronavirus spike protein or biological function thereof in vivo reduces the amount of the coronavirus spike protein on cell surfaces of the subject and/or on viral envelopes. In some embodiments, oligomerization of the polypeptide and the coronavirus spike protein regulates the coronavirus spike protein or biological function thereof to treat or prevent the coronavirus infection. In some embodiments, formation and translocation of the coronavirus spike protein to cell surfaces of the subject and/or to viral envelopes is inhibited. In some embodiments, amount of the coronavirus spike protein on cell surfaces of the subject and/or on viral envelopes is reduced.

In some embodiments, the coronavirus comprises SARS-CoV, SARS-CoV-2, MERS-CoV, HCoV-229E, HCoV-NL63, HCoV-OC43, or HCoV-HKU1. In some embodiments, the coronavirus comprises SARS-CoV, and the coronavirus spike protein comprises a SARS-CoV spike protein. In some embodiments, the coronavirus comprises SARS-CoV-2, and the coronavirus spike protein comprises a SARS-CoV-2 spike protein. In some embodiments, the coronavirus comprises MERS-CoV, and the coronavirus spike protein comprises a MERS-CoV spike protein. In some embodiments, the coronavirus comprises HCoV-229E, and the coronavirus spike protein comprises a HCoV-229E spike protein. In some embodiments, the coronavirus comprises HCoV-NL63, and the coronavirus spike protein comprises a HCoV-NL63 spike protein. In some embodiments, the coronavirus comprises HCoV-OC43, and the coronavirus spike protein comprises a HCoV-OC43 spike protein. In some embodiments, the coronavirus comprises HCoV-HKU1, and the coronavirus spike protein comprises a HCoV-HKU1 spike protein.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:2. In some embodiments, the polypeptide has at least 80% identity with SEQ ID NO:2. In some embodiments, the polypeptide has at least 85% identity with SEQ ID NO:2. In some embodiments, the polypeptide has at least 90% identity with SEQ ID NO:2. In some embodiments, the polypeptide has at least 95% identity with SEQ ID NO:2. In some embodiments, the polypeptide comprises SEQ ID NO:2.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:4. In some embodiments, the polypeptide has at least 80% identity with SEQ ID NO:4. In some embodiments, the polypeptide has at least 85% identity with SEQ ID NO:4. In some embodiments, the polypeptide has at least 90% identity with SEQ ID NO:4. In some embodiments, the polypeptide has at least 95% identity with SEQ ID NO:4. In some embodiments, the polypeptide comprises SEQ ID NO:4.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:6. In some embodiments, the polypeptide has at least 80% identity with SEQ ID NO:6. In some embodiments, the polypeptide has at least 85% identity with SEQ ID NO:6. In some embodiments, the polypeptide has at least 90% identity with SEQ ID NO:6. In some embodiments, the polypeptide has at least 95% identity with SEQ ID NO:6. In some embodiments, the polypeptide comprises SEQ ID NO:6.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:8. In some embodiments, the polypeptide has at least 80% identity with SEQ ID NO:8. In some embodiments, the polypeptide has at least 85% identity with SEQ ID NO:8. In some embodiments, the polypeptide has at least 90% identity with SEQ ID NO:8. In some embodiments, the polypeptide has at least 95% identity with SEQ ID NO:8. In some embodiments, the polypeptide comprises SEQ ID NO:8.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:10. In some embodiments, the polypeptide has at least 80% identity with SEQ ID NO:10. In some embodiments, the polypeptide has at least 85% identity with SEQ ID NO:10. In some embodiments, the polypeptide has at least 90% identity with SEQ ID NO:10. In some embodiments, the polypeptide has at least 95% identity with SEQ ID NO:10. In some embodiments, the polypeptide comprises SEQ ID NO:10.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:12. In some embodiments, the polypeptide has at least 80% identity with SEQ ID NO:12. In some embodiments, the polypeptide has at least 85% identity with SEQ ID NO:12. In some embodiments, the polypeptide has at least 90% identity with SEQ ID NO:12. In some embodiments, the polypeptide has at least 95% identity with SEQ ID NO:12. In some embodiments, the polypeptide comprises SEQ ID NO:12.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:14. In some embodiments, the polypeptide has at least 80% identity with SEQ ID NO:14. In some embodiments, the polypeptide has at least 85% identity with SEQ ID NO:14. In some embodiments, the polypeptide has at least 90% identity with SEQ ID NO:14. In some embodiments, the polypeptide has at least 95% identity with SEQ ID NO:14. In some embodiments, the polypeptide comprises SEQ ID NO:14.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:16. In some embodiments, the polypeptide has at least 80% identity with SEQ ID NO:16. In some embodiments, the polypeptide has at least 85% identity with SEQ ID NO:16. In some embodiments, the polypeptide has at least 90% identity with SEQ ID NO:16. In some embodiments, the polypeptide has at least 95% identity with SEQ ID NO:16. In some embodiments, the polypeptide comprises SEQ ID NO:16.

In some embodiments, the methods for regulating a coronavirus spike protein or biological function thereof in vivo and/or the methods for treating or preventing a coronavirus infection further comprise: diagnosing the subject with the coronavirus infection; diagnosing the subject as having symptoms of the coronavirus infection; or diagnosing the subject as being at risk of having the coronavirus infection. In some embodiments, the subject is at high risk for having a coronavirus infection. In some embodiments, the subject does not have a coronavirus infection. In some embodiments, the subject has tested negative for a coronavirus infection. In some embodiments, the subject was diagnosed as having a coronavirus infection. In some embodiments, the coronavirus infection causes severe acute respiratory syndrome (SARS), Middle East respiratory syndrome, or a respiratory infection. In some embodiments, the coronavirus infection causes COVID-19. In some embodiments, the coronavirus infection is prevented, reduced in severity, and/or delayed in onset.

In some embodiments, the subject is provided an effective amount of a second therapy for the coronavirus infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-SARS-CoV-2 drug. In some embodiments, the anti-SARS-CoV-2 drug is selected from the group consisting of steroids, zinc, vitamin C, Remdesivir, Tocilizumab, Anakinra, Beclomethasone, Betamethasone, Budesonide Cortisone, Dexamethasone, Hydrocortisone, Methylprednisolone, Prednisolone, Prednisone, Triamcinolone, Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine, Hydroxychloroquine, CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/MlN128), FK-506 (Tacrolimus), Ternatin 4 (DA3), Tigecycline, Tomivosertib (eFT-508), Verdinexor, WDB002, Zotatifin (eFT226), and a combination thereof.

Disclosed herein in some aspects, is:

a method for regulating an HIV spike protein or biological function thereof in vivo in a subject comprising contacting the HIV spike protein or a portion thereof with an effective amount of a polypeptide comprising an amino acid sequence having at least 10% identity with SEQ ID NO:18;

a method for regulating an HIV spike protein or biological function thereof in vivo in a subject comprising contacting the HIV spike protein or a portion thereof with an effective amount of a polypeptide having an amino acid sequence corresponding to an oligomerization domain of the HIV spike protein and having at least 10% identity with SEQ ID NO:18, wherein the polypeptide oligomerizes with the oligomerization domain of the HIV spike protein to form a non-native protein complex, thereby regulating the HIV spike protein or biological function thereof in vivo;

a method for treating or preventing an HIV infection in a subject comprising administering to the subject a therapeutically effective amount of a composition comprising a polypeptide comprising an amino acid sequence having at least 10% identity with SEQ ID NO:18; and

a method for treating or preventing an HIV infection in a subject comprising administering to the subject a therapeutically effective amount of a composition comprising a polypeptide having an amino acid sequence corresponding to an oligomerization domain of an HIV spike protein and having at least 10% identity with SEQ ID NO:18, wherein the polypeptide oligomerizes with the oligomerization domain of the HIV spike protein to form a non-native protein complex.

In some embodiments, the non-native protein complex is subjected to proteasomal degradation. In some embodiments, regulating the HIV spike protein or biological function thereof in vivo treats or prevents an HIV infection in the subject. In some embodiments, regulating the HIV spike protein or biological function thereof in vivo comprises inhibition of formation and translocation of the HIV spike protein to cell surfaces of the subject and/or to viral envelopes. In some embodiments, regulating the HIV spike protein or biological function thereof in vivo reduces the amount of the HIV spike protein on cell surfaces of the subject and/or on viral envelopes. In some embodiments, oligomerization of the polypeptide and the HIV spike protein regulates the HIV spike protein or biological function thereof to treat or prevent the HIV infection. In some embodiments, formation and translocation of the HIV spike protein to cell surfaces of the subject and/or to viral envelopes is inhibited. In some embodiments, amount of the HIV spike protein on cell surfaces of the subject and/or on viral envelopes is reduced.

In some embodiments, the HIV spike protein comprises an HIV gp160 spike protein.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:18. In some embodiments, the polypeptide has at least 80% identity with SEQ ID NO:18. In some embodiments, the polypeptide has at least 85% identity with SEQ ID NO:18. In some embodiments, the polypeptide has at least 90% identity with SEQ ID NO:18. In some embodiments, the polypeptide has at least 95% identity with SEQ ID NO:18. In some embodiments, the polypeptide comprises SEQ ID NO:18.

In some embodiments, the methods for regulating an HIV spike protein or biological function thereof in vivo and/or the methods for treating or preventing an HIV infection further comprise: diagnosing the subject with the HIV infection; diagnosing the subject as having symptoms of the HIV infection; or diagnosing the subject as being at risk of having the HIV infection. In some embodiments, the subject is at high risk for having an HIV infection. In some embodiments, the subject does not have an HIV infection. In some embodiments, the subject has tested negative for an HIV infection. In some embodiments, the subject was diagnosed as having an HIV infection. In some embodiments, the HIV infection is prevented, reduced in severity, and/or delayed in onset.

In some embodiments, the subject is provided an effective amount of a second therapy for the HIV infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-HIV drug. In some embodiments, the anti-HIV drug is selected from the group consisting of efavirenz (Sustiva), rilpivirine (Edurant), etravirine (Intelence), delavirdine (Rescriptor), nevirapine (Viramune, Viramune XR), doravirine (Pifeltroz), abacavir (Ziagen), tenofovir alafenamide fumarate (Vemlidy), tenofovir (Viread), emtricitabine (Emtriva), lamivudine (Epivir), zidovudine (Retrovir), abacavir/lamivudine/zidovudine (Trizivir), abacavir/lamivudine (Epzicom), emtricitabine/tenofovir (Truvada), abacavir/lamivudine (Epzicom), lamivudine/tenofovir disoproxil fumarate (Cimduo, Temixys), lamivudine/zidovudine (Combivir), emtricitabine/tenofovir alafenamide (Descovy), didanosine (Videx, Videx EC), stavudine (Zerit), atazanavir (Reyataz), darunavir (Prezista), fosamprenavir (Lexiva), ritonavir (Norvir), tipranavir (Aptivus), lopinavir/ritonavir (Kaletra), atazanavir/cobicistat (Evotaz), darunavir/cobicistat (Prezcobix), indinavir (Crixivan), nelfinavir (Viracept), saquinavir (Invirase), bictegravir sodium/emtricitabine/tenofovir alafenamide fumar (Biktarvy), raltegravir (Isentress), elvitegravir (Genvoya and Stribild), dolutegravir (Tivicay), cobicistat (Tybost), ritonavir (Norvir), enfuvirtide (Fuzeon), maraviroc (Selzentry), ibalizumab-uiyk (Trogarzo), maraviroc (Selzentry), fostemsavir (Rukobia), doravirine/lamivudine/tenofovir disoproxil fumarate (Delstrigo), efavirenz/lamivudine/tenofovir disoproxil fumarate (Symfi), efavirenz/lamivudine/tenofovir disoproxil fumarate (Symfi Lo), efavirenz/emtricitabine/tenofovir disoproxil fumarate (Atripla), emtricitabine/rilpivirine/tenofovir alafenamide fumarate (Odefsey), emtricitabine/rilpivirine/tenofovir disoproxil fumarate (Complera), elvitegravir/cobicistat/emtricitabine/tenofovir disoproxil fumarate (Stribild), elvitegravir/cobicistat/emtricitabine/tenofovir alafenamide fumarate (Genvoya), abacavir/dolutegravir/lamivudine (Triumeq), bictegravir/emtricitabine/tenofovir alafenamide fumarate (Biktarvy), dolutegravir/lamivudine (Dovato), dolutegravir/rilpivirine (Juluca), darunavir/cobicistat/emtricitabine/tenofovir alafenamide fumarate (Symtuza), acetyl-L-carnitine, whey protein, L-glutamine, L-arginine, hydroxymethylbutyrate (HMB), probiotics, vitamins and minerals, and a combination thereof.

Disclosed herein, in some aspects, is:
a method for regulating an Ebola glycoprotein or biological function thereof in vivo in a subject comprising contacting the Ebola glycoprotein or a portion thereof with an effective amount of a polypeptide comprising an amino acid sequence having at least 10% identity with SEQ ID NO:20;
a method for regulating an Ebola glycoprotein or biological function thereof in vivo in a subject comprising contacting the Ebola glycoprotein or a portion thereof with an effective amount of a polypeptide having an amino acid sequence corresponding to an oligomerization domain of the Ebola glycoprotein and having at least 10% identity with SEQ ID NO:20, wherein the polypeptide oligomerizes with the oligomerization domain of the Ebola glycoprotein to form a non-native protein complex, thereby inhibiting the Ebola glycoprotein or biological function thereof in vivo;
a method for treating or preventing an Ebola infection in a subject comprising administering to the subject a therapeutically effective amount of a composition comprising a polypeptide comprising an amino acid sequence having at least 10% identity with SEQ ID NO:20; and
a method for treating or preventing an Ebola infection in a subject comprising administering to the subject a therapeutically effective amount of a composition comprising a polypeptide having an amino acid sequence corresponding to an oligomerization domain of an Ebola glycoprotein and having at least 10% identity with SEQ ID NO:20, wherein the polypeptide oligomerizes with the oligomerization domain of the Ebola glycoprotein to form a non-native protein complex.

In some embodiments, the non-native protein complex is subjected to proteasomal degradation. In some embodiments, regulating the Ebola glycoprotein or biological function thereof in vivo treats or prevents an Ebola infection in the subject. In some embodiments, regulating the Ebola glycoprotein or biological function thereof in vivo comprises inhibition of formation and translocation of the Ebola glycoprotein to cell surfaces of the subject and/or to viral envelopes. In some embodiments, regulating the Ebola glycoprotein or biological function thereof in vivo reduces the amount of the Ebola glycoprotein on cell surfaces of the subject and/or on viral envelopes. In some embodiments, oligomerization of the polypeptide and the Ebola glycoprotein regulates the Ebola glycoprotein or biological function thereof to treat or prevent the Ebola infection. In some embodiments, formation and translocation of the Ebola glycoprotein to cell surfaces of the subject and/or to viral envelopes is inhibited. In some embodiments, amount of the Ebola glycoprotein on cell surfaces of the subject and/or on viral envelopes is reduced. In some embodiments, the Ebola infection is prevented, reduced in severity, and/or delayed in onset.

In some embodiments, the Ebola glycoprotein comprises an Ebola GP glycoprotein.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:20. In some embodiments, the polypeptide has at least 80% identity with SEQ ID NO:20. In some embodiments, the polypeptide has at least 85% identity with SEQ ID NO:20. In some embodiments, the polypeptide has at least 90% identity with SEQ ID NO:20. In some embodiments, the polypeptide has at least 95% identity with SEQ ID NO:20. In some embodiments, the polypeptide comprises SEQ ID NO:20.

In some embodiments, the methods for regulating an Ebola glycoprotein or biological function thereof in vivo and/or the methods for treating or preventing an Ebola infection further comprise: diagnosing the subject with the Ebola infection; diagnosing the subject as having symptoms of the Ebola infection; or diagnosing the subject as being at risk of having the Ebola infection. In some embodiments, the subject is at high risk for having an Ebola infection. In some embodiments, the subject does not have an Ebola infection. In some embodiments, the subject has tested negative for an Ebola infection. In some embodiments, the subject was diagnosed as having an Ebola infection.

In some embodiments, the subject is provided an effective amount of a second therapy for the Ebola infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-Ebola drug. In some embodiments, the anti-Ebola drug is selected from the group consisting of atoltivimab/maftivimab/odesivimab-ebgn (Inmazeb), ansuvimab-zykl (Ebanga), Favipiravir (Avigan), Ribavirin, BCX4430, Brincidofovir, TKM-Ebola, AVI-7537, JK-05, and a combination thereof.

Disclosed herein, in some aspects, is:
a method for regulating an influenza virus spike protein or biological function thereof in vivo in a subject comprising contacting the influenza virus spike protein or a portion thereof with an effective amount of a polypeptide comprising an amino acid sequence having at least 10% identity with SEQ ID NO:25;
a method for regulating an influenza virus spike protein or biological function thereof in vivo in a subject comprising contacting the influenza virus spike protein or a portion thereof with an effective amount of a polypeptide having an amino acid sequence corresponding to an oligomerization domain of the influenza virus spike protein and having at least 10% identity with SEQ ID NO:25, wherein the polypeptide oligomerizes with the oligomerization domain of the influenza virus spike protein to form a non-native protein complex, thereby regulating the influenza virus spike protein or biological function thereof in vivo;
a method for treating or preventing an influenza infection in a subject comprising administering to the subject a therapeutically effective amount of a composition comprising a polypeptide comprising an amino acid sequence having at least 10% identity with SEQ ID NO:25; and
a method for treating or preventing an influenza infection in a subject comprising administering to the subject a therapeutically effective amount of a composition comprising a polypeptide having an amino acid sequence corresponding to an oligomerization domain of an influenza virus spike protein and having at least 10% identity with SEQ ID NO:25, wherein the polypeptide oligomerizes with the oligomerization domain of the influenza virus spike protein to form a non-native protein complex.

In some embodiments, the non-native protein complex is subjected to proteasomal degradation. In some embodiments, regulating the influenza virus spike protein or biological function thereof in vivo treats or prevents an influenza infection in the subject. In some embodiments, regulating the influenza virus spike protein or biological function thereof in vivo comprises inhibition of formation and translocation of the influenza virus spike protein to cell surfaces of the subject and/or to viral envelopes. In some embodiments, regulating the influenza virus spike protein or biological function thereof in vivo reduces the amount of the influenza virus spike protein on cell surfaces of the subject and/or on viral envelopes. In some embodiments, oligomerization of the polypeptide and the influenza virus spike protein regulates the influenza virus spike protein or biological function thereof to treat or prevent the influenza infection. In some embodiments, formation and translocation of the influenza virus spike protein to cell surfaces of the subject and/or to viral envelopes is inhibited. In some embodiments, amount of the influenza virus spike protein on cell surfaces of the subject and/or on viral envelopes is reduced.

In some embodiments, the influenza comprises influenza virus A, and the influenza virus spike protein comprises an influenza virus A spike protein. In some embodiments, the influenza virus A comprises influenza virus A/H1, and the influenza virus spike protein comprises an influenza virus A/H1 HA spike protein. In some embodiments, the influenza virus A comprises influenza virus A/H3, and the influenza virus spike protein comprises an influenza virus A/H3 HA spike protein. In some embodiments, the influenza comprises influenza virus B, and the influenza virus spike protein comprises an influenza virus B spike protein. In some embodiments, the influenza virus B comprises influenza virus B/Victoria, and the influenza virus spike protein comprises an influenza virus B/Victoria HA spike protein. In some embodiments, the influenza virus B comprises influenza virus B/Yamagata, and the influenza virus spike protein comprises an influenza virus B/Yamagata HA spike protein.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:25. In some embodiments, the polypeptide has at least 80% identity with SEQ ID NO:25. In some embodiments, the polypeptide has at least 85% identity with SEQ ID NO:25. In some embodiments, the polypeptide has at least 90% identity with SEQ ID NO:25. In some embodiments, the polypeptide has at least 95% identity with SEQ ID NO:25. In some embodiments, the polypeptide comprises SEQ ID NO:25.

In some embodiments, the methods for regulating an influenza virus spike protein or biological function thereof in vivo and/or the methods for treating or preventing an influenza virus infection further comprise: diagnosing the subject with the influenza infection; diagnosing the subject as having symptoms of the influenza infection; or diagnosing the subject as being at risk of having the influenza infection. In some embodiments, the subject is at high risk for having an influenza infection. In some embodiments, the subject does not have an influenza infection. In some embodiments, the subject has tested negative for an influenza infection. In some embodiments, the subject was diagnosed as having an influenza infection. In some embodiments, the influenza infection is prevented, reduced in severity, and/or delayed in onset.

In some embodiments, the subject is provided an effective amount of a second therapy for the influenza infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-influenza drug. In some embodiments, the anti-influenza drug is selected from the group consisting of oseltamivir phosphate (Tamiflu), zanamivir (Relenza), peramivir (Rapivab), baloxavir marboxil (Xofluza), amantadine, rimantadine (Flumadine), umifenovir (Arbidol), moroxydine, fluticare, acetaminophen, chlorpheniramine, dextromethorphan, pseudoephedrine, and a combination thereof.

Disclosed herein, in some aspects, is:
a method for regulating an RSV glycoprotein or biological function thereof in vivo in a subject comprising contacting the RSV glycoprotein or a portion thereof with an effective amount of a polypeptide comprising an amino acid sequence having at least 10% identity with SEQ ID NO:34;
a method for regulating an RSV glycoprotein or biological function thereof in vivo in a subject comprising contacting the RSV glycoprotein or a portion thereof with an effective amount of a polypeptide having an amino acid sequence corresponding to an oligomerization domain of the RSV glycoprotein and having at least 10% identity with SEQ ID NO:34, wherein the polypeptide oligomerizes with the oligomerization domain of the RSV glycoprotein to form a non-native protein complex, thereby inhibiting the RSV glycoprotein or biological function thereof in vivo;
a method for treating or preventing an RSV infection in a subject comprising administering to the subject a therapeutically effective amount of a composition comprising a polypeptide comprising an amino acid sequence having at least 10% identity with SEQ ID NO:34; and
a method for treating or preventing an RSV infection in a subject comprising administering to the subject a therapeutically effective amount of a composition comprising a polypeptide having an amino acid sequence corresponding to an oligomerization domain of an RSV glycoprotein and having at least 10% identity with SEQ ID NO:34, wherein the polypeptide oligomerizes with the oligomerization domain of the Ebola glycoprotein to form a non-native protein complex.

In some embodiments, the non-native protein complex is subjected to proteasomal degradation. In some embodiments, regulating the RSV glycoprotein or biological function thereof in vivo treats or prevents an RSV infection in the subject. In some embodiments, regulating the RSV glycoprotein or biological function thereof in vivo comprises inhibition of formation and translocation of the RSV glycoprotein to cell surfaces of the subject and/or to viral envelopes. In some embodiments, regulating the RSV glycoprotein or biological function thereof in vivo reduces the amount of the RSV glycoprotein on cell surfaces of the subject and/or on viral envelopes. In some embodiments, oligomerization of the polypeptide and the RSV glycoprotein regulates the RSV glycoprotein or biological function thereof to treat or prevent the RSV infection. In some embodiments, formation and translocation of the RSV glycoprotein to cell surfaces of the subject and/or to viral envelopes is inhibited. In some embodiments, amount of the RSV glycoprotein on cell surfaces of the subject and/or on viral envelopes is reduced. In some embodiments, the RSV infection is prevented, reduced in severity, and/or delayed in onset.

In some embodiments, the RSV glycoprotein comprises an RSV F glycoprotein.

In some embodiments, the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:34. In some embodiments, the polypeptide has at least 80% identity with SEQ ID NO:34. In some embodiments, the polypeptide has at least 85% identity with SEQ ID NO:34. In some embodiments, the polypeptide has at least 90% identity with SEQ ID NO:34. In some embodiments, the polypeptide has at least 95% identity with SEQ ID NO:34. In some embodiments, the polypeptide comprises SEQ ID NO:34.

In some embodiments, the methods for regulating an RSV glycoprotein or biological function thereof in vivo and/or the methods for treating or preventing an RSV infection further comprise: diagnosing the subject with the RSV infection; diagnosing the subject as having symptoms of the RSV infection; or diagnosing the subject as being at risk of having the RSV infection. In some embodiments, the subject is at high risk for having an RSV infection. In some embodiments, the subject does not have an RSV infection. In some embodiments, the subject has tested negative for an RSV infection. In some embodiments, the subject was diagnosed as having an RSV infection.

In some embodiments, the subject is provided an effective amount of a second therapy for the RSV infection. In some embodiments, the second therapy comprises an anti-RSV drug.

In some embodiments of the methods disclosed herein, a dose of between 0.1 to 1000 mg/kg body weight of the subject of the polypeptide is administered to the subject. In some embodiments, a dose of between 0.1 to 1000 μg/kg body weight of the subject of the polypeptide is administered to the subject.

In some embodiments, the polypeptide is expressed from a vector encoding the polypeptide. In some embodiments, the vector is a viral vector or a non-viral vector. In some embodiments, the vector is a minicircle DNA vector. In some embodiments, a dose of between 1×10⁸ to 1×10¹⁸ vector copies/kg body weight of the subject is administered to the subject. In some embodiments, a dose of about 1×10¹¹ to about 1×10¹⁴ vector copies/kg body weight of the subject is administered to the subject. In some embodiments, a dose of about 1×10¹² to about 1×10¹⁵ vector/kg body weight of the subject is administered to the subject. In some embodiments, the vector transduces cells of the subject, and wherein the cells of the subject express the polypeptide.

In some embodiments, the composition further comprises a pharmaceutically acceptable carrier. In some embodiments, the pharmaceutically acceptable carrier comprises liposomes, polymeric micelles, microspheres, or nanoparticles.

In some embodiments, a single dose of the composition is administered. In some embodiments, multiple doses of the composition are administered. In some embodiments, the composition is delivered to the subject once a day, more than once a day, more than once a week, more than once a month, or more than once a year. In some embodiments, the composition is delivered systemically or locally. In some embodiments, the composition is administered to the subject intranasally, intravenously, intraperitoneally, intratracheally, intramuscularly, endoscopically, percutaneously, subcutaneously, regionally, intracranially, by inhalation, by injection, by infusion, or by perfusion. In some embodiments, the composition is administered to the subject by inhalation. In some embodiments, the composition is administered to the subject intranasally.

Disclosed herein, in some aspects, is a pharmaceutical composition comprising a vector encoding a polypeptide having an amino acid sequence corresponding to a sequence of a target protein, wherein the length of the polypeptide is greater than 30 amino acids, and wherein the polypeptide has at least 10% sequence identity with the sequence of the target protein.

In some embodiments, the polypeptide having an amino acid sequence corresponding to a sequence of the target protein has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with the sequence of the target protein. In some embodiments, the polypeptide having an amino acid sequence corresponding to a sequence of the target protein has at least 10% sequence identity with the sequence of the target protein. In some embodiments, the polypeptide having an amino acid sequence corresponding to a sequence of the target protein has at least 20% identity with the sequence of the target protein. In some embodiments, the polypeptide having an amino acid sequence corresponding to a sequence of the target protein has at least 30% identity with the sequence of the target protein. In some embodiments, the polypeptide having an amino acid sequence corresponding to a sequence of the target protein has at least 40% identity with the sequence of the target protein. In some embodiments, the polypeptide having an amino acid sequence corresponding to a sequence of the target protein has at least 50% identity with the sequence of the target protein. In some embodiments, the polypeptide having an amino acid sequence corresponding to a sequence of the target protein has at least 60% identity with the sequence of the target protein. In some embodiments, the polypeptide having an amino acid sequence corresponding to a sequence of the target protein has at least 70% identity with the sequence of the target protein. In some embodiments, the polypeptide having an amino acid sequence corresponding to a sequence of the target protein has at least 80% identity with the sequence of the target protein. In some embodiments, the polypeptide having an amino acid sequence corresponding to a sequence of the target protein has at least 85% identity with the sequence of the target protein. In some embodiments, the polypeptide having an amino acid sequence corresponding to a sequence of the target protein has at least 90% identity with the sequence of the target protein. In some embodiments, the polypeptide having an amino acid sequence corresponding to a sequence of the target protein has at least 95% identity with the sequence of the target protein. In some embodiments, the polypeptide having an amino acid sequence corresponding to a sequence of the target protein comprises the corresponding sequence of the target protein.

Disclosed herein, in some aspects, is a pharmaceutical composition comprising a vector encoding a polypeptide having an amino acid sequence corresponding to an oligomerization domain of a target protein, wherein the length of the polypeptide is greater than 30 amino acids, and wherein the polypeptide has at least 10% sequence identity with the oligomerization domain of the target protein.

In some embodiments, the polypeptide having an amino acid sequence corresponding to an oligomerization domain of the target protein has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with the oligomerization domain of the target protein. In some embodiments, the polypeptide having an amino acid sequence corresponding to an oligomerization domain of the target protein has at least 10% identity with the oligomerization domain of the target protein. In some embodiments, the polypeptide having an amino acid sequence corresponding to an oligomerization domain of the target protein has at least 20% identity with the oligomerization domain of the target protein. In some embodiments, the polypeptide having an amino acid sequence corresponding to an oligomerization domain of the target protein has at least 30% identity with the oligomerization domain of the target protein. In some embodiments, the polypeptide having an amino acid sequence corresponding to an oligomerization domain of the target protein has at least 40% identity with the oligomerization domain of the target protein. In some embodiments, the polypeptide having an amino acid sequence corresponding to an oligomerization domain of the target protein has at least 50% identity with the oligomerization domain of the target protein. In some embodiments, the polypeptide having an amino acid sequence corresponding to an oligomerization domain of the target protein has at least 60% identity with the oligomerization domain of the target protein. In some embodiments, the polypeptide having an amino acid sequence corresponding to an oligomerization domain of the target protein has at least 70% identity with the oligomerization domain of the target protein. In some embodiments, the polypeptide having an amino acid sequence corresponding to an oligomerization domain of the target protein has at least 80% identity with the oligomerization domain of the target protein. In some embodiments, the polypeptide having an amino acid sequence corresponding to an oligomerization domain of the target protein has at least 85% identity with the oligomerization domain of the target protein. In some embodiments, the polypeptide having an amino acid sequence corresponding to an oligomerization domain of the target protein has at least 90% identity with the oligomerization domain of the target protein. In some embodiments, the polypeptide having an amino acid sequence corresponding to an oligomerization domain of the target protein has at least 95% identity with the oligomerization domain of the target protein. In some embodiments, the polypeptide having an amino acid sequence corresponding to an oligomerization domain of the target protein comprises the oligomerization domain of the target protein.

In some embodiments, the target protein comprises a viral protein. In some embodiments, the target protein comprises a viral glycoprotein. In some embodiments, the target protein comprises a viral spike protein.

Disclosed herein, in some aspects, is: a pharmaceutical composition comprising a vector encoding a polypeptide comprising an amino acid sequence having at least 10% identity with SEQ ID NOS:2, 4, 6, 8, 10, 12, 14, or 16; and a pharmaceutical composition comprising a vector encoding a polypeptide having an amino acid sequence corresponding to an oligomerization domain of a coronavirus spike protein and having at least 10% identity with SEQ ID NOS:2, 4, 6, 8, 10, 12, 14, or 16.

In some embodiments, the coronavirus spike protein comprises a SARS-CoV spike protein. In some embodiments, the coronavirus spike protein comprises a SARS-CoV-2 spike protein. In some embodiments, the coronavirus spike protein comprises a MERS-CoV spike protein. In some embodiments, the coronavirus spike protein comprises a HCoV-229E spike protein. In some embodiments, the coronavirus spike protein comprises a HCoV-NL63 spike protein. In some embodiments, the coronavirus spike protein comprises a HCoV-OC43 spike protein. In some embodiments, the coronavirus spike protein comprises a HCoV-HKU1 spike protein.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:2. In some embodiments, the polypeptide has at least 80% identity with SEQ ID NO:2. In some embodiments, the polypeptide has at least 85% identity with SEQ ID NO:2. In some embodiments, the polypeptide has at least 90% identity with SEQ ID NO:2. In some embodiments, the polypeptide has at least 95% identity with SEQ ID NO:2. In some embodiments, the polypeptide comprises SEQ ID NO:2.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:4. In some embodiments, the polypeptide has at least 80% identity with SEQ ID NO:4. In some embodiments, the polypeptide has at least 85% identity with SEQ ID NO:4. In some embodiments, the polypeptide has at least 90% identity with SEQ ID NO:4. In some embodiments, the polypeptide has at least 95% identity with SEQ ID NO:4. In some embodiments, the polypeptide comprises SEQ ID NO:4.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:6. In some embodiments, the polypeptide has at least 80% identity with SEQ ID NO:6. In some embodiments, the polypeptide has at least 85% identity with SEQ ID NO:6. In some embodiments, the polypeptide has at least 90% identity with SEQ ID NO:6. In some embodiments, the polypeptide has at least 95% identity with SEQ ID NO:6. In some embodiments, the polypeptide comprises SEQ ID NO:6.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:8. In some embodiments, the polypeptide has at least 80% identity with SEQ ID NO:8. In some embodiments, the polypeptide has at least 85% identity with SEQ ID NO:8. In some embodiments, the polypeptide has at least 90% identity with SEQ ID NO:8. In some embodiments, the polypeptide has at least 95% identity with SEQ ID NO:8. In some embodiments, the polypeptide comprises SEQ ID NO:8.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:10. In some embodiments, the polypeptide has at least 80% identity with SEQ ID NO:10. In some embodiments, the polypeptide has at least 85% identity with SEQ ID NO:10. In some embodiments, the polypeptide has at least 90% identity with SEQ ID NO:10. In some embodiments, the polypeptide has at least 95% identity with SEQ ID NO:10. In some embodiments, the polypeptide comprises SEQ ID NO:10.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:12. In some embodiments, the polypeptide has at least 80% identity with SEQ ID NO:12. In some embodiments, the polypeptide has at least 85% identity with SEQ ID NO:12. In some embodiments, the polypeptide has at least 90% identity with SEQ ID NO:12. In some embodiments, the polypeptide has at least 95% identity with SEQ ID NO:12. In some embodiments, the polypeptide comprises SEQ ID NO:12.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:14. In some embodiments, the polypeptide has at least 80% identity with SEQ ID NO:14. In some embodiments, the polypeptide has at least 85% identity with SEQ ID NO:14. In some embodiments, the polypeptide has at least 90% identity with SEQ ID NO:14. In some embodiments, the polypeptide has at least 95% identity with SEQ ID NO:14. In some embodiments, the polypeptide comprises SEQ ID NO:14.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:16. In some embodiments, the polypeptide has at least 80% identity with SEQ ID NO:16. In some embodiments, the polypeptide has at least 85% identity with SEQ ID NO:16. In some embodiments, the polypeptide has at least 90% identity with SEQ ID NO:16. In some embodiments, the polypeptide has at least 95% identity with SEQ ID NO:16. In some embodiments, the polypeptide comprises SEQ ID NO:16.

In some embodiments, the composition further comprises a second therapy for a coronavirus infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-SARS-CoV-2 drug. In some embodiments, the anti-SARS-CoV-2 drug is selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/MlN128), FK-506 (Tacrolimus), Ternatin 4 (DA3), Tigecycline, Tomivosertib (eFT-508), Verdinexor, WDB002, Zotatifin (eFT226), and a combination thereof.

Disclosed herein, in some aspects, is a pharmaceutical composition comprising a vector encoding a polypeptide comprising an amino acid sequence having at least 10% identity with SEQ ID NO:18; and a pharmaceutical composition comprising a vector encoding a polypeptide having an amino acid sequence corresponding to an oligomerization domain of an HIV spike protein and having at least 10% identity with SEQ ID NO:18.

In some embodiments, the HIV spike protein comprises an HIV gp160 spike protein.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:18. In some embodiments, the polypeptide has at least 80% identity with SEQ ID NO:18. In some embodiments, the polypeptide has at least 85% identity with SEQ ID NO:18. In some embodiments, the polypeptide has at least 90% identity with SEQ ID NO:18. In some embodiments, the polypeptide has at least 95% identity with SEQ ID NO:18. In some embodiments, the polypeptide comprises SEQ ID NO:18.

In some embodiments, the composition further comprises a second therapy for an HIV infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-HIV drug. In some embodiments, the anti-HIV drug is selected from the group consisting of efavirenz (Sustiva), rilpivirine (Edurant), etravirine (Intelence), delavirdine (Rescriptor), nevirapine (Viramune, Viramune XR), doravirine (Pifeltroz), abacavir (Ziagen), tenofovir alafenamide fumarate (Vemlidy), tenofovir (Viread), emtricitabine (Emtriva), lamivudine (Epivir), zidovudine (Retrovir), abacavir/lamivudine/zidovudine (Trizivir), abacavir/lamivudine (Epzicom), emtricitabine/tenofovir (Truvada), abacavir/lamivudine (Epzicom), lamivudine/tenofovir disoproxil fumarate (Cimduo, Temixys), lamivudine/zidovudine (Combivir), emtricitabine/tenofovir alafenamide (Descovy), didanosine (Videx, Videx EC), stavudine (Zerit), atazanavir (Reyataz), darunavir (Prezista), fosamprenavir (Lexiva), ritonavir (Norvir), tipranavir (Aptivus), lopinavir/ritonavir (Kaletra), atazanavir/cobicistat (Evotaz), darunavir/cobicistat (Prezcobix), indinavir (Crixivan), nelfinavir (Viracept), saquinavir (Invirase), bictegravir sodium/emtricitabine/tenofovir alafenamide fumar (Biktarvy), raltegravir (Isentress), elvitegravir (Genvoya and Stribild), dolutegravir (Tivicay), cobicistat (Tybost), ritonavir (Norvir), enfuvirtide (Fuzeon), maraviroc (Selzentry), ibalizumab-uiyk (Trogarzo), maraviroc (Selzentry), fostemsavir (Rukobia), doravirine/lamivudine/tenofovir disoproxil fumarate (Delstrigo), efavirenz/lamivudine/tenofovir disoproxil fumarate (Symfi), efavirenz/lamivudine/tenofovir disoproxil fumarate (Symfi Lo), efavirenz/emtricitabine/tenofovir disoproxil fumarate (Atripla), emtricitabine/rilpivirine/tenofovir alafenamide fumarate (Odefsey), emtricitabine/rilpivirine/tenofovir disoproxil fumarate (Complera), elvitegravir/cobicistat/emtricitabine/tenofovir disoproxil fumarate (Stribild), elvitegravir/cobicistat/emtricitabine/tenofovir alafenamide fumarate (Genvoya), abacavir/dolutegravir/lamivudine (Triumeq), bictegravir/emtricitabine/tenofovir alafenamide fumarate (Biktarvy), dolutegravir/lamivudine (Dovato), dolutegravir/rilpivirine (Juluca), darunavir/cobicistat/emtricitabine/tenofovir alafenamide fumarate (Symtuza), acetyl-L-carnitine, whey protein, L-glutamine, L-arginine, hydroxymethylbutyrate (HMB), probiotics, vitamins and minerals, and a combination thereof.

Disclosed herein, in some aspects, is a pharmaceutical composition comprising a vector encoding a polypeptide comprising an amino acid sequence having at least 10% identity with SEQ ID NO:20; and a pharmaceutical composition comprising a vector encoding a polypeptide having an amino acid sequence corresponding to an oligomerization domain of an Ebola glycoprotein and having at least 10% identity with SEQ ID NO:20.

In some embodiments, the Ebola virus glycoprotein comprises an Ebola GP spike protein.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:20. In some embodiments, the polypeptide has at least 80% identity with SEQ ID NO:20. In some embodiments, the polypeptide has at least 85% identity with SEQ ID NO:20. In some embodiments, the polypeptide has at least 90% identity with SEQ ID NO:20. In some embodiments, the polypeptide has at least 95% identity with SEQ ID NO:20. In some embodiments, the polypeptide comprises SEQ ID NO:20.

In some embodiments, the composition further comprises a second therapy for an Ebola infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-Ebola drug. In some embodiments, the anti-Ebola drug is selected from the group consisting of atoltivimab/maftivimab/odesivimab-ebgn (Inmazeb), ansuvimab-zykl (Ebanga), Favipiravir (Avigan), Ribavirin, BCX4430, Brincidofovir, TKM-Ebola, AVI-7537, JK-05, and a combination thereof.

Disclosed herein, in some aspects, is a pharmaceutical composition comprising a vector encoding a polypeptide comprising an amino acid sequence having at least 10% identity with SEQ ID NO:25; and a pharmaceutical composition comprising a vector encoding a polypeptide having an amino acid sequence corresponding to an oligomerization domain of an influenza virus spike protein and having at least 10% identity with SEQ ID NO:25.

In some embodiments, the influenza comprises influenza virus A, and the influenza virus spike protein comprises an influenza virus A spike protein.

In some embodiments, the influenza virus A comprises influenza virus A/H1, and the influenza virus spike protein comprises an influenza virus A/H1 HA spike protein. In some embodiments, the influenza virus A comprises influenza virus A/H3, and the influenza virus spike protein comprises an influenza virus A/H3 HA spike protein. In some embodiments, the influenza comprises influenza virus B, and the influenza virus spike protein comprises an influenza virus B spike protein. In some embodiments, the influenza virus B comprises influenza virus B/Victoria, and the influenza virus spike protein comprises an influenza virus B/Victoria HA spike protein. In some embodiments, the influenza virus B comprises influenza virus B/Yamagata, and the influenza virus spike protein comprises an influenza virus B/Yamagata HA spike protein.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:25. In some embodiments, the polypeptide has at least 80% identity with SEQ ID NO:25. In some embodiments, the polypeptide has at least 85% identity with SEQ ID NO:25. In some embodiments, the polypeptide has at least 90% identity with SEQ ID NO:25. In some embodiments, the polypeptide has at least 95% identity with SEQ ID NO:25. In some embodiments, the polypeptide comprises SEQ ID NO:25.

In some embodiments, the composition further comprises a second therapy for an influenza infection. In some embodiments, the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. In some embodiments, the second therapy comprises an anti-influenza drug. In some embodiments, the anti-influenza drug is selected from the group consisting of oseltamivir phosphate (Tamiflu), zanamivir (Relenza), peramivir (Rapivab), baloxavir marboxil (Xofluza), amantadine, rimantadine (Flumadine), umifenovir (Arbidol), moroxydine, fluticare, acetaminophen, chlorpheniramine, dextromethorphan, pseudoephedrine, and a combination thereof.

Disclosed herein, in some aspects, is a pharmaceutical composition comprising a vector encoding a polypeptide comprising an amino acid sequence having at least 10% identity with SEQ ID NO:34; and a pharmaceutical composition comprising a vector encoding a polypeptide having an amino acid sequence corresponding to an oligomerization domain of an RSV glycoprotein and having at least 10% identity with SEQ ID NO:34.

In some embodiments, the RSV glycoprotein comprises an RSV F glycoprotein.

In some embodiments, the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:34. In some embodiments, the polypeptide has at least 80% identity with SEQ ID NO:34. In some embodiments, the polypeptide has at least 85% identity with SEQ ID NO:34. In some embodiments, the polypeptide has at least 90% identity with SEQ ID NO:34. In some embodiments, the polypeptide has at least 95% identity with SEQ ID NO:34. In some embodiments, the polypeptide comprises SEQ ID NO:34.

In some embodiments, the composition further comprises a second therapy for an RSV infection. In some embodiments, the second therapy comprises an anti-RSV drug.

In some embodiments of the compositions disclosed herein, the vector is a viral vector or a non-viral vector. In some embodiments, the vector is a minicircle DNA vector. In some embodiments, a dose of between 1×10⁸ to 1×10¹⁸ vector genomes/kg body weight of the subject is administered to the subject. In some embodiments, a dose of about 1×10¹¹ to about 1×10¹⁴ vector genomes/kg body weight of the subject is administered to the subject. In some embodiments, a dose of about 1×10¹² to about 1×10¹⁵ vector genomes/kg body weight of the subject is administered to the subject.

In some embodiments of the compositions disclosed herein, the compositions further comprise a pharmaceutically acceptable carrier. In some embodiments, the pharmaceutically acceptable carrier comprises liposomes, polymeric micelles, microspheres, or nanoparticles.

In some embodiments of the compositions disclosed herein, a single dose of the composition is administered. In some embodiments, multiple doses of the composition are administered. In some embodiments, the composition is delivered to the subject once a day, more than once a day, more than once a week, more than once a month, or more than once a year. In some embodiments, the composition is delivered systemically or locally. In some embodiments, the composition is administered to the subject intranasally, intravenously, intraperitoneally, intratracheally, intramuscularly, endoscopically, percutaneously, subcutaneously, regionally, intracranially, by inhalation, by injection, by infusion, or by perfusion. In some embodiments, the composition is administered to the subject by inhalation. In some embodiments, the composition is administered to the subject intranasally.

“Individual, “subject,” and “patient” are used interchangeably and can refer to a human or non-human.

As used herein, “treat,” “treating,” or “treatment” or equivalent terminology refer to both therapeutic treatment and prophylactic or preventative measures, wherein the object is to prevent or slow down (lessen) an undesired physiological change or disorder, such as the growth, development, or spread of a disease, including but not limited to a virus. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The results of treatment can be determined by methods known in the art, such as determination of reduction of viral load, determination of restoration of function, or other methods known in the art.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary, Detailed Description, Claims, and Description of the Drawings.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C. The concept of polypeptide-based protein inhibition. FIG. 1A. Domain organization of COVID-19 SARS2-S, mutations in SARS2-S variants, and design of inhibitory polypeptides CoV-F1 (F1) and CoV-F2 (F2). SP: Signal peptide; NTD: N-terminal domain; RBD: receptor-binding domain; SD1: subdomain 1; SD2: subdomain 2; FP: fusion peptide; HR1: heptad repeat 1; HR2: heptad repeat 2; TM: transmembrane domain; CT: Cytoplasmic tail. The cleavage at S1/S2 (red arrow) gives rise to N-terminal S1 fragment and C-terminal S2 fragment. The signal peptide sequence at the extreme N-termini of F1 and F2 allowed the polypeptides to be translocated in the same way as COVID-19 SARS2-S. At the extreme C-terminus, SARS2-S has a C9 epitope recognized by a C9-rhodopsin antibody 1D4, while both the F1 and F2 polypeptides have a FLAG-tag. F1 encompasses SP, HR1, HR2, TM, and CT (residues 911-1273; SEQ ID NO:2), while F2 contains SP, HR2, TM, and CT (residues 985-1273; SEQ ID NO:27). FIG. 1B. Proposed mode of action for polypeptide-based inhibition targeting coronavirus spike proteins. Top row: in normal situations, the spike proteins are synthesized, folded as monomers, and assembled into native spike oligomers, which are anchored on virions. Bottom row: inhibitory polypeptides interfere with the assembly step, resulting in non-native oligomers with wild-type spike proteins. The net outcome is a reduced number of native spike oligomers on new virions.

FIGS. 2A-2C. F1 polypeptide expressed form plasmid DNA significantly reduced expression and surface translocation of three coronavirus spike glycoproteins. FIG. 2A. COVID-19 SARS2-S. The amount of SARS2-S protein in whole-cell lysate (left) or cell-surface fraction (right) were compared for HEK293T cells transfected with SARS2-S-coding plasmid only (column #1), or together with two-fold (column #2) or ten-fold molar ratio (column #3) of F1-coding plasmid, or with two-fold (column #4) or ten-fold molar ratio (column #5) of F2-coding plasmid. FIG. 2B. 2002 SARS-S. The amount of SARS-S protein in whole-cell lysate (left) or cell-surface fraction (right) were compared for HEK293T cells transfected with SARS-S-coding plasmid only (column #1), or together with two-fold (column #2) or ten-fold molar ratio (column #3) of F1-coding plasmid. FIG. 2C. 2012 MERS-S. The amount of MERS-S protein in whole-cell lysate (left) or cell-surface fraction (right) were compared for HEK293T cells transfected with MERS-S-coding plasmid only (column #1), or together with two-fold (column #2) or ten-fold molar ratio (column #3) of F1-coding plasmid. For all panels, the uncleaved S and the cleaved S2 proteins were detected by C9-rhodopsin antibody 1D4. Endogenous membrane protein CNPase was detected by anti-CNPase antibody and served as an internal control. Shown were representative results from at least two independent experiments.

FIGS. 3A-3B. Sequence alignment of coronavirus spike proteins. FIG. 3A. The alignment of SARS2-S(SARS2), SARS-S(SARS), and SARS-CoV-2 B.1.1.7-S(SARS2a) spike proteins. The range of residues included in the F1 polypeptide is highlighted in blue boxes. FIG. 3B. The alignment of SARS2-S(SARS2) and MERS-S (MERS) proteins. The range of residues included in the F1 polypeptide is highlighted in blue boxes. The cleavage site between S1 and S2 is shown by a red arrow. Within Si, the boundaries between NTD and RBD domains are indicated by green vertical bars. Multiple sequence alignment was performed using CLUSTAL Omega²⁹. The sequences are: SARS2-S (GenBank accession number AFR58740.1), SARS-S (GenBank accession number QHD43416.1), B.1.1.7-S(SARS2α-S) (GenBank accession number QQH18545.1), and MERS-S (GenBank accession number QBM11748.1).

FIGS. 4A-4D. Sequence conservation of representative viral glycoproteins. FIG. 4A. Comparison of SARS-S and MERS-S proteins with full-length SARS2-S. FIG. 4B. Comparison of representative human coronavirus spike proteins with SARS2-S F1 polypeptide (residues 911-1273). FIG. 4C. Comparison of A/H1N1, B/Vic and B/YM HA proteins with full-length A/H3N2 HA. FIG. 4D. Comparison of A/H1N1, B/Vic and B/YM HA with A/H3N2 HAi polypeptide. In all panels, shown are the number of residues that are identical (Identity), the total residues compared (after “/”), and the corresponding percentage (in parenthesis). The total residues vary slightly due to various gaps in sequence alignment. The sequences are: SARS2-S (GenBank accession number AFR58740.1), SARS-S (GenBank accession number QHD43416.1), SARS2α-S (GenBank accession number QQH18545.1), MERS-S (GenBank accession number QBM11748.1), HCoV-HKU1-S (Uniprot accession number QOZME7), HCoV-OC43-S (Uniprot accession number P36334), HCoV-NL63-S (Uniprot accession number Q6Q1S2), and HCoV-229E-S (Uniprot accession number P15423). In panels c) and d), the HA sequences were from the four flu vaccine strains in 2019-2020, A/Hawaii/70/2019 (A/H1N1), A/Hong Kong/45/2019 (A/H3N2), B/Washington/02/2019 (B/Vic), and B/Phuket/3073/2013 (B/YM).

FIGS. 5A-5C. Protein expression levels of F1 and F2 polypeptides when F1- or F2-coding plasmid was co-transfected with coronavirus spike protein-coding plasmids for COVID-19 SARS2-S (FIG. 5A); 2002 SARS-S (FIG. 5B); and 2012 MERS-S (FIG. 5C). Endogenous membrane-anchored protein CNPase detected by anti-CNPase antibody was used as an internal control. Representative results from two or more independent experiments.

FIGS. 6A-6E. F1 polypeptide expressed from minicircle DNA interacted with coronavirus spike proteins at the protein level. The mRNA levels of spike proteins COVID-19 SARS2-S (FIG. 6A), 2002 SARS-S (FIG. 6B), and 2012 MERS-S (FIG. 6C) were measured in the absence or presence of F1-coding plasmid. FIG. 6D. Diagram for the FRET setup, where CFP and YFP were each tagged to the extreme C-terminus of SARS2-S and F1, resulting in SARS2-C and F1Y, respectively. FIG. 6E. FRET ratio of SARS2-C with F1Y (at a 1:1 molar ratio for transfection), compared to that of F1Y only. ****, p<0.0001 in unpaired t-test comparing FRET ratio of SARS2C+F1Y versus same-day F1Y only for each set of experiments on a given day. Numbers in parentheses indicate the number of cells included in each analysis. Shown are results from three independent experiments.

FIGS. 7A-7C. The mRNA level of F1 and F2 when co-transfected with coronavirus spike protein-coding plasmids for COVID-19 SARS2-S (FIG. 7A); 2002 SARS-S (FIG. 7B); and 2012 MERS-S (FIG. 7C). The mRNA levels of corresponding proteins in the absence of inhibitory polypeptide-coding plasmids were treated as a baseline against which the mRNA levels of other samples were normalized. The error bars represent standard deviations (SD) from two or more independent experiments.

FIGS. 8A-8D. F1 polypeptide expressed from minicircle DNA significantly reduced expression and surface translocation of three coronavirus spike glycoproteins. FIG. 8A Diagram for the production of F1 minicircles used in this study. FIGS. 8B-8D. Results for COVID-19 SARS2-S (FIG. 8B); 2002 SARS-S (FIG. 8C); and 2012 MERS-S (FIG. 8D). The amount of uncleaved S protein and the cleaved S2 protein in whole-cell lysate (left) or cell-surface fraction (right) are compared for HEK293T cells transfected with S-coding plasmid only, or together with 4.5-fold or 22.5-fold molar ratio of F1 minicircles. Endogenous membrane-anchored protein CNPase detected by anti-CNPase antibody was used as an internal control. Representative results from two or more independent experiments.

FIGS. 9A-9B. F1 polypeptide expressed from minicircle DNA significantly reduced the amount of SARS2-S on intact pseudoviruses and impaired pseudovirus infectivity. FIG. 9A. The amount of cleaved S2 protein was compared for intact pseudovirus produced from HEK293T cells transfected with SARS2-S-coding plasmid and different ratios of empty minicircle control, MN501A (columns #1-4), or together with different ratios of F1 minicircle (columns #5-8).). p24 detected by anti-p24 antibody was used as an internal control. Representative results from two independent experiments. FIG. 9B. Pseudovirus generated in the presence of two-fold molar ratio of F1 minicircle lost the ability to infect hACE2-expressing HEK293T cells. The infectivity of pseudovirus generated in the presence of MN501A minicircle at a two-fold molar ratio was considered as 100% for each co-transfection ratio. Shown are representative results from at least two independent experiments. The error bars represent standard deviations (SD) from two independent experiments.

FIG. 10. Diagram of generalized polypeptide-based protein inhibition. FIG. 10A. For homo-oligomers, in normal situations, proteins are first synthesized, folded as monomers, and then multiple monomers (shown in blue and green colors but in the same shape for different monomers of the same protein species) are assembled into native protein complexes. In polypeptide-based protein interference situations, an interfering polypeptide (shown in blue color), which is derived from or contain one or more segments of the target protein (shown in blue color) in the native protein complex or its homologues, competes with the target protein to interact with the native interacting partner of the target protein (identical to the target protein in sequence and structure, shown in green color) and blocks the native protein complex formation, resulting in non-native protein complexes, thus regulating the level of assembled native protein complex. FIG. 10B. For hetero-oligomers, in normal situations, proteins are first synthesized, folded, and then multiple protein species (shown in blue and orange colors and different shapes for different protein species) are assembled into native protein complexes. In polypeptide-based protein interference situations, an interfering polypeptide (shown in blue color), which is derived from or contains one or more segments of the target protein (shown in blue color) in the native protein complex or its homologues, competes with the target protein to interact with the native interacting partner of the target protein (shown in orange color) and blocks the native protein complex formation, resulting in non-native protein complexes, thus regulating the level of assembled native protein complex. FIG. 10C. For some types of hetero-oligomeric native protein complexes comprising multiple copies of the target protein (shown in blue and green colors) and other protein species (shown in orange color), in normal situations, proteins are first synthesized, folded, and then assembled into native protein complexes. In polypeptide-based protein interference situations, an interfering polypeptide (shown in blue color), which is derived from or contains one or more segments of the target protein (shown in blue color) in the native protein complex or its homologues, competes with the target protein to interact with the native interacting partners of the target protein (shown in green and orange colors) and blocks the native protein complex formation, resulting in non-native protein complexes, thus regulating the level of assembled native protein complex. Polypeptide-based protein interference can lead to down- or up-regulation of the target pathway.

FIG. 11. Inhibitory polypeptide gp160i significantly reduced expression of HIV-1 gp160 glycoprotein. The amount of uncleaved gp160 protein and the cleaved gp41 protein in whole-cell lysate was compared for HEK293T cells transfected with gp160-coding plasmid only, or together with 5-fold of plasmid encoding gp160i. Uncleaved gp160 and cleaved gp41 were detected by C9-rhodopsin antibody 1D4, and endogenous membrane protein CNPase was detected by anti-CNPase antibody and served as an internal control. Representative results from two or more independent experiments.

FIG. 12. Inhibitory polypeptide GPi significantly reduced expression and translocation of Ebola GP glycoprotein. The amount of uncleaved GP protein and the cleaved GP2 protein in whole-cell lysate (left) and cell surface (right) was compared for HEK293T cells transfected with GP-containing plasmid only, or together with 5-fold or 15-fold molar ratio of plasmid encoding GPi. Uncleaved GP and cleaved GP2 were detected by C9-rhodopsin antibody 1D4, and endogenous membrane protein CNPase was detected by anti-CNPase antibody and served as an internal control. Representative results from two or more independent experiments.

FIG. 13. Inhibitory polypeptide HAi significantly reduced expression and translocation of influenza virus type A and type B glycoprotein hemagglutinin (HA). The amount of uncleaved HA protein in whole-cell lysate (left) and cell surface (right) was compared for HEK293T cells transfected with HA-coding plasmid only, or together with 5-fold plasmid encoding HAi. Uncleaved HA was detected by C9-rhodopsin antibody 1D4, and endogenous membrane protein CNPase was detected by anti-CNPase antibody and served as an internal control. The results for A/H1 HA are shown as an example. Representative results from two or more independent experiments.

FIG. 14. Inhibitory polypeptide Fi significantly reduced expression and translocation of RSV type A glycoprotein F. The amount of uncleaved RSV F protein in whole-cell lysate (left) and cell surface (right) was compared for HEK293T cells transfected with RSV F-coding plasmid only, or together with 5-fold or 15-fold molar ratio plasmid encoding Fi. Uncleaved RSV F was detected by C9-rhodopsin antibody 1D4, and endogenous membrane protein CNPase was detected by anti-CNPase antibody and served as an internal control. Representative results from two or more independent experiments.

FIGS. 15A-15D. F1 polypeptide expressed from minicircle DNA effectively blocked the replication of authentic SARS-CoV-2 virus in hACE2-mice. FIG. 15A. The experimental setup of in vivo protection test of F1 polypeptide in hACE2-mice. FIG. 15B. The body weight changes of the two mouse groups. FIG. 15C. Box plot showing the loads of SARS-CoV-2 virus in mouse lungs in the two experimental groups. Each dot represents one mouse, “x” marks the mean. The p-value is reported for unpaired two-tailed t-test comparing the viral loads of the two groups (n=6). FIG. 15D. H&E staining of mouse lung tissue sections at 3 days post-inoculation comparing the two experimental groups.

FIGS. 16A-16E. Amino-acid sequences of CoV-F1 (F1) (FIG. 16A), CoV-F2 (F2) (FIG. 16B), HIV gp160i (FIG. 16C), Ebola GPi (FIG. 16D), RSV Fi (FIG. 16E), and Influenza HAi (FIG. 16F) used herein. Single-letter codes are used for amino acids. The sequences were derived from: SARS2-S (GenBank accession number AFR58740.1), HIV-1 gp160 (GenBank accession number NP_057856.1); Zaire EBOV GP (GenBank accession number AAN37507.1); RSV type A F (GenBank accession number QKN22797.1); and influenza virus A/HongKong/45/2019 (H3N2) HA. The signal peptide sequences are highlighted in boldface at the N-terminus. FLAG-tag at the extreme C-terminus of each polypeptide includes a FLAG-tag and a twin-strep tag preceding the FLAG-tag (not shown). The underlined residues in RSV Fi represent an introduced linker.

### DETAILED DESCRIPTION

The present disclosure fulfills certain needs in the fields of medicine and virology by providing compositions and methods for polypeptide-based inhibition and for utilizing polypeptide-based inhibition in treatment and prevention of diseases included but not limited to viral infections (e.g., a coronavirus infection, an HIV infection, an Ebola infection, an RSV infection, or an influenza infection) and/or post-viral infection syndromes and is based, at least in part, on the surprising discovery that polypeptide-based inhibition is a powerful innovative strategy to reduce cell-surface translocation of viral spike proteins and to impair the ability of virus progenies to infect host cells. The novel compositions and method involve design regulatory polypeptides against a protein and are widely applicable as an innovative and efficient solution for post-translational regulation of protein levels in vitro and in vivo without irreversible genetic modifications.

As described herein, inhibitory polypeptides designed based on COVID-19 SARS-CoV-2 and influenza A/H3N2 virus glycoprotein sequences were unexpectedly found to be equally effective against glycoproteins of other coronavirus and influenza strains, respectively, despite as low as 27% sequence identity, underscoring their high insensitivity to mutations and potential activity of the inhibitory polypeptides on glycoproteins from viruses of different lineages. In fact, it has been surprisingly shown that in hACE2-mice infected with authentic SARS-CoV-2 virus, only one dose of inhibitory polypeptide-coding DNA effectively prevented viral replication in mouse lungs. Moreover, at least in part due to their reduced sequence size, minicircles encoding the inhibitory polypeptides may survive the shear forces in generating aerosols to allow convenient and effective nasal spray delivery²⁷. Therefore, in some embodiments, the compositions and related methods disclosed herein can provide the basis for highly effective broad-spectrum antiviral therapeutics. Furthermore, these approaches represent a general way to alter expression of essentially any proteins post-translationally, thus providing a straightforward and high-precision approach for treating infectious or non-infectious diseases.

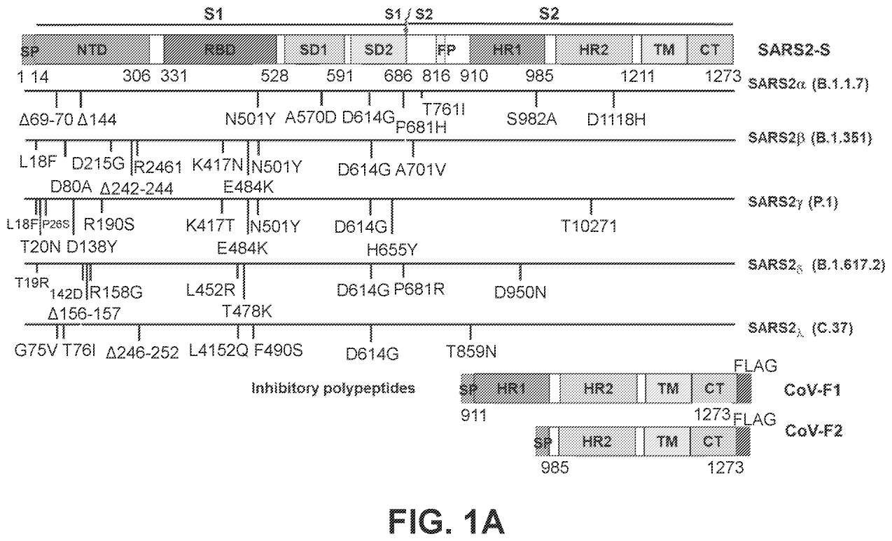

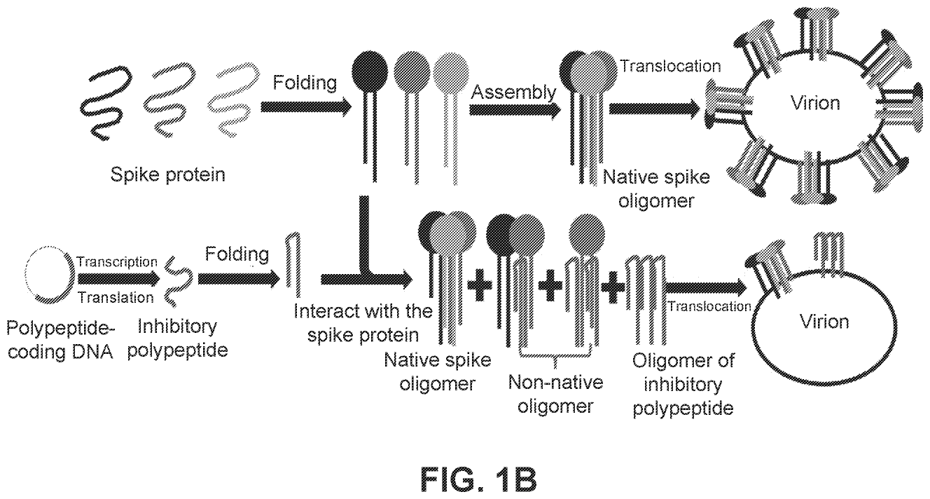

For RNA viruses including coronaviruses, the high mutational rate of the viruses is a powerful weapon for repeated infection among human populations. As exemplified by recent SARS2-S variants^{3, 4, 7, 8, 9}, mutations accumulated in the S1 region allow evasion from neutralizing antibodies elicited by natural infection or vaccines (FIG. 1A). The S2 region of these spike proteins is generally more invariant in evolution. Therefore, polypeptide-based inhibition that utilizes the highly conserved sequences of the viral proteins can be employed for novel therapeutics with high efficacy and target specificity, wide-spectrum coverage, and adaptability to other viral proteins, including but not limited to other viral membrane fusion glycoproteins, to combat viral pathogens.

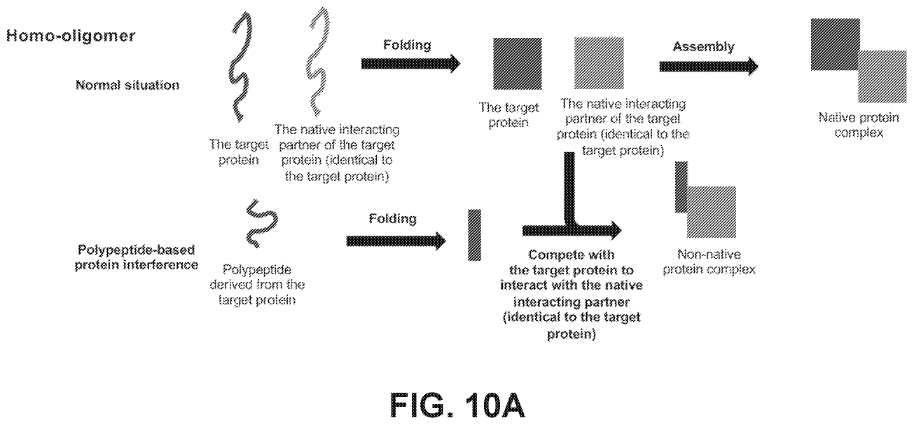

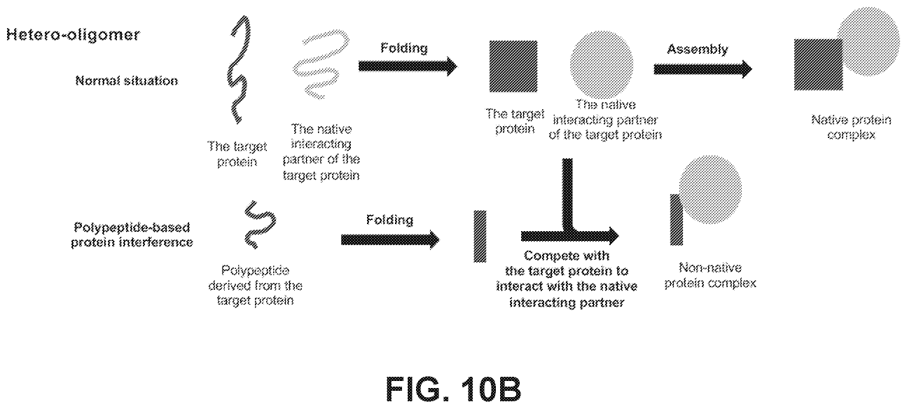

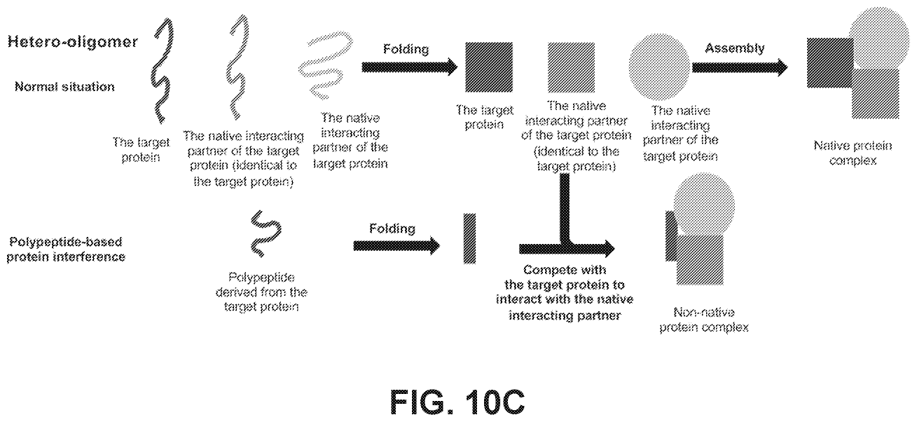

Furthermore, the polypeptide-based protein inhibition described herein offers a general method for post-translational regulation of native protein complexes where interactions within the complex can be regulated with a polypeptide derived from or containing one or more segments of the target protein in the native protein complex or its homologues (FIG. 10), for example HIV, Ebola, RSV, and influenza virus spike protein complexes. Due to the relatively larger interacting surface of the polypeptide compared to smaller organic compounds, such polypeptide-based inhibition provides high efficacy and specificity for therapeutics, and the polypeptides can be easily designed or selected in the absence of structural information. In addition, the polypeptides derived from one or more segments of a host protein are essentially immunogenically inert.

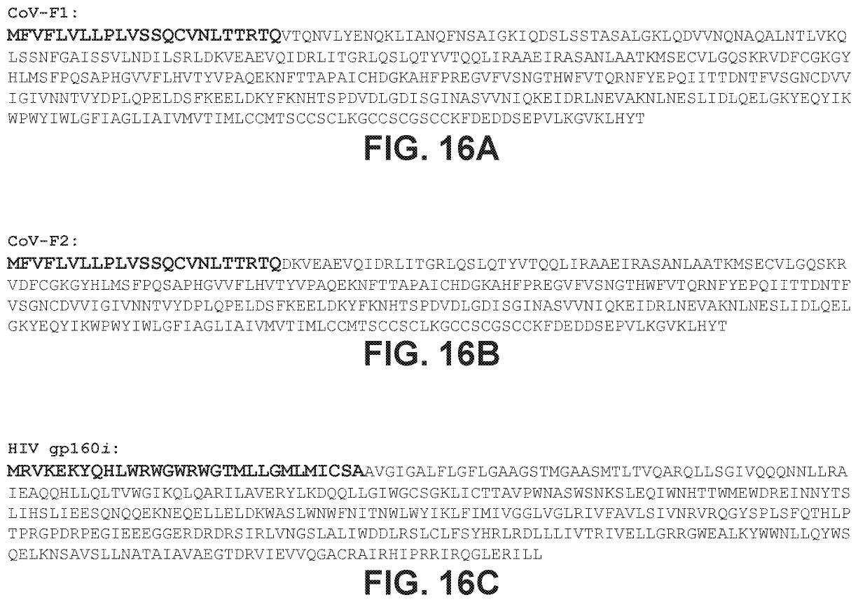

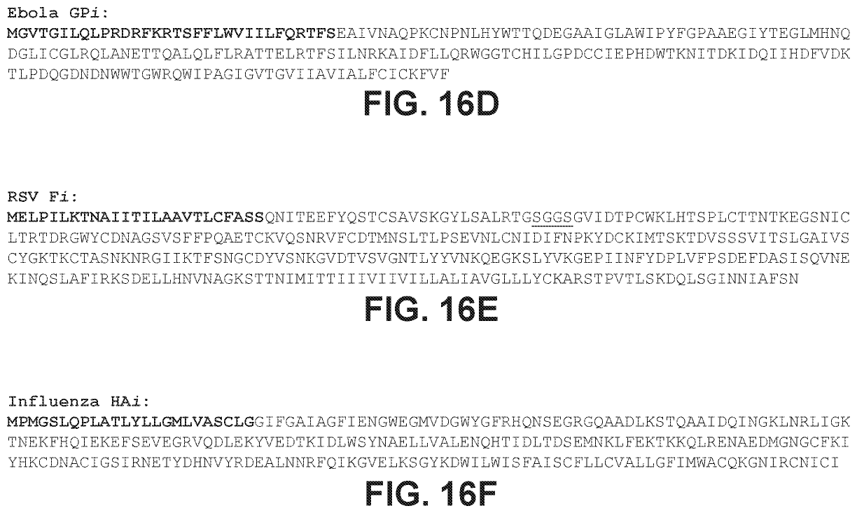

Aspects of the disclosure are based on compositions comprising protein-interacting polypeptides (also referred to herein as “interfering polypeptides” or “protein-interfering polypeptides”) such as those characterized by FIGS. 16A-16E that interact, and in some cases interfere, with proteins, like viral proteins such as viral spike proteins including but not limited to coronavirus spike proteins, HIV spike proteins, Ebola virus glycoproteins, RSV glycoproteins, and influenza virus spike proteins, and methods of administration of the polypeptides; however, polypeptides that interact with other viral and non-viral proteins are also contemplated and within the scope of the disclosure. Certain polypeptides of the disclosure inhibit formation and translocation of the virus spike protein to cell surfaces of the subject and/or to viral envelopes. Certain polypeptides of the disclosure oligomerize with conserved oligomerization domains of virus spike proteins to inhibit formation and translocation of the virus spike protein to cell surfaces of the subject and/or to viral envelopes. Certain polypeptides of the disclosure reduce the amount of the virus spike protein on cell surfaces of the subject and/or on viral envelopes. Certain polypeptides of the disclosure oligomerize with conserved oligomerization domains of virus spike proteins to reduce the amount of the virus spike protein on cell surfaces of the subject and/or on viral envelopes. In some cases, inhibition of formation and translocation of the virus spike protein to cell surfaces of the subject and/or to viral envelopes and/or a reduction of the amount of the virus spike protein on cell surfaces of the subject and/or on viral envelopes treats or prevents the viral infections and/or post-viral infection syndromes. Thus, such polypeptides are useful, for example, in methods for treatment and prevention of viral infections, such as a coronavirus infection (e.g., MERS-CoV, SARS-CoV, and SARS-CoV-2 infection) and/or post-coronavirus infection syndrome, an HIV infection, an Ebola infection, an RSV infection, and/or an influenza infection, or other diseases unrelated to viral infections for which polypeptide-based protein inhibition would be beneficial.

### I. Protein-Interacting Polypeptide Compositions

Aspects of the disclosure relate to polypeptides that interact with, and in some cases inhibit or interfere with, a target protein and/or a native interacting partner thereof (“protein-interacting polypeptides” or “inhibitory polypeptides” or “interfering polypeptides” or “interacting polypeptides” or “protein-interfering polypeptides”), including but not limited to viral proteins like viral spike proteins, in vivo, compositions comprising such polypeptides, and methods of use of the polypeptides for treatment and prevention of a disease or condition in a subject, including but not limited to a viral (e.g., coronavirus, HIV, Ebola, RSV, influenza) infection.

As disclosed herein, “interact with” describes any contact between a protein-interacting polypeptide and a target protein and/or a native interacting partner of the target protein at an interacting surface, including but not limited to oligomerization of the protein-interacting polypeptide with a corresponding oligomerization domain of the target protein and/or the native interacting partner of the target protein. For example, in some cases, “interact with” may describe any contact between a protein-interacting polypeptide and a target protein, a protein-interacting polypeptide and a native interacting partner of a target protein, and/or a protein-interacting polypeptide, a target protein, and a native interacting partner of a target protein. In some cases, “interact with” may describe oligomerization of a protein-interacting polypeptide and a target protein, a protein-interacting polypeptide and a native interacting partner of a target protein, and/or a protein-interacting polypeptide, a target protein, and a native interacting partner of a target protein. In certain embodiments, “interact with” may describe any contact between a viral protein-interacting polypeptide and a viral protein, including but not limited to oligomerization of the viral protein-interacting polypeptide with a corresponding oligomerization domain of viral protein.

In some cases, such interaction between a protein-interacting polypeptide and a target protein and/or a native interacting partner of the target protein at an interacting surface may be inhibition or interference. As disclosed herein, “inhibit,” “inhibitory,” “inhibition,” “interference,” “interfere with,” and related terms and phrases describe contact between a protein-interacting polypeptide, a target protein, and/or a native interacting partner of the target protein that prevents native protein complex formation, including but not limited to competition of the protein-interacting polypeptide with the target protein for interaction with the native interacting partner of the target protein. In some cases, “inhibit,” “inhibitory,” “inhibition,” “interference,” “interfere with,” and related terms and phrases may describe oligomerization of a protein-interacting polypeptide with a corresponding oligomerization domain of the target protein and/or the native interacting partner of the target protein that prevents native oligomeric protein complex formation, including but not limited to competition of the protein-interacting polypeptide with the target protein for interaction with a corresponding oligomerization domain of the native interacting partner of the target protein. In certain embodiments, “inhibit,” “inhibitory,” “inhibition,” “interfere with” may describe contact between a viral protein-interacting polypeptide and a viral protein such that the viral protein-interacting polypeptide competes with a corresponding oligomerization domain of the viral protein.

As disclosed herein, a “protein-interacting polypeptide” describes any polypeptide capable of interacting with and in some cases inhibiting or interfering with a target protein and/or a native interacting partner of the target protein. For example, a protein-interacting polypeptide may be a viral protein-interacting polypeptide that interacts with, inhibits, or interferes with viral proteins, such as viral spike glycoproteins and/or other viral proteins.

As disclosed herein, a “target protein” refers to a protein from which the amino acid sequence of a protein-interacting polypeptide disclosed herein is derived having at least 10% amino acid sequence identity with the protein-interacting polypeptide.

In some cases, a “target protein” is a monomer of a homo-oligomeric protein complex, and interaction, or contact, of a protein-interacting polypeptide with the protein monomer inhibits native homo-oligomeric complex formation (see, e.g., FIG. 10A). In such cases, the protein-interacting polypeptide competes with monomers to prevent native homo-oligomeric complex formation by the targeted monomer and additional monomers, thereby regulating the monomer and/or biological function thereof.

In some cases, a “target protein” is a native protein, and interaction, or contact, of a protein-interacting polypeptide with a native interacting partner of the native protein prevents further interaction of the native protein with the native interacting partner of the native protein (see, e.g., FIG. 10B). In such cases, the protein-interacting polypeptide competes with the native protein for binding to the native interacting partner of the native protein to prevent native hetero-oligomer complex formation by the native protein, thereby regulating the native protein and/or biological function thereof.

In some cases, a “target protein” is a native protein, and interaction, or contact, of a protein-interacting polypeptide with the native protein and/or a native interacting partner of the native protein prevents further interaction of the native protein with other native proteins and/or the native interacting partner of the native protein (see, e.g., FIG. 10C). In such cases, the protein-interacting polypeptide competes with the native protein for binding to the native protein and/or the native interacting partner of the native protein to prevent native hetero-oligomer complex formation by the native protein, thereby regulating the native protein and/or biological function thereof.

The amino acid sequences of the protein-interacting polypeptides disclosed herein may be derived from one or more “target proteins” to achieve regulation of a native protein complex. Furthermore, one or more “protein-interacting polypeptides” may be used to regulate a native protein complex, and one or more “protein-interacting polypeptides” may be used to regulate one or more native protein complexes.

As disclosed herein, a “native interacting partner of a target protein,” “native interacting partner of the native protein,” and the like refer to one or more proteins that associate with a native protein in a cell to form native protein complexes.

A protein-interacting polypeptide can block and inhibit or interfere with a target protein's homo-oligomerization, or interaction with itself, upon binding of the interacting polypeptide at an interacting surface, and/or a protein-interacting polypeptide can block and inhibit or interfere with a target protein's hetero-oligomerization, or interaction with a native interacting partner of the target protein, upon binding of the interacting polypeptide at an interacting surface. As a result of such interaction, the polypeptide, which can be derived from or contain one or more segments of the target protein in the native protein complex or its homologues, competes with the target protein and results in non-native protein complexes, thus regulating the level of assembled native protein complex. Such polypeptide-based protein inhibition or interference can lead to down- or up-regulation of the target pathway.

As used herein, “regulating,” “regulate,” “regulation,” and grammatical equivalents refer to regulation of a target protein and/or biological function thereof, which allows the cell to regulate not only the amounts but also the activities of its protein constituents. Native protein complexes consist of multiple independent polypeptide subunits. In some homo-oligomeric embodiments, the protein subunits of native protein complexes are identical; in other hetero-oligomeric embodiments, the protein subunits of native protein complexes are two or more distinct polypeptides. In either case, interactions between the polypeptides are important in regulation of protein activity.

In some embodiments, “regulation” of a target protein and/or biological function thereof comprises inhibition of the target protein and/or biological function thereof. As used herein, the terms “reduce,” “inhibit,” “diminish,” “suppress,” and grammatical equivalents (including “lower,” “smaller,” etc.) refer to a measurable decrease, in some cases, a statistically significant decrease, in occurrence or activity, including full blocking of an occurrence or activity. For example, activity can refer to one or more biological activities of the native protein and/or native protein complex, and occurrence can refer to formation of native protein complexes. For example, “inhibition” can refer to a decrease of about 10%, 20%, 30%, 40%, 50%, 60%, 70%, 80%, 90% or 100% in activity or occurrence. These terms can be used relative to a “control” that has not been subjected to a particular treatment, e.g., a method of the disclosure. In one example, the control can be an untreated sample or subject. In another example, the control can be a sample or subject that has received a different treatment from the treated sample or subject. In some embodiments, a “treated” sample or subject is one that has been subjected to a method of the disclosure.

In some embodiments, “regulation” and/or “inhibition” of a target protein and/or biological function thereof comprises impaired biological activity of the target protein and/or native protein complex upon formation of non-native protein complexes. In some embodiments, “regulation” and/or “inhibition” of a target protein and/or biological function thereof comprises proteasomal degradation of non-native protein complexes, which are loosely packed and fail the quality control system of cells, upon formation of the non-native protein complexes.

Thus, in some embodiments, regulation of a target protein, including but not limited to a viral protein such as a coronavirus spike protein, an HIV spike protein, an Ebola glycoprotein, an RSV glycoprotein, and/or an influenza virus spike protein, or biological function thereof in vivo comprises inhibition of the target protein, including but not limited to a viral protein such as a coronavirus spike protein, an HIV spike protein, an Ebola glycoprotein, an RSV glycoprotein, and/or an influenza virus spike protein, or biological function thereof in vivo. In some embodiments, inhibition of the target protein, including but not limited to a viral protein such as a coronavirus spike protein, an HIV spike protein, an Ebola glycoprotein, an RSV glycoprotein, and/or an influenza virus spike protein, or biological function thereof in vivo comprises impairment of the biological activity of the target protein and/or the native protein complex comprising the target protein upon formation of non-native protein complexes. In some embodiments, inhibition of the target protein, including but not limited to a viral protein such as a coronavirus spike protein, an HIV spike protein, an Ebola glycoprotein, an RSV glycoprotein, and/or an influenza virus spike protein, or biological function thereof in vivo comprises proteasomal degradation of the target protein and/or the native protein complex comprising the target protein upon formation of non-native protein complexes.

Certain aspects relate to polypeptides having an amino acid sequence corresponding to a sequence of a target protein, and the amino acid sequence has at least 10% sequence identity with the corresponding sequence of the target protein. Certain aspects relate to polypeptides having an amino acid sequence corresponding to an oligomerization domain of a target protein, and the amino acid sequence has at least 10% sequence identity with the oligomerization domain of the target protein.

Certain aspects relate to polypeptides having an amino acid sequence having at least 10-80% identity with SEQ ID NOS:2, 4, 6, 8, 10, 12, 14, or 16. Certain aspects relate to polypeptides having an amino acid sequence corresponding to an oligomerization domain of a coronavirus spike protein, and the amino acid sequence has at least 10-80% identity with SEQ ID NOS:2, 4, 6, 8, 10, 12, 14, or 16.

Certain aspects relate to polypeptides having an amino acid sequence having at least 10-80% identity with SEQ ID NO:18. Certain aspects relate to polypeptides having an amino acid sequence corresponding to a sequence of an HIV spike protein, and the amino acid sequence has at least 10-80% identity with SEQ ID NO:18.

Certain aspects relate to polypeptides having an amino acid sequence having at least 10-80% identity with SEQ ID NO:20. Certain aspects relate to polypeptides having an amino acid sequence corresponding to a sequence of an Ebola virus glycoprotein, and the amino acid sequence has at least 10-80% identity with SEQ ID NO:20.

Certain aspects relate to polypeptides having an amino acid sequence having at least 10-80% identity with SEQ ID NO: 25. Certain aspects relate to polypeptides having an amino acid sequence corresponding to a sequence of an influenza virus HA spike protein, and the amino acid sequence has at least 10-80% identity with SEQ ID NO:25.

Certain aspects relate to polypeptides having an amino acid sequence having at least 10-80% identity with SEQ ID NO: 33. Certain aspects relate to polypeptides having an amino acid sequence corresponding to a sequence of an RSV glycoprotein, and the amino acid sequence has at least 10-80% identity with SEQ ID NO:34.

In some embodiments, the polypeptide has an amino acid sequence corresponding to a sequence of a target protein, the amino acid sequence having at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or more amino acids, or any value derivable therein, and having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the sequence of the target protein or fragments or functional derivatives thereof. In some embodiments, the polypeptide has an amino acid sequence corresponding to a sequence of a target protein, the amino acid sequence having at least 30 amino acids and having 10% sequence identity with the sequence of the target protein or fragments or functional derivatives thereof. In some embodiments, the polypeptide has an amino acid sequence corresponding to a sequence of a target protein, the amino acid sequence having at least 30 amino acids and having 20% sequence identity with the sequence of the target protein or fragments or functional derivatives thereof. In some embodiments, the polypeptide has an amino acid sequence corresponding to a sequence of a target protein, the amino acid sequence having at least 30 amino acids and having 30% sequence identity with the sequence of the target protein or fragments or functional derivatives thereof. In some embodiments, the polypeptide has an amino acid sequence corresponding to a sequence of a target protein, the amino acid sequence having at least 30 amino acids and having 40% sequence identity with the sequence of the target protein or fragments or functional derivatives thereof. In some embodiments, the polypeptide has an amino acid sequence corresponding to a sequence of a target protein, the amino acid sequence having at least 30 amino acids and having 50% sequence identity with the sequence of the target protein or fragments or functional derivatives thereof. In some embodiments, the polypeptide has an amino acid sequence corresponding to a sequence of a target protein, the amino acid sequence having at least 30 amino acids and having 60% sequence identity with the sequence of the target protein or fragments or functional derivatives thereof. In some embodiments, the polypeptide has an amino acid sequence corresponding to a sequence of a target protein, the amino acid sequence having at least 30 amino acids and having 70% sequence identity with the sequence of the target protein or fragments or functional derivatives thereof. In some embodiments, the polypeptide has an amino acid sequence corresponding to a sequence of a target protein, the amino acid sequence having at least 30 amino acids and having at least 75% sequence identity with the sequence of the target protein or fragments or functional derivatives thereof. In some embodiments, the polypeptide has an amino acid sequence corresponding to a sequence of a target protein, the amino acid sequence having at least 30 amino acids and having at least 80% sequence identity with the sequence the of target protein or fragments or functional derivatives thereof. In some embodiments, the polypeptide has an amino acid sequence corresponding to a sequence of a target protein, the amino acid sequence having at least 30 amino acids and having at least 85% sequence identity with the sequence of the target protein or fragments or functional derivatives thereof. In some embodiments, the polypeptide has an amino acid sequence corresponding to a sequence of a target protein, the amino acid sequence having at least 30 amino acids and having at least 90% sequence identity with the sequence of the target protein or fragments or functional derivatives thereof. In some embodiments, the polypeptide has an amino acid sequence corresponding to a sequence of a target protein, the amino acid sequence having at least 30 amino acids and having at least 95% sequence identity with the sequence of the target protein or fragments or functional derivatives thereof. In some embodiments, the polypeptide having an amino acid sequence corresponding to a sequence of a target protein comprises, consists of, or consists essentially of the sequence of the target protein or fragments or functional derivatives thereof.

In some embodiments, the polypeptide has an amino acid sequence corresponding to an oligomerization domain of a target protein, the amino acid sequence having at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or more amino acids, or any value derivable therein, and having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 10% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 20% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 30% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 40% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 50% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 60% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having 70% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having at least 75% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having at least 80% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having at least 85% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having at least 90% sequence identity with the oligomerization domain of the target protein or fragments or functional derivatives thereof. In some embodiments, the polypeptide has an amino acid sequence corresponding to an oligomerization domain of a target protein, the amino acid sequence having at least 30 amino acids and having at least 95% sequence identity with the oligomerization domain of the target protein or fragments or functional derivatives thereof. In some embodiments, the polypeptide having an amino acid sequence corresponding to an oligomerization domain of a target protein comprises, consists of, or consists essentially of the oligomerization domain of the target protein or fragments or functional derivatives thereof.

In some embodiments, the polypeptide that interacts with a protein, for example, a coronavirus spike protein, comprises an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NOS:2, 4, 6, 8, 10, 12, 14, or 16, or a fragment or functional derivative thereof.

In some embodiments, the polypeptide that interacts with a protein, for example, a coronavirus spike protein, comprises an amino acid sequence having at least 10% sequence identity with SEQ ID NO:2 or a fragment or functional derivative thereof. In some embodiments, the polypeptide that interacts with a protein, for example, a coronavirus spike protein, comprises an amino acid sequence having at least 20% sequence identity with SEQ ID NO:2 or a fragment or functional derivative thereof. In some embodiments, the polypeptide that interacts with a protein, for example, a coronavirus spike protein, comprises an amino acid sequence having at least 30% sequence identity with SEQ ID NO:2 or a fragment or functional derivative thereof. In some embodiments, the polypeptide that interacts with a protein, for example, a coronavirus spike protein, comprises an amino acid sequence having at least 40% sequence identity with SEQ ID NO:2 or a fragment or functional derivative thereof. In some embodiments, the polypeptide that interacts with a protein, for example, a coronavirus spike protein, comprises an amino acid sequence having at least 50% sequence identity with SEQ ID NO:2 or a fragment or functional derivative thereof. In some embodiments, the polypeptide that interacts with a protein, for example, a coronavirus spike protein, comprises an amino acid sequence having at least 60% sequence identity with SEQ ID NO:2 or a fragment or functional derivative thereof. In some embodiments, the polypeptide that interacts with a protein, for example, a coronavirus spike protein, comprises an amino acid sequence having at least 70% sequence identity with SEQ ID NO:2 or a fragment or functional derivative thereof. In some embodiments, the polypeptide that interacts with a protein, for example, a coronavirus spike protein, comprises an amino acid sequence having at least 75% sequence identity with SEQ ID NO:2 or a fragment or functional derivative thereof. In some embodiments, the polypeptide that interacts with a protein, for example, a coronavirus spike protein, comprises an amino acid sequence having at least 80% sequence identity with SEQ ID NO:2 or a fragment or functional derivative thereof. In some embodiments, the polypeptide that interacts with a protein, for example, a coronavirus spike protein, comprises an amino acid sequence having at least 85% sequence identity with SEQ ID NO:2 or a fragment or functional derivative thereof. In some embodiments, the polypeptide that interacts with a protein, for example, a coronavirus spike protein, comprises an amino acid sequence having at least 90% sequence identity with SEQ ID NO:2 or a fragment or functional derivative thereof. In some embodiments, the polypeptide that interacts with a protein, for example, a coronavirus spike protein, comprises an amino acid sequence having at least 95% sequence identity with SEQ ID NO:2 or a fragment or functional derivative thereof. In some embodiments, the polypeptide that interacts with a protein, for example, a coronavirus spike protein, comprises, consists of, or consists essentially of SEQ ID NO:2 or a fragment or functional derivative thereof.

In some embodiments, the polypeptide that interacts with a protein, for example, a coronavirus spike protein, comprises an amino acid sequence having at least 10% sequence identity with SEQ ID NO:4 or a fragment or functional derivative thereof. In some embodiments, the polypeptide that interacts with a protein, for example, a coronavirus spike protein, comprises an amino acid sequence having at least 20% sequence identity with SEQ ID NO:4 or a fragment or functional derivative thereof. In some embodiments, the polypeptide that interacts with a protein, for example, a coronavirus spike protein, comprises an amino acid sequence having at least 30% sequence identity with SEQ ID NO:4 or a fragment or functional derivative thereof. In some embodiments, the polypeptide that interacts with a protein, for example, a coronavirus spike protein, comprises an amino acid sequence having at least 40% sequence identity with SEQ ID NO:4 or a fragment or functional derivative thereof. In some embodiments, the polypeptide that interacts with a protein, for example, a coronavirus spike protein, comprises an amino acid sequence having at least 50% sequence identity with SEQ ID NO:4 or a fragment or functional derivative thereof. In some embodiments, the polypeptide that interacts with a protein, for example, a coronavirus spike protein, comprises an amino acid sequence having at least 60% sequence identity with SEQ ID NO:4 or a fragment or functional derivative thereof. In some embodiments, the polypeptide that interacts with a protein, for example, a coronavirus spike protein, comprises an amino acid sequence having at least 70% sequence identity with SEQ ID NO:4 or a fragment or functional derivative thereof. In some embodiments, the polypeptide that interacts with a protein, for example, a coronavirus spike protein, comprises an amino acid sequence having at least 75% sequence identity with SEQ ID NO:4 or a fragment or functional derivative thereof. In some embodiments, the polypeptide that interacts with a protein, for example, a coronavirus spike protein, comprises an amino acid sequence having at least 80% sequence identity with SEQ ID NO:4 or a fragment or functional derivative thereof. In some embodiments, the polypeptide that interacts with a protein, for example, a coronavirus spike protein, comprises an amino acid sequence having at least 85% sequence identity with SEQ ID NO:4 or a fragment or functional derivative thereof. In some embodiments, the polypeptide that interacts with a protein, for example, a coronavirus spike protein, comprises an amino acid sequence having at least 90% sequence identity with SEQ ID NO:4 or a fragment or functional derivative thereof. In some embodiments, the polypeptide that interacts with a protein, for example, a coronavirus spike protein, comprises an amino acid sequence having at least 95% sequence identity with SEQ ID NO:4 or a fragment or functional derivative thereof. In some embodiments, the polypeptide that interacts with a protein, for example, a coronavirus spike protein, comprises, consists of, or consists essentially of SEQ ID NO:4 or a fragment or functional derivative thereof.

In some embodiments, the polypeptide that interacts with a protein, for example, a coronavirus spike protein, comprises an amino acid sequence having at least 10% sequence identity with SEQ ID NO:6 or a fragment or functional derivative thereof. In some embodiments, the polypeptide that interacts with a protein, for example, a coronavirus spike protein, comprises an amino acid sequence having at least 20% sequence identity with SEQ ID NO:6 or a fragment or functional derivative thereof. In some embodiments, the polypeptide that interacts with a protein, for example, a coronavirus spike protein, comprises an amino acid sequence having at least 30% sequence identity with SEQ ID NO:6 or a fragment or functional derivative thereof. In some embodiments, the polypeptide that interacts with a protein, for example, a coronavirus spike protein, comprises an amino acid sequence having at least 40% sequence identity with SEQ ID NO:6 or a fragment or functional derivative thereof. In some embodiments, the polypeptide that interacts with a protein, for example, a coronavirus spike protein, comprises an amino acid sequence having at least 50% sequence identity with SEQ ID NO:6 or a fragment or functional derivative thereof. In some embodiments, the polypeptide that interacts with a protein, for example, a coronavirus spike protein, comprises an amino acid sequence having at least 60% sequence identity with SEQ ID NO:6 or a fragment or functional derivative thereof. In some embodiments, the polypeptide that interacts with a protein, for example, a coronavirus spike protein, comprises an amino acid sequence having at least 70% sequence identity with SEQ ID NO:6 or a fragment or functional derivative thereof. In some embodiments, the polypeptide that interacts with a protein, for example, a coronavirus spike protein, comprises an amino acid sequence having at least 75% sequence identity with SEQ ID NO:6 or a fragment or functional derivative thereof. In some embodiments, the polypeptide that interacts with a protein, for example, a coronavirus spike protein, comprises an amino acid sequence having at least 80% sequence identity with SEQ ID NO:6 or a fragment or functional derivative thereof. In some embodiments, the polypeptide that interacts with a protein, for example, a coronavirus spike protein, comprises an amino acid sequence having at least 85% sequence identity with SEQ ID NO:6 or a fragment or functional derivative thereof. In some embodiments, the polypeptide that interacts with a protein, for example, a coronavirus spike protein, comprises an amino acid sequence having at least 90% sequence identity with SEQ ID NO:6 or a fragment or functional derivative thereof. In some embodiments, the polypeptide that interacts with a protein, for example, a coronavirus spike protein, comprises an amino acid sequence having at least 95% sequence identity with SEQ ID NO:6 or a fragment or functional derivative thereof. In some embodiments, the polypeptide that interacts with a protein, for example, a coronavirus spike protein, comprises, consists of, or consists essentially of SEQ ID NO:6 or a fragment or functional derivative thereof.

In some embodiments, the polypeptide that interacts with a protein, for example, a coronavirus spike protein, comprises an amino acid sequence having at least 10% sequence identity with SEQ ID NO:8 or a fragment or functional derivative thereof. In some embodiments, the polypeptide that interacts with a protein, for example, a coronavirus spike protein, comprises an amino acid sequence having at least 20% sequence identity with SEQ ID NO:8 or a fragment or functional derivative thereof. In some embodiments, the polypeptide that interacts with a protein, for example, a coronavirus spike protein, comprises an amino acid sequence having at least 30% sequence identity with SEQ ID NO:8 or a fragment or functional derivative thereof. In some embodiments, the polypeptide that interacts with a protein, for example, a coronavirus spike protein, comprises an amino acid sequence having at least 40% sequence identity with SEQ ID NO:8 or a fragment or functional derivative thereof. In some embodiments, the polypeptide that interacts with a protein, for example, a coronavirus spike protein, comprises an amino acid sequence having at least 50% sequence identity with SEQ ID NO:8 or a fragment or functional derivative thereof. In some embodiments, the polypeptide that interacts with a protein, for example, a coronavirus spike protein, comprises an amino acid sequence having at least 60% sequence identity with SEQ ID NO:8 or a fragment or functional derivative thereof. In some embodiments, the polypeptide that interacts with a protein, for example, a coronavirus spike protein, comprises an amino acid sequence having at least 70% sequence identity with SEQ ID NO:8 or a fragment or functional derivative thereof. In some embodiments, the polypeptide that interacts with a protein, for example, a coronavirus spike protein, comprises an amino acid sequence having at least 75% sequence identity with SEQ ID NO:8 or a fragment or functional derivative thereof. In some embodiments, the polypeptide that interacts with a protein, for example, a coronavirus spike protein, comprises an amino acid sequence having at least 80% sequence identity with SEQ ID NO:8 or a fragment or functional derivative thereof. In some embodiments, the polypeptide that interacts with a protein, for example, a coronavirus spike protein, comprises an amino acid sequence having at least 85% sequence identity with SEQ ID NO:8 or a fragment or functional derivative thereof. In some embodiments, the polypeptide that interacts with a protein, for example, a coronavirus spike protein, comprises an amino acid sequence having at least 90% sequence identity with SEQ ID NO:8 or a fragment or functional derivative thereof. In some embodiments, the polypeptide that interacts with a protein, for example, a coronavirus spike protein, comprises an amino acid sequence having at least 95% sequence identity with SEQ ID NO:8 or a fragment or functional derivative thereof. In some embodiments, the polypeptide that interacts with a protein, for example, a coronavirus spike protein, comprises, consists of, or consists essentially of SEQ ID NO:8 or a fragment or functional derivative thereof.

In some embodiments, the polypeptide that interacts with a protein, for example, a coronavirus spike protein, comprises an amino acid sequence having at least 10% sequence identity with SEQ ID NO:10 or a fragment or functional derivative thereof. In some embodiments, the polypeptide that interacts with a protein, for example, a coronavirus spike protein, comprises an amino acid sequence having at least 20% sequence identity with SEQ ID NO:10 or a fragment or functional derivative thereof. In some embodiments, the polypeptide that interacts with a protein, for example, a coronavirus spike protein, comprises an amino acid sequence having at least 30% sequence identity with SEQ ID NO:10 or a fragment or functional derivative thereof. In some embodiments, the polypeptide that interacts with a protein, for example, a coronavirus spike protein, comprises an amino acid sequence having at least 40% sequence identity with SEQ ID NO:10 or a fragment or functional derivative thereof. In some embodiments, the polypeptide that interacts with a protein, for example, a coronavirus spike protein, comprises an amino acid sequence having at least 50% sequence identity with SEQ ID NO:10 or a fragment or functional derivative thereof. In some embodiments, the polypeptide that interacts with a protein, for example, a coronavirus spike protein, comprises an amino acid sequence having at least 60% sequence identity with SEQ ID NO:10 or a fragment or functional derivative thereof. In some embodiments, the polypeptide that interacts with a protein, for example, a coronavirus spike protein, comprises an amino acid sequence having at least 70% sequence identity with SEQ ID NO:10 or a fragment or functional derivative thereof. In some embodiments, the polypeptide that interacts with a protein, for example, a coronavirus spike protein, comprises an amino acid sequence having at least 75% sequence identity with SEQ ID NO:10 or a fragment or functional derivative thereof. In some embodiments, the polypeptide that interacts with a protein, for example, a coronavirus spike protein, comprises an amino acid sequence having at least 80% sequence identity with SEQ ID NO:10 or a fragment or functional derivative thereof. In some embodiments, the polypeptide that interacts with a protein, for example, a coronavirus spike protein, comprises an amino acid sequence having at least 85% sequence identity with SEQ ID NO:10 or a fragment or functional derivative thereof. In some embodiments, the polypeptide that interacts with a protein, for example, a coronavirus spike protein, comprises an amino acid sequence having at least 90% sequence identity with SEQ ID NO:10 or a fragment or functional derivative thereof. In some embodiments, the polypeptide that interacts with a protein, for example, a coronavirus spike protein, comprises an amino acid sequence having at least 95% sequence identity with SEQ ID NO:10 or a fragment or functional derivative thereof. In some embodiments, the polypeptide that interacts with a protein, for example, a coronavirus spike protein, comprises, consists of, or consists essentially of SEQ ID NO:10 or a fragment or functional derivative thereof.

In some embodiments, the polypeptide that interacts with a protein, for example, a coronavirus spike protein, comprises an amino acid sequence having at least 10% sequence identity with SEQ ID NO:12 or a fragment or functional derivative thereof. In some embodiments, the polypeptide that interacts with a protein, for example, a coronavirus spike protein, comprises an amino acid sequence having at least 20% sequence identity with SEQ ID NO:12 or a fragment or functional derivative thereof. In some embodiments, the polypeptide that interacts with a protein, for example, a coronavirus spike protein, comprises an amino acid sequence having at least 30% sequence identity with SEQ ID NO:12 or a fragment or functional derivative thereof. In some embodiments, the polypeptide that interacts with a protein, for example, a coronavirus spike protein, comprises an amino acid sequence having at least 40% sequence identity with SEQ ID NO:12 or a fragment or functional derivative thereof. In some embodiments, the polypeptide that interacts with a protein, for example, a coronavirus spike protein, comprises an amino acid sequence having at least 50% sequence identity with SEQ ID NO:12 or a fragment or functional derivative thereof. In some embodiments, the polypeptide that interacts with a protein, for example, a coronavirus spike protein, comprises an amino acid sequence having at least 60% sequence identity with SEQ ID NO:12 or a fragment or functional derivative thereof. In some embodiments, the polypeptide that interacts with a protein, for example, a coronavirus spike protein, comprises an amino acid sequence having at least 70% sequence identity with SEQ ID NO:12 or a fragment or functional derivative thereof. In some embodiments, the polypeptide that interacts with a protein, for example, a coronavirus spike protein, comprises an amino acid sequence having at least 75% sequence identity with SEQ ID NO:12 or a fragment or functional derivative thereof. In some embodiments, the polypeptide that interacts with a protein, for example, a coronavirus spike protein, comprises an amino acid sequence having at least 80% sequence identity with SEQ ID NO:12 or a fragment or functional derivative thereof. In some embodiments, the polypeptide that interacts with a protein, for example, a coronavirus spike protein, comprises an amino acid sequence having at least 85% sequence identity with SEQ ID NO:12 or a fragment or functional derivative thereof. In some embodiments, the polypeptide that interacts with a protein, for example, a coronavirus spike protein, comprises an amino acid sequence having at least 90% sequence identity with SEQ ID NO:12 or a fragment or functional derivative thereof. In some embodiments, the polypeptide that interacts with a protein, for example, a coronavirus spike protein, comprises an amino acid sequence having at least 95% sequence identity with SEQ ID NO:12 or a fragment or functional derivative thereof. In some embodiments, the polypeptide that interacts with a protein, for example, a coronavirus spike protein, comprises, consists of, or consists essentially of SEQ ID NO:12 or a fragment or functional derivative thereof.

In some embodiments, the polypeptide that interacts with a protein, for example, a coronavirus spike protein, comprises an amino acid sequence having at least 10% sequence identity with SEQ ID NO:14 or a fragment or functional derivative thereof. In some embodiments, the polypeptide that interacts with a protein, for example, a coronavirus spike protein, comprises an amino acid sequence having at least 20% sequence identity with SEQ ID NO:14 or a fragment or functional derivative thereof. In some embodiments, the polypeptide that interacts with a protein, for example, a coronavirus spike protein, comprises an amino acid sequence having at least 30% sequence identity with SEQ ID NO:14 or a fragment or functional derivative thereof. In some embodiments, the polypeptide that interacts with a protein, for example, a coronavirus spike protein, comprises an amino acid sequence having at least 40% sequence identity with SEQ ID NO:14 or a fragment or functional derivative thereof. In some embodiments, the polypeptide that interacts with a protein, for example, a coronavirus spike protein, comprises an amino acid sequence having at least 50% sequence identity with SEQ ID NO:14 or a fragment or functional derivative thereof. In some embodiments, the polypeptide that interacts with a protein, for example, a coronavirus spike protein, comprises an amino acid sequence having at least 60% sequence identity with SEQ ID NO:14 or a fragment or functional derivative thereof. In some embodiments, the polypeptide that interacts with a protein, for example, a coronavirus spike protein, comprises an amino acid sequence having at least 70% sequence identity with SEQ ID NO:14 or a fragment or functional derivative thereof. In some embodiments, the polypeptide that interacts with a protein, for example, a coronavirus spike protein, comprises an amino acid sequence having at least 75% sequence identity with SEQ ID NO:14 or a fragment or functional derivative thereof. In some embodiments, the polypeptide that interacts with a protein, for example, a coronavirus spike protein, comprises an amino acid sequence having at least 80% sequence identity with SEQ ID NO:14 or a fragment or functional derivative thereof. In some embodiments, the polypeptide that interacts with a protein, for example, a coronavirus spike protein, comprises an amino acid sequence having at least 85% sequence identity with SEQ ID NO:14 or a fragment or functional derivative thereof. In some embodiments, the polypeptide that interacts with a protein, for example, a coronavirus spike protein, comprises an amino acid sequence having at least 90% sequence identity with SEQ ID NO:14 or a fragment or functional derivative thereof. In some embodiments, the polypeptide that interacts with a protein, for example, a coronavirus spike protein, comprises an amino acid sequence having at least 95% sequence identity with SEQ ID NO:14 or a fragment or functional derivative thereof. In some embodiments, the polypeptide that interacts with a protein, for example, a coronavirus spike protein, comprises, consists of, or consists essentially of SEQ ID NO:14 or a fragment or functional derivative thereof.

In some embodiments, the polypeptide that interacts with a protein, for example, a coronavirus spike protein, comprises an amino acid sequence having at least 10% sequence identity with SEQ ID NO:16 or a fragment or functional derivative thereof. In some embodiments, the polypeptide that interacts with a protein, for example, a coronavirus spike protein, comprises an amino acid sequence having at least 20% sequence identity with SEQ ID NO:16 or a fragment or functional derivative thereof. In some embodiments, the polypeptide that interacts with a protein, for example, a coronavirus spike protein, comprises an amino acid sequence having at least 30% sequence identity with SEQ ID NO:16 or a fragment or functional derivative thereof. In some embodiments, the polypeptide that interacts with a protein, for example, a coronavirus spike protein, comprises an amino acid sequence having at least 40% sequence identity with SEQ ID NO:16 or a fragment or functional derivative thereof. In some embodiments, the polypeptide that interacts with a protein, for example, a coronavirus spike protein, comprises an amino acid sequence having at least 50% sequence identity with SEQ ID NO:16 or a fragment or functional derivative thereof. In some embodiments, the polypeptide that interacts with a protein, for example, a coronavirus spike protein, comprises an amino acid sequence having at least 60% sequence identity with SEQ ID NO:16 or a fragment or functional derivative thereof. In some embodiments, the polypeptide that interacts with a protein, for example, a coronavirus spike protein, comprises an amino acid sequence having at least 70% sequence identity with SEQ ID NO:16 or a fragment or functional derivative thereof. In some embodiments, the polypeptide that interacts with a protein, for example, a coronavirus spike protein, comprises an amino acid sequence having at least 75% sequence identity with SEQ ID NO:16 or a fragment or functional derivative thereof. In some embodiments, the polypeptide that interacts with a protein, for example, a coronavirus spike protein, comprises an amino acid sequence having at least 80% sequence identity with SEQ ID NO:16 or a fragment or functional derivative thereof. In some embodiments, the polypeptide that interacts with a protein, for example, a coronavirus spike protein, comprises an amino acid sequence having at least 85% sequence identity with SEQ ID NO:16 or a fragment or functional derivative thereof. In some embodiments, the polypeptide that interacts with a protein, for example, a coronavirus spike protein, comprises an amino acid sequence having at least 90% sequence identity with SEQ ID NO:16 or a fragment or functional derivative thereof. In some embodiments, the polypeptide that interacts with a protein, for example, a coronavirus spike protein, comprises an amino acid sequence having at least 95% sequence identity with SEQ ID NO:16 or a fragment or functional derivative thereof. In some embodiments, the polypeptide that interacts with a protein, for example, a coronavirus spike protein, comprises, consists of, or consists essentially of SEQ ID NO:16 or a fragment or functional derivative thereof.

In some embodiments, the polypeptide that interacts with a protein, for example, an HIV spike protein, comprises an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:18, or a fragment or functional derivative thereof.

In some embodiments, the polypeptide that interacts with a protein, for example, an HIV spike protein, comprises an amino acid sequence having at least 10% sequence identity with SEQ ID NO:18 or a fragment or functional derivative thereof. In some embodiments, the polypeptide that interacts with a protein, for example, an HIV spike protein, comprises an amino acid sequence having at least 20% sequence identity with SEQ ID NO:18 or a fragment or functional derivative thereof. In some embodiments, the polypeptide that interacts with a protein, for example, an HIV spike protein, comprises an amino acid sequence having at least 30% sequence identity with SEQ ID NO:18 or a fragment or functional derivative thereof. In some embodiments, the polypeptide that interacts with a protein, for example, an HIV spike protein, comprises an amino acid sequence having at least 40% sequence identity with SEQ ID NO:18 or a fragment or functional derivative thereof. In some embodiments, the polypeptide that interacts with a protein, for example, an HIV spike protein, comprises an amino acid sequence having at least 50% sequence identity with SEQ ID NO:18 or a fragment or functional derivative thereof. In some embodiments, the polypeptide that interacts with a protein, for example, an HIV spike protein, comprises an amino acid sequence having at least 60% sequence identity with SEQ ID NO:18 or a fragment or functional derivative thereof. In some embodiments, the polypeptide that interacts with a protein, for example, an HIV spike protein, comprises an amino acid sequence having at least 70% sequence identity with SEQ ID NO:18 or a fragment or functional derivative thereof. In some embodiments, the polypeptide that interacts with a protein, for example, an HIV spike protein, comprises an amino acid sequence having at least 75% sequence identity with SEQ ID NO:18 or a fragment or functional derivative thereof. In some embodiments, the polypeptide that interacts with a protein, for example, an HIV spike protein, comprises an amino acid sequence having at least 80% sequence identity with SEQ ID NO:18 or a fragment or functional derivative thereof. In some embodiments, the polypeptide that interacts with a protein, for example, an HIV spike protein, comprises an amino acid sequence having at least 85% sequence identity with SEQ ID NO:18 or a fragment or functional derivative thereof. In some embodiments, the polypeptide that interacts with a protein, for example, an HIV spike protein, comprises an amino acid sequence having at least 90% sequence identity with SEQ ID NO:18 or a fragment or functional derivative thereof. In some embodiments, the polypeptide that interacts with a protein, for example, an HIV spike protein, comprises an amino acid sequence having at least 95% sequence identity with SEQ ID NO:18 or a fragment or functional derivative thereof. In some embodiments, the polypeptide that interacts with a protein, for example, an HIV spike protein, comprises, consists of, or consists essentially of SEQ ID NO:18 or a fragment or functional derivative thereof.

In some embodiments, the polypeptide that interacts with a protein, for example, an Ebola virus glycoprotein, comprises an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:20, or a fragment or functional derivative thereof.

In some embodiments, the polypeptide that interacts with a protein, for example, an Ebola virus glycoprotein, comprises an amino acid sequence having at least 10% sequence identity with SEQ ID NO:20 or a fragment or functional derivative thereof. In some embodiments, the polypeptide that interacts with a protein, for example, an Ebola virus glycoprotein, comprises an amino acid sequence having at least 20% sequence identity with SEQ ID NO:20 or a fragment or functional derivative thereof. In some embodiments, the polypeptide that interacts with a protein, for example, an Ebola virus glycoprotein, comprises an amino acid sequence having at least 30% sequence identity with SEQ ID NO:20 or a fragment or functional derivative thereof. In some embodiments, the polypeptide that interacts with a protein, for example, an Ebola virus glycoprotein, comprises an amino acid sequence having at least 40% sequence identity with SEQ ID NO:20 or a fragment or functional derivative thereof. In some embodiments, the polypeptide that interacts with a protein, for example, an Ebola virus glycoprotein, comprises an amino acid sequence having at least 50% sequence identity with SEQ ID NO:20 or a fragment or functional derivative thereof. In some embodiments, the polypeptide that interacts with a protein, for example, an Ebola virus glycoprotein, comprises an amino acid sequence having at least 60% sequence identity with SEQ ID NO:20 or a fragment or functional derivative thereof. In some embodiments, the polypeptide that interacts with a protein, for example, an Ebola virus glycoprotein, comprises an amino acid sequence having at least 70% sequence identity with SEQ ID NO:20 or a fragment or functional derivative thereof. In some embodiments, the polypeptide that interacts with a protein, for example, an Ebola virus glycoprotein, comprises an amino acid sequence having at least 75% sequence identity with SEQ ID NO:20 or a fragment or functional derivative thereof. In some embodiments, the polypeptide that interacts with a protein, for example, an Ebola virus glycoprotein, comprises an amino acid sequence having at least 80% sequence identity with SEQ ID NO:20 or a fragment or functional derivative thereof. In some embodiments, the polypeptide that interacts with a protein, for example, an Ebola virus glycoprotein, comprises an amino acid sequence having at least 85% sequence identity with SEQ ID NO:20 or a fragment or functional derivative thereof. In some embodiments, the polypeptide that interacts with a protein, for example, an Ebola virus glycoprotein, comprises an amino acid sequence having at least 90% sequence identity with SEQ ID NO:20 or a fragment or functional derivative thereof. In some embodiments, the polypeptide that interacts with a protein, for example, an Ebola virus glycoprotein, comprises an amino acid sequence having at least 95% sequence identity with SEQ ID NO:20 or a fragment or functional derivative thereof. In some embodiments, the polypeptide that interacts with a protein, for example, an Ebola virus glycoprotein, comprises, consists of, or consists essentially of SEQ ID NO:20 or a fragment or functional derivative thereof.

In some embodiments, the polypeptide that interacts with a protein, for example, an influenza virus spike protein, comprises an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:25, or a fragment or functional derivative thereof.

In some embodiments, the polypeptide that interacts with a protein, for example, an influenza virus spike protein, comprises an amino acid sequence having at least 10% sequence identity with SEQ ID NO:25 or a fragment or functional derivative thereof. In some embodiments, the polypeptide that interacts with a protein, for example, an influenza virus spike protein, comprises an amino acid sequence having at least 20% sequence identity with SEQ ID NO:25 or a fragment or functional derivative thereof. In some embodiments, the polypeptide that interacts with a protein, for example, an influenza virus spike protein, comprises an amino acid sequence having at least 30% sequence identity with SEQ ID NO:25 or a fragment or functional derivative thereof. In some embodiments, the polypeptide that interacts with a protein, for example, an influenza virus spike protein, comprises an amino acid sequence having at least 40% sequence identity with SEQ ID NO:25 or a fragment or functional derivative thereof. In some embodiments, the polypeptide that interacts with a protein, for example, an influenza virus spike protein, comprises an amino acid sequence having at least 50% sequence identity with SEQ ID NO:25 or a fragment or functional derivative thereof. In some embodiments, the polypeptide that interacts with a protein, for example, an influenza virus spike protein, comprises an amino acid sequence having at least 60% sequence identity with SEQ ID NO:25 or a fragment or functional derivative thereof. In some embodiments, the polypeptide that interacts with a protein, for example, an influenza virus spike protein, comprises an amino acid sequence having at least 70% sequence identity with SEQ ID NO:25 or a fragment or functional derivative thereof. In some embodiments, the polypeptide that interacts with a protein, for example, an influenza virus spike protein, comprises an amino acid sequence having at least 75% sequence identity with SEQ ID NO:25 or a fragment or functional derivative thereof. In some embodiments, the polypeptide that interacts with a protein, for example, an influenza virus spike protein, comprises an amino acid sequence having at least 80% sequence identity with SEQ ID NO:25 or a fragment or functional derivative thereof. In some embodiments, the polypeptide that interacts with a protein, for example, an influenza virus spike protein, comprises an amino acid sequence having at least 85% sequence identity with SEQ ID NO:25 or a fragment or functional derivative thereof. In some embodiments, the polypeptide that interacts with a protein, for example, an influenza virus spike protein, comprises an amino acid sequence having at least 90% sequence identity with SEQ ID NO:25 or a fragment or functional derivative thereof. In some embodiments, the polypeptide that interacts with a protein, for example, an influenza virus spike protein, comprises an amino acid sequence having at least 95% sequence identity with SEQ ID NO:25 or a fragment or functional derivative thereof. In some embodiments, the polypeptide that interacts with a protein, for example, an influenza virus spike protein, comprises, consists of, or consists essentially of SEQ ID NO:25 or a fragment or functional derivative thereof.

In some embodiments, the polypeptide that interacts with a protein, for example, an RSV glycoprotein, comprises an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:34, or a fragment or functional derivative thereof.

In some embodiments, the polypeptide that interacts with a protein, for example, an RSV glycoprotein, comprises an amino acid sequence having at least 10% sequence identity with SEQ ID NO:34 or a fragment or functional derivative thereof. In some embodiments, the polypeptide that interacts with a protein, for example, an RSV glycoprotein, comprises an amino acid sequence having at least 20% sequence identity with SEQ ID NO:34 or a fragment or functional derivative thereof. In some embodiments, the polypeptide that interacts with a protein, for example, an RSV glycoprotein, comprises an amino acid sequence having at least 30% sequence identity with SEQ ID NO:34 or a fragment or functional derivative thereof. In some embodiments, the polypeptide that interacts with a protein, for example, an RSV glycoprotein, comprises an amino acid sequence having at least 40% sequence identity with SEQ ID NO:34 or a fragment or functional derivative thereof. In some embodiments, the polypeptide that interacts with a protein, for example, an RSV glycoprotein, comprises an amino acid sequence having at least 50% sequence identity with SEQ ID NO:34 or a fragment or functional derivative thereof. In some embodiments, the polypeptide that interacts with a protein, for example, an RSV glycoprotein, comprises an amino acid sequence having at least 60% sequence identity with SEQ ID NO:34 or a fragment or functional derivative thereof. In some embodiments, the polypeptide that interacts with a protein, for example, an RSV glycoprotein, comprises an amino acid sequence having at least 70% sequence identity with SEQ ID NO:34 or a fragment or functional derivative thereof. In some embodiments, the polypeptide that interacts with a protein, for example, an RSV glycoprotein, comprises an amino acid sequence having at least 75% sequence identity with SEQ ID NO:34 or a fragment or functional derivative thereof. In some embodiments, the polypeptide that interacts with a protein, for example, an RSV glycoprotein, comprises an amino acid sequence having at least 80% sequence identity with SEQ ID NO:34 or a fragment or functional derivative thereof. In some embodiments, the polypeptide that interacts with a protein, for example, an RSV glycoprotein, comprises an amino acid sequence having at least 85% sequence identity with SEQ ID NO:34 or a fragment or functional derivative thereof. In some embodiments, the polypeptide that interacts with a protein, for example, an RSV glycoprotein, comprises an amino acid sequence having at least 90% sequence identity with SEQ ID NO:34 or a fragment or functional derivative thereof. In some embodiments, the polypeptide that interacts with a protein, for example, an RSV glycoprotein, comprises an amino acid sequence having at least 95% sequence identity with SEQ ID NO:34 or a fragment or functional derivative thereof. In some embodiments, the polypeptide that interacts with a protein, for example, an RSV glycoprotein, comprises, consists of, or consists essentially of SEQ ID NO:34 or a fragment or functional derivative thereof.

In some embodiments, the polypeptide that interacts with a protein, for example, a viral protein, further comprises a signal peptide sequence. A signal peptide (sometimes referred to as signal sequence, targeting signal, localization signal, localization sequence, transit peptide, leader sequence or leader peptide) is a short peptide (e.g., 16-30 amino acids long) present at the N-terminus of the newly synthesized secretable proteins. Signal peptides function to prompt a cell to translocate a protein, usually to the cellular membrane. In some embodiments, the signal peptide comprises an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NOS:28-32, or a fragment or functional derivative thereof.

In some embodiments, the signal peptide comprises an amino acid sequence having at least 70% sequence identity with SEQ ID NO:28 or a fragment or functional derivative thereof. In some embodiments, the signal peptide comprises an amino acid sequence having at least 75% sequence identity with SEQ ID NO:28 or a fragment or functional derivative thereof. In some embodiments, the signal peptide comprises an amino acid sequence having at least 80% sequence identity with SEQ ID NO:28 or a fragment or functional derivative thereof. In some embodiments, the signal peptide comprises an amino acid sequence having at least 85% sequence identity with SEQ ID NO:28 or a fragment or functional derivative thereof. In some embodiments, the signal peptide comprises an amino acid sequence having at least 90% sequence identity with SEQ ID NO:28 or a fragment or functional derivative thereof. In some embodiments, the signal peptide comprises an amino acid sequence having at least 95% sequence identity with SEQ ID NO:28 or a fragment or functional derivative thereof. In some embodiments, the signal peptide comprises an amino acid sequence having at least 99% sequence identity with SEQ ID NO:28 or a fragment or functional derivative thereof. In some embodiments, the signal peptide comprises SEQ ID NO:28 or a fragment or functional derivative thereof.

In some embodiments, the polypeptide that interacts with a protein, for example, a coronavirus spike protein, comprising an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NOS:2, 4, 6, 8, 10, 12, 14, or 16, or a fragment or functional derivative thereof, further comprises a signal peptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:28, or a fragment or functional derivative thereof.

In some embodiments, the signal peptide comprises an amino acid sequence having at least 70% sequence identity with SEQ ID NO:29 or a fragment or functional derivative thereof. In some embodiments, the signal peptide comprises an amino acid sequence having at least 75% sequence identity with SEQ ID NO:29 or a fragment or functional derivative thereof. In some embodiments, the signal peptide comprises an amino acid sequence having at least 80% sequence identity with SEQ ID NO:29 or a fragment or functional derivative thereof. In some embodiments, the signal peptide comprises an amino acid sequence having at least 85% sequence identity with SEQ ID NO:29 or a fragment or functional derivative thereof. In some embodiments, the signal peptide comprises an amino acid sequence having at least 90% sequence identity with SEQ ID NO:29 or a fragment or functional derivative thereof. In some embodiments, the signal peptide comprises an amino acid sequence having at least 95% sequence identity with SEQ ID NO:29 or a fragment or functional derivative thereof. In some embodiments, the signal peptide comprises an amino acid sequence having at least 99% sequence identity with SEQ ID NO:29 or a fragment or functional derivative thereof. In some embodiments, the signal peptide comprises SEQ ID NO:29 or a fragment or functional derivative thereof.

In some embodiments, the polypeptide that interacts with a protein, for example, an HIV spike protein, comprising an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:18, or a fragment or functional derivative thereof, further comprises a signal peptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:29, or a fragment or functional derivative thereof.

In some embodiments, the signal peptide comprises an amino acid sequence having at least 70% sequence identity with SEQ ID NO:30 or a fragment or functional derivative thereof. In some embodiments, the signal peptide comprises an amino acid sequence having at least 75% sequence identity with SEQ ID NO:30 or a fragment or functional derivative thereof. In some embodiments, the signal peptide comprises an amino acid sequence having at least 80% sequence identity with SEQ ID NO:30 or a fragment or functional derivative thereof. In some embodiments, the signal peptide comprises an amino acid sequence having at least 85% sequence identity with SEQ ID NO:30 or a fragment or functional derivative thereof. In some embodiments, the signal peptide comprises an amino acid sequence having at least 90% sequence identity with SEQ ID NO:30 or a fragment or functional derivative thereof. In some embodiments, the signal peptide comprises an amino acid sequence having at least 95% sequence identity with SEQ ID NO:30 or a fragment or functional derivative thereof. In some embodiments, the signal peptide comprises an amino acid sequence having at least 99% sequence identity with SEQ ID NO:30 or a fragment or functional derivative thereof. In some embodiments, the signal peptide comprises SEQ ID NO:30 or a fragment or functional derivative thereof.

In some embodiments, the polypeptide that interacts with a protein, for example, an Ebola glycoprotein, comprising an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:20, or a fragment or functional derivative thereof, further comprises a signal peptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:30, or a fragment or functional derivative thereof.

In some embodiments, the signal peptide comprises an amino acid sequence having at least 70% sequence identity with SEQ ID NO:31 or a fragment or functional derivative thereof. In some embodiments, the signal peptide comprises an amino acid sequence having at least 75% sequence identity with SEQ ID NO:31 or a fragment or functional derivative thereof. In some embodiments, the signal peptide comprises an amino acid sequence having at least 80% sequence identity with SEQ ID NO:31 or a fragment or functional derivative thereof. In some embodiments, the signal peptide comprises an amino acid sequence having at least 85% sequence identity with SEQ ID NO:31 or a fragment or functional derivative thereof. In some embodiments, the signal peptide comprises an amino acid sequence having at least 90% sequence identity with SEQ ID NO:31 or a fragment or functional derivative thereof. In some embodiments, the signal peptide comprises an amino acid sequence having at least 95% sequence identity with SEQ ID NO:31 or a fragment or functional derivative thereof. In some embodiments, the signal peptide comprises an amino acid sequence having at least 99% sequence identity with SEQ ID NO:31 or a fragment or functional derivative thereof. In some embodiments, the signal peptide comprises SEQ ID NO:31 or a fragment or functional derivative thereof.

In some embodiments, the polypeptide that interacts with a protein, for example, an influenza virus spike protein, comprising an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:25, or a fragment or functional derivative thereof, further comprises a signal peptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:31, or a fragment or functional derivative thereof.

In some embodiments, the signal peptide comprises an amino acid sequence having at least 70% sequence identity with SEQ ID NO:32 or a fragment or functional derivative thereof. In some embodiments, the signal peptide comprises an amino acid sequence having at least 75% sequence identity with SEQ ID NO:32 or a fragment or functional derivative thereof. In some embodiments, the signal peptide comprises an amino acid sequence having at least 80% sequence identity with SEQ ID NO:32 or a fragment or functional derivative thereof. In some embodiments, the signal peptide comprises an amino acid sequence having at least 85% sequence identity with SEQ ID NO:32 or a fragment or functional derivative thereof. In some embodiments, the signal peptide comprises an amino acid sequence having at least 90% sequence identity with SEQ ID NO:32 or a fragment or functional derivative thereof. In some embodiments, the signal peptide comprises an amino acid sequence having at least 95% sequence identity with SEQ ID NO:32 or a fragment or functional derivative thereof. In some embodiments, the signal peptide comprises an amino acid sequence having at least 99% sequence identity with SEQ ID NO:32 or a fragment or functional derivative thereof. In some embodiments, the signal peptide comprises SEQ ID NO:32 or a fragment or functional derivative thereof.

In some embodiments, the polypeptide that interacts with a protein, for example, an RSV glycoprotein, comprising an amino acid sequence having at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:34, or a fragment or functional derivative thereof, further comprises a signal peptide comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:32, or a fragment or functional derivative thereof.

### A. Polypeptides

As used herein, “polypeptide,” “peptide,” or “protein” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

As used herein, a “proteinaceous molecule,” “proteinaceous composition,” “proteinaceous compound,” “proteinaceous chain” or “proteinaceous material” generally refers, but is not limited to, a protein of greater than about 30 amino acids or the full length endogenous sequence translated from a gene; a polypeptide of greater than about 100 amino acids; and/or a peptide of from about 30 to about 3000 amino acids. All the “proteinaceous” terms described above may be used interchangeably herein.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments, the size of a protein or polypeptide (wild-type or modified) may comprise, but is not limited to, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500, 2750, 3000, 3250, 3500, 3750, 4000, 4250, 4500, 4750, or 5000 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). A protein may comprise one or more polypeptides.

As used herein, the term “domain” refers to any distinct functional or structural unit of a protein or polypeptide, and generally refers to a sequence of amino acids with a structure or function recognizable by one skilled in the art. For example, domain may refer to an oligomerization domain of a protein or polypeptide. Collagen triple helices, coiled coils and other oligomerization domains mediate the subunit assembly of a large number of proteins. Oligomerization leads to functional advantages of multivalency and high binding strength, increased structure stabilization and combined functions of different domains. Domains, for example, the oligomerization domains described herein, may be conserved. In evolutionary biology, conserved sequences are identical or similar sequences in nucleic acids (DNA and RNA) or proteins across species (orthologous sequences), or within a genome (paralogous sequences), or between donor and receptor taxa (xenologous sequences). Conservation indicates that a sequence has been maintained by natural selection. A highly conserved sequence is one that has remained relatively unchanged far back up the phylogenetic tree.

In certain embodiments the proteinaceous composition comprises at least one protein, polypeptide or peptide. It is contemplated that virtually any protein, polypeptide, or peptide containing component described herein may be used in the compositions and methods disclosed herein. In further embodiments the proteinaceous composition comprises a biocompatible protein, polypeptide, or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein-, polypeptide-, or peptide-containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides, or peptides through standard molecular biological techniques, the isolation of proteinaceous compounds from natural sources, or the chemical synthesis of proteinaceous materials.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide, or peptide.

Proteins and peptides suitable for use in this invention may be autologous proteins or peptides, although the invention is clearly not limited to the use of such autologous proteins. As used herein, the term “autologous protein, polypeptide, or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, a prokaryotic organism, a virus, or a bacteriophage, with a selected animal or human subject being preferred. The “autologous protein, polypeptide or peptide” may then be used as a component of a composition intended for application to the selected animal or human subject.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### B. Nucleic Acids

In certain embodiments, nucleic acid sequences can exist in a variety of instances such as: isolated segments and recombinant vectors of incorporated sequences or recombinant polynucleotides encoding the polypeptides described herein and/or derivatives, muteins, or variants thereof, polynucleotides sufficient for use as hybridization probes, PCR primers or sequencing primers for identifying, analyzing, mutating or amplifying a polynucleotide encoding a polypeptide, anti-sense nucleic acids for regulating expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having different nucleic acid sequences but, nonetheless, encode the same or substantially similar protein.

In certain embodiments, there are polynucleotide variants having substantial identity to the sequences disclosed herein, for example, those comprising at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence described herein, over the entire length of the sequence, or a nucleotide sequence complementary to said isolated polynucleotide, and in some cases, the nucleotide sequence encoding a polypeptide that has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to an amino acid sequence described herein results in a polypeptide having a similar structure to the structure of a polypeptide described herein.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, translocation, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

Changes can be introduced by mutation into a nucleic acid, thereby leading to changes in the amino acid sequence of a polypeptide that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes (see, e.g., Romain Studer et al., Biochem. J. 449:581-594 (2013)). For example, the mutation can quantitatively or qualitatively change the biological activity. Examples of quantitative changes include increasing, reducing or eliminating the activity. Examples of qualitative changes include altering the antigen specificity of an antibody.

### C. Variant Polypeptides

As modifications and/or changes may be made in the proteins and/or polynucleotides encoding the proteins according to the present invention, while still obtaining molecules having similar or improved characteristics, such biologically functional equivalents are also encompassed within the present invention.

The biological functional equivalent may comprise a polynucleotide that has been engineered to contain distinct sequences while at the same time retaining the capacity to encode the “wild-type” or standard protein or peptide or “variant” protein or peptide. This can be accomplished to the degeneracy of the genetic code, i.e., the presence of multiple codons, which encode for the same amino acids.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) having substitutions or mutations in selected amino acids (or codons) that retain the ability to interact with target proteins in vivo, for example.

In general, the shorter the length of the molecule, the fewer changes that can be made within the molecule while retaining function. Longer polypeptides may have an intermediate number of changes. The full-length protein will have the most tolerance for a larger number of changes. However, it must be appreciated that certain molecules or domains that are highly dependent upon their structure may tolerate little or no modification. In one example, a polynucleotide may be (and encode) a biological functional equivalent with more significant changes. Certain amino acids may be substituted for other amino acids in a protein structure without appreciable loss of interactive binding capacity with structures such as, for example, oligomerization domains, binding sites on substrate molecules, receptors, and such like.

The following is a discussion of changing the amino acids of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 10%, 20%, 30%, 40%, 50%, 60%, 70%, 80%, or 90%, including all values and ranges there between, identical to any sequence provided or referenced herein. A variant can include 1, 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild-type protein. Individual residues can be deleted or a number of contiguous amino acids can be deleted. A stop codon may be introduced (by substitution or insertion) into an encoding nucleic acid sequence to generate a truncated protein. For example, it is contemplated that peptides may be mutated by truncation, or deletion of a number of contiguous amino acids, rendering them shorter than their corresponding endogenous form.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein. For example, it is contemplated that peptides might be altered by fusing or conjugating a heterologous protein or polypeptide sequence with a particular function (e.g., for targeting or localization, for enhanced activity, for purification purposes, etc.).

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties. So-called “conservative” changes do not disrupt the biological activity of the protein, as the structural change is not one that impinges of the protein's ability to carry out its designed function. It is thus contemplated by the inventors that various changes may be made in the sequence of genes and proteins disclosed herein, while still fulfilling the goals of the present invention.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Amino acid sequence variants of the disclosure can be substitutional, insertional, or deletion variants, for example. It is contemplated that a region or fragment of a polypeptide of the disclosure may have an amino acid sequence that has, has at least, or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or more amino acid substitutions, contiguous amino acid additions, or contiguous amino acid deletions with respect to any of SEQ ID NOS:1-25, 27, 33, and 34. Alternatively, a region or fragment of a polypeptide of the disclosure may have an amino acid sequence that comprises or consists of an amino acid sequence that is, is at least, or is at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOS:1-25, 27, 33, and 34.

Moreover, in some embodiments, a region or fragment comprises an amino acid region of 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or more in any of SEQ ID NOS:1-25, 27, 33, and 34 (where position 1 is at the N-terminus of the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or be at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 350, 400, or more contiguous amino acids, or any range derivable therein, of any of SEQ ID NOS:1-25, 27, 33, and 34.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 substitutions of any of SEQ ID NOS:1-25, 27, 33, and 34 (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222,223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of any of SEQ ID NOS:1-25, 27, 33, and 34 (or any derivable position therein).

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the disclosure, those that are within ±1 are included, and in other aspects of the disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of a polypeptide with respect to its three-dimensional structure. One skilled in the art may choose not to make changes to amino acid residues predicted to be on the surface of the protein, since such residues may be involved in important interactions with other molecules. Moreover, one skilled in the art may generate and test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the protein that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native protein).

As used herein, an “amino molecule” refers to any amino acid, amino acid derivative, or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties. Peptides and polypeptides include the twenty “natural” amino acids, and post-translational modifications thereof. However, in vitro peptide synthesis permits the use of modified and/or unusual amino acids.

Accordingly, the term “proteinaceous composition” encompasses amino molecule sequences comprising at least one of the 20 common amino acids in naturally synthesized proteins, or at least one modified or unusual amino acid, including but not limited to those shown in the Table below.

Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino- | AHyl | allo-Hydroxylysine |
| | propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of R-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al. (1998)). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids in Weisshoff et al. (1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. These structures render the peptide or protein more thermally stable, also increase resistance to proteolytic degradation.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

### II. Exogenous Delivery of Protein-Interacting Polypeptides

In some aspects, there are nucleic acid molecules encoding the polypeptides of the disclosure. In certain embodiments, nucleic acid vectors could be constructed to comprise exogenous nucleic acid sequences to allow cells to express the proteinaceous compositions and polypeptides disclosed herein. Details of components of these vectors and delivery methods are disclosed below.

In various embodiments a DNA construct or vector encoding the polypeptide sequences disclosed herein are provided. Genetic modification may also be introduced to cells. These modifications include, for example, transduction of cells with a vector encoding the polypeptide for the generation of cells which express the polypeptide.

Cells according to the present disclosure include any cell into which the proteinaceous compositions and polypeptide sequences disclosed herein. DNA constructs or vectors constructed to comprise exogenous nucleic acid sequences to allow cells to express the proteinaceous compositions and polypeptide sequences disclosed herein can be introduced and expressed as described herein. It is to be understood that the basic concepts of the present disclosure described herein are not limited by cell type. Cells according to the present disclosure include prokaryotic cells, eukaryotic cells, mammalian cells, animal cells, human cells, and the like. Further, cells include any in which it would be beneficial or desirable to regulate production of a functional protein.

### A. Vectors

In some embodiments, the polypeptide sequences disclosed herein are achieved by operably linking a nucleic acid encoding the polypeptide sequences or portions thereof to a promoter, and incorporating the construct into an expression vector, which is taken up and expressed by cells. The vectors can be suitable for replication and, in some cases, integration in eukaryotes. Typical cloning vectors contain transcription and translation terminators, initiation sequences, and promoters useful for regulation of the expression of the desired nucleic acid sequence. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193). In some embodiments, a suitable vector is capable of crossing the blood-brain barrier.

In certain embodiments the expression vector may be provided to a cell in the form of a non-viral vector. For example, the non-viral vector may comprise a minicircle vector. Minicircles are a type of newly developed DNA carriers for gene therapy. The main advantages of minicircles include a cleaner gene background with minimal viral or bacterial gene elements and sustained high-level protein expression, and the small size of the minicircle may allow for its use in aerosols for drug delivery. The non-viral vector may comprise a pcDNA3.1(+) vector. The non-viral vector may comprise a nucleotide sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with SEQ ID NO:26.

In certain embodiments the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals.

A number of viral based systems have been developed for gene transfer into mammalian cells. Viruses that are useful as vectors include, but are not limited to, retroviruses, adenoviruses, adeno-associated viruses, herpes viruses, and lentiviruses (including self-inactivating lentivirus vectors). For example, adenoviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Thus, in some embodiments, the nucleic acid encoding the polypeptide sequences is introduced into cells using a recombinant vector such as a viral vector including, for example, a lentivirus, a retrovirus, gamma-retroviruses, an adeno-associated virus (AAV), a herpesvirus, or an adenovirus.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

Eukaryotic expression cassettes included in the vectors particularly contain (in a 5′-to-3′ direction) regulatory elements including a eukaryotic transcriptional promoter operably linked to a protein-coding sequence, splice signals including intervening sequences, a transcriptional termination/polyadenylation sequence, post-transcriptional regulatory elements, and origins of replication.

### 1. Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the thymidine kinase promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each specifically incorporated by reference herein in its entirety). Furthermore, it is contemplated the control sequences that direct transcription and/or expression of sequences within non-nuclear organelles such as mitochondria and the like can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, specifically incorporated by reference herein in its entirety). The promoters employed may be constitutive, cell-specific, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, through world wide web at epd.isb-sib.ch/) could also be used to drive expression. Non-limiting examples of other potential promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

In some embodiments, expression of the polynucleotide is regulated by a constitutive promoter. In some embodiments, the constitutive promoter is CAG (also known as CAGGS or CBA), EF-1ALPHA, ubiquitin, or CMV.

In some embodiments, expression of the polynucleotide is regulated by a cell-specific promoter. In some embodiments the cell-specific promoter is a neuron-specific promoter. In some embodiments, the neuron-specific promoter comprises a human synapsin I (SYN) promoter, a mouse calcium/calmodulin-dependent protein kinase II (CaMKII) promoter, a rat tubulin alpha I (Tal), rat neuron-specific enolase (NSE) promoter, a human platelet-derived growth factor-beta chain (PDGF) promoter, or THY1 (CD90) promoter. In some embodiments, the cell-specific promoter is human synapsin I.

In some embodiments, expression of the polynucleotide is regulated by a tissue-specific promoter. In some embodiments the tissue-specific promoter is a choroid plexus-specific promoter. In some embodiments, the choroid plexus-specific promoter comprises a Prlr promoter, a Spint2 promoter, or a F5 promoter. In some embodiments, the tissue-specific promoter is a liver-specific promoter. Liver-specific promoters have been described, for example, in L. M. Kattenhorn et al., Hum. Gene Ther. 27(12):947-961 (2016), specifically incorporated by reference herein in its entirety.

### 2. Protease Cleavage Sites/Self-Cleaving Peptides and Internal Ribosome Binding Sites

Suitable protease cleavages sites and self-cleaving peptides are known to the skilled person (see, e.g., in Ryan et al., 1997; Scymczak et al., 2004). Examples of protease cleavage sites are the cleavage sites of furin proteases, potyvirus NIa proteases (e.g., tobacco etch virus protease), potyvirus HC proteases, potyvirus P1 (P35) proteases, byovirus N1a proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used. In some embodiments, the protease cleavage sites are the cleavage sites of furin proteases.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thosea asigna virus, and porcine teschovirus.

A specific initiation signal also may be used for efficient translation of coding sequences in a polycistronic message. These signals include the ATG initiation codon or adjacent sequences. For example, an initiation signal may comprise a Kozak consensus sequence having an amino acid sequence comprising GCCACCAUGGG. See Kozak, 1987; Harte et al., 2012. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### 3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, specifically incorporated by reference herein in their entirety). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

### 5. Termination Signals

The vectors or constructs may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 6. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Exemplary embodiments include the SV40 polyadenylation signal or the bovine growth hormone polyadenylation signal, convenient and known to function well in various target cells. Polyadenylation may increase the stability of the transcript or may facilitate cytoplasmic translocation.

### 7. Post-Transcriptional Regulatory Elements

A vector for use in the disclosure can also comprise one or more post-transcriptional regulatory elements (PREs). Examples of PREs include the woodchuck hepatitis virus PRE (WPRE), hepatitis B virus PRE, and Intron A of human cytomegalovirus immediate early gene. See Sun et al. 2009 and Mariati et al. 2010 for further examples and details. In a particular embodiment, the PRE is a WPRE. WPRE is a DNA sequence that, when transcribed, creates a tertiary structure to enhance expression of genes delivered by viral vectors.

### 8. Origins of Replication

In order to propagate a vector in a host cell, the vector may contain one or more origins of replication sites (often termed “ori”), for example, a nucleic acid sequence corresponding to oriP of EBV as described above or a genetically engineered oriP with a similar or elevated function in differentiation programming, which is a specific nucleic acid sequence at which replication is initiated. Alternatively a replication origin of other extra-chromosomally replicating virus as described above or an autonomously replicating sequence (ARS) can be employed.

### B. Vector Delivery

Genetic modification or introduction of exogenous nucleic acids into cells may use any suitable methods for nucleic acid delivery for transformation of a cell, as described herein or as would be known to one of ordinary skill in the art. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Such methods include, but are not limited to, direct delivery of DNA such as by ex vivo transfection (Wilson et al., 1989, Nabel et al, 1989); transduction; viral transduction; injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by nucleofection; by lipofection or liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile or nanoparticle bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985); thermal shock (Froger and Hall, 2007); and any combination of such methods. Through the application of techniques such as these, organelle(s), cell(s), tissue(s) or organism(s) may be stably or transiently transformed.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al. (2001) Molecular Cloning: A Laboratory Manual, Cold Spring Harbor Laboratory, New York).

Biological methods for introducing a polynucleotide of interest into a host cell can include the use of DNA and RNA vectors into which the polynucleotide of interest, or transgene, can be inserted. Viral vectors have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. Also contemplated are nanoparticles. An illustrative colloidal system for use as a delivery vehicle in vitro and in vivo is a liposome (e.g., an artificial membrane vesicle).

Gene therapy methods and methods of delivering genes to subjects, for example using adeno-associated viruses, are described in U.S. Pat. No. 6,967,018, WO2014/093622, US2008/0175845, US 2014/0100265, EP2432490, EP2352823, EP2384200, WO2014/127198, WO2005/122723, WO2008/137490, WO2013/1421 14, WO2006/128190, WO2009/134681, EP2341068, WO2008/027084, WO2009/054994, WO2014059031, U.S. Pat. No. 7,977,049 and WO 2014/059029, each of which are specifically incorporated herein by reference in their entirety.

### 1. Liposome-Mediated Transfection

One illustrative delivery vehicle is a lipid and/or a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain embodiment, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The amount of liposomes used may vary upon the nature of the liposome as well as the cell used, for example, about 5 to about 20 μg vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

In various embodiments lipids suitable for use can be obtained from commercial sources. For example, lipofectamine can be obtained from Thermo Fisher Scientific, Waltham, Mass.; dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### 2. Electroporation

In certain embodiments, a nucleic acid is introduced into a cell via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### 3. Calcium Phosphate

In other embodiments, a nucleic acid is introduced to the cells using calcium phosphate precipitation. Human KB cells have been transfected with adenovirus 5 DNA (Graham and Van Der Eb, 1973) using this technique. Also in this manner, mouse L (A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

### C. Selectable or Screenable Markers

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In certain embodiments, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain embodiments of the present invention utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., 0-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum (see Table 1 for non-limiting examples). Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

TABLE
Fluorescent Protein Properties

| | | | | | | Relative |
|---|---|---|---|---|---|---|
| | Excitation | Emission | | | | Brightness |
| Protein | Maximum | Maximum | Molar Extinction | Quantum | in vivo | (% of |
| (Acronym) | (nm) | (nm) | Coefficient | Yield | Structure | EGFP) |
| [symbol] GFP (wt) | 395/475 | 509 | 21,000 | 0.77 | Monomer* | 48 |

Green Fluorescent Proteins

| | | | | | | |
|---|---|---|---|---|---|---|
| .EGFP | 484 | 507 | 56,000 | 0.60 | Monomer* | 100 |
| .AcGFP | 480 | 505 | 50,000 | 0.55 | Monomer* | 82 |
| [symbol] TurboGFP | 482 | 502 | 70,000 | 0.53 | Monomer* | 110 |
| [symbol] Emerald | 487 | 509 | 57,500 | 0.68 | Monomer* | 116 |
| [symbol] Azami Green | 492 | 505 | 55,000 | 0.74 | Monomer | 121 |
| [symbol] ZsGreen | 493 | 505 | 43,000 | 0.91 | Tetramer | 117 |

Blue Fluorescent Proteins

| | | | | | | |
|---|---|---|---|---|---|---|
| [symbol] EBFP | 383 | 445 | 29,000 | 0.31 | Monomer* | 27 |
| [symbol] Sapphire | 399 | 511 | 29,000 | 0.64 | Monomer* | 55 |
| .T-Sapphire | 399 | 511 | 44,000 | 0.60 | Monomer* | 79 |

Cyan Fluorescent Proteins

| | | | | | | |
|---|---|---|---|---|---|---|
| [symbol] ECFP | 439 | 476 | 32,500 | 0.40 | Monomer* | 39 |
| [symbol] mCFP | 433 | 475 | 32,500 | 0.40 | Monomer | 39 |
| [symbol] Cerulean | 433 | 475 | 43,000 | 0.62 | Monomer* | 79 |
| [symbol] CyPet | 435 | 477 | 35,000 | 0.51 | Monomer* | 53 |
| [symbol] AmCyan1 | 458 | 489 | 44,000 | 0.24 | Tetramer | 31 |
| .Midori-Ishi | 472 | 495 | 27,300 | 0.90 | Dimer | 73 |
| Cyan | | | | | | |
| .mTFP1 | 462 | 492 | 64,000 | 0.85 | Monomer | 162 |
| (Teal) | | | | | | |

Yellow Fluorescent Proteins

| | | | | | | |
|---|---|---|---|---|---|---|
| [symbol] EYFP | 514 | 527 | 83,400 | 0.61 | Monomer* | 151 |
| .Topaz | 514 | 527 | 94,500 | 0.60 | Monomer* | 169 |
| [symbol] Venus | 515 | 528 | 92,200 | 0.57 | Monomer* | 156 |
| .mCitrine | 516 | 529 | 77,000 | 0.76 | Monomer | 174 |
| [symbol] YPet | 517 | 530 | 104,000 | 0.77 | Monomer* | 238 |
| .PhiYFP | 525 | 537 | 124,000 | 0.39 | Monomer* | 144 |
| [symbol] ZsYellow1 | 529 | 539 | 20,200 | 0.42 | Tetramer | 25 |
| [symbol] mBanana | 540 | 553 | 6,000 | 0.7 | Monomer | 13 |
| [symbol] Kusabira | 548 | 559 | 51,600 | 0.60 | Monomer | 92 |
| Orange | | | | | | |
| .mOrange | 548 | 562 | 71,000 | 0.69 | Monomer | 146 |
| [symbol] dTomato | 554 | 581 | 69,000 | 0.69 | Dimer | 142 |
| .dTomato- | 554 | 581 | 138,000 | 0.69 | Monomer | 283 |
| Tandem | | | | | | |
| .DsRed | 558 | 583 | 75,000 | 0.79 | Tetramer | 176 |
| .DsRed2 | 563 | 582 | 43,800 | 0.55 | Tetramer | 72 |
| [symbol] DsRed- | 555 | 584 | 38,000 | 0.51 | Tetramer | 58 |
| Express (T1) | | | | | | |
| [symbol] DsRed- | 556 | 586 | 35,000 | 0.10 | Monomer | 10 |
| Monomer | | | | | | |
| .mTangerine | 568 | 585 | 38,000 | 0.30 | Monomer | 34 |
| [symbol] mStrawberry | 574 | 596 | 90,000 | 0.29 | Monomer | 78 |
| [symbol] AsRed2 | 576 | 592 | 56,200 | 0.05 | Tetramer | 8 |
| .mRFP1 | 584 | 607 | 50,000 | 0.25 | Monomer | 37 |
| [symbol] JRed | 584 | 610 | 44,000 | 0.20 | Dimer | 26 |
| [symbol] mCherry | 587 | 610 | 72,000 | 0.22 | Monomer | 47 |
| .HcRed1 | 588 | 618 | 20,000 | 0.015 | Dimer | 1 |
| [symbol] mRaspberry | 598 | 625 | 86,000 | 0.15 | Monomer | 38 |
| .HcRed- | 590 | 637 | 160,000 | 0.04 | Monomer | 19 |
| Tandem | | | | | | |
| .mPlum | 590 | 649 | 41,000 | 0.10 | Monomer | 12 |
| [symbol] AQ143 | 595 | 655 | 90,000 | 0.04 | Tetramer | 11 |

.Orange and Red Fluorescent Proteins
*Weak Dimer

### III. Viruses

Aspects of the present disclosure relate to treatment or prevention of a virus. In some embodiments, disclosed are methods for treatment or prevention of a viral infection. In some embodiments, disclosed are compositions comprising one or more anti-viral agents.

### A. Coronaviruses

In particular embodiments, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry.

Several coronaviruses utilize animals as their primary hosts and have also evolved to infect humans. There are four main sub-groupings of coronaviruses, known as alpha, beta, gamma, and delta, and seven coronaviruses that can infect people. The four most common coronaviruses utilize humans as their natural host and include: HCoV-229E (alpha coronavirus); HCoV-NL63 (alpha coronavirus); HCoV-OC43 (beta coronavirus); HCoV-HKU1 (beta coronavirus). Three other human coronaviruses are: MERS-CoV (the beta coronavirus that causes MERS); SARS-CoV (the beta coronavirus that causes SARS); and SARS-CoV-2 (the novel coronavirus that causes coronavirus disease 2019, or COVID-19).

Coronaviruses have characteristic club-shaped spikes that project from their surface, which in electron micrographs create an image reminiscent of the solar corona, from which their name derives. The average diameter of the virus particles is around 120 nm (0.12 m). The diameter of the envelope is ˜80 nm (0.08 μm) and the spikes are ˜20 nm (0.02 μm) long. Beneath the spiked exterior of the virus is a round core shrouded in a viral envelope. The core contains genetic material that the virus can inject into cells to infect them.

The viral envelope consists of a lipid bilayer where the membrane (M), envelope (E), and spike (S) structural proteins are anchored. Inside the envelope, there is the nucleocapsid of helical symmetry which is formed from multiple copies of the nucleocapsid (N) protein, which are bound to the positive-sense single-stranded RNA genome in a continuous beads-on-a-string type conformation. The genome size of coronaviruses ranges from approximately 26 to 32 kilobases. The genome organization for a coronavirus is 5′-leader-UTR-replicase/transcriptase-spike (S)-envelope (E)-membrane (M)-nucleocapsid (N)-3′ UTR-poly (A) tail. The open reading frames 1a and 1b, which occupy the first two-thirds of the genome, encode the replicase/transcriptase polyprotein. The replicase/transcriptase polyprotein self cleaves to form nonstructural proteins. The later reading frames encode the four major structural proteins: spike, envelope, membrane, and nucleocapsid. Interspersed between these reading frames are the reading frames for the accessory proteins. The number of accessory proteins and their function is unique depending on the specific coronavirus.

The lipid bilayer envelope, membrane proteins, and nucleocapsid protect the virus when it is outside the host cell. The spike proteins extend from within the core to the viral surface and allow the virus to recognize and bind specific cells in the body. When the spike engages a receptor on a host cell, a cascade is triggered, resulting in the merger of the virus with the cell which allows the virus to release its genetic material and overtake the cell's processes to produce new viruses.

Infection begins when the viral spike (S) glycoprotein attaches to its complementary host cell receptor. After attachment, a protease of the host cell cleaves and activates the receptor-attached spike protein. Depending on the host cell protease available, cleavage and activation allows the virus to enter the host cell by endocytosis or direct fusion of the viral envelop with the host membrane. On entry into the host cell, the virus particle is uncoated, and its genome enters the cell cytoplasm. The coronavirus RNA genome has a 5′ methylated cap and a 3′ polyadenylated tail, which allows the RNA to attach to the host cell's ribosome for translation. The host ribosome translates the initial overlapping open reading frame of the virus genome and forms a long polyprotein. The polyprotein has its own proteases which cleave the polyprotein into multiple nonstructural proteins.

Viral entry is followed by replication of the virus. A number of the nonstructural proteins coalesce to form a multi-protein replicase-transcriptase complex (RTC). The main replicase-transcriptase protein is the RNA-dependent RNA polymerase (RdRp). It is directly involved in the replication and transcription of RNA from an RNA strand. The other nonstructural proteins in the complex assist in the replication and transcription process. The exoribonuclease nonstructural protein, for instance, provides extra fidelity to replication by providing a proofreading function which the RNA-dependent RNA polymerase lacks. One of the main functions of the complex is to replicate the viral genome. RdRp directly mediates the synthesis of negative-sense genomic RNA from the positive-sense genomic RNA. This is followed by the replication of positive-sense genomic RNA from the negative-sense genomic RNA. The other important function of the complex is to transcribe the viral genome. RdRp directly mediates the synthesis of negative-sense subgenomic RNA molecules from the positive-sense genomic RNA. This is followed by the transcription of these negative-sense subgenomic RNA molecules to their corresponding positive-sense mRNAs.

The replicated positive-sense genomic RNA becomes the genome of the progeny viruses. The mRNAs are gene transcripts of the last third of the virus genome after the initial overlapping reading frame. These mRNAs are translated by the host's ribosomes into the structural proteins and a number of accessory proteins. RNA translation occurs inside the endoplasmic reticulum. The viral structural proteins S, E, and M move along the secretory pathway into the Golgi intermediate compartment. There, the M proteins direct most protein-protein interactions required for assembly of viruses following its binding to the nucleocapsid. Progeny viruses are then released from the host cell by exocytosis through secretory vesicles.

The interaction of the coronavirus spike protein with its complement host cell receptor is central in determining the tissue tropism, infectivity, and species range of the virus. Coronaviruses mainly target epithelial cell receptors. They can be transmitted by aerosol, fomite, or fecal-oral routes, for example. Human coronaviruses infect the epithelial cells of the respiratory tract, while animal coronaviruses generally infect the epithelial cells of the digestive tract. For example, coronaviruses such as SARS-CoV-2 can infect, via an aerosol route, human epithelial cells of the lungs by binding of the spike protein receptor binding domain (RBD) to an angiotensin-converting enzyme 2 (ACE2) receptor on the cell surface.

The WHO has reported that the two groups most at risk of experiencing severe illness due to a coronavirus infection and/or post-coronavirus infection syndrome are adults aged 65 years or older and people who have other underlying health conditions including chronic lung disease, serious heart conditions, severe obesity, a compromised immune system, or diabetes. In humans, coronaviruses typically cause a respiratory infection with mild to severe flu-like symptoms, but the exact symptoms vary depending on the type of coronavirus. The four common human coronaviruses can cause people to develop a runny nose, headache, cough, sore throat and fever. In a subset of subjects, including those with cardiopulmonary disease or a weakened immune system, the viral infection can progress to a more severe lower-respiratory infection such as pneumonia or bronchitis. In comparison, severe MERS and SARS infections often progress to pneumonia. Other symptoms of MERS include fever, coughing, and shortness of breath, while SARS can cause fever, chills and body aches.

Coronaviruses cause a variety of symptoms, triggering fever, cough, and shortness of breath in most patients. Rarer symptoms include dizziness, tiredness, aches, chills, sore throat, loss of smell, loss of taste, headache, nausea, vomiting, and diarrhea. Emergency signs or symptoms can include trouble breathing, persistent chest pain or pressure, new confusion, and/or blue lips or face. Complications of coronavirus infections can include pneumonia, organ failure, respiratory failure, blood clots, heart conditions such as cardiomyopathies, acute kidney injury, and/or further viral and bacterial infections.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and the species severe acute respiratory syndrome-related coronavirus; the subgenus Embecovirus and the species human coronavirus HKU1; and the species Betacoronavirus 1. In specific embodiments, the disclosure encompasses treatment or prevention of infection of any virus in the species of severe acute respiratory syndrome-related coronavirus, including the strains Middle East respiratory syndrome coronavirus (MERS-CoV), human coronavirus 229E (HCoV-229E), human coronavirus NL63 (HCoV-NL63), human coronavirus OC43 (HCoV-OC43), human coronavirus HKU1 (HCoV-HKU1), severe acute respiratory syndrome coronavirus (SARS-CoV), and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of HCoV-229E, HCoV-NL63, HCoV-OC43, HCoV-HKU1, Middle East respiratory syndrome coronavirus or severe acute respiratory syndrome-related coronavirus, including at least SARS-CoV and SARS-CoV-2. In specific embodiments, the virus has a genome length between 29000 to 30000, between 29100 and 29900, between 29200 and 29900, between 29300 and 29900, between 29400 and 29900, between 29500 and 29900, between 29600 and 29900, between 29700 and 29900, between 29800 and 29900, or between 29780 and 29900 base pairs in length.

Aspects of the disclosure relate to polypeptides that interact with a coronavirus spike (S) protein, including but not limited to, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, disclosed are polypeptides that interact with a MERS-CoV S protein. In some embodiments, disclosed are polypeptides that interact with an HCoV-229E S protein. In some embodiments, disclosed are polypeptides that interact with an HCoV-NL63 S protein. In some embodiments, disclosed are polypeptides that interact with an HCoV-OC43S protein. In some embodiments, disclosed are polypeptides that interact with an HCoV-HKU1 S protein. In some embodiments, disclosed are polypeptides that interact with a SARS-CoV S protein. In some embodiments, disclosed are polypeptides that interact with a SARS-CoV-2 S protein.

Viral membrane fusion proteins such as coronavirus spike proteins are oligomeric Class-I transmembrane glycoproteins on the viral envelope. The coronavirus spike proteins are cleaved to give rise to an N-terminal S1 region and a C-terminal S2 region. The S1 region contains the NTD and RBD domains responsible for attachment to the cell-surface receptor ACE2, and the S2 regions trimerize to form an elongated “stem” domain mainly for inducing fusion of viral envelope and host membrane through a large-scale conformational change. The fragment of the S2 region responsible for membrane fusion is highly conserved in sequence among coronaviruses. Thus, in some embodiments, an S2 fragment is derived from a MERS-CoV S protein. In some embodiments, an S2 fragment is derived from an HCoV-229E S protein. In some embodiments, an S2 fragment is derived from an HCoV-NL63 S protein. In some embodiments, an S2 fragment is derived from an HCoV-OC43S protein. In some embodiments, an S2 fragment is derived from an HCoV-HKU1 S protein. In some embodiments, an S2 fragment is derived from a SARS-CoV S protein. In some embodiments, an S2 fragment is derived from a SARS-CoV-2 S protein.

The sequence of SARS-CoV-2 S protein with the S2 region bolded and the S2 fragment bolded and underlined is provided as SEQ ID NO:1:

(SEQ ID NO: 1)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT.

In some embodiments, the polypeptides of the disclosure are derived from the S2 region of the SARS-CoV-2 S protein. In some embodiments, the polypeptide derived from the S2 region of the SARS-CoV-2 S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:1 corresponding to SEQ ID NO:2, or a fragment or functional derivative thereof.

In some embodiments, the polypeptides derived from the S2 region of the SARS-CoV-2 S protein, for example, the bolded and underlined portion of SEQ ID NO:1 corresponding to SEQ ID NO:2, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the SARS-CoV-2 S protein interact with a MERS-CoV S protein. In some embodiments, the polypeptides derived from the S2 region of the SARS-CoV-2 S protein interact with an HCoV-229E S protein. In some embodiments, the polypeptides derived from the S2 region of the SARS-CoV-2 S protein interact with an HCoV-NL63 S protein. In some embodiments, the polypeptides derived from the S2 region of the SARS-CoV-2 S protein interact with an HCoV-OC43 S protein. In some embodiments, the polypeptides derived from the S2 region of the SARS-CoV-2 S protein interact with an HCoV-HKU1 S protein. In some embodiments, the polypeptides derived from the S2 region of the SARS-CoV-2 S protein interact with a SARS-CoV S protein. In some embodiments, the polypeptides derived from the S2 region of the SARS-CoV-2 S protein interact with a SARS-CoV-2 S protein.

In some embodiments, the polypeptides derived from the S2 region of the SARS-CoV-2 S protein, for example, the bolded and underlined portion of SEQ ID NO:1 corresponding to SEQ ID NO:2, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the SARS-CoV-2 S protein oligomerize with a MERS-CoV S protein. In some embodiments, the polypeptides derived from the S2 region of the SARS-CoV-2 S protein oligomerize with an HCoV-229E S protein. In some embodiments, the polypeptides derived from the S2 region of the SARS-CoV-2 S protein oligomerize with an HCoV-NL63 S protein. In some embodiments, the polypeptides derived from the S2 region of the SARS-CoV-2 S protein oligomerize with an HCoV-OC43 S protein. In some embodiments, the polypeptides derived from the S2 region of the SARS-CoV-2 S protein oligomerize with an HCoV-HKU1 S protein. In some embodiments, the polypeptides derived from the S2 region of the SARS-CoV-2 S protein oligomerize with a SARS-CoV S protein. In some embodiments, the polypeptides derived from the S2 region of the SARS-CoV-2 S protein oligomerize with a SARS-CoV-2 S protein.

The sequence of the SARS-CoV S protein with the S2 region bolded and the S2 fragment bolded and underlined is provided as SEQ ID NO:3:

(SEQ ID NO: 3)
MPMGSLQPLATLYLLGMLVASVLASGSDLDRCTTFDDVQAPNYTQHTSS
MRGVYYPDEIFRSDTLYLTQDLFLPFYSNVTGFHTINHTFGNPVIPFKDG
IYFAATEKSNVVRGWVFGSTMNNKSQSVIIINNSTNVVIRACNFELCDNP
FFAVSKPMGTQTHTMIFDNAFNCTFEYISDAFSLDVSEKSGNFKHLREFV
FKNKDGFLYVYKGYQPIDVVRDLPSGFNTLKPIFKLPLGINITNFRAILT
AFSPAQDIWGTSAAAYFVGYLKPTTFMLKYDENGTITDAVDCSQNPLAEL
KCSVKSFEIDKGIYQTSNFRVVPSGDVVRFPNITNLCPFGEVFNATKFPS
YVAWERKKISNCVADYSVLYNSTFFSTFKCYGVSATKLNDLCFSNVYADS
FVVKGDDVRQIAPGQTGVIADYNYKLPDDFMGCVLAWNTRNIDATSTGNY
NYKYRYLRHGKLRPFERDISNVPFSPDGKPCTPPALNCYWPLNDYGFYTT
TGIGYQPYRVVVLSFELLNAPATVCGPKLSTDLIKNQCVNFNFNGLTGTG
VLTPSSKRFQPFQQFGRDVSDFTDSVRDPKTSEILDISPCSFGGVSVITP
GTNASSEVAVLYQDVNCTDVSTAIHADQLTPAWRIYSTGNNVFQTQAGCL
IGAEHVDTSYECDIPIGAGICASYHTVSLLR
STSQKSIVAYTMSLGADSSIAYSNNTIAIP
TNFSISITTEVMPVSMAKTSVDCNMYICGD
STECANLLLQYGSFCTQLNRALSGIAAEQD
RNTREVFAQVKQMYKTPTLKYFGGFNFSQI
LPDPLKPTKRSFIEDLLFNKVTLADAGFMK
QYGECLGDINARDLICAQKFNGLTVLPPLL
TDDMIAAYTAALVSGTATAGWTFGAGAALQ
IPFAMQMAYRFNGIGVTQNVLYENQKQIAN
QFNKAISQIQESLTTTSTALGKLQDVVNQN
AQALNTLVKQLSSNFGAISSVLNDILSRLD
KVEAEVQIDRLITGRLQSLQTYVTQQLIRA
AEIRASANLAATKMSECVLGQSKRVDFCGK
GYHLMSFPQAAPHGVVFLHVTYVPSQERNF
TTAPAICHEGKAYFPREGVFVFNGTSWFIT
QRNFFSPQIITTDNTFVSGNCDVVIGIINN
TVYDPLQPELDSFKEELDKYFKNHTSPDVD
LGDISGINASVVNIQKEIDRLNEVAKNLNE
SLIDLQELGKYEQYIKWPWYVWLGFIAGLI
AIVMVTILLCCMTSCCSCLKGACSCGSCCK
FDEDDSEPVLKGVKLHYT.

In some embodiments, the polypeptides of the disclosure are derived from the S2 region of the SARS-CoV S protein. In some embodiments, the polypeptide derived from the S2 region of the SARS-CoV S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:3 corresponding to SEQ ID NO:4, or a fragment or functional derivative thereof.

In some embodiments, the polypeptides derived from the S2 region of the SARS-CoV S protein, for example, the bolded and underlined portion of SEQ ID NO:3 corresponding to SEQ ID NO:4, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the SARS-CoV S protein interact with a MERS-CoV S protein. In some embodiments, the polypeptides derived from the S2 region of the SARS-CoV S protein interact with an HCoV-229E S protein. In some embodiments, the polypeptides derived from the S2 region of the SARS-CoV S protein interact with an HCoV-NL63 S protein. In some embodiments, the polypeptides derived from the S2 region of the SARS-CoV S protein interact with an HCoV-OC43 S protein. In some embodiments, the polypeptides derived from the S2 region of the SARS-CoV S protein interact with an HCoV-HKU1 S protein. In some embodiments, the polypeptides derived from the S2 region of the SARS-CoV S protein interact with a SARS-CoV S protein. In some embodiments, the polypeptides derived from the S2 region of the SARS-CoV S protein interact with a SARS-CoV-2 S protein.

In some embodiments, the polypeptides derived from the S2 region of the SARS-CoV S protein, for example, the bolded and underlined portion of SEQ ID NO:3 corresponding to SEQ ID NO:4, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the SARS-CoV S protein oligomerize with a MERS-CoV S protein. In some embodiments, the polypeptides derived from the S2 region of the SARS-CoV S protein oligomerize with an HCoV-229E S protein. In some embodiments, the polypeptides derived from the S2 region of the SARS-CoV S protein oligomerize with an HCoV-NL63 S protein. In some embodiments, the polypeptides derived from the S2 region of the SARS-CoV S protein oligomerize with an HCoV-OC43 S protein. In some embodiments, the polypeptides derived from the S2 region of the SARS-CoV S protein oligomerize with an HCoV-HKU1 S protein. In some embodiments, the polypeptides derived from the S2 region of the SARS-CoV S protein oligomerize with a SARS-CoV S protein. In some embodiments, the polypeptides derived from the S2 region of the SARS-CoV S protein oligomerize with a SARS-CoV-2 S protein.

The sequence of the SARS-CoV-2 B.1.1.7 variant S protein with the S2 region bolded and the S2 fragment bolded and underlined is provided as SEQ ID NO:5:

(SEQ ID NO: 5)
MFVFLVLLPLVSSQCVNLTTRTQLPPAYTNSFTRGVYYPDKVFRSSVLHS
TQDLFLPFFSNVTWFHAISGTNGTKRFDNPVLPFNDGVYFASTEKSNIIR
GWIFGTTLDSKTQSLLIVNNATNVVIKVCEFQFCNDPFLGVYHKNNKSWM
ESEFRVYSSANNCTFEYVSQPFLMDLEGKQGNFKNLREFVFKNIDGYFKI
YSKHTPINLVRDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGENCYFPLQSYGFQPTYGV
GYQPYRVVVLSFELLHAPATVCGPKKSTNLVKNKCVNFNFNGLTGTGVLT
ESNKKFLPFQQFGRDIDDTTDAVRDPQTLEILDITPCSFGGVSVITPGTN
TSNQVAVLYQGVNCTEVPVAIHADQLTPTWRVYSTGSNVFQTRAGCLIGA
EHVNNSYECDIPIGAGICASYQTQTNSHRRAR
SVASQSIIAYTMSLGAENSVAYSNNSIAIP
INFTISVTTEILPVSMTKTSVDCTMYICGD
STECSNLLLQYGSFCTQLNRALTGIAVEQD
KNTQEVFAQVKQIYKTPPIKDFGGFNFSQI
LPDPSKPSKRSFIEDLLENKVTLADAGFIK
QYGDCLGDIAARDLICAQKFNGLTVLPPLL
TDEMIAQYTSALLAGTITSGWTFGAGAALQ
IPFAMQMAYRFNGIGVTQNVLYENQKLIAN
QFNSAIGKIQDSLSSTASALGKLQDVVNQN
AQALNTLVKQLSSNFGAISSVLNDILARLD
KVEAEVQIDRLITGRLQSLQTYVTQQLIRA
AEIRASANLAATKMSECVLGQSKRVDFCGK
GYHLMSFPQSAPHGVVFLHVTYVPAQEKNF
TTAPAICHDGKAHFPREGVFVSNGTHWFVT
QRNFYEPQIITTHNTFVSGNCDVVIGIVNN
TVYDPLQPELDSFKEELDKYFKNHTSPDVD
LGDISGINASVVNIQKEIDRLNEVAKNLNE
SLIDLQELGKYEQYIKWPWYIWLGFIAGLI
AIVMVTIMLCCMTSCCSCLKGCCSCGSCCK
FDEDDSEPVLKGVKLHYT.

In some embodiments, the polypeptides of the disclosure are derived from the S2 region of the SARS-CoV-2 B.1.1.7 variant S protein. In some embodiments, the polypeptide derived from the S2 region of the SARS-CoV-2 B.1.1.7 variant S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:5 corresponding to SEQ ID NO:6, or a fragment or functional derivative thereof.

In some embodiments, the polypeptides derived from the S2 region of the SARS-CoV-2 B.1.1.7 variant S protein, for example, the bolded and underlined portion of SEQ ID NO:5 corresponding to SEQ ID NO:6, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the SARS-CoV-2 B.1.1.7 variant S protein interact with a MERS-CoV S protein. In some embodiments, the polypeptides derived from the S2 region of the SARS-CoV-2 B.1.1.7 variant S protein interact with an HCoV-229E S protein. In some embodiments, the polypeptides derived from the S2 region of the SARS-CoV-2 B.1.1.7 variant S protein interact with an HCoV-NL63 S protein. In some embodiments, the polypeptides derived from the S2 region of the SARS-CoV-2 B.1.1.7 variant S protein interact with an HCoV-OC43 S protein. In some embodiments, the polypeptides derived from the S2 region of the SARS-CoV-2 B.1.1.7 variant S protein interact with an HCoV-HKU1 S protein. In some embodiments, the polypeptides derived from the S2 region of the SARS-CoV-2 B.1.1.7 variant S protein interact with a SARS-CoV S protein. In some embodiments, the polypeptides derived from the S2 region of the SARS-CoV-2 B.1.1.7 variant S protein interact with a SARS-CoV-2 S protein.

In some embodiments, the polypeptides derived from the S2 region of the SARS-CoV-2 B.1.1.7 variant S protein, for example, the bolded and underlined portion of SEQ ID NO:5 corresponding to SEQ ID NO:6, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the SARS-CoV-2 B.1.1.7 variant S protein oligomerize with a MERS-CoV S protein. In some embodiments, the polypeptides derived from the S2 region of the SARS-CoV-2 B.1.1.7 variant S protein oligomerize with an HCoV-229E S protein. In some embodiments, the polypeptides derived from the S2 region of the SARS-CoV-2 B.1.1.7 variant S protein oligomerize with an HCoV-NL63 S protein. In some embodiments, the polypeptides derived from the S2 region of the SARS-CoV-2 B.1.1.7 variant S protein oligomerize with an HCoV-OC43 S protein. In some embodiments, the polypeptides derived from the S2 region of the SARS-CoV-2 B.1.1.7 variant S protein oligomerize with an HCoV-HKU1 S protein. In some embodiments, the polypeptides derived from the S2 region of the SARS-CoV-2 B.1.1.7 variant S protein oligomerize with a SARS-CoV S protein. In some embodiments, the polypeptides derived from the S2 region of the SARS-CoV-2 B.1.1.7 variant S protein oligomerize with a SARS-CoV-2 S protein.

The sequence of the MERS-CoV S protein with the S2 region bolded and the S2 fragment bolded and underlined is provided as SEQ ID NO:7:

(SEQ ID NO: 7)
MIHSVFLLMFLLTPTESYVDVGPDSAKSACIEVDIQQTFFDKTWPRPIDV
SKADGIIYPQGRTYSNITITYQGLFPYQGDHGDMYVYSAGHATGTTPQKL
FVANYSQDVKQFANGFVVRIGAAANSTGTVIISPSTSAIIRKIYPAFMLG
SSVGNFSYGKMGRFFNHTLVLLPDGCGTLLRAFYCILEPRSGNYCPAGNS
YTSFATYHTPATDCSDGNYNRNASLNSFKEYFNLRNCTFMYTYNITEDEI
LEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCS
YESFDVESGVYSVSSFEAKPSGSVVEQAEGVECDFSPLLFGTPPQVYNFK
RLVFTNCNYNLTKLLSLFSVNDFTCSQISPAAIASNCYSSLILDYFSYPL
SMKSDLSVSSAGPISQFNYKQSFSNPTCLILATVPHNLTTITKPLKYSYI
NKCSRLLSDDRTEVLQLVNANQYSPCVSIVPSTVWEDGDYYRKQLSPLEG
GGWLVASGSTVAMTEQLQMGFGITVQYGTDTNSVCPKLEFANDTKIVSQL
GNCVEYSLYGVSGRGVFQNCTAVGVPQQRFVYDAYQNLVGYYSDDGNYYC
LRACVSVPVSVIYDKETKTHATLFGSVACEHISSTMSQYSRSTRSMLKRR
DSTYGPLQTPVGCVLGLVNSSLFVEDCKLPLGQSLCALPDTPSTLTPRSV
RSVPGEMRLASIAFNHPIQVDQLNSSYFKL
SIPTNFSFGVTQEYIQTTIQKVTVDCKQYV
CNGFQKCEQLLREYGQFCSKINQALHGANL
RQDDSVRNLFESVKSSQSSPIIPGFGGDFN
LTLLEPVSISTGSRSARSAIEDLLFDKVTI
ADPGYMQGYDDCMQQGPASARDLICAQYVA
GYKVLPPLMDVNMEAAYTSSLLGSIAGVGW
TAGLSSFAAIPFAQSIFYRLNGVGITQQVL
SENQKLIANKFNQALGAMQTGFTTTNEAFQ
KVQDAVNNNAQALSKLASELSNTFGAISAS
IGDIIQRLDVLEQDAQIDRLINGRLTTLNA
FVAQQLVRSESAALSAQLAKDKVNECVKAQ
SKRSGFCGQGTHIVSFVVNAPNGLYFMHVG
YYPSNHIEVVSAYGLCDSANPTNCIAPVNG
YFIKTNNTRIVDEWSYTGSSFYAPEPITSL
NTKYVAPQVTYQNISTNLPPPLLGNSTGID
FQDELDEFFKNVSTSIPNFGSLTQINTTLL
DLTYEMLSLQQVVKALNESYIDLKELGNYT
YYNKWPWYIWLSFIAGLVALALCVFFILCC
TGCGTNCMGKLKCNRCCDRYEEYDLEPHKV
HVH.

In some embodiments, the polypeptides of the disclosure are derived from the S2 region of the MERS-CoV S protein. In some embodiments, the polypeptide derived from the S2 region of the MERS-CoV S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:7 corresponding to SEQ ID NO:8, or a fragment or functional derivative thereof.

In some embodiments, the polypeptides derived from the S2 region of the MERS-CoV S protein, for example, the bolded and underlined portion of SEQ ID NO:7 corresponding to SEQ ID NO:8, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the MERS-CoV S protein interact with a MERS-CoV S protein. In some embodiments, the polypeptides derived from the S2 region of the MERS-CoV S protein interact with an HCoV-229E S protein. In some embodiments, the polypeptides derived from the S2 region of the MERS-CoV S protein interact with an HCoV-NL63 S protein. In some embodiments, the polypeptides derived from the S2 region of the MERS-CoV S protein interact with an HCoV-OC43 S protein. In some embodiments, the polypeptides derived from the S2 region of the MERS-CoV S protein interact with an HCoV-HKU1 S protein. In some embodiments, the polypeptides derived from the S2 region of the MERS-CoV S protein interact with a SARS-CoV S protein. In some embodiments, the polypeptides derived from the S2 region of the MERS-CoV S protein interact with a SARS-CoV-2 S protein.

In some embodiments, the polypeptides derived from the S2 region of the MERS-CoV S protein, for example, the bolded and underlined portion of SEQ ID NO:7 corresponding to SEQ ID NO:8, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the MERS-CoV S protein oligomerize with a MERS-CoV S protein. In some embodiments, the polypeptides derived from the S2 region of the MERS-CoV S protein oligomerize with an HCoV-229E S protein. In some embodiments, the polypeptides derived from the S2 region of the MERS-CoV S protein oligomerize with an HCoV-NL63 S protein. In some embodiments, the polypeptides derived from the S2 region of the MERS-CoV S protein oligomerize with an HCoV-OC43 S protein. In some embodiments, the polypeptides derived from the S2 region of the MERS-CoV S protein oligomerize with an HCoV-HKU1 S protein. In some embodiments, the polypeptides derived from the S2 region of the MERS-CoV S protein oligomerize with a SARS-CoV S protein. In some embodiments, the polypeptides derived from the S2 region of the MERS-CoV S protein oligomerize with a SARS-CoV-2 S protein.

The sequence of the HCoV-229E S protein with the S2 region bolded and the S2 fragment bolded and underlined is provided as SEQ ID NO:9:

(SEQ ID NO: 9)
MFVLLVAYALLHIAGCQTTNGLNTSYSVCNGCVGYSENVFAVESGGYIPS
DFAFNNWFLLTNTSSVVDGVVRSFQPLLLNCLWSVSGLRFTTGFVYFNGT
GRGDCKGFSSDVLSDVIRYNLNFEENLRRGTILFKTSYGVVVFYCTNNTL
VSGDAHIPFGTVLGNFYCFVNTTIGNETTSAFVGALPKTVREFVISRTGH
FYINGYRYFTLGNVEAVNFNVTTAETTDFCTVALASYADVLVNVSQTSIA
NIIYCNSVINRLRCDQLSFDVPDGFYSTSPIQSVELPVSIVSLPVYHKHT
FIVLYVDFKPQSGGGKCFNCYPAGVNITLANFNETKGPLCVDTSHFTTKY
VAVYANVGRWSASINTGNCPFSFGKVNNFVKFGSVCFSLKDIPGGCAMPI
VANWAYSKYYTIGSLYVSWSDGDGITGVPQPVEGVSSFMNVTLDKCTKYN
IYDVSGVGVIRVSNDTFLNGITYTSTSGNLLGFKDVTKGTIYSITPCNPP
DQLVVYQQAVVGAMLSENFTSYGFSNVVELPKFFYASNGTYNCTDAVLTY
SSFGVCADGSIIAVQPRNVSYDSVSAIVTA
NLSIPSNWTTSVQVEYLQITSTPIVVDCST
YVCNGNVRCVELLKQYTSACKTIEDALRNS
ARLESADVSEMLTFDKKAFTLANVSSFGDY
NLSSVIPSLPTSGSRVAGRSAIEDILFSKL
VTSGLGTVDADYKKCTKGLSIADLACAQYY
NGIMVLPGVADAERMAMYTGSLIGGIALGG
LTSAVSIPFSLAIQARLNYVALQTDVLQEN
QKILAASENKAMTNIVDAFTGVNDAITQTS
QALQTVATALNKIQDVVNQQGNSLNHLTSQ
LRQNFQAISSSIQAIYDRLDTIQADQQVDR
LITGRLAALNVFVSHTLTKYTEVRASRQLA
QQKVNECVKSQSKRYGFCGNGTHIFSIVNA
APEGLVFLHTVLLPTQYKDVEAWSGLCVDG
TNGYVLRQPNLALYKEGNYYRITSRIMFEP
RIPTMADFVQIENCNVTFVNISRSELQTIV
PEYIDVNKTLQELSYKLPNYTVPDLVVEQY
NQTILNLTSEISTLENKSAELNYTVQKLQT
LIDNINSTLVDLKWLNRVETYIKWPWWVWL
CISVVLIFVVSMLLLCCCSTGCCGFFSCFA
SSIRGCCESTKLPYYDVEKIHIQ.

In some embodiments, the polypeptides of the disclosure are derived from the S2 region of the HCoV-229E S protein. In some embodiments, the polypeptide derived from the S2 region of the HCoV-229E S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:9 corresponding to SEQ ID NO:10, or a fragment or functional derivative thereof.

In some embodiments, the polypeptides derived from the S2 region of the HCoV-229E S protein, for example, the bolded and underlined portion of SEQ ID NO:9 corresponding to SEQ ID NO:10, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the HCoV-229E S protein interact with a MERS-CoV S protein. In some embodiments, the polypeptides derived from the S2 region of the HCoV-229E S protein interact with an HCoV-229E S protein. In some embodiments, the polypeptides derived from the S2 region of the HCoV-229E S protein interact with an HCoV-NL63 S protein. In some embodiments, the polypeptides derived from the S2 region of the HCoV-229E S protein interact with an HCoV-OC43 S protein. In some embodiments, the polypeptides derived from the S2 region of the HCoV-229E S protein interact with an HCoV-HKU1 S protein. In some embodiments, the polypeptides derived from the S2 region of the HCoV-229E S protein interact with a SARS-CoV S protein. In some embodiments, the polypeptides derived from the S2 region of the HCoV-229E S protein interact with a SARS-CoV-2 S protein.

In some embodiments, the polypeptides derived from the S2 region of the HCoV-229E S protein, for example, the bolded and underlined portion of SEQ ID NO:9 corresponding to SEQ ID NO:10, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the HCoV-229E S protein oligomerize with a MERS-CoV S protein. In some embodiments, the polypeptides derived from the S2 region of the HCoV-229E S protein oligomerize with an HCoV-229E S protein. In some embodiments, the polypeptides derived from the S2 region of the HCoV-229E S protein oligomerize with an HCoV-NL63 S protein. In some embodiments, the polypeptides derived from the S2 region of the HCoV-229E S protein oligomerize with an HCoV-OC43 S protein. In some embodiments, the polypeptides derived from the S2 region of the HCoV-229E S protein oligomerize with an HCoV-HKU1 S protein. In some embodiments, the polypeptides derived from the S2 region of the HCoV-229E S protein oligomerize with a SARS-CoV S protein. In some embodiments, the polypeptides derived from the S2 region of the HCoV-229E S protein oligomerize with a SARS-CoV-2 S protein.

The sequence of the HCoV-NL63 S protein with the S2 region bolded and the S2 fragment bolded and underlined is provided as SEQ ID NO:11:

(SEQ ID NO: 11)
MKLFLILLVLPLASCFFTCNSNANLSMLQLGVPDNSSTIVTGLLPTHWFC
ANQSTSVYSANGFFYIDVGNHRSAFALHTGYYDANQYYIYVTNEIGLNAS
VTLKICKFSRNTTFDFLSNASSSFDCIVNLLFTEQLGAPLGITISGETVR
LHLYNVTRTFYVPAAYKLTKLSVKCYFNYSCVFSVVNATVTVNVTTHNGR
VVNYTVCDDCNGYTDNIFSVQQDGRIPNGFPFNNWFLLTNGSTLVDGVSR
LYQPLRLTCLWPVPGLKSSTGFVYFNATGSDVNCNGYQHNSVVDVMRYNL
NFSANSLDNLKSGVIVFKTLQYDVLFYCSNSSSGVLDTTIPFGPSSQPYY
CFINSTINTTHVSTFVGILPPTVREIVVARTGQFYINGFKYFDLGFIEAV
NFNVTTASATDFWTVAFATFVDVLVNVSATNIQNLLYCDSPFEKLQCEHL
QFGLQDGFYSANFLDDNVLPETYVALPIYYQHTDINFTATASFGGSCYVC
KPHQVNISLNGNTSVCVRTSHFSIRYIYNRVKSGSPGDSSWHIYLKSGTC
PFSFSKLNNFQKFKTICFSTVEVPGSCNFPLEATWHYTSYTIVGALYVTW
SEGNSITGVPYPVSGIREFSNLVLNNCTKYNIYDYVGTGIIRSSNQSLAG
GITYVSNSGNLLGFKNVSTGNIFIVTPCNQPDQVAVYQQSIIGAMTAVNE
SRYGLQNLLQLPNFYYVSNGGNNCTTAVMTYSNFGICADGSLIPVRPR
NSSDNGISAIITANLSIPSNWTTSVQVEYL
QITSTPIVVDCATYVCNGNPRCKNLLKQYT
SACKTIEDALRLSAHLETNDVSSMLTFDSN
AFSLANVTSFGDYNLSSVLPQRNIRSSRIA
GRSALEDLLFSKVVTSGLGTVDVDYKSCTK
GLSIADLACAQYYNGIMVLPGVADAERMAM
YTGSLIGGMVLGGLTSAAAIPFSLALQARL
NYVALQTDVLQENQKILAASFNKAINNIVA
SFSSVNDAITQTAEAIHTVTIALNKIQDVV
NQQGSALNHLTSQLRHNFQAISNSIQAIYD
RLDSIQADQQVDRLITGRLAALNAFVSQVL
NKYTEVRGSRRLAQQKINECVKSQSNRYGF
CGNGTHIFSIVNSAPDGLLFLHTVLLPTDY
KNVKAWSGICVDGIYGYVLRQPNLVLYSDN
GVFRVTSRVMFQPRLPVLSDFVQIYNCNVT
FVNISRVELHTVIPDYVDVNKTLQEFAQNL
PKYVKPNFDLTPFNLTYLNLSSELKQLEAK
TASLFQTTVELQGLIDQINSTYVDLKLLNR
FENYIKWPWWVWLIISVVFVVLLSLLVFKC
CLSTGCCGCCNCLTSSMRGCCDCGSTLPYY
EFEKVHVQ.

In some embodiments, the polypeptides of the disclosure are derived from the S2 region of the HCoV-NL63 S protein. In some embodiments, the polypeptide derived from the S2 region of the HCoV-NL63 S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:11 corresponding to SEQ ID NO:12, or a fragment or functional derivative thereof.

In some embodiments, the polypeptides derived from the S2 region of the HCoV-NL63 S protein, for example, the bolded and underlined portion of SEQ ID NO:11 corresponding to SEQ ID NO:12, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the HCoV-NL63 S protein interact with a MERS-CoV S protein. In some embodiments, the polypeptides derived from the S2 region of the HCoV-NL63 S protein interact with an HCoV-229E S protein. In some embodiments, the polypeptides derived from the S2 region of the HCoV-NL63 S protein interact with an HCoV-NL63 S protein. In some embodiments, the polypeptides derived from the S2 region of the HCoV-NL63 S protein interact with an HCoV-OC43 S protein. In some embodiments, the polypeptides derived from the S2 region of the HCoV-NL63 S protein interact with an HCoV-HKU1 S protein. In some embodiments, the polypeptides derived from the S2 region of the HCoV-NL63 S protein interact with a SARS-CoV S protein. In some embodiments, the polypeptides derived from the S2 region of the HCoV-NL63 S protein interact with a SARS-CoV-2 S protein.

In some embodiments, the polypeptides derived from the S2 region of the HCoV-NL63 S protein, for example, the bolded and underlined portion of SEQ ID NO:11 corresponding to SEQ ID NO:12, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the HCoV-NL63 S protein oligomerize with a MERS-CoV S protein. In some embodiments, the polypeptides derived from the S2 region of the HCoV-NL63 S protein oligomerize with an HCoV-229E S protein. In some embodiments, the polypeptides derived from the S2 region of the HCoV-NL63 S protein oligomerize with an HCoV-NL63 S protein. In some embodiments, the polypeptides derived from the S2 region of the HCoV-NL63 S protein oligomerize with an HCoV-OC43 S protein. In some embodiments, the polypeptides derived from the S2 region of the HCoV-NL63 S protein oligomerize with an HCoV-HKU1 S protein. In some embodiments, the polypeptides derived from the S2 region of the HCoV-NL63 S protein oligomerize with a SARS-CoV S protein. In some embodiments, the polypeptides derived from the S2 region of the HCoV-NL63 S protein oligomerize with a SARS-CoV-2 S protein.

The sequence of the HCoV-HKU1 S protein with the S2 region bolded and the S2 fragment bolded and underlined is provided as SEQ ID NO:13:

(SEQ ID NO: 13)
MFLIIFILPTTLAVIGDFNCTNSFINDYNKTIPRISEDVVDVSLGLGTYY
VLNRVYLNTTLLFTGYFPKSGANFRDLALKGSIYLSTLWYKPPFLSDFNN
GIFSKVKNTKLYVNNTLYSEFSTIVIGSVFVNTSYTIVVQPHNGILEITA
CQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTD
NETLEYWVTPLSRRQYLLNFDEHGVITNAVDCSSSFLSEIQCKTQSFAPN
TGVYDLSGFTVKPVATVYRRIPNLPDCDIDNWLNNVSVPSPLNWERRIFS
NCNFNLSTLLRLVHVDSFSCNNLDKSKIFGSCFNSITVDKFAIPNRRRDD
LQLGSSGFLQSSNYKIDISSSSCQLYYSLPLVNVTINNFNPSSWNRRYGF
GSFNLSSYDVVYSDHCFSVNSDFCPCADPSVVNSCAKSKPPSAICPAGTK
YRHCDLDTTLYVKNWCRCSCLPDPISTYSPNTCPQKKVVVGIGEHCPGLG
INEEKCGTQLNHSSCFCSPDAFLGWSFDSCISNNRCNIFSNFIFNGINSG
TTCSNDLLYSNTEISTGVCVNYDLYGITGQGIFKEVSAAYYNNWQNLLYD
SNGNIIGFKDFLINKTYTILPCYSGRVSAAFYQNSSSPALLYRNLKCSYV
LNNISFISQPFYFDSYLGCVLNAVNLTSYSVSSCDLRMGSGFCIDYALPS
SRRKRRGISSPYRFVTFEPFNVSFVNDSVE
TVGGLFEIQIPTNFTIAGHEEFIQTSSPKV
TIDCSAFVCSNYAACHDLLSEYGTFCDNIN
SILNEVNDLLDITQLQVANALMQGVTLSSN
LNTNLHSDVDNIDFKSLLGCLGSQCGSSSR
SLLEDLLFNKVKLSDVGFVEAYNNCTGGSE
IRDLLCVQSFNGIKVLPPILSETQISGYTT
AATVAAMFPPWSAAAGVPFSLNVQYRINGL
GVTMDVLNKNQKLIANAFNKALLSIQNGFT
ATNSALAKIQSVVNANAQALNSLLQQLFNK
FGAISSSLQEILSRLDNLEAQVQIDRLING
RLALNAYVSQQLSDITLIKAGASRAIEKVN
TECVKSQSPRINFCGNGNHILSLVQNAPYG
LLFIHFSYKPTSFKTVLVSPGLCLSGDRGI
APKQGYFIKQNDSWMFTGSSYYYPEPISDK
NVVFMNSCSVNFTKAPFIYLNNSIPNLSDF
EAELSLWFKNHTSIAPNLTFNSHINATFLD
LYYEMNVIQESIKSLNSSFINLKEIGTYEM
YVKWPWYIWLLIVILFIIFLMILFFICCCT
GCGSACFSKCHNCCDEYGGHNDFVIKASHD
D.

In some embodiments, the polypeptides of the disclosure are derived from the S2 region of the HCoV-HKU1 S protein. In some embodiments, the polypeptide derived from the S2 region of the HCoV-HKU1 S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:13 corresponding to SEQ ID NO:14, or a fragment or functional derivative thereof.

In some embodiments, the polypeptides derived from the S2 region of the HCoV-HKU1 S protein, for example, the bolded and underlined portion of SEQ ID NO:13 corresponding to SEQ ID NO:14, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the HCoV-HKU1 S protein interact with a MERS-CoV S protein. In some embodiments, the polypeptides derived from the S2 region of the HCoV-HKU1 S protein interact with an HCoV-229E S protein. In some embodiments, the polypeptides derived from the S2 region of the HCoV-HKU1 S protein interact with an HCoV-NL63 S protein. In some embodiments, the polypeptides derived from the S2 region of the HCoV-HKU1 S protein interact with an HCoV-OC43 S protein. In some embodiments, the polypeptides derived from the S2 region of the HCoV-HKU1 S protein interact with an HCoV-HKU1 S protein. In some embodiments, the polypeptides derived from the S2 region of the HCoV-HKU1 S protein interact with a SARS-CoV S protein. In some embodiments, the polypeptides derived from the S2 region of the HCoV-HKU1 S protein interact with a SARS-CoV-2 S protein.

In some embodiments, the polypeptides derived from the S2 region of the HCoV-HKU1 S protein, for example, the bolded and underlined portion of SEQ ID NO:13 corresponding to SEQ ID NO:14, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the HCoV-HKU1 S protein oligomerize with a MERS-CoV S protein. In some embodiments, the polypeptides derived from the S2 region of the HCoV-HKU1 S protein oligomerize with an HCoV-229E S protein. In some embodiments, the polypeptides derived from the S2 region of the HCoV-HKU1 S protein oligomerize with an HCoV-NL63 S protein. In some embodiments, the polypeptides derived from the S2 region of the HCoV-HKU1 S protein oligomerize with an HCoV-OC43 S protein. In some embodiments, the polypeptides derived from the S2 region of the HCoV-HKU1 S protein oligomerize with an HCoV-HKU1 S protein. In some embodiments, the polypeptides derived from the S2 region of the HCoV-HKU1 S protein oligomerize with a SARS-CoV S protein. In some embodiments, the polypeptides derived from the S2 region of the HCoV-HKU1 S protein oligomerize with a SARS-CoV-2 S protein.

The sequence of the HCoV-OC43 S protein with the S2 region bolded and the S2 fragment bolded and underlined is provided as SEQ ID NO: 15:

(SEQ ID NO: 15)
MFLILLISLPTAFAVIGDLKCTSDNINDKDTGPPPISTDTVDVTNGLGTY
YVLDRVYLNTTLFLNGYYPTSGSTYRNMALKGSVLLSRLWFKPPFLSDFI
NGIFAKVKNTKVIKDRVMYSEFPAITIGSTFVNTSYSVVVQPRTINSTQD
GDNKLQGLLEVSVCQYNMCEYPQTICHPNLGNHRKELWHLDTGVVSCLYK
RNFTYDVNADYLYFHFYQEGGTFYAYFTDTGVVTKFLFNVYLGMALSHYY
VMPLTCNSKLTLEYWVTPLTSRQYLLAFNQDGIIFNAEDCMSDFMSEIKC
KTQSIAPPTGVYELNGYTVQPIADVYRRKPNLPNCNIEAWLNDKSVPSPL
NWERKTFSNCNFNMSSLMSFIQADSFTCNNIDAAKIYGMCFSSITIDKFA
IPNGRKVDLQLGNLGYLQSFNYRIDTTATSCQLYYNLPAANVSVSRFNPS
TWNKRFGFIEDSVFKPRPAGVLTNHDVVYAQHCFKAPKNFCPCKLNGSCV
GSGPGKNNGIGTCPAGTNYLTCDNLCTPDPITFTGTYKCPQTKSLVGIGE
HCSGLAVKSDYCGGNSCTCRPQAFLGWSADSCLQGDKCNIFANFILHDVN
SGLTCSTDLQKANTDIILGVCVNYDLYGILGQGIFVEVNATYYNSWQNLL
YDSNGNLYGFRDYIINRTFMIRSCYSGRVSAAFHANSSEPALLFRNIKCN
YVFNNSLTRQLQPINYFDSYLGCVVNAYNSTAISVQTCDLTVGSGYCVDY
SKNRRSRGAITTGYRFTNFEPFTVNSVNDS
LEPVGGLYEIQIPSEFTIGNMVEFIQTSSP
KVTIDCAAFVCGDYAACKSQLVEYGSFCDN
INAILTEVNELLDTTQLQVANSLMNGVTLS
TKLKDGVNFNVDDINFSPVLGCLGSECSKA
SSRSAIEDLLFDKVKLSDVGFVEAYNNCTG
GAEIRDLICVQSYKGIKVLPPLLSENQISG
YTLAATSASLFPPWTAAAGVPFYLNVQYRI
NGLGVTMDVLSQNQKLIANAFNNALYAIQE
GFDATNSALVKIQAVVNANAEALNNLLQQL
SNRFGAISASLQEILSRLDALEAEAQIDRL
INGRLTALNAYVSQQLSDSTLVKFSAAQAM
EKVNECVKSQSSRINFCGNGNHIISLVQNA
PYGLYFIHFSYVPTKYVTARVSPGLCIAGD
RGIAPKSGYFVNVNNTWMYTGSGYYYPEPI
TENNVVVMSTCAVNYTKAPYVMLNTSIPNL
PDFKEELDQWFKNQTSVAPDLSLDYINVTF
LDLQVEMNRLQEAIKVLNQSYINLKDIGTY
EYYVKWPWYVWLLICLAGVAMLVLLFFICC
CTGCGTSCFKKCGGCCDDYTGYQELVIKTS
HDD.

In some embodiments, the polypeptides of the disclosure are derived from the S2 region of the HCoV-OC43 S protein. In some embodiments, the polypeptide derived from the S2 region of the HCoV-OC43 S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the underlined portion of SEQ ID NO:15 corresponding to SEQ ID NO:16, or a fragment or functional derivative thereof.

In some embodiments, the polypeptides derived from the S2 region of the HCoV-OC43 S protein, for example, the bolded and underlined portion of SEQ ID NO:15 corresponding to SEQ ID NO:16, interact with a coronavirus spike (S) protein, including but not limited to a MERS-CoV S protein, a HCoV-229E S protein, a HCoV-NL63 S protein, HCoV-OC43, a HCoV-HKU1 S protein, a SARS-CoV S protein, and/or a SARS-CoV-2 S protein. In some embodiments, the polypeptides derived from the S2 region of the HCoV-OC43 S protein interact with a MERS-CoV S protein. In some embodiments, the polypeptides derived from the S2 region of the HCoV-OC43 S protein interact with an HCoV-229E S protein. In some embodiments, the polypeptides derived from the S2 region of the HCoV-OC43 S protein interact with an HCoV-NL63 S protein. In some embodiments, the polypeptides derived from the S2 region of the HCoV-OC43 S protein interact with an HCoV-OC43 S protein. In some embodiments, the polypeptides derived from the S2 region of the HCoV-OC43 S protein interact with an HCoV-HKU1 S protein. In some embodiments, the polypeptides derived from the S2 region of the HCoV-OC43 S protein interact with a SARS-CoV S protein. In some embodiments, the polypeptides derived from the S2 region of the HCoV-OC43 S protein interact with a SARS-CoV-2 S protein.

In some embodiments, the polypeptides derived from the S2 region of the HCoV-OC43 S protein, for example, the bolded and underlined portion of SEQ ID NO:15 corresponding to SEQ ID NO:16, oligomerize with a coronavirus spike (S) protein, for example, a MERS-CoV S protein, an HCoV-229E S protein, an HCoV-NL63 S protein, HCoV-OC43, an HCoV-HKU1 S protein, a SARS-CoV S protein, and/or a SARS-CoV-2 S protein. In some embodiments, the polypeptides derived from the S2 region of the HCoV-OC43 S protein oligomerize with a MERS-CoV S protein. In some embodiments, the polypeptides derived from the S2 region of the HCoV-OC43 S protein oligomerize with an HCoV-229E S protein. In some embodiments, the polypeptides derived from the S2 region of the HCoV-OC43 S protein oligomerize with an HCoV-NL63 S protein. In some embodiments, the polypeptides derived from the S2 region of the HCoV-OC43 S protein oligomerize with an HCoV-OC43 S protein. In some embodiments, the polypeptides derived from the S2 region of the HCoV-OC43 S protein oligomerize with an HCoV-HKU1 S protein. In some embodiments, the polypeptides derived from the S2 region of the HCoV-OC43 S protein oligomerize with a SARS-CoV S protein. In some embodiments, the polypeptides derived from the S2 region of the HCoV-OC43 S protein oligomerize with a SARS-CoV-2 S protein.

### B. Retroviruses

In particular embodiments, the virus is from the family Retroviridae. Retrovirus is the common name for Retroviridae. Retroviridae is a family of enveloped, positive-sense, single-stranded, linear RNA viruses. Retroviruses are classified into three sub-families: Oncoviruses, Lentiviruses, and Spumaviruses. Retroviruses are also classified based on their morphological types in the electron microscope as A-type, B-type, C-type, and D-type. The A-type viruses bud intracellularly, either into the cytoplasm or within endoplasmic reticulum, are not considered to be infectious, and have an electron lucent core. These are endogenous viruses and some animal species have thousands of copies of these A-type viruses in their chromosomal DNA. Their function remains unknown. B-type viruses have an eccentric core and the mammary tumor viruses exclusively have this structure. These viruses exist as endogenous and exogenous viruses in some animals and when expressed can cause mammary tumors. C-type viruses have a central electron-dense core, and most of the oncoviruses and endogenous viruses are of this type. The D-type viruses have a rod-shaped core and Lentiviruses are of this type.

Virions of retroviruses consist of enveloped particles about 100 nm in diameter. The outer lipid envelope consists of glycoprotein. The virions also contain two identical single-stranded RNA molecules 7-10 kilobases in length. The two molecules are present as a dimer, formed by base pairing between complementary sequences. Interaction sites between the two RNA molecules have been identified as a kissing stem-loop.

Although virions of different retroviruses do not have the same morphology or biology, all the virion components are very similar. The main virion components are:

Envelope: composed of lipids (obtained from the host plasma membrane during the budding process) as well as glycoprotein encoded by the env gene. The retroviral envelope serves three distinct functions: protection from the extracellular environment via the lipid bilayer, enabling the retrovirus to enter/exit host cells through endosomal membrane trafficking, and the ability to directly enter cells by fusing with their membranes.

RNA: consists of a dimer RNA. It has a cap at the 5′ end and a poly(A) tail at the 3′ end. Genomic RNA (gRNA) is produced as a result of host RNA polymerase II (Pol II) activity and by adding a 5′ methyl cap and a 3′ poly-A tail is processed as a host mRNA. The RNA genome also has terminal noncoding regions, which are important in replication, and internal regions that encode virion proteins for gene expression. The 5′ end includes four regions, which are R, U5, PBS, and L. The R region is a short repeated sequence at each end of the genome used during the reverse transcription to ensure correct end-to-end transfer in the growing chain. U5, on the other hand, is a short unique sequence between R and PBS. PBS (primer binding site) consists of 18 bases complementary to 3′ end of tRNA primer. L region is an untranslated leader region that gives the signal for packaging of the genome RNA. The 3′ end includes 3 regions, which are PPT (polypurine tract), U3, and R. The PPT is a primer for plus-strand DNA synthesis during reverse transcription. U3 is a sequence between PPT and R, which serves as a signal that the provirus can use in transcription. R is the terminal repeated sequence at 3′ end.

Proteins: consisting of gag proteins, protease (PR), pol proteins, and env proteins. Group-specific antigen (gag) proteins are major components of the viral capsid, which are about 2000-4000 copies per virion. Gag possesses two nucleic acid binding domains, including matrix (MA) and nucleocapsid (NC). Specifically recognizing, binding, and packaging the retroviral genomic RNA into assembling virions is one of the important functions of Gag protein. Gag interactions with cellular RNAs also regulate aspects of assembly. The expression of gag alone gives rise to assembly of immature virus-like particles that bud from the plasma membrane. In all retroviruses the Gag protein is the precursor to the internal structural protein. Protease (pro) is expressed differently in different viruses. It functions in proteolytic cleavages during virion maturation to make mature gag and pol proteins. Retroviral Gag proteins are responsible for coordinating many aspects of virion assembly. Pol proteins are responsible for synthesis of viral DNA and integration into host DNA after infection. Env proteins play a role in association and entry of virions into the host cell. Possessing a functional copy of an env gene is what makes retroviruses distinct from retroelements. The ability of the retrovirus to bind to its target host cell using specific cell-surface receptors is given by the surface component (SU) of the Env protein, while the ability of the retrovirus to enter the cell via membrane fusion is imparted by the membrane-anchored trans-membrane component (TM). Thus, it is the Env protein that enables the retrovirus to be infectious.

The retroviral genome is packaged as viral particles. These viral particles are dimers of single-stranded, positive-sense, linear RNA molecules. Retroviruses (and orterviruses in general) follow a layout of 5′-gag-pro-pol-env-3′ in the RNA genome. gag and pol encode polyproteins, each managing the capsid and replication. The pol region encodes enzymes necessary for viral replication, such as reverse transcriptase, protease and integrase. Depending on the virus, the genes may overlap or fuse into larger polyprotein chains.

A retrovirus has a membrane containing glycoproteins, which are able to bind to a receptor protein on a host cell. There are two strands of RNA within the cell that have three enzymes: protease, reverse transcriptase, and integrase. The first step of replication is the binding of the glycoprotein to the receptor protein. Once these have been bound, the cell membrane degrades, becoming part of the host cell, and the RNA strands and enzymes enter the cell. Within the cell, reverse transcriptase creates a complementary strand of DNA from the retrovirus RNA and the RNA is degraded; this strand of DNA is known as cDNA. The cDNA is then replicated, and the two strands form a weak bond and enter the nucleus. Once in the nucleus, the DNA is integrated into the host cell's DNA with the help of integrase. This cell can either stay dormant, or RNA may be synthesized from the DNA and used to create the proteins for a new retrovirus. Ribosome units are used to translate the mRNA of the virus into the amino acid sequences which can be made into proteins in the rough endoplasmic reticulum. This step will also make viral enzymes and capsid proteins. Viral RNA will be made in the nucleus. These pieces are then gathered together and are pinched off of the cell membrane as a new retrovirus.

While transcription was classically thought to occur only from DNA to RNA, reverse transcriptase transcribes RNA into DNA. The term “retro” in retrovirus refers to this reversal (making DNA from RNA) of the usual direction of transcription. It still obeys the central dogma of molecular biology, which states that information can be transferred from nucleic acid to nucleic acid but cannot be transferred back from protein to either protein or nucleic acid. Reverse transcriptase activity outside of retroviruses has been found in almost all eukaryotes, enabling the generation and insertion of new copies of retrotransposons into the host genome. These inserts are transcribed by enzymes of the host into new RNA molecules that enter the cytosol. Next, some of these RNA molecules are translated into viral proteins. In the rough endoplasmic reticulum glycosylation begins and the env gene is translated from spliced mRNAs in the rough endoplasmic reticulum, into molecules of the envelope protein. When the envelope protein molecules are carried to the Golgi complex, they are divided into surface glycoprotein and transmembrane glycoprotein by a host protease. These two glycoprotein products stay in close affiliation, and they are transported to the plasma membrane after further glycosylation.

More than 35 million people worldwide are infected with the retrovirus human immunodeficiency virus (HIV), the virus that causes AIDS. Once in the body, HIV attacks and destroys immune cells, which normally protect the body from infection. Current treatments help to prevent the virus from multiplying.

In the absence of treatment, HIV typically progress through three stages. Stage 1 corresponds to acute HIV Infection. People have a large amount of HIV in their blood and are very contagious. Some people have flu-like symptoms within 2 to 4 weeks after infection (called acute HIV infection). These symptoms may last for a few days or several weeks. Possible symptoms include fever, headache, chills, rash, night sweats, muscle aches and joint pain, sore throat, fatigue, diarrhea, weight loss, cough, swollen lymph nodes, and mouth ulcers. Only antigen/antibody tests or nucleic acid tests (NATs) can diagnose acute infection.

Stage 2 corresponds to chronic HIV infection. This stage is also called asymptomatic HIV infection or clinical latency. HIV is still active but reproduces at very low levels. People may not have any symptoms or get sick during this phase. Others may progress to symptomatic HIV and have symptoms including fever, fatigue, swollen lymph nodes, diarrhea, weight loss, oral yeast infection (thrush), shingles (herpes zoster), and pneumonia. Without taking HIV medicine, this period may last a decade or longer, but some may progress faster. People can transmit HIV in this phase. At the end of this phase, the amount of HIV in the blood (called viral load) goes up and the CD4 cell count goes down. The person may have symptoms as the virus levels increase in the body, and the person moves into Stage 3.

Stage 3 corresponds to Acquired Immunodeficiency Syndrome (AIDS), the most severe phase of HIV infection. People with AIDS have such badly damaged immune systems that they get an increasing number of severe illnesses, called opportunistic infections. The signs and symptoms of some of these infections may include: sweats, chills, recurring fever, chronic diarrhea, swollen lymph glands, persistent white spots or unusual lesions on the tongue or in the mouth, persistent, unexplained fatigue, weakness, weight loss, and skin rashes or bumps. People receive an AIDS diagnosis when their CD4 cell count drops below 200 cells/mm, or if they develop certain opportunistic infections. People with AIDS can have a high viral load and be very infectious. Without treatment, people with AIDS typically survive about three years.

HIV-1 testing is initially done using an enzyme-linked immunosorbent assay (ELISA) to detect antibodies to HIV-1. Specimens with a non-reactive result from the initial ELISA are considered HIV-negative, unless new exposure to an infected partner or partner of unknown HIV status has occurred. Specimens with a reactive ELISA result are retested in duplicate. If the result of either duplicate test is reactive, the specimen is reported as repeatedly reactive and undergoes confirmatory testing with a more specific supplemental test (e.g., a polymerase chain reaction (PCR), western blot or, less commonly, an immunofluorescence assay (IFA)). Specimens that are repeatedly reactive by ELISA and positive by IFA or PCR or reactive by western blot are considered HIV-positive and indicative of HIV infection. Specimens that are repeatedly ELISA-reactive occasionally provide an indeterminate western blot result, which may be either an incomplete antibody response to HIV in an infected person or nonspecific reactions in an uninfected person.

To become infected with HIV, infected blood, semen or vaginal secretions must enter the body. This can happen in several ways. Infection may occur as a result of vaginal, anal, or oral sex with an infected partner whose blood, semen or vaginal secretions enter the body. The virus can enter the body through mouth sores or small tears that sometimes develop in the rectum or vagina during sexual activity. Infection may occur as a result of sharing contaminated IV drug paraphernalia (needles and syringes). Infection may occur as a result of transmission through blood transfusions. Infection may occur as a result of pregnancy or delivery or through breast-feeding. Infected mothers can pass the virus on to their babies.

Anyone of any age, race, sex or sexual orientation can be infected with HIV/AIDS. Unprotected sex, sexually-transmitted diseases, and IV drug use are all factors that increase the risk of HIV infection.

The present disclosure encompasses treatment or prevention of infection of any virus in the Retroviridae family. In certain embodiments, the disclosure encompasses treatment or prevention of infection of any virus in the subfamily Orthoretrovirinae and including the five genera, Alpharetrovirus, Betaretrovirus, Deltaretrovirus, Epsilonretrovirus, Gammaretrovirus, and Lentivirus. In specific embodiments, the disclosure encompasses treatment or prevention of infection of any virus in the genus of Lentivirus, including the species human immunodeficiency virus 1 and human immunodeficiency virus 2 that infect humans and over time cause acquired immunodeficiency syndrome (AIDS).

Aspects of the disclosure relate to polypeptides that interact with an HIV spike (S) protein. A major target for potential HIV therapeutics is a spike-shaped virus protein known as Env. Env extends from the surface of the HIV virus particle. The protein is trimeric structure of three heterodimers made of three cap-like subunits called glycoprotein 120 (gp120) and three stem-like subunits called glycoprotein 41 (gp41) that anchor Env in the viral membrane. Analysis of the structure and sequence of several different env genes suggests that Env proteins are type 1 fusion machines. Type 1 fusion machines initially bind a receptor on the target cell surface, which triggers a conformational change, allowing for binding of the fusion protein. The fusion peptide inserts itself in the host cell membrane and brings the host cell membrane very close to the viral membrane to facilitate membrane fusion.

The env gene codes for the gp160 protein which forms a homotrimer, and is cleaved into gp120 and gp41 by the host cell protease, furin. To form an active fusion protein, surface protein gp120 and transmembrane protein gp41 polypeptides remain non-covalently bound together, but this interaction is often not stable, leading to shed, soluble gp120 and membrane-bound, gp41 stumps.

Env expression is regulated by the gene product of rev. Experimental deletion of rev resulted in the inability to detect the Env protein and levels of env mRNA in the cell cytoplasm were significantly diminished. However, when total cellular RNA was analyzed, env RNA totals were not significantly difference in the presence and absence of rev coexpression. It was found that without rev expression, there was a marked increase in nuclear env RNA, which suggests that rev plays an important role in the nuclear export of env mRNA. The role of rev was further elucidated when it was found that Rev acts in trans to target a specific sequence present in the env gene of HIV-1 to initiate export of incompletely spliced HIV-1 RNA from the nucleus.

Exposed on the surface of the viral envelope, the glycoprotein gp120 binds to the CD4 receptor on any target cell that has such a receptor, particularly the helper T-cell. Prior to binding the host cell, gp120 remains effectively hidden from antibodies because it is buried in the protein and shielded by sugars. Gp120 is only exposed when in close proximity to a host cell and the space between the viral and host cell membranes is small enough to sterically hinder the binding of antibodies.

The glycoprotein gp41 is non-covalently bound to gp120, and provides the second step by which HIV enters the cell. It is originally buried within the viral envelope, but when gp120 binds to a CD4 receptor, gp120 changes its conformation causing gp41 to become exposed, where it can assist in fusion with the host cell.

The sequence of HIV gp160 S protein with the gp41 fragment bolded and underlined is provided as SEQ ID NO:17:

(SEQ ID NO: 17)
MRVKEKYQHLWRWGWRWGTMLLGMLMICSATEKLWVTVYYGVPVWKEATT
TLFCASDAKAYDTEVHNVWATHACVPTDPNPQEVVLVNVTENFNMWKNDM
VEQMHEDIISLWDQSLKPCVKLTPLCVSLKCTDLKNDTNTNSSSGRMIME
KGEIKNCSFNISTSIRGKVQKEYAFFYKLDIIPIDNDTTSYKLTSCNTSV
ITQACPKVSFEPIPIHYCAPAGFAILKCNNKTFNGTGPCTNVSTVQCTHG
IRPVVSTQLLLNGSLAEEEVVIRSVNFTDNAKTIIVQLNTSVEINCTRPN
NNTRKRIRIQRGPGRAFVTIGKIGNMRQAHCNISRAKWNNTLKQIASKLR
EQFGNNKTIIFKQSSGGDPEIVTHSFNCGGEFFYCNSTQLFNSTWFNSTW
STEGSNNTEGSDTITLPCRIKQIINMWQKVGKAMYAPPISGQIRCSSNIT
GLLLTRDGGNSNNESEIFRPGGGDMRDNWRSELYKYKVVKIEPLGVAPTK
AKRRVVQREKRAVGIGALFLGFLGAAGSTM
GAASMTLTVQARQLLSGIVQQQNNLLRAIE
AQQHLLQLTVWGIKQLQARILAVERYLKDQ
QLLGIWGCSGKLICTTAVPWNASWSNKSLE
QIWNHTTWMEWDREINNYTSLIHSLIEESQ
NQQEKNEQELLELDKWASLWNWFNITNWLW
YIKLFIMIVGGLVGLRIVFAVLSIVNRVRQ
GYSPLSFQTHLPTPRGPDRPEGIEEEGGER
DRDRSIRLVNGSLALIWDDLRSLCLESYHR
LRDLLLIVTRIVELLGRRGWEALKYWWNLL
YQWSQELKNSAVSLLNATAIAVAEGTDRVI
EVVQGACRAIRHIPRRIRQGLERILL.

In some embodiments, the polypeptides of the disclosure are derived from the HIV gp160 S protein. In some embodiments, the polypeptide derived from the HIV gp160 S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the bolded and underlined portion of SEQ ID NO:17 corresponding to SEQ ID NO:18, or a fragment or functional derivative thereof.

In some embodiments, the polypeptides derived from the HIV gp160 S protein, for example, the gp41 fragment of the HIV gp160 S protein corresponding to SEQ ID NO:18, interact with a retroviral spike (S) protein. In some embodiments, the polypeptides derived from the HIV gp160 S protein, for example, the gp41 fragment of the HIV gp160 S protein corresponding to SEQ ID NO:18, interact with an HIV spike (S) protein. In some embodiments, the polypeptides derived from the HIV gp160 S protein, for example, the gp41 fragment of the the HIV gp160 S protein corresponding to SEQ ID NO:18, oligomerize with a retroviral spike (S) protein. In some embodiments, the polypeptides derived from the HIV gp160 S protein, for example, the gp41 fragment of the HIV gp160 S protein corresponding to SEQ ID NO:18, oligomerize with an HIV spike (S) protein.

### C. Ebolavirus

In particular embodiments, the virus is from the family Filoviridae. Filoviridae is a family of enveloped negative-sense, single-stranded, RNA viruses. Filoviridae are classified into six genera, including Cuevavirus, Dianlovirus, Ebolavirus, Marburgvirus, Striavirus, and Thamnovirus.

The filovirus life cycle begins with virion attachment to specific cell-surface receptors, followed by fusion of the virion envelope with cellular membranes and the concomitant release of the virus nucleocapsid into the cytosol. The viral RNA-dependent RNA polymerase (RdRp, or RNA replicase) partially uncoats the nucleocapsid and transcribes the genes into positive-stranded mRNAs, which are then translated into structural and nonstructural proteins. Filovirus RdRps bind to a single promoter located at the 3′ end of the genome. Transcription either terminates after a gene or continues to the next gene downstream. This means that genes close to the 3′ end of the genome are transcribed in the greatest abundance, whereas those toward the 5′ end are least likely to be transcribed. The gene order is therefore a simple but effective form of transcriptional regulation. The most abundant protein produced is the nucleoprotein, whose concentration in the cell determines when the RdRp switches from gene transcription to genome replication. Replication results in full-length, positive-stranded antigenomes that are in turn transcribed into negative-stranded virus progeny genome copies. Newly synthesized structural proteins and genomes self-assemble and accumulate near the inside of the cell membrane. Virions bud off from the cell, gaining their envelopes from the cellular membrane they bud from. The mature progeny particles then infect other cells to repeat the cycle.

Ebola, also known as Ebola virus disease (EVD) or Ebola hemorrhagic fever (EHF), is a viral hemorrhagic fever of humans and other primates caused by ebolaviruses from the family Filoviridae. EVD most commonly affects people and nonhuman primates (such as monkeys, gorillas, and chimpanzees). It is caused by an infection with a group of viruses within the genus Ebolavirus: Ebola virus (species Zaire ebolavirus), Sudan virus (species Sudan ebolavirus), Taï Forest virus (species Taï Forest ebolavirus, formerly Côte d'Ivoire ebolavirus), Bundibugyo virus (species Bundibugyo ebolavirus), Reston virus (species Reston ebolavirus), and Bombali virus (species Bombali ebolavirus). Of these, only four (Ebola, Sudan, Tai Forest, and Bundibugyo viruses) are known to cause disease in people. Reston virus is known to cause disease in nonhuman primates and pigs, but not in people. It is unknown if Bombali virus, which was recently identified in bats, causes disease in either animals or people.

Ebolaviruses contain single-stranded, non-infectious RNA genomes. Ebolavirus genomes contain seven genes including 3′-UTR-NP-VP35-VP40-GP-VP30-VP24-L-5′-UTR. The genomes of the five different ebolaviruses (BDBV, EBOV, RESTV, SUDV and TAFV) differ in sequence and the number and location of gene overlaps. As with all filoviruses, ebolavirus virions are filamentous particles that may appear in the shape of a shepherd's crook, of a “U” or of a “6,” and they may be coiled, toroid or branched. In general, ebolavirions are 80 nanometers (nm) in width and may be as long as 14,000 nm.

Signs and symptoms typically start between two days and three weeks after contracting the virus with a fever, sore throat, muscular pain, weakness, fatigue, and headaches. Vomiting, diarrhea, abdominal pain, and rash usually follow, along with decreased function of the liver and kidneys. At this time, some people begin to bleed both internally and externally. The disease has a high risk of death, killing 25% to 90% of those infected, with an average of about 50%. This is often due to shock from fluid loss, and typically follows six to 16 days after symptoms appear.

The virus spreads through direct contact with body fluids, such as blood from infected humans or other animals. Spread may also occur from contact with items recently contaminated with bodily fluids. Spread of the disease through the air between primates, including humans, has not been documented in either laboratory or natural conditions. Semen or breast milk of a person after recovery from EVD may carry the virus for several weeks to months. Fruit bats are believed to be the normal carrier in nature, able to spread the virus without being affected by it.

Possible non-specific laboratory indicators of EVD include a low platelet count; an initially decreased white blood cell count followed by an increased white blood cell count; elevated levels of the liver enzymes alanine aminotransferase (ALT) and aspartate aminotransferase (AST); and abnormalities in blood clotting often consistent with disseminated intravascular coagulation (DIC) such as a prolonged prothrombin time, partial thromboplastin time, and bleeding time. Filovirions such as EBOV may be identified by their unique filamentous shapes in cell cultures examined with electron microscopy.

The specific diagnosis of EVD is confirmed by isolating the virus, detecting its RNA or proteins, or detecting antibodies against the virus in a person's blood. Isolating the virus by cell culture, detecting the viral RNA by polymerase chain reaction (PCR) and detecting proteins by enzyme-linked immunosorbent assay (ELISA) are methods best used in the early stages of the disease and also for detecting the virus in human remains. Detecting antibodies against the virus is most reliable in the later stages of the disease and in those who recover. IgM antibodies are detectable two days after symptom onset and IgG antibodies can be detected six to 18 days after symptom onset.

Health workers who do not use proper infection control while caring for Ebola patients, and family and friends in close contact with Ebola patients, are at the highest risk of getting sick. Ebola can spread when people come into contact with infected blood or body fluids. Ebola poses little risk to travelers or the general public who have not cared for or been in close contact (within 3 feet or 1 meter) with someone sick with Ebola. The virus can remain in areas of the body that are immunologically privileged sites after acute infection. These are sites where viruses and pathogens, like the Ebola virus, are shielded from the survivor's immune system, even after being cleared elsewhere in the body. These areas include the testes, interior of the eyes, placenta, and central nervous system, particularly the cerebrospinal fluid.

The present disclosure encompasses treatment or prevention of infection of any virus in the Filoviridae family and including the six genera, Cuevavirus, Dianlovirus, Ebolavirus, Marburgvirus, Striavirus, and Thamnovirus. In specific embodiments, the disclosure encompasses treatment or prevention of infection of any virus in the genus of Ebolavirus, including the species Ebola virus (species Zaire ebolavirus), Sudan virus (species Sudan ebolavirus), Tai Forest virus (species TaY Forest ebolavirus, formerly Côe d'Ivoire ebolavirus), Bundibugyo virus (species Bundibugyo ebolavirus) that infect humans.

Aspects of the disclosure relate to polypeptides that interact with an Ebola virus (EBOV) spike (S) protein. EBOV is thought to infect humans through contact with mucous membranes or skin breaks. After infection, endothelial cells (cells lining the inside of blood vessels), liver cells, and several types of immune cells such as macrophages, monocytes, and dendritic cells are the main targets of attack. Following infection, immune cells carry the virus to nearby lymph nodes where further reproduction of the virus takes place. From there the virus can enter the bloodstream and lymphatic system and spread throughout the body. Macrophages are the first cells infected with the virus, and this infection results in programmed cell death. Other types of white blood cells, such as lymphocytes, also undergo programmed cell death leading to an abnormally low concentration of lymphocytes in the blood. This contributes to the weakened immune response seen in those infected with EBOV.

Endothelial cells may be infected within three days after exposure to the virus. The breakdown of endothelial cells leading to blood vessel injury can be attributed to EBOV glycoproteins. This damage occurs due to the synthesis of Ebola virus glycoprotein (GP), which reduces the availability of specific integrins responsible for cell adhesion to the intercellular structure and causes liver damage, leading to improper clotting. The widespread bleeding that occurs in affected people causes swelling and shock due to loss of blood volume. The dysfunctional bleeding and clotting commonly seen in EVD has been attributed to increased activation of the extrinsic pathway of the coagulation cascade due to excessive tissue factor production by macrophages and monocytes.

After infection, a secreted glycoprotein, small soluble glycoprotein (sGP or GP) is synthesized. EBOV replication overwhelms protein synthesis of infected cells and the host immune defenses. The GP forms a trimeric complex, which tethers the virus to the endothelial cells. The sGP forms a dimeric protein that interferes with the signaling of neutrophils, another type of white blood cell. This enables the virus to evade the immune system by inhibiting early steps of neutrophil activation.

The sequence of Ebola virus glycoprotein with the GP fragment bolded and underlined is provided as SEQ ID NO:19:

(SEQ ID NO: 19)
MGVTGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEELSFTVVSNGAKNISGQSPARTSSDPGTNTTTEDHKIMASENSSAM
VQVHSQGREAAVSHLTTLATISTSPQSLTTKPGPDNSTHNTPVYKLDISE
ATQVEQHHRRTDNDSTASDTPSATTAAGPPKAENTNTSKSTDFLDPATTT
SPQNHSETAGNNNTHHQDTGEESASSGKLGLITNTIAGVAGLITGGRRTR
REAIVNAQPKCNPNLHYWTTQDEGAAIGLA
WIPYFGPAAEGIYTEGLMHNQDGLICGLRQ
LANETTQALQLFLRATTELRTFSILNRKAI
DFLLQRWGGTCHILGPDCCIEPHDWTKNIT
DKIDQIIHDFVDKTLPDQGDNDNWWTGWRQ
WIPAGIGVTGVIIAVIALFCICKFVF.

In some embodiments, the polypeptides of the disclosure are derived from the Ebola virus glycoprotein. In some embodiments, the polypeptide derived from the Ebola virus glycoprotein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the bolded and underlined portion of SEQ ID NO:19 corresponding to SEQ ID NO:20, or a fragment or functional derivative thereof.

In some embodiments, the polypeptides derived from the Ebola virus glycoprotein, for example, the GP fragment of the Ebola virus glycoprotein corresponding to SEQ ID NO:20, interact with a Filoviridae glycoprotein. In some embodiments, the polypeptides derived from the Ebola virus glycoprotein, for example, the GP fragment of the Ebola virus glycoprotein corresponding to SEQ ID NO:20, interact with an Ebola glycoprotein. In some embodiments, the polypeptides derived from the Ebola virus glycoprotein, for example, the GP fragment of the Ebola virus glycoprotein corresponding to SEQ ID NO:20, oligomerize with a Filoviridae glycoprotein. In some embodiments, the polypeptides derived from the Ebola virus glycoprotein, for example, the GP fragment of the Ebola virus glycoprotein corresponding to SEQ ID NO:20, oligomerize with an Ebola virus glycoprotein.

### D. Orthomyxoviruses

In particular embodiments, the virus is from the family Orthomyxoviridae. Orthomyxoviridae is a family of enveloped negative-sense, single-stranded, linear RNA viruses. It includes seven genera: Alphainfluenzavirus, Betainfluenzavirus, Deltainfluenzavirus, Gammainfluenzavirus, Isavirus, Thogotovirus, and Quaranjavirus. Alphainfluenzavirus, Betainfluenzavirus, Deltainfluenzavirus, and Gammainfluenzavirus contain viruses that cause influenza in birds and mammals, including humans.

Alphainfluenzavirus, Betainfluenzavirus, and Gammainfluenzavirus contain viruses that cause influenza in humans.

Viruses of the family Orthomyxoviridae contain six to eight segments of linear negative-sense single stranded RNA. They have a total genome length that is 10,000-14,600 nucleotides (nt). The influenza virus A genome, for instance, has eight pieces of segmented negative-sense RNA (13.5 kilobases total). The genome sequence has terminal repeated sequences; repeated at both ends. Terminal repeats at the 5′-end 12-13 nucleotides long. Nucleotide sequences of 3′-terminus identical; the same in genera of same family; most on RNA (segments), or on all RNA species. Terminal repeats at the 3′-end 9-11 nucleotides long. Encapsidated nucleic acid is solely genomic. Each virion may contain defective interfering copies. The M and NS genes produce two different genes via alternative splicing.

The influenzavirus virion is pleomorphic; the viral envelope can occur in spherical and filamentous forms. In general, the virus's morphology is ellipsoidal with particles 100-120 nm in diameter, or filamentous with particles 80-100 nm in diameter and up to 20 μm long. There are approximately 500 distinct spike-like surface projections in the envelope each projecting 10-14 nm from the surface with varying surface densities. The major glycoprotein (HA) spike is interposed irregularly by clusters of neuraminidase (NA) spikes, with a ratio of HA to NA of about 10 to 1.

The viral envelope composed of a lipid bilayer membrane in which the glycoprotein spikes are anchored encloses the nucleocapsids; nucleoproteins of different size classes with a loop at each end; the arrangement within the virion is uncertain. The ribonuclear proteins are filamentous and fall in the range of 50-130 nm long and 9-15 nm in diameter with helical symmetry.

Typically, influenza is transmitted from infected mammals through the air by coughs or sneezes, creating aerosols containing the virus, and from infected birds through their droppings. Influenza can also be transmitted by saliva, nasal secretions, feces and blood. Infections occur through contact with these bodily fluids or with contaminated surfaces. Out of a host, flu viruses can remain infectious for about one week at human body temperature, over 30 days at 0° C. (32° F.), and indefinitely at very low temperatures. They can be inactivated easily by disinfectants and detergents.

The viruses bind to a cell through interactions between its hemagglutinin glycoprotein and sialic acid sugars on the surfaces of epithelial cells in the lung and throat. The cell imports the virus by endocytosis. In the acidic endosome, part of the hemagglutinin protein fuses the viral envelope with the vacuole's membrane, releasing the viral RNA (vRNA) molecules, accessory proteins and RNA-dependent RNA polymerase into the cytoplasm. These proteins and vRNA form a complex that is transported into the cell nucleus, where the RNA-dependent RNA polymerase begins transcribing complementary positive-sense cRNA. The cRNA is either exported into the cytoplasm and translated or remains in the nucleus. Newly synthesized viral proteins are either secreted through the Golgi apparatus onto the cell surface (in the case of neuraminidase and hemagglutinin) or transported back into the nucleus to bind vRNA and form new viral genome particles.

Negative-sense vRNAs that form the genomes of future viruses, RNA-dependent RNA transcriptase, and other viral proteins are assembled into a virion. Hemagglutinin and neuraminidase molecules cluster into a bulge in the cell membrane. The vRNA and viral core proteins leave the nucleus and enter this membrane protrusion. The mature virus buds off from the cell in a sphere of host phospholipid membrane, acquiring hemagglutinin and neuraminidase with this membrane coat. As before, the viruses adhere to the cell through hemagglutinin; the mature viruses detach once their neuraminidase has cleaved sialic acid residues from the host cell. After the release of new influenza virus, the host cell dies.

Orthomyxoviridae viruses are one of two RNA viruses that replicate in the nucleus (the other being retroviridae). This is because the machinery of orthomyxo viruses cannot make their own mRNAs. They use cellular RNAs as primers for initiating the viral mRNA synthesis in a process known as cap snatching. Once in the nucleus, the RNA Polymerase Protein PB2 finds a cellular pre-mRNA and binds to its 5′ capped end. Then RNA Polymerase PA cleaves off the cellular mRNA near the 5′ end and uses this capped fragment as a primer for transcribing the rest of the viral RNA genome in viral mRNA. This is due to the need of mRNA to have a 5′ cap in order to be recognized by the cell's ribosome for translation.

There are four genera of influenza virus, each containing only a single species, or type. Influenza A and C infect a variety of species (including humans), while influenza virus B almost exclusively infects humans, and influenza D infects cattle and pigs.

Influenza A viruses are negative-sense, single-stranded, segmented RNA viruses. The entire Influenza A virus genome is 13,588 bases long and is contained on eight RNA segments that code for at least 10 but up to 14 proteins, depending on the strain. The virus particle (also called the virion) is 80-120 nanometers in diameter such that the smallest virions adopt an elliptical shape. The length of each particle varies considerably, owing to the fact that influenza is pleomorphic, and can be in excess of many tens of micrometers, producing filamentous virions. Despite these varied shapes, the virions of all influenza type A viruses are similar in composition. They are all made up of a viral envelope containing two main types of proteins, wrapped around a central core.

The two large proteins found on the outside of viral particles are hemagglutinin (HA) and neuraminidase (NA). HA is a protein that mediates binding of the virion to target cells and entry of the viral genome into the target cell. NA is involved in release from the abundant non-productive attachment sites present in mucus as well as the release of progeny virions from infected cells. These proteins are usually the targets for antiviral drugs. Furthermore, they are also the antigen proteins to which a host's antibodies can bind and trigger an immune response. Influenza type A viruses are categorized into subtypes based on the type of these two proteins on the surface of the viral envelope. There are 16 subtypes of HA and 9 subtypes of NA known, but only H 1, 2 and 3, and N 1 and 2 are commonly found in humans.

The central core of a virion contains the viral genome and other viral proteins that package and protect the genetic material. The influenza type A virus genome is not a single piece of RNA; instead, it consists of segmented pieces of negative-sense RNA, each piece containing either one or two genes which code for a gene product (protein). The term negative-sense RNA as used herein refers to the fact that the RNA genome cannot be translated into protein directly; it must first be transcribed to positive-sense RNA before it can be translated into protein products. The segmented nature of the genome allows for the exchange of entire genes between different viral strains.

Influenza A viruses that cause infections may be treated or prevented by the polypeptides disclosed herein include: Influenza A virus subtype H1N1, Influenza A virus subtype H1N2, Influenza A virus subtype H2N2, Influenza A virus subtype H2N3, Influenza A virus subtype H3N1, Influenza A virus subtype H3N2, Influenza A virus subtype H3N8, Influenza A virus subtype H5N1, Influenza A virus subtype H5N2, Influenza A virus subtype H5N3, Influenza A virus subtype H5N6, Influenza A virus subtype H5N8, Influenza A virus subtype H5N9, Influenza A virus subtype H6N1, Influenza A virus subtype H6N2, Influenza A virus subtype H7N1, Influenza A virus subtype H7N2, Influenza A virus subtype H7N3, Influenza A virus subtype H7N4, Influenza A virus subtype H7N7, Influenza A virus subtype H7N9, Influenza A virus subtype H9N2, Influenza A virus subtype H10N7, Influenza A virus subtype H10N8, Influenza A virus subtype H11N2, Influenza A virus subtype H11N9, Influenza A virus subtype H17N10, Influenza A virus subtype H18N11, or a combination thereof.

The Influenza B virus capsid is enveloped while its virion consists of an envelope, a matrix protein, a nucleoprotein complex, a nucleocapsid, and a polymerase complex. It is sometimes spherical and sometimes filamentous. Its 500 or so surface projections are made of hemagglutinin and neuraminidase. The Influenza B virus genome is 14,548 nucleotides long and consists of eight segments of linear negative-sense, single-stranded RNA. The multipartite genome is encapsidated, each segment in a separate nucleocapsid, and the nucleocapsids are surrounded by one envelope. There are two known circulating lineages of Influenza B virus based on the antigenic properties of the surface glycoprotein hemagglutinin. The lineages are termed B/Yamagata/16/88-like and B/Victoria/2/87-like viruses, and infections due to these Influenza B virus lineages may be treated or prevented by the polypeptides disclosed herein.

Influenza C virus has 7 RNA segments and encodes 9 proteins, while Types A and B have 8 RNA segments and encode at least 10 proteins. Influenza C virus has only one glycoprotein: hemagglutinin-esterase fusion (HEF). Unlike influenza virus A and influenza virus B, influenza virus C also expresses the enzyme esterase. This enzyme is similar to the enzyme neuraminidase produced by Types A and B in that they both function in destroying the host cell receptors.

The time period between exposure to the influenza virus and development of symptoms, called the incubation period, is 1-4 days, most commonly 1-2 days. Many infections, however, are asymptomatic. The onset of symptoms is sudden, and initial symptoms are predominately non-specific, including fever, chills, headaches, muscle pain or aching, a feeling of discomfort, loss of appetite, lack of energy/fatigue, and confusion. These symptoms are usually accompanied by respiratory symptoms such as a dry cough, sore or dry throat, hoarse voice, and a stuffy or runny nose. Coughing is the most common symptom. Gastrointestinal symptoms may also occur, including nausea, vomiting, diarrhea, and gastroenteritis. The standard influenza symptoms typically last for 2-8 days.

People who are infected can transmit influenza viruses through breathing, talking, coughing, and sneezing, which spread respiratory droplets and aerosols that contain virus particles into the air. A person susceptible to infection can then contract influenza by coming into contact with these particles. Respiratory droplets are relatively large and travel less than two meters before falling onto nearby surfaces. Aerosols are smaller and remain suspended in the air longer, so they take longer to settle and can travel further than respiratory droplets. Inhalation of aerosols can lead to infection, but most transmission is in the area about two meters around an infected person via respiratory droplets that come into contact with mucosa of the upper respiratory tract. Transmission through contact with a person, bodily fluids, or intermediate objects (fomites) can also occur, such as through contaminated hands and surfaces since influenza viruses can survive for hours on non-porous surfaces. If one's hands are contaminated, then touching one's face can cause infection. Influenza is usually transmissible from one day before the onset of symptoms to 5-7 days after. In healthy adults, the virus is shed for up to 3-5 days.

People who are at risk of exposure to influenza include health care workers, social care workers, and those who live with or care for people vulnerable to influenza. In long-term care facilities, the flu can spread rapidly after it is introduced. A variety of factors likely encourage influenza transmission, including lower temperature, lower absolute and relative humidity, less ultraviolet radiation from the sun, and crowding.

Diagnosis based on symptoms is fairly accurate in otherwise healthy people during seasonal epidemics and should be suspected in cases of pneumonia, ARDS, sepsis, or if encephalitis, myocarditis, and rhabdomyolysis occur. Because influenza is similar to other viral respiratory tract illnesses, laboratory diagnosis is necessary for confirmation. Common ways of collecting samples for testing include nasal and throat swabs. Samples may be taken from the lower respiratory tract if infection has cleared the upper but not lower respiratory tract. Influenza testing is recommended for anyone hospitalized with symptoms resembling influenza during flu season or who is connected to an influenza case. For severe cases, earlier diagnosis improves patient outcome. Diagnostic methods that can identify influenza include viral cultures, antibody- and antigen-detecting tests, and nucleic acid-based tests.

Viruses can be grown in a culture of mammalian cells or embryonated eggs for 3-10 days to monitor cytopathic effect. Final confirmation can then be done via antibody staining, hemadsorption using erythrocytes, or immunofluorescence microscopy. Shell vial cultures, which can identify infection via immunostaining before a cytopathic effect appears, are more sensitive than traditional cultures with results in 1-3 days.

Serological assays can be used to detect an antibody response to influenza after natural infection or vaccination. Common serological assays include hemagglutination inhibition assays that detect HA-specific antibodies, virus neutralization assays that check whether antibodies have neutralized the virus, and enzyme-linked immunoabsorbant assays.

Direct fluorescent or immunofluorescent antibody (DFA/IFA) tests involve staining respiratory epithelial cells in samples with fluorescently-labeled influenza-specific antibodies, followed by examination under a fluorescent microscope. They can differentiate between IAV and IBV but can't subtype A.

Nucleic acid-based tests (NATs) amplify and detect viral nucleic acid. Among NATs, reverse transcription polymerase chain reaction is the most traditional and considered the gold standard for diagnosing influenza because it is fast and can subtype IAV. Other NATs that have been used include loop-mediated isothermal amplification-based assay, simple amplification-based assay, and nucleic acid sequence-based amplification. Nucleic acid sequencing methods can identify infection by obtaining the nucleic acid sequence of viral samples to identify the virus and antiviral drug resistance.

The present disclosure encompasses treatment or prevention of infection of any virus in the Orthomyxoviridae family and including the seven genera, Alphainfluenzavirus, Betainfluenzavirus, Deltainfluenzavirus, Gammainfluenzavirus, Isavirus, Thogotovirus, and Quaranjavirus. In specific embodiments, the disclosure encompasses treatment or prevention of infection of any virus in the genera of Orthomyxoviridae family that infect vertebrates, including Alphainfluenzavirus, Betainfluenzavirus, Deltainfluenzavirus, and Gammainfluenzavirus. In specific embodiments, the disclosure encompasses treatment or prevention of infection of any virus in the genera of Orthomyxoviridae family that infect humans, including the Alphainfluenzavirus, Betainfluenzavirus, and Gammainfluenzavirus. In specific embodiments, the disclosure encompasses treatment or prevention of infection of any virus in the genera of Alphainfluenzavirus, including the species influenza virus A and any influenza virus A strains, subtypes, or lineages. In specific embodiments, the disclosure encompasses treatment or prevention of infection of any virus in the genera of Betainfluenzavirus, including the species influenza virus B and any influenza virus B strains, subtypes, or lineages. In specific embodiments, the disclosure encompasses treatment or prevention of infection of any virus in the genera of Gammainfluenzavirus, including the species influenza virus C and any influenza virus C strains, subtypes, or lineages.

Aspects of the disclosure relate to polypeptides that interact with an influenza virus spike (S) protein. In humans, influenza viruses first cause infection by infecting epithelial cells in the respiratory tract. Illness during infection is primarily the result of lung inflammation and compromise caused by epithelial cell infection and death, combined with inflammation caused by the immune system's response to infection. Non-respiratory organs can become involved, but the mechanisms by which influenza is involved in these cases is unknown. Severe respiratory illness can be caused by multiple, non-exclusive mechanisms, including obstruction of the airways, loss of alveolar structure, loss of lung epithelial integrity due to epithelial cell infection and death, and degradation of the extracellular matrix that maintains lung structure. In particular, alveolar cell infection appears to drive severe symptoms since this results in impaired gas exchange and enables viruses to infect endothelial cells, which produce large quantities of pro-inflammatory cytokines.

The pathophysiology of influenza is significantly influenced by which receptors influenza viruses bind to during entry into cells. Mammalian influenza viruses preferentially bind to sialic acids connected to the rest of the oligosaccharide by an α-2,6 link, most commonly found in various respiratory cells, such as respiratory and retinal epithelial cells. Avian influenza viruses (AIVs) prefer sialic acids with an α-2,3 linkage, which are most common in birds in gastrointestinal epithelial cells and in humans in the lower respiratory tract. Furthermore, cleavage of the HA protein into HA1, the binding subunit, and HA2, the fusion subunit, is performed by different proteases, affecting which cells can be infected. For mammalian influenza viruses and low pathogenic AIVs, cleavage is extracellular, which limits infection to cells that have the appropriate proteases, whereas for highly pathogenic AIVs, cleavage is intracellular and performed by ubiquitous proteases, which allows for infection of a greater variety of cells, thereby contributing to more severe disease.

The sequence of Influenza A/H1 HA S protein with the HA2 fragment bolded and underlined is provided as SEQ ID NO:21:

(SEQ ID NO: 21)
MPMGSLQPLATLYLLGMLVASCLGTANADTLCIGYHANNSTDTVDTVLEK
NVTVTHSVNLLEDKHNGKLCKLRGVAPLHLGKCNIAGWILGNPECESLST
ARSWSYIVETSNSDNGTCYPGDFINYEELREQLSSVSSFERFEIFPKTSS
WPNHDSDKGVTAACPHAGAKSFYKNLIWLVKKGNSYPKLNQTYINDKGKE
VLVLWGIHHPPTIAAQESLYQNADAYVFVGTSRYSKKFKPEIATRPKVRD
QEGRMNYYWTLVEPGDKITFEATGNLVVPRYAFTMERDAGSGIIISDTPV
HDCNTTCQTPEGAINTSLPFQNVHPITIGKCPKYVKSTKLRLATGLRNVP
SIQSRGLFGAIAGFIEGGWTGMVDGWYGYH
HQNEQGSGYAADLKSTQNAIDKITNKVNSV
IEKMNTQFTAVGKEFNHLEKRIENLNKKVD
DGFLDIWTYNAELLVLLENERTLDYHDSNV
KNLYEKVRNQLKNNAKEIGNGCFEFYHKCD
NTCMESVKNGTYDYPKYSEEAKLNREKIDG
VKLESTRIYQILAIYSTVASSLVLVVSLGA
ISFWMCSNGSLQCRICI.

The sequence of Influenza A/H3 HA S protein with the HA2 fragment bolded and underlined is provided as SEQ ID NO:22:

(SEQ ID NO: 22)
MPMGSLQPLATLYLLGMLVASCLGQKIPGNDNSTATLCLGHHAVPNGTIV
KTITNDRIEVTNATELVQNSSIGEICDSPHQILDGGNCTLIDALLGDPQC
DGFQNKKWDLFVERSRAYSNCYPYDVPDYASLRSLVASSGTLEFKNESFN
WAGVTQNGKSFSCIRGSSSSFFSRLNWLTHLNYTYPALNVTMPNKEQFDK
LYIWGVHHPGTDKDQISLYAQSSGRITVSTKRSQQAVIPNIGSRPRIRDI
PSRISIYWTIVKPGDILLINSTGNLIAPRGYFKIRSGKSSIMRSDAPIGK
CKSECITPNGSIPNDKPFQNVNRITYGACPRYVKQNTLKLATGMRNVPEK
QTRGIFGAIAGFIENGWEGMVDGWYGFRHQ
NSEGRGQAADLKSTQAAIDQINGKLNRLIG
KTNEKFHQIEKEFSEVEGRVQDLEKYVEDT
KIDLWSYNAELLVALENQHTIDLTDSEMNK
LFEKTKKQLRENAEDMGNGCFKIYHKCDNA
CIGSIRNETYDHNVYRDEALNNRFQIKGVE
LKSGYKDWILWISFAISCFLLCVALLGFIM
WACQKGNIRCNICI.

The sequence of Influenza B/Victoria HA S protein with the HA2 fragment bolded and underlined is provided as SEQ ID NO:23:

(SEQ ID NO: 23)
MPMGSLQPLATLYLLGMLVASCLGTSNADRICTGITSSNSPHVVKTATQG
EVNVTGVIPLTTTPTKSHFANLKGTETRGKLCPKCLNCTDLDVALGRPKC
TGKIPSARVSILHEVRPVTSGCFPIMHDRTKIRQLPNLLRGYEHVRLSTH
NVINAEDAPGRPYEIGTSGSCPNITNGNGFFATMAWAVPKNKTATNPLTI
EVPYICTEGEDQITVWGFHSDSETQMAKLYGDSKPQKFTSSANGVTTHYV
SQIGGFPNQTEDGGLPQSGRIVVDYMVQKSGKTGTITYQRGILLPQKVWC
ASGRSKVIKGSLPLIGEADCLHEKYGGLNKSKPYYTGEHAKAIGNCPIWV
KTPLKLANGTKYRPPAKLLKERGFFGAIAG
FLEGGWEGMIAGWHGYTSHGAHGVAVAADL
KSTQEAINKITKNLNSLSELEVKNLQRLSG
AMDELHNEILELDEKVDDLRADTISSQIEL
AVLLSNEGIINSEDEHLLALERKLKKMLGP
SAVEIGNGCFETKHKCNQTCLDRIAAGTFD
AGEFSLPTFDSLNITAASLNDDGLDNHTIL
LYYSTAASSLAVTLMIAIFVVYMVSRDNVS
CSICL.

The sequence of Influenza B/Yamagata HA S protein with the HA2 fragment bolded and underlined is provided as SEQ ID NO:24:

(SEQ ID NO: 24)
MPMGSLQPLATLYLLGMLVASCLGTSNADRICTGITSSNSPHVVKTATQG
EVNVTGVIPLTTTPTKSHFANLKGTETRGKLCPKCLNCTDLDVALGRPKC
TGKIPSARVSILHEVRPVTSGCFPIMHDRTKIRQLPNLLRGYEHVRLSTH
NVINAEDAPGRPYEIGTSGSCPNITNGNGFFATMAWAVPKNKTATNPLTI
EVPYICTEGEDQITVWGFHSDSETQMAKLYGDSKPQKFTSSANGVTTHYV
SQIGGFPNQTEDGGLPQSGRIVVDYMVQKSGKTGTITYQRGILLPQKVWC
ASGRSKVIKGSLPLIGEADCLHEKYGGLNKSKPYYTGEHAKAIGNCPIWV
KTPLKLANGTKYRPPAKLLKERGFFGAIAG
FLEGGWEGMIAGWHGYTSHGAHGVAVAADL
KSTQEAINKITKNLNSLSELEVKNLQRLSG
AMDELHNEILELDEKVDDLRADTISSQIEL
AVLLSNEGIINSEDEHLLALERKLKKMLGP
SAVEIGNGCFETKHKCNQTCLDRIAAGTFD
AGEFSLPTFDSLNITAASLNDDGLDNHTIL
LYYSTAASSLAVTLMIAIFVVYMVSRDNVS
CSICL.

In some embodiments, the polypeptides of the disclosure are derived from the influenza virus HA S protein. In some embodiments, the polypeptide derived from the influenza virus HA S protein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with bolded and SEQ ID NO:25, or a fragment or functional derivative thereof.

In some embodiments, the polypeptides derived from the Influenza HA S protein, for example, the HA2 fragment of the Influenza HA S protein corresponding to SEQ ID NO:25, interact with an influenza virus HA spike (S) protein, for example, an influenza virus A S protein, an influenza virus B S protein, or an influenza virus C S protein. In some embodiments, the influenza virus HA S protein comprises an influenza virus A/H1 HA S protein, an influenza virus A/H3 HA S protein, an influenza virus B/Victoria HA S protein, and/or an influenza virus B/Yamagata HA S protein. In some embodiments, the polypeptides derived from the influenza virus HA S protein interact with an influenza virus A S protein, for example, an influenza virus A/H1 HA S protein and/or an influenza virus A/H3 HA S protein. In some embodiments, the polypeptides derived from the influenza virus HA S protein interact with an influenza virus B S protein, for example, an influenza virus B/Victoria HA S protein and/or an influenza virus B/Yamagata HA S protein. In some embodiments, the polypeptides derived from the influenza virus HA S protein interact with an influenza virus C S protein.

In some embodiments, the polypeptides derived from the influenza virus HA S protein, for example, the HA2 fragment of the Influenza HA S protein corresponding to SEQ ID NO:25, oligomerize with an influenza virus HA spike (S) protein, for example, an influenza virus A S protein, an influenza virus B S protein, or an influenza virus C S protein. In some embodiments, the influenza virus HA S protein comprises an influenza virus A/H1 HA S protein, an influenza virus A/H3 HA S protein, an influenza virus B/Victoria HA S protein, and/or an influenza virus B/Yamagata HA S protein. In some embodiments, the polypeptides derived from the influenza virus HA S protein oligomerize with an influenza virus A S protein, for example, an influenza virus A/H1 HA S protein and/or an influenza virus A/H3 HA S protein. In some embodiments, the polypeptides derived from the influenza virus HA S protein oligomerize with an influenza virus B S protein, for example, an influenza virus B/Victoria HA S protein and/or an influenza virus B/Yamagata HA S protein. In some embodiments, the polypeptides derived from the influenza virus HA S protein oligomerize with an influenza virus C S protein.

### E. Respiratory Syncytial Virus

In particular embodiments, the virus is from the family Pneumoviridae. Pneumoviridae is a family of negative-strand RNA viruses in the order Mononegavirales. Pneumoviridae are classified into two genera, including Metapneumovirus and Orthopneumovirus.

Pneumoviruses replicate in the cytoplasm of the host cell. First, the virus binds to HN glycoprotein receptors expressed on the surface of the cell. Then, through the action of the fusion protein, the virus fuses to the host plasma membrane and the nucleocapsid is released. Prior to undergoing replication, mRNA is transcribed and viral proteins are translated. Transcription is dependent on virally encoded RNA-dependent-RNA-polymerase, which binds the genome at the 3′ leader region and then sequentially transcribes each gene. Translation of viral proteins is carried out by host cell ribosomes. Once sufficient P, N, L, and M2 proteins are available to create a capsid around the newly replicated genome, the virus undergoes replication. After replication, the P, L, and M proteins participate in forming the ribonucleocapsid. Once virion assembly is complete, the virion egresses by budding out of the cell.

Respiratory Syncytial Virus (RSV), also called human respiratory syncytial virus (hRSV) and human orthopneumovirus, is a common, contagious virus that causes infections of the respiratory tract. In some aspects, the RSV is RSV type A. In some aspects, the RSV is RSV type B. It is a negative-sense, single-stranded RNA virus, and its name is derived from the large cells known as syncytia that form when infected cells fuse. RSV is a common cause of respiratory hospitalization in infants, and reinfection remains common in later life such that it is an important pathogen in all age groups. Infection rates are typically higher during the cold winter months, causing bronchiolitis in infants, common colds in adults, and more serious respiratory illnesses such as pneumonia in the elderly and immunocompromised.

RSV is a medium-sized (˜150 nm) enveloped virus. The genome rests within a helical nucleocapsid and is surrounded by matrix protein and an envelope containing viral glycoproteins. The genome is linear and approximately 15,000 nucleotides in length. While most particles are spherical, filamentous species have also been identified. RSV genomes contain 10 genes encoding for 11 proteins: lipid envelope proteins (glycoprotein (G), fusion protein (F), small hydrophobic protein (SH)), inner envelope face matrix protein (M), ribonucleocapsid proteins (nucleoprotein (N), phosphoprotein (P), large protein (L), M2-1), regulatory M2-2, and nonstructural proteins (NS-1, and NS-2). The gene order is NS1-NS2-N-P-M-SH-G-F-M2-L.

RSV infection can present with a wide variety of signs and symptoms that range from mild upper respiratory tract infections to severe and potentially life-threatening lower respiratory tract infections (e.g., bronchiolitis, viral pneumonia, or croup) requiring hospitalization and mechanical ventilation. Most RSV infections include common cold, sinus, and/or upper respiratory tract signs and symptoms, such as nasal congestion, runny nose, cough, malaise, sore throat, and low-grade fever. Inflammation of the nasal mucosa (rhinitis) and throat (pharyngitis), as well as redness of the eyes (conjunctival infection), may be seen on exam.

RSV can spread when an infected person coughs or sneezes, and droplets are passed into the eyes, nose, or mouth of another individual, or the individual touches a surface with the virus on it and then touches their face before washing their hands, or the individual directly contacts the virus. People infected with RSV are usually contagious for 3 to 8 days, and up to 4 weeks in some cases. People at highest risk for severe disease include premature infants, young children with congenital (from birth) heart or chronic lung disease, young children with compromised (weakened) immune systems due to a medical condition or medical treatment, adults with compromised immune systems, and older adults, especially those with underlying heart or lung disease.

A variety of laboratory tests are available for the diagnosis of RSV infection, including, without limitation, antigen testing, molecular testing, viral culture, and serologic testing. Chest x-rays may also be performed to identify perihilar markings, patchy hyperinflation, and/or atelectasis.

The present disclosure encompasses treatment or prevention of infection of any virus in the Pneumoviridae family and including the two genera, Metapneumovirus and Orthopneumovirus. In specific embodiments, the disclosure encompasses treatment or prevention of infection of any virus in the genus of Orthopneumovirus, including the species human orthopneumovirus (RSV type A or B) that infects humans.

Aspects of the disclosure relate to polypeptides that interact with an RSV glycoprotein. Following transmission through the nose or eyes, RSV infects ciliated columnar epithelial cells of the upper and lower airway. RSV continues to replicate within these bronchial cells for about 8 days. After the first several days, RSV-infected cells will become more rounded and ultimately slough into the smaller bronchioles of the lower airway. This sloughing mechanism is also thought to be responsible for the spread of virus from the upper to lower respiratory tract. Infection causes generalized inflammation within the lungs, including the migration and infiltration of inflammatory cells (such as monocytes and T-cells), necrosis of the epithelial cell wall, edema, and increased mucous production. Together, the sloughed epithelial cells, mucous plugs, and accumulated immune cells cause obstruction of the lower airway.

Glycoprotein F (surface fusion protein) is responsible for fusion of viral & host cell membranes, as well as syncytium formation between viral particles. F protein exists in multiple conformational forms. In the prefusion state (PreF), the protein exists in a trimeric form. After binding to its target on the host cell surface, PreF undergoes a conformational change that enables the protein to insert itself into the host cell membrane and leads to fusion of the viral and host cell membranes. A final conformational shift results in a more stable and elongated form of the protein (postfusion, PostF). RSV F protein also binds to and activates toll-like receptor 4, initiating the innate immune response and signal transduction.

Following fusion of the viral and host cell membranes, the viral nucleocapsid (containing the viral genome) and the associated viral polymerase are delivered into the host cell cytoplasm. Transcription and translation both occur within the cytoplasm. RNA-dependent RNA polymerase transcribes the genome into 10 segments of messenger RNA (mRNA) which is translated into structural proteins by host cell machinery. During replication of the negative-sense viral genome, RNA-dependent RNA polymerase synthesizes a positive-sense complement called the antigenome. This complementary strand is used as a template to construct genomic negative-sense RNA, which is packaged into nucleocapsids and transported to the plasma membrane for assembly and particle budding.

The sequence of RSV F glycoprotein with the F fragment portions bolded and underlined is provided as SEQ ID NO:33:

(SEQ ID NO: 33)
MELPILKTNAIITILAAVTLCFASS
QNITEEFYQSTCSAVSKGYLSALRTG
WYTSVITIELSNIKENKCNGTDAKVKLIKQELDKYKN
AVTELQLLMQSTPAANSRARRELPRFMNYTLNNTKNT
NVTLSKKRKRRFLGFLLGVGSAIASGIAVSKVLHLEG
EVNKIKSALLSTNKAVVSLSNGVSVLTSKVLDLKNYI
DKQLLPIVNKQSCSISNIETVIEFQQKNNRLLEITRE
FSVNAGVTTPVSTYMLTNSELLSLINDMPITNDQKKL
MSSNVQIVRQQSYSIMSIIKEEVLAYVVQLPLY
GVIDTPCWKLHTSPLCTTNTKEGSNICLTR
TDRGWYCDNAGSVSFFPQAETCKVQSNRVF
CDTMNSLTLPSEVNLCNIDIFNPKYDCKIM
TSKTDVSSSVITSLGAIVSCYGKTKCTASN
KNRGIIKTFSNGCDYVSNKGVDTVSVGNTL
YYVNKQEGKSLYVKGEPIINFYDPLVFPSD
EFDASISQVNEKINQSLAFIRKSDELLHNV
NAGKSTTNIMITTIIIVIIVILLALIAVGL
LLYCKARSTPVTLSKDQLSGINNIAFSN.

In some embodiments, the polypeptides of the disclosure are derived from the RSV glycoprotein. In some embodiments, the polypeptide derived from the RSV glycoprotein has at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 52%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, or any value derivable therein, with the bolded and underlined portions of SEQ ID NO:33 that are comprised in SEQ ID NO:34, or a fragment or functional derivative thereof.

In some embodiments, polypeptides derived from the RSV glycoprotein, for example, SEQ ID NO:34, interact with a Pneumoviridae glycoprotein. In some embodiments, polypeptides derived from the RSV glycoprotein, for example, SEQ ID NO:34, interact with an RSV glycoprotein. In some embodiments, polypeptides derived from the RSV glycoprotein, for example, SEQ ID NO:34, oligomerize with a Pneumoviridae glycoprotein. In some embodiments, polypeptides derived from the RSV glycoprotein, for example, SEQ ID NO:34, oligomerize with an RSV glycoprotein.

### IV. Anti-Viral Treatment

Compositions (e.g., viral protein-interacting polypeptides or polynucleotides encoding viral protein-interacting polypeptides) or methods described herein may be administered to any subject having a condition in which interacting with, and in some cases inhibiting or interfering with, viral proteins, such as virus spike proteins, may have therapeutic benefit. Conditions in which targeting viral proteins may have a therapeutic benefit include, for example, a condition associated with binding of viral particles to cells and entry of viral particles into cells. Such conditions include, for example, coronavirus infections or post-coronavirus infections syndrome, HIV infections, Ebola infections, RSV infections, and/or influenza infections.

As used herein, “coronavirus infection” refers to an infection caused by any Coronaviridae family member. For example, coronavirus infections can include but are not limited to MERS-CoV infections, HCoV-229E infections, HCoV-NL63 infections, HCoV-OC43 infections, HCoV-HKU1 infections, SARS-CoV infections, and SARS-CoV-2 infections. Thus, aspects of the present disclosure are directed to methods comprising treatment of a subject suffering from, or suspected of having, or at risk of having, a coronavirus infection. In some embodiments, the coronavirus infection is a MERS-CoV infection. In some embodiments, the coronavirus infection is an HCoV-229E infection. In some embodiments, the coronavirus infection is an HCoV-NL63 infection. In some embodiments, the coronavirus infection is an HCoV-OC43 infection. In some embodiments, the coronavirus infection is an HCoV-HKU1 infection. In some embodiments, the coronavirus infection is a SARS-CoV infection. In some embodiments, the coronavirus infection is a SARS-CoV-2 infection.

As used herein, “HIV infection” refers to an infection caused by the retrovirus human immunodeficiency virus. Thus, aspects of the present disclosure are directed to methods comprising treatment of a subject suffering from, or suspected of having, or at risk of having, an HIV infection.

As used herein, “Ebola infection” refers to an infection caused by an Ebolavirus. For example, the Ebola infections can include but are not limited to Ebola virus infections (species Zaire ebolavirus), Sudan virus infections, Tai Forest virus infections, and/or Bundibugyo virus infections. In some embodiments, the Ebola infection is an Ebola virus infection. In some embodiments, the Ebola infection is a Sudan virus infection. In some embodiments, the Ebola infection is a Tai Forest virus infection. In some embodiments, the Ebola infection is a Bundibugyo virus infection. Thus, aspects of the present disclosure are directed to methods comprising treatment of a subject suffering from, or suspected of having, an Ebola infection.

As used herein, “influenza infection” refers to an infection caused by an influenza virus. For example, the influenza virus can include any virus in the genera of Orthomyxoviridae family that infects humans, including Alphainfluenzaviruses, Betainfluenzaviruses, and Gammainfluenzaviruses. In specific embodiments, the influenza virus comprises an Alphainfluenzavirus, including the species influenza virus A and any influenza virus A strains, subtypes, or lineages. In specific embodiments, the influenza virus A comprises Influenza A virus subtype H1N1, Influenza A virus subtype H1N2, Influenza A virus subtype H2N2, Influenza A virus subtype H2N3, Influenza A virus subtype H3N1, Influenza A virus subtype H3N2, Influenza A virus subtype H3N8, Influenza A virus subtype H5N1, Influenza A virus subtype H5N2, Influenza A virus subtype H5N3, Influenza A virus subtype H5N6, Influenza A virus subtype H5N8, Influenza A virus subtype H5N9, Influenza A virus subtype H6N1, Influenza A virus subtype H6N2, Influenza A virus subtype H7N1, Influenza A virus subtype H7N2, Influenza A virus subtype H7N3, Influenza A virus subtype H7N4, Influenza A virus subtype H7N7, Influenza A virus subtype H7N9, Influenza A virus subtype H9N2, Influenza A virus subtype H10N7, Influenza A virus subtype H10N8, Influenza A virus subtype H11N2, Influenza A virus subtype H11N9, Influenza A virus subtype H17N10, Influenza A virus subtype H18N11, or a combination thereof. In specific embodiments, the influenza virus comprises a Betainfluenzavirus, including the species influenza virus B and any influenza virus B strains, subtypes, or lineages. In specific embodiments, the influenza virus B comprises influenza virus B/Yamagata and/or influenza virus B/Victoria. In specific embodiments, the influenza virus comprises a Gammainfluenzavirus, including the species influenza virus C and any influenza virus C strains, subtypes, or lineages. Thus, aspects of the present disclosure are directed to methods comprising treatment of a subject suffering from, or suspected of having, or at risk of having, an influenza infection.

As used herein, “RSV infection” refers to an infection caused by the human orthopneumovirus. Thus, aspects of the present disclosure are directed to methods comprising treatment of a subject suffering from, or suspected of having, or at risk of having, an RSV infection.

In specific embodiments, the methods and compositions comprise treating, preventing, delaying onset of, and/or reducing severity of viral infections, for example, coronavirus infections, HIV infections, Ebola infections, RSV infections, and/or influenza infections, in a subject in need thereof by administering an effective amount of a viral protein-interacting polypeptide therapy or a polynucleotide encoding a protein-interacting polypeptide. In specific embodiments, the effective amount is effective to treat, prevent, delay onset of, and/or reduce severity of a viral infection in the subject. In specific embodiments, the methods and compositions further comprise increasing the survival rate of a subject infected with a virus. In specific embodiments, the methods and compositions further comprise reducing the recovery time of a subject infected with a virus. In specific embodiments, the methods and compositions further comprise treating, preventing, delaying onset of, and/or reducing severity of the symptoms of a virus infection in a subject. In specific embodiments, the methods and compositions further comprise treating, preventing, delaying onset of, and/or reducing severity of cellular, tissue, organ, or system damage caused by a virus infection in a subject.

In specific embodiments, the methods and compositions further comprise treating, preventing, delaying onset of, and/or reducing severity of post-viral infection syndrome in a subject in need thereof by administering an effective amount of a protein-interacting polypeptide therapy or a polynucleotide encoding a protein-interacting polypeptide. In specific embodiments, the effective amount is effective to treat, prevent, delay onset of, and/or reduce severity of post-viral infection syndrome in the individual. In specific embodiments, the effective amount is effective to treat, prevent, delay onset of, and/or reduce severity of persistent symptoms of a viral infection in a subject. In specific embodiments, the effective amount is effective to treat, prevent, delay onset of, and/or reduce severity of chronic effects of cellular, tissue, organ, or system damage caused by a viral infection in the individual. In specific embodiments, the methods and compositions further comprise increasing the survival rate of a subject with post-viral infection syndrome. In specific embodiments, the methods and compositions further comprise increasing the survival rate of a subject with persistent symptoms of a viral infection. In specific embodiments, the methods and compositions further comprise increasing the survival rate of a subject with chronic effects of cellular, tissue, organ, or system damage caused by a viral infection. In specific embodiments, the methods and compositions further comprise reducing the recovery time of a subject with post-viral infection syndrome. In specific embodiments, the methods and compositions further comprise reducing the recovery time of a subject with persistent symptoms of a viral infection. In specific embodiments, the methods and compositions further comprise reducing the recovery time of a subject with chronic effects of cellular, tissue, organ, or system damage caused by a viral infection.

In some embodiments of the methods disclosed herein, the subject is at high risk for having a viral infection. In some embodiments, the subject does not have a viral infection or has tested negative for a viral infection. In some embodiments, the subject was diagnosed as having a viral infection. In some embodiments, the subject is diagnosed as having symptoms of the viral infection. In some embodiments, the subject is diagnosed as being at risk of having the viral infection.

In specific embodiments, the methods and compositions comprise treating, preventing, delaying onset of, and/or reducing severity of coronavirus infections in a subject in need thereof by administering an effective amount of a coronavirus S protein-interacting polypeptide therapy or a polynucleotide encoding a coronavirus S protein-interacting polypeptide. In specific embodiments, the effective amount is effective to treat, prevent, delay onset of, and/or reduce severity of a coronavirus infection in the subject. In specific embodiments, the methods and compositions further comprise increasing the survival rate of a subject infected with a coronavirus. In specific embodiments, the methods and compositions further comprise reducing the recovery time of a subject infected with a coronavirus. In specific embodiments, the methods and compositions further comprise treating, preventing, delaying onset of, and/or reducing severity of the symptoms of a coronavirus infection in a subject. In specific embodiments, the methods and compositions further comprise treating, preventing, delaying onset of, and/or reducing severity of cellular, tissue, organ, or system damage caused by a coronavirus infection in a subject.

In specific embodiments, the methods and compositions further comprise treating, preventing, delaying onset of, and/or reducing severity of post-coronavirus infection syndrome in a subject in need thereof by administering an effective amount of a protein-interacting polypeptide therapy, such as a coronavirus S protein-interacting polypeptide therapy, or a polynucleotide encoding a protein-interacting polypeptide, such as a coronavirus S protein-interacting polypeptide. In specific embodiments, the effective amount is effective to treat, prevent, delay onset of, and/or reduce severity of post-coronavirus infection syndrome in the individual. In specific embodiments, the effective amount is effective to treat, prevent, delay onset of, and/or reduce severity of persistent symptoms of a coronavirus infection in a subject. In specific embodiments, the effective amount is effective to treat, prevent, delay onset of, and/or reduce severity of chronic effects of cellular, tissue, organ, or system damage caused by a coronavirus infection in the individual. In specific embodiments, the methods and compositions further comprise increasing the survival rate of a subject with post-coronavirus infection syndrome. In specific embodiments, the methods and compositions further comprise increasing the survival rate of a subject with persistent symptoms of a coronavirus infection. In specific embodiments, the methods and compositions further comprise increasing the survival rate of a subject with chronic effects of cellular, tissue, organ, or system damage caused by a coronavirus infection. In specific embodiments, the methods and compositions further comprise reducing the recovery time of a subject with post-coronavirus infection syndrome. In specific embodiments, the methods and compositions further comprise reducing the recovery time of a subject with persistent symptoms of a coronavirus infection. In specific embodiments, the methods and compositions further comprise reducing the recovery time of a subject with chronic effects of cellular, tissue, organ, or system damage caused by a coronavirus infection.

A subject in need thereof may be a subject having one or more symptoms of infection by a virus of the Coronaviridae family, such as HCoV-229E, HCoV-NL63, HCoV-OC43, HCoV-HKU1, SARS-CoV, SARS-CoV-2, or MERS-CoV. Common initial signs and symptoms of HCoV-229E, HCoV-NL63, HCoV-OC43, HCoV-HKU1, SARS-CoV, SARS-CoV-2, and/or MERS-CoV infections may include fever, cough, shortness of breath or difficulty breathing, tiredness, aches, chills, sore throat, loss of smell, loss of taste headache, diarrhea, or vomiting. As the viral infection progresses, the individual may develop pneumonia or acute respiratory distress syndrome (ARDS). In some embodiments, the virus is SARS-CoV-2, and in certain embodiments the virus is not SARS-CoV, MERS-CoV, HCoV-229E, HCoV-NL63, HCoV-OC43, or HCoV-HKU1. In some embodiments, the virus is SARS-CoV, and in certain embodiments the virus is not SARS-CoV-2, MERS-CoV, HCoV-229E, HCoV-NL63, HCoV-OC43, or HCoV-HKU1. In some embodiments, the virus is MERS-CoV, and in certain embodiments the virus is not SARS-CoV, SARS-CoV-2, HCoV-229E, HCoV-NL63, HCoV-OC43, or HCoV-HKU1. In some embodiments, the virus is HCoV-229E, and in certain embodiments the virus is not SARS-CoV, SARS-CoV-2, MERS-CoV, HCoV-NL63, HCoV-OC43, or HCoV-HKU1. In some embodiments, the virus is HCoV-NL63, and in certain embodiments the virus is not SARS-CoV, SARS-CoV-2, MERS-CoV, HCoV-229E, HCoV-OC43, or HCoV-HKU1. In some embodiments, the virus is HCoV-OC43, and in certain embodiments the virus is not SARS-CoV, SARS-CoV-2, MERS-CoV, HCoV-229E, HCoV-NL63, or HCoV-HKU1. In some embodiments, the virus is HCoV-HKU1, and in certain embodiments the virus is not SARS-CoV, SARS-CoV-2, MERS-CoV, HCoV-229E, HCoV-NL63, or HCoV-OC43.

In some embodiments of the methods disclosed herein, the subject is at high risk for having coronavirus infection and/or post-coronavirus infection syndrome. In some embodiments, the subject does not have a coronavirus infection or has tested negative for a coronavirus infection. In some embodiments, the subject was diagnosed as having a coronavirus infection and/or post-coronavirus infection syndrome. In some embodiments, the subject is diagnosed as having symptoms of the coronavirus infection and/or post-coronavirus infection syndrome. In some embodiments, the subject is diagnosed as being at risk of having the coronavirus infection and/or post-coronavirus infection syndrome. In some embodiments, the subject has severe acute respiratory syndrome (SARS), Middle East respiratory syndrome, or a respiratory infection. In some embodiments, the subject has COVID-19.

In specific embodiments, the methods and compositions comprise treating, preventing, delaying onset of, and/or reducing severity of HIV infections in a subject in need thereof by administering an effective amount of an HIV S protein-interacting polypeptide therapy or a polynucleotide encoding an HIV S protein-interacting polypeptide. In specific embodiments, the effective amount is effective to treat, prevent, delay onset of, and/or reduce severity of an HIV infection in the subject. In specific embodiments, the methods and compositions further comprise increasing the survival rate of a subject infected with HIV. In specific embodiments, the methods and compositions further comprise reducing the recovery time of a subject infected with HIV. In specific embodiments, the methods and compositions further comprise treating, preventing, delaying onset of, and/or reducing severity of the symptoms of an HIV infection in a subject. In specific embodiments, the methods and compositions further comprise treating, preventing, delaying onset of, and/or reducing severity of cellular, tissue, organ, or system damage caused by HIV infection in a subject.

A subject in need thereof may be a subject having one or more symptoms of HIV infection. Common initial signs and symptoms of HIV infection may include fever, headache, chills, rash, night sweats, muscle aches and joint pain, sore throat, fatigue, diarrhea, weight loss, cough, swollen lymph nodes, mouth ulcers, oral yeast infection (thrush), shingles (herpes zoster), and pneumonia. As the viral infection progresses, the individual may develop sweats, chills, recurring fever, chronic diarrhea, swollen lymph glands, persistent white spots or unusual lesions on the tongue or in the mouth, persistent, unexplained fatigue, weakness, weight loss, and skin rashes or bumps.

In some embodiments of the methods disclosed herein, the subject is at high risk for having HIV infection. In some embodiments, the subject does not have an HIV infection or has tested negative for an HIV infection. In some embodiments, the subject was diagnosed as having HIV. In some embodiments, the subject is diagnosed as having symptoms of the HIV infection. In some embodiments, the subject is diagnosed as being at risk of having the HIV infection.

In specific embodiments, the methods and compositions comprise treating, preventing, delaying onset of, and/or reducing severity of Ebola infections in a subject in need thereof by administering an effective amount of an Ebola virus glycoprotein-interacting polypeptide therapy or a polynucleotide encoding an Ebola virus glycoprotein-interacting polypeptide. In specific embodiments, the effective amount is effective to treat, prevent, delay onset of, and/or reduce severity of an Ebola infection in the subject. In specific embodiments, the methods and compositions further comprise increasing the survival rate of a subject infected with Ebola. In specific embodiments, the methods and compositions further comprise reducing the recovery time of a subject infected with Ebola. In specific embodiments, the methods and compositions further comprise treating, preventing, delaying onset of, and/or reducing severity of the symptoms of an Ebola infection in a subject. In specific embodiments, the methods and compositions further comprise treating, preventing, delaying onset of, and/or reducing severity of cellular, tissue, organ, or system damage caused by Ebola infection in a subject.

A subject in need thereof may be a subject having one or more symptoms of infection by an Ebolavirus. Common initial signs and symptoms of an Ebola infection may include fever, sore throat, muscular pain, weakness, fatigue, and headaches. Vomiting, diarrhea, abdominal pain, and rash usually follow, along with decreased function of the liver and kidneys. At this time, some people begin to bleed both internally and externally.

In some embodiments of the methods disclosed herein, the subject is at high risk for having Ebola infection. In some embodiments, the subject does not have an Ebola infection or has tested negative for an Ebola infection. In some embodiments, the subject was diagnosed as having Ebola. In some embodiments, the subject is diagnosed as having symptoms of the Ebola infection. In some embodiments, the subject is diagnosed as being at risk of having the Ebola infection.

In specific embodiments, the methods and compositions comprise treating, preventing, delaying onset of, and/or reducing severity of influenza infections in a subject in need thereof by administering an effective amount of an influenza viral S protein-interacting polypeptide therapy or a polynucleotide encoding an influenza viral S protein-interacting polypeptide. In specific embodiments, the effective amount is effective to treat, prevent, delay onset of, and/or reduce severity of an influenza infection in the subject. In specific embodiments, the methods and compositions further comprise increasing the survival rate of a subject infected with influenza. In specific embodiments, the methods and compositions further comprise reducing the recovery time of a subject infected with influenza. In specific embodiments, the methods and compositions further comprise treating, preventing, delaying onset of, and/or reducing severity of the symptoms of an influenza infection in a subject. In specific embodiments, the methods and compositions further comprise treating, preventing, delaying onset of, and/or reducing severity of cellular, tissue, organ, or system damage caused by influenza infection in a subject.

A subject in need thereof may be a subject having one or more symptoms of infection by a virus of the Orthomyxoviridae family, such as influenza virus A, influenza virus B, or influenza virus C virus. Common signs and symptoms of influenza infections may include fever, chills, headaches, muscle pain or aching, a feeling of discomfort, loss of appetite, lack of energy/fatigue, and confusion. These symptoms are usually accompanied by respiratory symptoms such as a dry cough, sore or dry throat, hoarse voice, and a stuffy or runny nose. Coughing is the most common symptom. Gastrointestinal symptoms may also occur, including nausea, vomiting, diarrhea, and gastroenteritis.

In some embodiments of the methods disclosed herein, the subject is at high risk for having influenza infection. In some embodiments, the subject does not have an influenza infection or has tested negative for an influenza infection. In some embodiments, the subject was diagnosed as having influenza. In some embodiments, the subject is diagnosed as having symptoms of the influenza infection. In some embodiments, the subject is diagnosed as being at risk of having the influenza infection.

In specific embodiments, the methods and compositions comprise treating, preventing, delaying onset of, and/or reducing severity of RSV infections in a subject in need thereof by administering an effective amount of an RSV glycoprotein-interacting polypeptide therapy or a polynucleotide encoding an RSV glycoprotein-interacting polypeptide. In specific embodiments, the effective amount is effective to treat, prevent, delay onset of, and/or reduce severity of an RSV infection in the subject. In specific embodiments, the methods and compositions further comprise increasing the survival rate of a subject infected with RSV. In specific embodiments, the methods and compositions further comprise reducing the recovery time of a subject infected with RSV. In specific embodiments, the methods and compositions further comprise treating, preventing, delaying onset of, and/or reducing severity of the symptoms of an RSV infection in a subject. In specific embodiments, the methods and compositions further comprise treating, preventing, delaying onset of, and/or reducing severity of cellular, tissue, organ, or system damage caused by RSV infection in a subject.

A subject in need thereof may be a subject having one or more symptoms of RSV infection. Common initial signs and symptoms of RSV infection may include common cold, sinus, and/or upper respiratory tract signs and symptoms, such as nasal congestion, runny nose, cough, malaise, sore throat, and low-grade fever.

In some embodiments of the methods disclosed herein, the subject is at high risk for having RSV infection. In some embodiments, the subject does not have an RSV infection or has tested negative for an RSV infection. In some embodiments, the subject was diagnosed as having RSV. In some embodiments, the subject is diagnosed as having symptoms of the RSV infection. In some embodiments, the subject is diagnosed as being at risk of having the RSV infection.

In specific embodiments, a subject may be diagnosed with a viral infection based on the onset of symptoms of the viral infection; and/or based on a positive biological test for a current viral infection. In specific embodiments, the biological test for a current viral infection is an assay for the virus. In specific embodiments, a subject may be considered recovered from a viral infection based on the amount of time which has passed since the onset of symptoms of the viral infection, the amount of time which has passed without a fever in the absence of use of fever-reducing medication, and the improvement of other symptoms of the viral infection; and/or two consecutive negative biological tests for a current viral infection taken at least a certain time period apart. In specific embodiments, a subject may be considered recovered from a virus infection if at least 10 days have passed since virus infection symptoms first appeared, at least 24 hours have passed with no fever without the use of fever-reducing medications, and other symptoms of virus infection are improving; and/or two biological tests for a current virus infection taken at least 24 hours apart are both negative. In specific embodiments, a subject may confirm a previous viral infection based on a biological test for a past viral infection. In specific embodiments, the biological test for a past viral infection is an assay for viral antibodies. In specific embodiments, a subject considered recovered from a viral infection may be diagnosed with a post-viral infection syndrome based on persistent symptoms of the viral infection and/or chronic effects of cellular, tissue, organ, or system damage caused by the viral infection. In specific embodiments, persistent symptoms of a coronavirus infection and/or chronic effects of cellular, tissue, organ, or system damage caused a coronavirus infection include persistent fever, cough, shortness of breath, difficulty breathing, tiredness, aches, chills, sore throat, loss of smell, loss of taste, headache, diarrhea, vomiting, pneumonia, acute respiratory distress syndrome (ARDS), dizziness, mood disorders, cognitive impairment, muscle weakness, nerve damage, joint pain, chest pain, palpitations, rash, hair loss, worsened quality of life, lung damage, heart damage, heart swelling, kidney damage, or liver damage. In specific embodiments, a subject in need thereof may be a subject having one or more persistent symptoms of a viral infection. Common persistent symptoms of a viral infections and/or chronic effects of chronic effects of cellular, tissue, organ, or system damage caused by a viral infection may include persistent fever, cough, shortness of breath, difficulty breathing, tiredness, aches, chills, sore throat, loss of smell, loss of taste, headache, diarrhea, vomiting, pneumonia, acute respiratory distress syndrome (ARDS), dizziness, mood disorders, cognitive impairment, muscle weakness, nerve damage, joint pain, chest pain, palpitations, rash, hair loss, worsened quality of life, lung damage, heart damage, heart swelling, kidney damage, or liver damage.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance.

The therapy provided herein may comprise administration of a composition comprising a therapeutic agent (e.g., viral protein-interacting polypeptides or polynucleotides encoding viral protein-interacting polypeptides). In some embodiments, therapy provided herein comprises administration of coronavirus S protein-interacting polypeptides and a pharmaceutically acceptable excipient. In some embodiments, therapy provided herein comprises administration of a nucleic acid encoding for the coronavirus S protein-interacting polypeptides and a pharmaceutically acceptable excipient. In some embodiments, therapy provided herein comprises administration of a vector comprising the nucleic acid encoding for the coronavirus S protein-interacting polypeptides and a pharmaceutically acceptable excipient. In some embodiments, therapy provided herein comprises administration of HIV S protein-interacting polypeptides and a pharmaceutically acceptable excipient. In some embodiments, therapy provided herein comprises administration of a nucleic acid encoding for the HIV S protein-interacting polypeptides and a pharmaceutically acceptable excipient. In some embodiments, therapy provided herein comprises administration of a vector comprising the nucleic acid encoding for the HIV S protein-interacting polypeptides and a pharmaceutically acceptable excipient. In some embodiments, therapy provided herein comprises administration of Ebola virus glycoprotein-interacting polypeptides and a pharmaceutically acceptable excipient. In some embodiments, therapy provided herein comprises administration of a nucleic acid encoding for the Ebola virus glycoprotein-interacting polypeptides and a pharmaceutically acceptable excipient. In some embodiments, therapy provided herein comprises administration of a vector comprising the nucleic acid encoding for the Ebola virus glycoprotein-interacting polypeptides and a pharmaceutically acceptable excipient. In some embodiments, therapy provided herein comprises administration of influenza viral S protein-interacting polypeptides and a pharmaceutically acceptable excipient. In some embodiments, therapy provided herein comprises administration of a nucleic acid encoding for the influenza viral S protein-interacting polypeptides and a pharmaceutically acceptable excipient. In some embodiments, therapy provided herein comprises administration of a vector comprising the nucleic acid encoding for the influenza viral S protein-interacting polypeptides and a pharmaceutically acceptable excipient. In some embodiments, therapy provided herein comprises administration of RSV glycoprotein-interacting polypeptides and a pharmaceutically acceptable excipient. In some embodiments, therapy provided herein comprises administration of a nucleic acid encoding for the RSV glycoprotein-interacting polypeptides and a pharmaceutically acceptable excipient. In some embodiments, therapy provided herein comprises administration of a vector comprising the nucleic acid encoding for the RSV glycoprotein-interacting polypeptides and a pharmaceutically acceptable excipient.

As disclosed herein, a “viral protein-interacting polypeptide” (also “virus protein-interacting polypeptide”) describes any polypeptide capable of interacting with a viral protein. For example, a viral protein-interacting polypeptide may be a “virus glycoprotein-interacting polypeptide” (also “virus glycoprotein-interacting polypeptide”) and may interact with virus glycoproteins. In some cases, such interaction with virus glycoproteins may be inhibition or interference with virus spike proteins. In specific examples, a viral protein-interacting polypeptide is a “virus spike protein-interacting polypeptide” (also “virus S protein-interacting polypeptide”) and may interact with virus spike proteins. In some cases, such interaction with virus spike proteins may be inhibition or interference with virus spike proteins.

As disclosed herein, a “coronavirus S protein-interacting polypeptide” (also “coronavirus spike protein-interacting polypeptide”) describes any polypeptide capable of interacting with a coronavirus spike (S) protein. In some cases, such interaction with a coronavirus spike protein may be inhibition or interference with a coronavirus spike protein. For example, a coronavirus S protein-interacting polypeptide may interact with an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus comprises SARS-CoV, the coronavirus spike protein comprises a SARS-CoV S protein, and the coronavirus S protein-interacting polypeptide is derived from an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus comprises SARS-CoV-2, the coronavirus spike protein comprises a SARS-CoV-2 S protein, and the coronavirus S protein-interacting polypeptide is derived from an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus comprises MERS-CoV, the coronavirus spike protein comprises a MERS-CoV S protein, and the coronavirus S protein-interacting polypeptide is derived from an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus comprises HCoV-229E, the coronavirus spike protein comprises a HCoV-229E S protein, and the coronavirus S protein-interacting polypeptide is derived from a HCoV-229E S protein, a HCoV-NL63 S protein, a HCoV-OC43 S protein, a HCoV-HKU1 S protein, a MERS-CoV S protein, a SARS-CoV S protein, and/or a SARS-CoV-2 S protein. In some embodiments, the coronavirus comprises HCoV-NL63, the coronavirus spike protein comprises an HCoV-NL63 S protein, and the coronavirus S protein-interacting polypeptide is derived from an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus comprises HCoV-OC43, the coronavirus spike protein comprises an HCoV-OC43 S protein, and the coronavirus S protein-interacting polypeptide is derived from an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus comprises HCoV-HKU1, the coronavirus spike protein comprises an HCoV-HKU1 S protein, and the coronavirus S protein-interacting polypeptide is derived from an HCoV-229E S protein, an HCoV-NL63 S protein, an HCoV-OC43 S protein, an HCoV-HKU1 S protein, a MERS-CoV S protein, a SARS-CoV S protein, and/or a SARS-CoV-2 S protein. In some embodiments, the coronavirus S protein-interacting polypeptide is capable of interacting with a SARS-CoV spike protein, a SARS-CoV-2 spike protein, a MERS-CoV spike protein, an HCoV-229E spike protein, an HCoV-NL63 spike protein, an HCoV-OC43 spike protein, an HCoV-HKU1 spike protein, or a combination thereof.

As disclosed herein, a “HIV S protein-interacting polypeptide” (also “HIV spike protein-interacting polypeptide”) describes any polypeptide capable of interacting with an HIV spike (S) protein. In some cases, such interaction with an HIV spike protein may be inhibition or interference with an HIV spike protein. In some embodiments, the HIV S protein comprises HIV-1 gp160, and the HIV S protein-interacting polypeptide is derived from the HIV-1 gp160 S protein.

As disclosed herein, an “Ebola virus glycoprotein-interacting polypeptide” (also “Ebola virus glycoprotein-interacting polypeptide”) describes any polypeptide capable of interacting with an Ebola glycoprotein. In some cases, such interaction with an Ebola glycoprotein may be inhibition or interference with an Ebola glycoprotein. In some embodiments, the Ebola virus glycoprotein comprises Ebola virus GP, and the Ebola virus glycoprotein-interacting polypeptide is derived from the Ebola virus GP protein.

As disclosed herein, an “influenza viral S protein-interacting polypeptide” (also “influenza virus spike protein-interacting polypeptide”) describes any polypeptide capable of interacting with an influenza virus spike (S) protein. In some cases, such interaction with an influenza virus S protein may be inhibition or interference with an influenza virus S protein. For example, an influenza viral S protein-interacting polypeptide may interact with an influenza virus A protein, an influenza virus B protein, and/or an influenza virus C protein. In some embodiments, the influenza comprises influenza virus A, the influenza virus spike protein comprises an influenza virus A S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein. In some embodiments, the influenza comprises influenza virus A/H1, the influenza virus spike protein comprises an influenza virus A/H1 HA S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein. In some embodiments, the influenza comprises influenza virus A/H3, the influenza virus spike protein comprises an influenza virus A/H3 HA S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein.

In some embodiments, the influenza comprises influenza virus B, the influenza virus spike protein comprises an influenza virus B S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein. In some embodiments, the influenza comprises influenza virus B/Victoria, the influenza virus spike protein comprises an influenza virus B/Victoria HA S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein. In some embodiments, the influenza comprises influenza virus B/Yamagata, the influenza virus spike protein comprises an influenza virus B/Yamagata HA S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein.

In some embodiments, the influenza comprises influenza virus C, the influenza virus spike protein comprises an influenza virus C S protein, and the influenza viral S protein-interacting polypeptide is derived from an influenza virus A/H1 HA S protein, an influenza virus A/H3 HA S protein, an influenza virus B/Victoria HA S protein, or an influenza virus B/Yamagata HA S protein.

As disclosed herein, an “RSV glycoprotein-interacting polypeptide” (also “RSV-interacting polypeptide”) describes any polypeptide capable of interacting with an RSV glycoprotein. In some cases, such interaction with an RSV glycoprotein may be inhibition or interference with an RSV glycoprotein. In some embodiments, the RSV glycoprotein comprises RSV F protein, and the RSV glycoprotein-interacting polypeptide is derived from the RSV F protein.

In some embodiments, the disclosed methods comprise treating a subject suffering from a viral infection, e.g., a coronavirus infection, an HIV infection, an Ebola infection, an RSV infection, and/or an influenza infection, and/or post-viral infection syndrome with viral protein-interacting polypeptides or polynucleotides encoding viral protein-interacting polypeptides. As disclosed herein in some embodiments, administration of a viral protein-interacting polypeptide can inhibit formation and translocation of the viral proteins, such as viral spike proteins and/or viral glycoproteins, to cell surfaces of the subject and/or to viral envelopes and/or reduce the amount of the viral proteins, such as viral spike proteins and/or viral glycoproteins, on cell surfaces of the subject and/or on viral envelopes.

Accordingly, in some embodiments, disclosed is a method for regulating a viral protein, such as a glycoprotein and/or spike protein, or biological function thereof, in vivo in a subject comprising contacting the viral protein or a portion thereof with an effective amount of a polypeptide comprising an amino acid corresponding to a sequence of the viral protein, the amino acid sequence having at least 10% sequence identity with the corresponding sequence of the viral protein. In some embodiments, disclosed is a method for treating or preventing a viral infection, e.g., a coronavirus infection, an HIV infection, an Ebola infection, an RSV infection, and/or an influenza infection, and/or post-viral infection syndrome in a subject comprising administering to the subject a therapeutically effective amount of a composition comprising a polypeptide comprising an amino acid corresponding to a sequence of the viral protein, the amino acid sequence having at least 10% sequence identity with the corresponding sequence of the viral protein. In some cases, contacting the viral protein or a portion thereof with an effective amount of a polypeptide comprising an amino acid corresponding to a sequence of the viral protein comprises competition of the viral protein-interacting polypeptide with a corresponding oligomerization domain of the viral protein.

In some cases, the inhibition of formation and translocation of viral proteins, such as viral spike proteins and/or viral glycoproteins, to cell surfaces of the subject and/or to viral envelopes and/or the reduction in the amount of the viral proteins, such as viral spike proteins and/or viral glycoproteins, on cell surfaces of the subject and/or on viral envelopes is due to oligomerization of the polypeptide and the viral protein to inhibit the viral protein or biological function thereof. In some cases, oligomerization of the polypeptide and the viral proteins, such as viral spike proteins and/or viral glycoproteins, inhibits the viral protein and results in formation of a non-native protein complex. In some cases, oligomerization of the viral protein-interacting polypeptide with a corresponding oligomerization domain of the viral protein comprises competition of the viral protein-interacting polypeptide with a corresponding oligomerization domain of the viral protein, and competition of the polypeptide with the viral proteins, such as viral spike proteins and/or viral glycoproteins, inhibits the viral protein and results in formation of a non-native protein complex.

Accordingly, in some embodiments, disclosed is a method for regulating a viral protein, such as viral spike proteins and/or viral glycoproteins, or biological function thereof, in vivo in a subject comprising contacting the viral protein or a portion thereof with an effective amount of a polypeptide having an amino acid sequence corresponding to an oligomerization domain of the virus spike protein and having at least 10% identity with the oligomerization domain, wherein the polypeptide oligomerizes with the oligomerization domain of the viral protein to form a non-native protein complex, thereby regulating the viral protein or biological function thereof in vivo. In some embodiments, disclosed is a method for treating or preventing a viral infection and/or post-viral infection syndrome in a subject comprising administering to the subject a therapeutically effective amount of a composition comprising a polypeptide having an amino acid sequence corresponding to an oligomerization domain of a viral protein, such as viral spike proteins and/or viral glycoproteins, and having at least 10% identity with the oligomerization domain, wherein the polypeptide oligomerizes with the oligomerization domain of the viral protein to form a non-native protein complex. In some cases, contacting the viral protein or a portion thereof with an effective amount of a polypeptide comprising an amino acid corresponding to a sequence of the viral protein comprises competition of the viral protein-interacting polypeptide with a corresponding oligomerization domain of the viral protein.

In some embodiments, the disclosed methods comprise treating a subject suffering from a coronavirus infection, e.g., a MERS-CoV infection, an HCoV-229E infection, an HCoV-NL63 infection, an HCoV-OC43 infection, an HCoV-HKU1 infection, a SARS-CoV infection, and/or a SARS-CoV-2 infection, and/or post-coronavirus infection syndrome with coronavirus S protein-interacting polypeptides or polynucleotides encoding coronavirus S protein-interacting polypeptides. As disclosed herein, administration of a coronavirus S protein-interacting polypeptide can inhibit formation and translocation of the coronavirus spike protein to cell surfaces of the subject and/or to viral envelopes and/or reduce the amount of the coronavirus spike protein on cell surfaces of the subject and/or on viral envelopes.

Accordingly, in some embodiments, disclosed is a method for regulating a coronavirus spike protein or biological function thereof in vivo in a subject comprising contacting the coronavirus spike protein or a portion thereof with an effective amount of a polypeptide comprising an amino acid sequence having at least 10-80% identity, for example, 10% identity, 20% identity, 30% identity, 40% identity, 50% identity, 60% identity, 70% identity, or 80% identity, with SEQ ID NOS:2, 4, 6, 8, 10, 12, 14, or 16. In some embodiments, the polypeptide comprises an amino acid sequence with greater than 80% identity, for example, 85% identity, 90% identity, 95% identity, or 99% identity, with SEQ ID NOS:2, 4, 6, 8, 10, 12, 14, or 16. In some embodiments, the polypeptide comprises SEQ ID NO:2, 4, 6, 8, 10, 12, 14, or 16. In some cases, contacting the coronavirus spike protein or a portion thereof with an effective amount of a polypeptide comprising an amino acid sequence corresponding to SEQ ID NOS:2, 4, 6, 8, 10, 12, 14, or 16 comprises competition of the polypeptide with a corresponding oligomerization domain of the coronavirus spike protein. In some embodiments, disclosed is a method for treating or preventing a coronavirus infection, e.g., a MERS-CoV infection, a HCoV-229E infection, a HCoV-NL63 infection, a HCoV-OC43 infection, a HCoV-HKU1 infection, a SARS-CoV infection, and/or a SARS-CoV-2 infection, and/or post-coronavirus infection syndrome in a subject comprising administering to the subject a therapeutically effective amount of a composition comprising a polypeptide comprising an amino acid sequence having at least 10-80% identity, for example, 10% identity, 20% identity, 30% identity, 40% identity, 50% identity, 60% identity, 70% identity, or 80% identity, with SEQ ID NOS:2, 4, 6, 8, 10, 12, 14, or 16. In some embodiments, the polypeptide comprises an amino acid sequence with greater than 80% identity 12, 14, or 16, for example, 85% identity, 90% identity, 95% identity, or 99% identity, with SEQ ID NOS:2, 4, 6, 8, 10, 12, 14, or 16. In some embodiments, the polypeptide comprises SEQ ID NO:2, 4, 6, 8, 10, 12, 14, or 16.

In some cases, the inhibition of formation and translocation of the coronavirus spike protein to cell surfaces of the subject and/or to viral envelopes and/or the reduction in the amount of the coronavirus spike protein on cell surfaces of the subject and/or on viral envelopes is due to oligomerization of the polypeptide and the coronavirus spike protein to inhibit the coronavirus spike protein or biological function thereof. In some cases, oligomerization of the polypeptide and the coronavirus spike protein inhibits the coronavirus spike protein and results in formation of a non-native protein complex. In some cases, oligomerization of the polypeptide with a corresponding oligomerization domain of the coronavirus spike protein comprises competition of the polypeptide with a corresponding oligomerization domain of the coronavirus spike protein, and competition of the polypeptide with the coronavirus spike protein inhibits the coronavirus spike protein and results in formation of a non-native protein complex.

Accordingly, in some embodiments, disclosed is a method for regulating a coronavirus spike protein or biological function thereof in vivo in a subject comprising contacting the coronavirus spike protein or a portion thereof with an effective amount of a polypeptide having an amino acid sequence corresponding to an oligomerization domain of the coronavirus spike protein and having 10-80% identity, for example, 10% identity, 20% identity, 30% identity, 40% identity, 50% identity, 60% identity, 70% identity, or 80% identity, with SEQ ID NOS:2, 4, 6, 8, 10, 12, 14, or 16, wherein the polypeptide oligomerizes with the oligomerization domain of the coronavirus spike protein to form a non-native protein complex, thereby regulating the coronavirus spike protein or biological function thereof in vivo. In some embodiments, the polypeptide comprises an amino acid sequence with greater than 80% identity, for example, 85% identity, 90% identity, 95% identity, or 99% identity, with SEQ ID NOS:2, 4, 6, 8, 10, 12, 14, or 16. In some embodiments, the polypeptide comprises SEQ ID NO:2, 4, 6, 8, 10, 12, 14, or 16.

In some embodiments, disclosed is a method for treating or preventing a coronavirus infection and/or post-coronavirus infection syndrome in a subject comprising administering to the subject a therapeutically effective amount of a composition comprising a polypeptide having an amino acid sequence corresponding to an oligomerization domain of a coronavirus spike protein and having 10-80% identity, for example, 10% identity, 20% identity, 30% identity, 40% identity, 50% identity, 60% identity, 70% identity, or 80% identity, with SEQ ID NOS:2, 4, 6, 8, 10, 12, 14, or 16, wherein the polypeptide oligomerizes with the oligomerization domain of the coronavirus spike protein to form a non-native protein complex. In some embodiments, the polypeptide comprises an amino acid sequence with greater than 80% identity, for example, 85% identity, 90% identity, 95% identity, or 99% identity, with SEQ ID NOS:2, 4, 6, 8, 10, 12, 14, or 16. In some embodiments, the polypeptide comprises SEQ ID NO:2, 4, 6, 8, 10, 12, 14, or 16.

In some embodiments, the disclosed methods comprise treating a subject suffering from an HIV infection with HIV S protein-interacting polypeptides or polynucleotides encoding HIV S protein-interacting polypeptides. As disclosed herein, administration of an HIV S protein-interacting polypeptide can inhibit formation and translocation of the HIV spike protein to cell surfaces of the subject and/or to viral envelopes and/or reduce the amount of the HIV spike protein on cell surfaces of the subject and/or on viral envelopes.

Accordingly, in some embodiments, disclosed is a method for regulating an HIV spike protein or biological function thereof in vivo in a subject comprising contacting the HIV spike protein or a portion thereof with an effective amount of a polypeptide comprising an amino acid sequence having 10-80% identity, for example, 10% identity, 20% identity, 30% identity, 40% identity, 50% identity, 60% identity, 70% identity, or 80% identity, with SEQ ID NO:18. In some embodiments, the polypeptide comprises an amino acid sequence with greater than 80% identity, for example, 85% identity, 90% identity, 95% identity, or 99% identity, with SEQ ID NO:18. In some embodiments, the polypeptide comprises SEQ ID NO:18. In some cases, contacting the HIV spike protein or a portion thereof with an effective amount of a polypeptide comprising an amino acid sequence corresponding to SEQ ID NO:18 comprises competition of the polypeptide with a corresponding oligomerization domain of the HIV spike protein.

In some embodiments, disclosed is a method for treating or preventing an HIV infection in a subject comprising administering to the subject a therapeutically effective amount of a composition comprising a polypeptide comprising an amino acid sequence having 10-80% identity, for example, 10% identity, 20% identity, 30% identity, 40% identity, 50% identity, 60% identity, 70% identity, or 80% identity, with SEQ ID NO:18. In some embodiments, the polypeptide comprises an amino acid sequence with greater than 80% identity, for example, 85% identity, 90% identity, 95% identity, or 99% identity, with SEQ ID NO:18. In some embodiments, the polypeptide comprises SEQ ID NO:18.

In some cases, the inhibition of formation and translocation of the HIV spike protein to cell surfaces of the subject and/or to viral envelopes and/or the reduction in the amount of the HIV spike protein on cell surfaces of the subject and/or on viral envelopes is due to oligomerization of the polypeptide and the HIV spike protein to inhibit the HIV spike protein or biological function thereof. In some cases, oligomerization of the polypeptide and the HIV spike protein inhibits the HIV spike protein and results in formation of a non-native protein complex. In some cases, oligomerization of the polypeptide with a corresponding oligomerization domain of the HIV spike protein comprises competition of the polypeptide with a corresponding oligomerization domain of the HIV spike protein, and competition of the polypeptide with the HIV spike protein inhibits the HIV spike protein and results in formation of a non-native protein complex.

Accordingly, in some embodiments, disclosed is a method for regulating an HIV spike protein or biological function thereof in vivo in a subject comprising contacting the HIV spike protein or a portion thereof with an effective amount of a polypeptide having an amino acid sequence corresponding to an oligomerization domain of the HIV spike protein and having 10-80% identity, for example, 10% identity, 20% identity, 30% identity, 40% identity, 50% identity, 60% identity, 70% identity, or 80% identity, with SEQ ID NO:18, wherein the polypeptide oligomerizes with the oligomerization domain of the HIV spike protein to form a non-native protein complex, thereby regulating the HIV spike protein or biological function thereof in vivo. In some embodiments, the polypeptide comprises an amino acid sequence with greater than 80% identity, for example, 85% identity, 90% identity, 95% identity, or 99% identity, with SEQ ID NO:18. In some embodiments, the polypeptide comprises SEQ ID NO:18.

In some embodiments, disclosed is a method for treating or preventing an HIV infection in a subject comprising administering to the subject a therapeutically effective amount of a composition comprising a polypeptide having an amino acid sequence corresponding to an oligomerization domain of an HIV spike protein and having 10-80% identity, for example, 10% identity, 20% identity, 30% identity, 40% identity, 50% identity, 60% identity, 70% identity, or 80% identity, with SEQ ID NO:18, wherein the polypeptide oligomerizes with the oligomerization domain of the HIV spike protein to form a non-native protein complex. In some embodiments, the polypeptide comprises an amino acid sequence with greater than 80% identity, for example, 85% identity, 90% identity, 95% identity, or 99% identity, with SEQ ID NO:18. In some embodiments, the polypeptide comprises SEQ ID NO:18.

In some embodiments, the disclosed methods comprise treating a subject suffering from an Ebola infection with Ebola virus glycoprotein-interacting polypeptides or polynucleotides encoding Ebola virus glycoprotein-interacting polypeptides. As disclosed herein, administration of an Ebola virus glycoprotein-interacting polypeptide can inhibit formation and translocation of the Ebola virus glycoprotein to cell surfaces of the subject and/or to viral envelopes and/or reduce the amount of the Ebola virus glycoprotein on cell surfaces of the subject and/or on viral envelopes.

Accordingly, in some embodiments, disclosed is a method for regulating an Ebola virus glycoprotein or biological function thereof in vivo in a subject comprising contacting the Ebola virus glycoprotein or a portion thereof with an effective amount of a polypeptide comprising an amino acid sequence having 10-80% identity, for example, 10% identity, 20% identity, 30% identity, 40% identity, 50% identity, 60% identity, 70% identity, or 80% identity, with SEQ ID NO:20. In some embodiments, the polypeptide comprises an amino acid sequence with greater than 80% identity, for example, 85% identity, 90% identity, 95% identity, or 99% identity, with SEQ ID NO:20. In some embodiments, the polypeptide comprises SEQ ID NO:20. In some cases, contacting the Ebola virus glycoprotein or a portion thereof with an effective amount of a polypeptide comprising an amino acid sequence corresponding to SEQ ID NO:20 comprises competition of the polypeptide with a corresponding oligomerization domain of the Ebola virus glycoprotein.

In some embodiments, disclosed is a method for treating or preventing an Ebola infection in a subject comprising administering to the subject a therapeutically effective amount of a composition comprising a polypeptide comprising an amino acid sequence having 10-80% identity, for example, 10% identity, 20% identity, 30% identity, 40% identity, 50% identity, 60% identity, 70% identity, or 80% identity, with SEQ ID NO:20. In some embodiments, the polypeptide comprises an amino acid sequence with greater than 80% identity, for example, 85% identity, 90% identity, 95% identity, or 99% identity, with SEQ ID NO:20. In some embodiments, the polypeptide comprises SEQ ID NO:20.

In some cases, the inhibition of formation and translocation of the Ebola virus glycoprotein to cell surfaces of the subject and/or to viral envelopes and/or the reduction in the amount of the Ebola virus glycoprotein on cell surfaces of the subject and/or on viral envelopes is due to oligomerization of the polypeptide and the Ebola virus glycoprotein to inhibit the Ebola virus glycoprotein or biological function thereof. In some cases, oligomerization of the polypeptide and the Ebola virus glycoprotein inhibits the Ebola virus glycoprotein and results in formation of a non-native protein complex. In some cases, oligomerization of the polypeptide with a corresponding oligomerization domain of the Ebola virus glycoprotein comprises competition of the polypeptide with a corresponding oligomerization domain of the Ebola virus glycoprotein, and competition of the polypeptide with the Ebola virus glycoprotein inhibits the Ebola virus glycoprotein and results in formation of a non-native protein complex.

Accordingly, in some embodiments, disclosed is a method for regulating an Ebola virus glycoprotein or biological function thereof in vivo in a subject comprising contacting the Ebola virus glycoprotein or a portion thereof with an effective amount of a polypeptide having an amino acid sequence corresponding to an oligomerization domain of the Ebola virus glycoprotein and having 10-80% identity, for example, 10% identity, 20% identity, 30% identity, 40% identity, 50% identity, 60% identity, 70% identity, or 80% identity, with SEQ ID NO:20, wherein the polypeptide oligomerizes with the oligomerization domain of the Ebola virus glycoprotein to form a non-native protein complex, thereby inhibiting the Ebola virus glycoprotein or biological function thereof in vivo. In some embodiments, the polypeptide comprises an amino acid sequence with greater than 80% identity, for example, 85% identity, 90% identity, 95% identity, or 99% identity, with SEQ ID NO:20. In some embodiments, the polypeptide comprises SEQ ID NO:20.

In some embodiments, disclosed is a method for treating or preventing an Ebola infection in a subject comprising administering to the subject a therapeutically effective amount of a composition comprising a polypeptide having an amino acid sequence corresponding to an oligomerization domain of an Ebola virus glycoprotein and having 10-80% identity, for example, 10% identity, 20% identity, 30% identity, 40% identity, 50% identity, 60% identity, 70% identity, or 80% identity, with SEQ ID NO:20, wherein the polypeptide oligomerizes with the oligomerization domain of the Ebola virus glycoprotein to form a non-native protein complex. In some embodiments, the polypeptide comprises an amino acid sequence with greater than 80% identity, for example, 85% identity, 90% identity, 95% identity, or 99% identity, with SEQ ID NO:20. In some embodiments, the polypeptide comprises SEQ ID NO:20.

In some embodiments, the disclosed methods comprise treating a subject suffering from an influenza infection with influenza viral S protein-interacting polypeptides or polynucleotides encoding influenza viral S protein-interacting polypeptides. As disclosed herein, administration of an influenza viral S protein-interacting polypeptide can inhibit formation and translocation of the influenza virus spike protein to cell surfaces of the subject and/or to viral envelopes and/or reduce the amount of the influenza virus spike protein on cell surfaces of the subject and/or on viral envelopes.

Accordingly, in some embodiments, disclosed is a method for regulating an influenza virus spike protein or biological function thereof in vivo in a subject comprising contacting the influenza virus spike protein or a portion thereof with an effective amount of a polypeptide comprising an amino acid sequence having 10-80% identity, for example, 10% identity, 20% identity, 30% identity, 40% identity, 50% identity, 60% identity, 70% identity, or 80% identity, with SEQ ID NO:25. In some embodiments, the polypeptide comprises an amino acid sequence with greater than 80% identity, for example, 85% identity, 90% identity, 95% identity, or 99% identity, with SEQ ID NO:25. In some embodiments, the polypeptide comprises SEQ ID NO:25. In some cases, contacting the influenza virus spike protein or a portion thereof with an effective amount of a polypeptide comprising an amino acid sequence corresponding to SEQ ID NO:25 comprises competition of the polypeptide with a corresponding oligomerization domain of the influenza virus spike protein.

In some embodiments, disclosed is a method for treating or preventing an influenza infection in a subject comprising administering to the subject a therapeutically effective amount of a composition comprising a polypeptide comprising an amino acid sequence having 10-80% identity, for example, 10% identity, 20% identity, 30% identity, 40% identity, 50% identity, 60% identity, 70% identity, or 80% identity, with SEQ ID NO:25. In some embodiments, the polypeptide comprises an amino acid sequence with greater than 80% identity, for example, 85% identity, 90% identity, 95% identity, or 99% identity, with SEQ ID NO:25. In some embodiments, the polypeptide comprises SEQ ID NO:25.

In some cases, the inhibition of formation and translocation of the influenza virus spike protein to cell surfaces of the subject and/or to viral envelopes and/or the reduction in the amount of the influenza virus spike protein on cell surfaces of the subject and/or on viral envelopes is due to oligomerization of the polypeptide and the influenza virus spike protein to inhibit the influenza virus spike protein or biological function thereof. In some cases, oligomerization of the polypeptide and the influenza virus spike protein inhibits the influenza virus spike protein and results in formation of a non-native protein complex. In some cases, oligomerization of the polypeptide with a corresponding oligomerization domain of the influenza virus spike protein comprises competition of the polypeptide with a corresponding oligomerization domain of the influenza virus spike protein, and competition of the polypeptide with the influenza virus spike protein inhibits the influenza virus spike protein and results in formation of a non-native protein complex.

Accordingly, in some embodiments, disclosed is a method for regulating an influenza virus spike protein or biological function thereof in vivo in a subject comprising contacting the influenza virus spike protein or a portion thereof with an effective amount of a polypeptide having an amino acid sequence corresponding to an oligomerization domain of the influenza virus spike protein and having 10-80% identity, for example, 10% identity, 20% identity, 30% identity, 40% identity, 50% identity, 60% identity, 70% identity, or 80% identity, with SEQ ID NO:25, wherein the polypeptide oligomerizes with the oligomerization domain of the influenza virus spike protein to form a non-native protein complex, thereby inhibiting the influenza virus spike protein or biological function thereof in vivo. In some embodiments, the polypeptide comprises an amino acid sequence with greater than 80% identity, for example, 85% identity, 90% identity, 95% identity, or 99% identity, with SEQ ID NO:25. In some embodiments, the polypeptide comprises SEQ ID NO:25.

In some embodiments, disclosed is a method for treating or preventing an influenza infection in a subject comprising administering to the subject a therapeutically effective amount of a composition comprising a polypeptide having an amino acid sequence corresponding to an oligomerization domain of an influenza virus spike protein and having 10-80% identity, for example, 10% identity, 20% identity, 30% identity, 40% identity, 50% identity, 60% identity, 70% identity, or 80% identity, with SEQ ID NO:25, wherein the polypeptide oligomerizes with the oligomerization domain of the influenza virus spike protein to form a non-native protein complex. In some embodiments, the polypeptide comprises an amino acid sequence with greater than 80% identity, for example, 85% identity, 90% identity, 95% identity, or 99% identity, with SEQ ID NO:25.

In some embodiments, the disclosed methods comprise treating a subject suffering from an RSV infection with RSV glycoprotein-interacting polypeptides or polynucleotides encoding RSV glycoprotein-interacting polypeptides. As disclosed herein, administration of an RSV glycoprotein-interacting polypeptide can inhibit formation and translocation of the RSV glycoprotein to cell surfaces of the subject and/or to viral envelopes and/or reduce the amount of the RSV glycoprotein on cell surfaces of the subject and/or on viral envelopes.

Accordingly, in some embodiments, disclosed is a method for regulating an RSV glycoprotein or biological function thereof in vivo in a subject comprising contacting the RSV glycoprotein or a portion thereof with an effective amount of a polypeptide comprising an amino acid sequence having 10-80% identity, for example, 10% identity, 20% identity, 30% identity, 40% identity, 50% identity, 60% identity, 70% identity, or 80% identity, with SEQ ID NO:34. In some embodiments, the polypeptide comprises an amino acid sequence with greater than 80% identity, for example, 85% identity, 90% identity, 95% identity, or 99% identity, with SEQ ID NO:34. In some embodiments, the polypeptide comprises SEQ ID NO:34. In some cases, contacting the RSV glycoprotein or a portion thereof with an effective amount of a polypeptide comprising an amino acid sequence corresponding to SEQ ID NO:34 comprises competition of the polypeptide with a corresponding oligomerization domain of the RSV glycoprotein.

In some embodiments, disclosed is a method for treating or preventing an RSV in a subject comprising administering to the subject a therapeutically effective amount of a composition comprising a polypeptide comprising an amino acid sequence having 10-80% identity, for example, 10% identity, 20% identity, 30% identity, 40% identity, 50% identity, 60% identity, 70% identity, or 80% identity, with SEQ ID NO:34. In some embodiments, the polypeptide comprises an amino acid sequence with greater than 80% identity, for example, 85% identity, 90% identity, 95% identity, or 99% identity, with SEQ ID NO:34. In some embodiments, the polypeptide comprises SEQ ID NO:34.

In some cases, the inhibition of formation and translocation of the RSV glycoprotein to cell surfaces of the subject and/or to viral envelopes and/or the reduction in the amount of the RSV glycoprotein on cell surfaces of the subject and/or on viral envelopes is due to oligomerization of the polypeptide and the RSV glycoprotein to inhibit the RSV glycoprotein or biological function thereof. In some cases, oligomerization of the polypeptide and the RSV glycoprotein inhibits the RSV glycoprotein and results in formation of a non-native protein complex. In some cases, oligomerization of the polypeptide with a corresponding oligomerization domain of the RSV glycoprotein comprises competition of the polypeptide with a corresponding oligomerization domain of the RSV glycoprotein, and competition of the polypeptide with the RSV glycoprotein inhibits the RSV glycoprotein and results in formation of a non-native protein complex.

Accordingly, in some embodiments, disclosed is a method for regulating an RSV glycoprotein or biological function thereof in vivo in a subject comprising contacting the RSV glycoprotein or a portion thereof with an effective amount of a polypeptide having an amino acid sequence corresponding to an oligomerization domain of the RSV glycoprotein and having 10-80% identity, for example, 10% identity, 20% identity, 30% identity, 40% identity, 50% identity, 60% identity, 70% identity, or 80% identity, with SEQ ID NO:34, wherein the polypeptide oligomerizes with the oligomerization domain of the RSV glycoprotein to form a non-native protein complex, thereby inhibiting the RSV glycoprotein or biological function thereof in vivo. In some embodiments, the polypeptide comprises an amino acid sequence with greater than 80% identity, for example, 85% identity, 90% identity, 95% identity, or 99% identity, with SEQ ID NO:34. In some embodiments, the polypeptide comprises SEQ ID NO:34.

In some embodiments, disclosed is a method for treating or preventing an RSV in a subject comprising administering to the subject a therapeutically effective amount of a composition comprising a polypeptide having an amino acid sequence corresponding to an oligomerization domain of an RSV glycoprotein and having 10-80% identity, for example, 10% identity, 20% identity, 30% identity, 40% identity, 50% identity, 60% identity, 70% identity, or 80% identity, with SEQ ID NO:34, wherein the polypeptide oligomerizes with the oligomerization domain of the RSV glycoprotein to form a non-native protein complex. In some embodiments, the polypeptide comprises an amino acid sequence with greater than 80% identity, for example, 85% identity, 90% identity, 95% identity, or 99% identity, with SEQ ID NO:34. In some embodiments, the polypeptide comprises SEQ ID NO:34.

In some embodiments, therapy provided herein comprises administration of a combination of therapeutic agents, such as viral protein-interacting polypeptides and an additional therapeutic agent. In some embodiments, the additional therapeutic comprises agents for treating a viral infection, which include but are not limited to steroids, zinc, vitamin C, convalescent serum, Remdesivir, Tocilizumab, Anakinra, Beclomethasone, Betamethasone, Budesonide Cortisone, Dexamethasone, Hydrocortisone, Methylprednisolone, Prednisolone, Prednisone, Triamcinolone, Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine, Hydroxychloroquine, CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), or a combination thereof.

In some embodiments, the agents for treating a viral infection further include but are not limited to antiretroviral therapies (ARTs), such as: non-nucleoside reverse transcriptase inhibitors (NNRTIs), which include but are not limited to efavirenz (Sustiva), rilpivirine (Edurant), etravirine (Intelence), delavirdine (Rescriptor), nevirapine (Viramune, Viramune XR), and doravirine (Pifeltro); nucleoside or nucleotide reverse transcriptase inhibitors (NRTIs), which include but are not limited to abacavir (Ziagen), tenofovir alafenamide fumarate (Vemlidy), tenofovir (Viread), emtricitabine (Emtriva), lamivudine (Epivir), zidovudine (Retrovir), abacavir/lamivudine/zidovudine (Trizivir), abacavir/lamivudine (Epzicom), emtricitabine/tenofovir (Truvada), abacavir/lamivudine (Epzicom), lamivudine/tenofovir disoproxil fumarate (Cimduo, Temixys), lamivudine/zidovudine (Combivir), emtricitabine/tenofovir alafenamide (Descovy), didanosine (Videx, Videx EC), and stavudine (Zerit); protease inhibitors (PIs), which include atazanavir (Reyataz), darunavir (Prezista), fosamprenavir (Lexiva), ritonavir (Norvir), tipranavir (Aptivus), lopinavir/ritonavir (Kaletra), atazanavir/cobicistat (Evotaz, also a cytochrome P450 inhibitor), darunavir/cobicistat (Prezcobix, also a cytochrome P450 inhibitor), indinavir (Crixivan), nelfinavir (Viracept), and saquinavir (Invirase); integrase strand transfer inhibitors (INSTIs), which include but are not limited to bictegravir sodium/emtricitabine/tenofovir alafenamide fumar (Biktarvy), raltegravir (Isentress), elvitegravir (Genvoya and Stribild), and dolutegravir (Tivicay); cytochrome p450 inhibitors, which include but are not limited to cobicistat (Tybost) and ritonavir (Norvir); and entry or fusion inhibitors, which include but are not limited to enfuvirtide (Fuzeon) and maraviroc (Selzentry); post-attachment inhibitors, which include but are not limited to ibalizumab-uiyk (Trogarzo); chemokine coreceptor antagonists, which include but are not limited to maraviroc (Selzentry); gp120 attachment inhibitors, which include but are not limited to fostemsavir (Rukobia); combination NNRTIs and NRTIs, which include but are not limited to doravirine/lamivudine/tenofovir disoproxil fumarate (Delstrigo), efavirenz/lamivudine/tenofovir disoproxil fumarate (Symfi), efavirenz/lamivudine/tenofovir disoproxil fumarate (Symfi Lo), efavirenz/emtricitabine/tenofovir disoproxil fumarate (Atripla), emtricitabine/rilpivirine/tenofovir alafenamide fumarate (Odefsey), emtricitabine/rilpivirine/tenofovir disoproxil fumarate (Complera); combination NRTI, INSTI, and cytochrome p450 inhibitors, which include but are not limited to elvitegravir/cobicistat/emtricitabine/tenofovir disoproxil fumarate (Stribild) and elvitegravir/cobicistat/emtricitabine/tenofovir alafenamide fumarate (Genvoya); combination NRTIs and INSTIs, which include but are not limited to abacavir/dolutegravir/lamivudine (Triumeq), bictegravir/emtricitabine/tenofovir alafenamide fumarate (Biktarvy), and dolutegravir/lamivudine (Dovato); combination NNRTIs and INSTIs, which include but are not limited to dolutegravir/rilpivirine (Juluca); combination NRTIs, PIs, and cytochrome p450 inhibitors, which include but are not limited to darunavir/cobicistat/emtricitabine/tenofovir alafenamide fumarate (Symtuza); acetyl-L-carnitine; whey protein; L-glutamine; L-arginine; hydroxymethylbutyrate (HMB); probiotics; vitamins and minerals; or combinations thereof.

In some embodiments, the agents for treating a viral infection further include but are not limited to atoltivimab/maftivimab/odesivimab-ebgn (Inmazeb), ansuvimab-zykl (Ebanga), Favipiravir (Avigan), Ribavirin, BCX4430, Brincidofovir, TKM-Ebola, AVI-7537, JK-05, or combinations thereof.

In some embodiments, the agents for treating a viral infection further include but are not limited to oseltamivir phosphate (Tamiflu), zanamivir (Relenza), peramivir (Rapivab), baloxavir marboxil (Xofluza), amantadine, rimantadine (Flumadine), umifenovir (Arbidol), moroxydine, fluticare, acetaminophen, chlorpheniramine, dextromethorphan, pseudoephedrine, or combinations thereof.

### V. Pharmaceutical Compositions

Also disclosed in some embodiments are pharmaceutical compositions comprising protein-interacting polypeptides or polynucleotides encoding protein-interacting polypeptides. For example, disclosed in some embodiments are pharmaceutical compositions comprising viral protein-interacting polypeptides or polynucleotides encoding viral protein-interacting polypeptides. In some embodiments, the viral protein-interacting polypeptides or polynucleotides encoding viral protein-interacting polypeptides comprise viral S protein-interacting polypeptides or polynucleotides encoding viral S protein-interacting polypeptides. In some embodiments, the viral S protein-interacting polypeptides or polynucleotides encoding viral S protein-interacting polypeptides comprise coronavirus S protein-interacting polypeptides or polynucleotides encoding coronavirus S protein-interacting polypeptides. In some embodiments, the viral S protein-interacting polypeptides or polynucleotides encoding viral S protein-interacting polypeptides comprise HIV S protein-interacting polypeptides or polynucleotides encoding HIV S protein-interacting polypeptides. In some embodiments, the viral S protein-interacting polypeptides or polynucleotides encoding viral S protein-interacting polypeptides comprise Ebola virus glycoprotein-interacting polypeptides or polynucleotides encoding Ebola virus glycoprotein-interacting polypeptides. In some embodiments, the viral S protein-interacting polypeptides or polynucleotides encoding viral S protein-interacting polypeptides comprise influenza viral S protein-interacting polypeptides or polynucleotides encoding influenza viral S protein-interacting polypeptides. In some embodiments, the viral S protein-interacting polypeptides or polynucleotides encoding viral S protein-interacting polypeptides comprise RSV glycoprotein-interacting polypeptides or polynucleotides encoding RSV glycoprotein-interacting polypeptides.

Therefore, disclosed in some embodiments are pharmaceutical compositions comprising coronavirus S protein-interacting polypeptides or polynucleotides encoding coronavirus S protein-interacting polypeptides. Disclosed in some embodiments are pharmaceutical compositions comprising HIV S protein-interacting polypeptides or polynucleotides encoding HIV S protein-interacting polypeptides. Disclosed in some embodiments are pharmaceutical compositions comprising Ebola virus glycoprotein-interacting polypeptides or polynucleotides encoding Ebola virus glycoprotein-interacting polypeptides. Disclosed in some embodiments are pharmaceutical compositions comprising influenza viral S protein-interacting polypeptides or polynucleotides encoding influenza viral S protein-interacting polypeptides. Disclosed in some embodiments are pharmaceutical compositions RSV glycoprotein-interacting polypeptides or polynucleotides encoding RSV glycoprotein-interacting polypeptides. In some embodiments, the pharmaceutical compositions can further comprise one or more additional therapeutics, for example, agents for treating viral infections, including but not limited to the agents disclosed herein. The compositions of the disclosure may be used for in vivo, in vitro, or ex vivo administration.

In some embodiments, the therapy comprises a protein-based therapy, which may be a protein-interacting polypeptide therapy. In some embodiments, the therapy comprises a polynucleotide-based therapy, which may be a therapy including a vector encoding a protein-interacting polypeptide or a fragment or functional derivative thereof. For example, in some embodiments, the therapy comprises a viral protein-based therapy, which may be a viral protein-interacting polypeptide therapy. In some embodiments, the viral protein-interacting polypeptide therapy is a viral S protein-interacting polypeptide therapy. In some embodiments, the viral S protein-interacting polypeptide therapy is a coronavirus S protein-interacting polypeptide therapy. In some embodiments, the viral S protein-interacting polypeptide therapy is an HIV S protein-interacting polypeptide therapy. In some embodiments, the viral S protein-interacting polypeptide therapy is an Ebola virus glycoprotein-interacting polypeptide therapy. In some embodiments, the viral S protein-interacting polypeptide therapy is an influenza viral S protein-interacting polypeptide therapy. In some embodiments, the viral S protein-interacting polypeptide therapy is an RSV glycoprotein-interacting polypeptide therapy. In some embodiments, the viral S protein-interacting polypeptide therapy is a combination of two or more protein-interacting polypeptide therapies, including but not limited to, coronavirus S protein-interacting polypeptide therapy, HIV S protein-interacting polypeptide therapy, Ebola virus glycoprotein-interacting polypeptide therapy, RSV glycoprotein-interacting polypeptide therapy, and/or influenza viral S protein-interacting polypeptide therapy.

Therefore, in some embodiments, the therapy comprises a coronavirus S protein-based therapy, which may be a coronavirus S protein-interacting polypeptide therapy. In some embodiments, the therapy comprises an HIV S protein-based therapy, which may be an HIV S protein-interacting polypeptide therapy. In some embodiments, the therapy comprises an Ebola virus glycoprotein-based therapy, which may be an Ebola virus glycoprotein-interacting polypeptide therapy. In some embodiments, the therapy comprises an influenza viral S protein-based therapy, which may be an influenza viral S protein-interacting polypeptide therapy. In some embodiments, the therapy comprises an RSV glycoprotein-based therapy, which may be an Ebola virus glycoprotein-interacting polypeptide therapy. In some embodiments, the therapy comprises a polynucleotide-based therapy, which may be a therapy including a vector encoding a viral protein-interacting polypeptide or a fragment or functional derivative thereof, for example, a coronavirus S protein-interacting polypeptide or a fragment or functional derivative thereof; a vector encoding an HIV S protein-interacting polypeptide or a fragment or functional derivative thereof; a vector encoding an Ebola virus glycoprotein-interacting polypeptide or a fragment or functional derivative thereof; a vector encoding an influenza viral S protein-interacting polypeptide or a fragment or functional derivative thereof; a vector encoding an RSV glycoprotein-interacting polypeptide or a fragment or functional derivative thereof; or a combination thereof.

In some embodiments, the therapy comprises one or more disease medications, for example, one or more anti-viral medications. Any of these disease therapies may be included. Any of these disease therapies may be excluded. Combinations of these therapies may also be administered.

The therapy provided herein may comprise administration of a combination of therapeutic compositions, such as a first disease therapy (e.g., a protein-interacting polypeptide therapy, such as a viral protein-interacting polypeptide therapy, or a polynucleotide encoding a protein-interacting polypeptide, such as a viral protein-interacting polypeptide) and one or more additional disease therapies (e.g., anti-viral medications). The therapies may be administered in any suitable manner known in the art. For example, the therapies may be administered sequentially (at different times) or concurrently (at the same time or approximately the same time; also “simultaneously” or “substantially simultaneously”). In some embodiments, the therapies may be administered in a separate composition. In some embodiments, the therapies may be in the same composition. The different therapies may be administered in one composition or in more than one composition, such as 2 compositions, 3 compositions, or 4 compositions. Various combinations of the agents may be employed.

In some embodiments, the composition(s) comprising a protein-interacting polypeptide therapy, such as a viral protein-interacting polypeptide therapy, or a polynucleotide encoding a protein-interacting polypeptide, such as a viral protein-interacting polypeptide, and the one or more additional disease medications are administered substantially simultaneously. In some embodiments, the composition(s) comprising a protein-interacting polypeptide therapy, such as a viral protein-interacting polypeptide therapy, or a polynucleotide encoding a protein-interacting polypeptide, such as a viral protein-interacting polypeptide, and the one or more additional disease medications are administered sequentially. In some embodiments, the composition(s) comprising a protein-interacting polypeptide therapy, such as a viral protein-interacting polypeptide therapy, or a polynucleotide encoding a protein-interacting polypeptide, such as a viral protein-interacting polypeptide, is administered before administering the one or more additional disease medications. In some embodiments, the composition(s) comprising a protein-interacting polypeptide therapy, such as a viral protein-interacting polypeptide therapy, or a polynucleotide encoding a protein-interacting polypeptide, such as a viral protein-interacting polypeptide, is administered after administering the one or more additional disease medications.

In some embodiments, the composition(s) comprising a protein-interacting polypeptide therapy, such as a viral protein-interacting polypeptide therapy, or a polynucleotide encoding a protein-interacting polypeptide, such as a viral protein-interacting polypeptide, is delivered to the subject a single time. In some embodiments, the composition comprising a protein-interacting polypeptide therapy, such as a viral protein-interacting polypeptide therapy, or a polynucleotide encoding a protein-interacting polypeptide, such as a viral protein-interacting polypeptide, is delivered to the subject multiple times, such as once a day, more than once a day, once a week, more than once a week, once a month, more than once a month, once a year, or more than once a year. In some embodiments, a protein-interacting polypeptide therapy, such as a viral protein-interacting polypeptide therapy, or a polynucleotide encoding a protein-interacting polypeptide, such as a viral protein-interacting polypeptide, is administered to the subject multiple times. In some embodiments, a protein-interacting polypeptide therapy, such as a viral protein-interacting polypeptide therapy, or a polynucleotide encoding a protein-interacting polypeptide, such as a viral protein-interacting polypeptide, is administered to the subject a single time. Multiple treatments may or may not have the same formulations and/or routes of administration(s).

In some embodiments, the composition comprising a protein-interacting polypeptide therapy, such as a viral protein-interacting polypeptide therapy, or a polynucleotide encoding a protein-interacting polypeptide, such as a viral protein-interacting polypeptide, is delivered after onset of a disease, for example, a viral infection and/or post-viral infection syndrome. In some embodiments, the composition comprising a protein-interacting polypeptide therapy, such as a viral protein-interacting polypeptide therapy, or a polynucleotide encoding a protein-interacting polypeptide, such as a viral protein-interacting polypeptide, is delivered before onset of a disease, for example, a viral infection and/or post-viral infection syndrome.

### A. Carriers

In some embodiments, pharmaceutical compositions of the present disclosure comprise an effective amount of one or more compositions comprising a protein-interacting polypeptide therapy, such as a viral protein-interacting polypeptide therapy, or a polynucleotide encoding a protein-interacting polypeptide, such as a viral protein-interacting polypeptide, are dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to a subject, such as, for example, a human, as appropriate, and do not interfere with the therapeutic methods of the disclosure. The preparation of a pharmaceutical composition that contains at least one protein-interacting polypeptide therapy, such as a viral protein-interacting polypeptide therapy, or polynucleotide encoding a protein-interacting polypeptide, such as a viral protein-interacting polypeptide, and/or an additional active ingredient will be known to those of skill in the art in light of the present disclosure, as exemplified by Remington: The Science and Practice of Pharmacy, 21st Ed. Lippincott Williams and Wilkins, 2005, specifically incorporated by reference herein in its entirety. Moreover, for administration to a subject, it will be understood that preparations should meet sterility, pyrogenicity, general safety, and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, specifically incorporated by reference herein in its entirety). Except insofar as any conventional carrier is incompatible with the active ingredient, its use in the pharmaceutical compositions is contemplated. The compositions comprising a protein-interacting polypeptide therapy, such as a viral protein-interacting polypeptide therapy, or a polynucleotide encoding a protein-interacting polypeptide, such as a viral protein-interacting polypeptide may comprise different types of carriers depending on whether it is to be administered in solid, liquid or aerosol form, and whether it needs to be sterile for such routes of administration, such as injection.

Further in accordance with the present disclosure, the composition of the present disclosure suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in practicing the methods of the present disclosure is appropriate. Examples of carriers or diluents include fats, oils, water, saline solutions, lipids, liposomes, resins, binders, fillers, alcohols, and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art. The compositions comprising a protein-interacting polypeptide therapy, such as a viral protein-interacting polypeptide therapy, or a polynucleotide encoding a protein-interacting polypeptide, such as a viral protein-interacting polypeptide, may be lyophilized.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in the composition include buffers, amino acids such as glycine and lysine, carbohydrates such as dextrose, mannose, galactose, fructose, lactose, sucrose, maltose, sorbitol, mannitol, etc.

In further embodiments, the present disclosure may include the use of a pharmaceutical lipid vehicle compositions that incorporate compositions comprising a protein-interacting polypeptide therapy, such as a viral protein-interacting polypeptide therapy, or a polynucleotide encoding a protein-interacting polypeptide, such as a viral protein-interacting polypeptide, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the composition(s) comprising a protein-interacting polypeptide therapy, such as a viral protein-interacting polypeptide therapy, or a polynucleotide encoding a protein-interacting polypeptide, such as a viral protein-interacting polypeptide, may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The composition(s) comprising a protein-interacting polypeptide therapy, such as a viral protein-interacting polypeptide therapy, or a polynucleotide encoding a protein-interacting polypeptide, such as a viral protein-interacting polypeptide, may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

### B. Routes of Administration

The therapeutic agents of the disclosure may be administered by the same route of administration or by different routes of administration. The route of administration of the composition may be, for example, intranasally, intravenously, intracerebrally, intracranially, intramuscularly, subcutaneously, topically, orally, mucosally, intradermally, transdermally, intraperitoneally, intraarterially, intraorbitally, by implantation, intravaginally, intrarectally, intrathecally, intraarticularly, intraventricularly, or intrasynovially; by inhalation, injection, infusion, continuous infusion, localized perfusion bathing target cells directly, via a catheter, via a lavage; in creams or in lipid compositions (e.g., liposomes); by other method or any combination of the forgoing as would be known to one of ordinary skill in the art (see, for example, Remington's Pharmaceutical Sciences, 18th Ed. Mack Printing Company, 1990, specifically incorporated by reference herein in its entirety).

In some embodiments, the composition(s) comprising a protein-interacting polypeptide therapy, such as a viral protein-interacting polypeptide therapy, or a polynucleotide encoding a protein-interacting polypeptide, such as a viral protein-interacting polypeptide, is delivered systemically or locally. In some embodiments, the composition comprising a protein-interacting polypeptide therapy, such as a viral protein-interacting polypeptide therapy, or a polynucleotide encoding a protein-interacting polypeptide, such as a viral protein-interacting polypeptide, is delivered by inhalation. In some embodiments, the composition comprising a protein-interacting polypeptide therapy, such as a viral protein-interacting polypeptide therapy, or a polynucleotide encoding a protein-interacting polypeptide, such as a viral protein-interacting polypeptide, is delivered intranasally.

In some embodiments, compositions comprising a protein-interacting polypeptide therapy, such as a viral protein-interacting polypeptide therapy, or a polynucleotide encoding a protein-interacting polypeptide, such as a viral protein-interacting polypeptide, as disclosed herein may be formulated so as to enhance the stability in vivo and/or uptake or absorption by cells, as explained in A. L. Lewis and J. Richard, Therapeutic Delivery 6(2):149-163 (2015), specifically incorporated by reference herein in its entirety. For example, compositions may be formulated with an absorption enhancer, e.g., acyl carnitine, sodium octanoate, sodium caprate, SNAC, SNAD, 5-CNAC, to increase absorption of the protein-interacting polypeptide therapy, such as a viral protein-interacting polypeptide therapy, or a polynucleotide encoding a protein-interacting polypeptide, such as a viral protein-interacting polypeptide, by cells. In some embodiments, formulations to enhance the stability in vivo and/or uptake or absorption by cells will depend on the route of administration of the compositions, for example, intranasally, orally, or by injection.

### 1. Parenteral Routes

Thus, in some embodiments, the composition(s) may be administered via a parenteral route. As used herein, the term “parenteral” includes routes that bypass the alimentary tract. Specifically, the pharmaceutical compositions disclosed herein may be administered for example, but not limited to retro-orbitally, intracerebrally, intracranially, intravenously, intradermally, intramuscularly, intraarterially, intrathecally, subcutaneous, or intraperitoneally U.S. Pat. Nos. 6,737,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions may be prepared by incorporating the active compounds in the required amount in the appropriate solvent with various of the other ingredients enumerated above, as required, followed by filtered sterilization, for example. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### 2. Alimentary Routes

In particular embodiments of the present disclosure, the compositions are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the composition(s) may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10% (by weight), and preferably about 1% to about 2% (by weight).

### 3. Miscellaneous Routes

In other embodiments of the disclosure, the composition(s) may be formulated for administration via various miscellaneous routes, for example, administration by inhalation, topical (i.e., transdermal) administration, and/or mucosal administration (intranasal, vaginal, etc.).

In certain embodiments, the pharmaceutical composition(s) may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

### C. Dosing

The appropriate dosage amount of a composition(s) of the present disclosure administered to the subject can be determined by physical and physiological factors such as body weight, severity and course of condition, the type of disease being treated, the clinical condition of the individual, previous or concurrent therapeutic interventions, the individual's clinical history and response to the treatment, idiopathy of the subject, the route of administration, and the discretion of the attending physician. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at most or least about 0.000001 to at most or at least about 10% (by weight) of an active compound. In other embodiments, the active compound may comprise between about 0.001% to about 1% of the weight of the unit, or about 0.01% to about 0.1%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. Furthermore, such doses can be administered at multiple times during a day, and/or on multiple days, weeks, or months.

In some embodiments, a single dose of a protein-interacting polypeptide therapy, such as a coronavirus S protein-interacting polypeptide therapy, is administered. In some embodiments, multiple doses of a protein-interacting polypeptide therapy, such as a coronavirus S protein-interacting polypeptide therapy, are administered. In some embodiments, an effective dose of a protein-interacting polypeptide therapy, such as a coronavirus S protein-interacting polypeptide therapy, is administered. In some embodiments, a protein-interacting polypeptide therapy, such as a coronavirus S protein-interacting polypeptide therapy, is administered at a dose of at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein. In certain embodiments, the effective dose of a protein-interacting polypeptide therapy, such as a coronavirus S protein-interacting polypeptide therapy, is one which can provide a blood level of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain embodiments, a protein-interacting polypeptide therapy, such as a coronavirus S protein-interacting polypeptide therapy, that is administered to a subject is metabolized in the body to a metabolized therapeutic agent, in which case the blood levels may refer to the amount of that agent. Alternatively, to the extent a protein-interacting polypeptide therapy, such as a coronavirus S protein-interacting polypeptide therapy, is not metabolized by a subject, the blood levels discussed herein may refer to the unmetabolized protein-interacting polypeptide therapy, such as a coronavirus S protein-interacting polypeptide therapy.

In some embodiments, a single dose of a polynucleotide that encodes a protein-interacting polypeptide therapy, such as a coronavirus S protein-interacting polypeptide therapy, is administered. In some embodiments, multiple doses of the polynucleotide that encodes a protein-interacting polypeptide therapy, such as a viral protein-interacting polypeptide therapy, or a polynucleotide encoding a protein-interacting polypeptide, such as a viral protein-interacting polypeptide, are administered. In some embodiments, an effective dose of the polynucleotide that encodes a protein-interacting polypeptide therapy, such as a viral protein-interacting polypeptide therapy, or a polynucleotide encoding a protein-interacting polypeptide, such as a viral protein-interacting polypeptide, is administered. In some embodiments, the polynucleotide that encodes a protein-interacting polypeptide therapy, such as a viral protein-interacting polypeptide therapy, or a polynucleotide encoding a protein-interacting polypeptide, such as a viral protein-interacting polypeptide, is administered at a dose of at least, at most, or about 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, or 1×10¹⁸ polynucleotide copies/kg body weight of the subject, or any range or value derivable therein. In some embodiments, the polynucleotide that encodes a protein-interacting polypeptide therapy, such as a viral protein-interacting polypeptide therapy, or a polynucleotide encoding a protein-interacting polypeptide, such as a viral protein-interacting polypeptide, is administered at a dose of between 1×10⁸ to 1×10¹⁸ polynucleotide copies/kg body weight of the subject. In some embodiments, the polynucleotide that encodes a protein-interacting polypeptide therapy, such as a viral protein-interacting polypeptide therapy, or a polynucleotide encoding a protein-interacting polypeptide, such as a viral protein-interacting polypeptide, is administered at a dose of between 1×10¹¹ to 1×10¹⁴ polynucleotide copies/kg body weight of the subject. In some embodiments, the polynucleotide that encodes a protein-interacting polypeptide therapy, such as a viral protein-interacting polypeptide therapy, or a polynucleotide encoding a protein-interacting polypeptide, such as a viral protein-interacting polypeptide, is administered at a dose of between 1×10¹² to 1×10¹⁵ polynucleotide copies/kg body weight of the subject.

In some embodiments, the polynucleotide that encodes a protein-interacting polypeptide therapy, such as a viral protein-interacting polypeptide therapy, or a polynucleotide encoding a protein-interacting polypeptide, such as a viral protein-interacting polypeptide, is comprised in a vector. In some embodiments, an effective dose of the vector comprising the polynucleotide that encodes a protein-interacting polypeptide therapy, such as a viral protein-interacting polypeptide therapy, or a polynucleotide encoding a protein-interacting polypeptide, such as a viral protein-interacting polypeptide, is administered. In some embodiments, the vector is administered at a dose of at least, at most, or about 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, or 1×10¹⁸ vector copies/kg body weight of the subject, or any range or value derivable therein. In some embodiments, the vector is administered at a dose of between 1×10⁸ to 1×10¹⁸ vector copies/kg body weight of the subject. In some embodiments, the vector is administered at a dose of between 1×10¹¹ to 1×10¹⁴ vector copies/kg body weight of the subject. In some embodiments, the vector is administered at a dose of between 1×10¹² to 1×10¹⁵ vector copies/kg body weight of the subject.

In some embodiments, a single dose of one or more additional disease medications is administered. In some embodiments, multiple doses of the one or more additional disease medications are administered. In some embodiments, an effective dose of the one or more additional disease medications is administered. In some embodiments, the one or more additional disease medications are administered at a dose of at least, at most, or about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range or value derivable therein. In certain embodiments, the effective dose of the one or more additional disease medications is one which can provide a blood level of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the one or more additional disease medications that is administered to a subject is metabolized in the body to a metabolized therapeutic agent, in which case the blood levels may refer to the amount of that agent. Alternatively, to the extent the one or more additional disease medications is not metabolized by a subject, the blood levels discussed herein may refer to the unmetabolized disease medications.

In some embodiments, the therapeutically effective or sufficient amount of composition that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VI. Kits

Certain aspects of the present disclosure also concern kits containing compositions of the disclosure or compositions to implement methods of the disclosure. In some embodiments, kits can be used to neutralize a virus in a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of interacting with one or more virus proteins, such as virus spike proteins, including polypeptides disclosed herein. For example, a kit may comprise at least 1, 2, 3, 4, 5, 6, 7, 8, 9, 10 or more polypeptides disclosed herein that interact with and neutralize a virus protein, such as a virus spike protein.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, synthetic polypeptides, nonsynthetic polypeptides, and/or inhibitors of the disclosure for prognostic or diagnostic applications are included as part of the disclosure. In certain aspects, negative and/or positive control nucleic acids, polypeptides, probes, and inhibitors are included in some kit embodiments.

Kits may further comprise instructions for use. For example, in some embodiments, a kit comprises instructions for detecting a virus antibody in a sample. In some embodiments, a kit comprises instructions for neutralizing a virus in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate embodiments of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific embodiments which are disclosed and still obtain a like or similar result without departing from the spirit and scope of the disclosure.

### Example 1

### The Concept of Polypeptide-Based Inhibition

Viral glycoproteins (e.g., membrane fusion proteins) such as coronavirus spike proteins, HIV-1 gp160, Ebola GP, and influenza virus HA, are oligomeric Class-I transmembrane glycoproteins on the viral envelope.

Coronavirus spike (S) proteins are cleaved to give rise to N-terminal S1 regions and C-terminal S2 regions (FIG. 1A). The N-terminal S1 regions contain N-terminal domains (NTDs) and receptor-binding domains (RBDs) responsible for attachment to cell-surface receptors, and the C-terminal S2 regions trimerize to form an elongated “stem” domain mainly for inducing fusion of viral envelope with the host membrane through a large-scale conformational change¹⁷⁻²⁰. The S1 regions are the major target for neutralizing antibodies elicited by natural infection or vaccination and are therefore under constant positive selection for escape variants. For instance, since the onset of COVID-19, six major variants of SARS-CoV-2 with extensive mutations have emerged, including B.1.1.7, B.1.351, P.1, B.1.617 2, and C.37 variant strains (FIG. 1A). The mutations in the SARS-CoV-2 spike (SARS2-S) proteins are concentrated in the S1 region (FIG. 1A). On the other hand, the C-terminal S2 regions responsible for oligomerization and membrane fusion are more conserved among different coronavirus strains. Similar to SARS2-S, HIV-1 gp160, Ebola GP, and influenza virus HA are all cleaved to yield an N-terminal globular domain responsible for receptor binding and a C-terminal elongated domain mainly for membrane fusion.

Upon entry into host cell, the viral genome guides the synthesis of new coronavirus spike proteins by ribosomes, which are then folded, assembled, and translocated into endoplasmic reticulum (ER) membranes, after which the spike proteins transit through the ER to the Golgi intermediate compartment for interaction with newly-replicated genomic RNA to produce new virions.

Without wishing to be bound by theory, polypeptides comprising fragments of coronavirus spike proteins, e.g., polypeptides derived from the SARS2-S S2 region, that maintain the same oligomeric interface as the native, wild-type spike proteins, can form non-native protein complexes with native, wild-type spike proteins upon oligomerization of the polypeptide with the native, wild-type spike proteins. For example, a polypeptide comprising a segment of the SARS2-S S2 region comprising the same oligomeric interface as the wild-type SARS2-S S2 region can form a non-native protein complex with wild-type SARS2-S by competing with the wild-type SARS2-S at the S2 region. Similarly, polypeptides comprising a segment of the HIV-1 gp160, Ebola GP, and influenza virus HA membrane fusion domains and comprising the same oligomeric interface as the wild-type proteins can form non-native protein complexes with the wild-type proteins at the oligomerization interface of the polypeptides and the wild-type proteins. These loosely-packed non-native protein complexes would fail the quality control system and result in proteasomal degradation of the non-native protein complexes. The net outcome is a significantly lowered amount of viral proteins on host cell membranes and on the envelope of newly generated virus progenies, thus impairing their infectivity (FIG. 1B). Also, the polypeptide comprising a fragment of the conserved S2 region or other conserved viral glycoprotein region would be more resistant to mutations and could potentially be “universal” against different lineages of viruses (i.e., a pan-virus inhibitor).

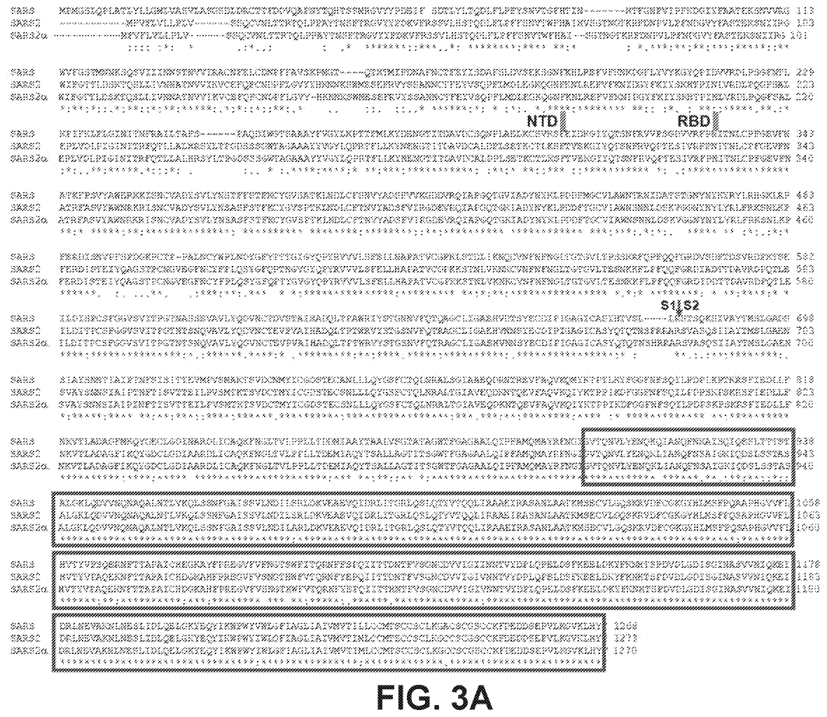

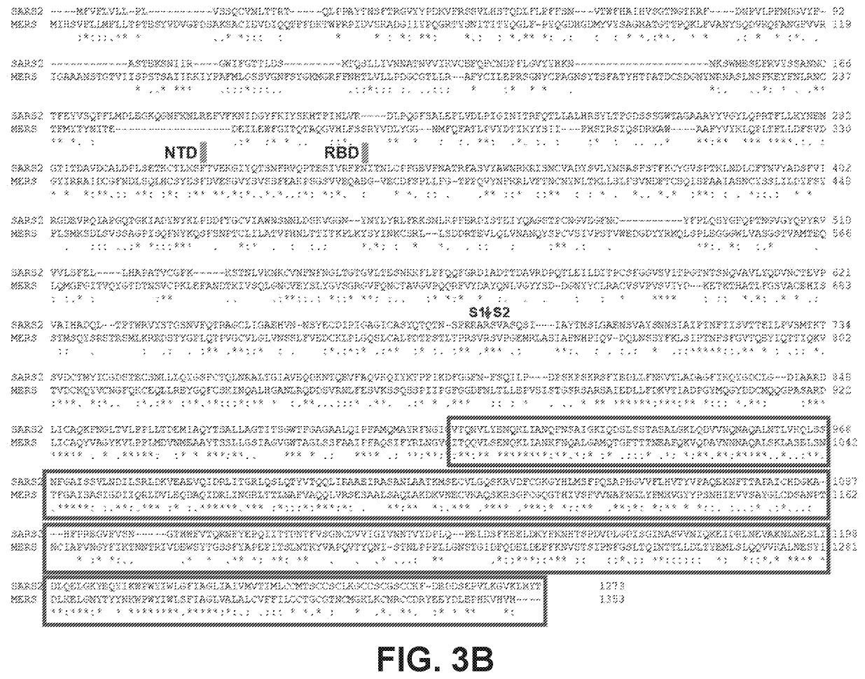

To test this “polypeptide-based inhibition” strategy, two polypeptides, CoV-F1 (hereinafter, F1; SEQ ID NO:43) and CoV-F2 (hereinafter, F2; SEQ ID NO:44) (FIG. 1A, FIG. 3A, FIG. 16A, FIG. 16B), were derived and generated from the SARS2-S S2 protein sequence. F1 (SEQ ID NO:2) encompasses residues 911-1273 of the wild-type SARS2-S and is slightly longer than F2 (SEQ ID NO:27), which includes residues 985-1273 of the wild-type SARS2-S. Both polypeptides contain a C-terminal FLAG epitope for ease of identification and an N-terminal signal peptide (SP) from wild-type SARS2-S for cell-surface translocation.

### Example 2

### F1 Polypeptide Potently Inhibits Expression and Surface Translocation of Multiple Coronavirus Spike Glycoproteins

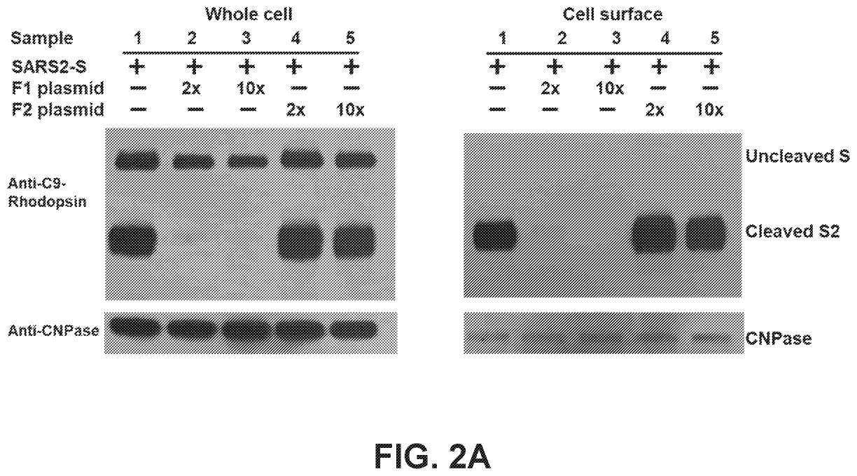

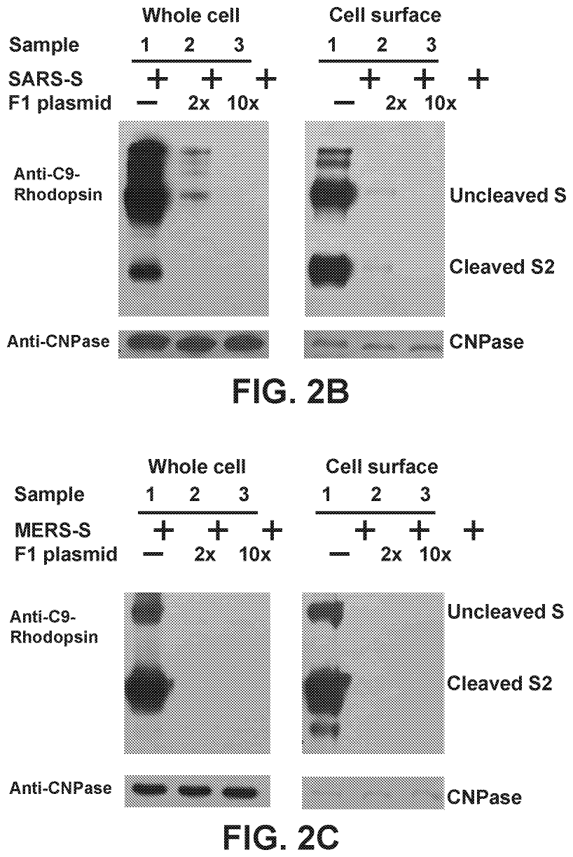

The impacts of F1 and F2 on the expression and cell surface translocation of SARS2-S protein to plasma membrane was investigated. Transient transfection of SARS2-S-coding plasmid resulted in a good level of proteins detected in HEK293T whole cell lysate, with most of the expressed proteins cleaved (FIG. 2A). This high-efficiency cleavage of SARS2-S proteins agreed with the novel polybasic cleavage site at the S1/S2 boundary^{17,18,24, 25}. Moreover, only the S2 fragments of the cleaved SARS2-S protein were labeled by biotin, affinity-purified by anti-biotin antibody, and detected in the cell surface fraction (FIG. 2A), suggesting that only properly cleaved SARS2-S proteins were translocated to the cell surface. When F1-coding plasmid was co-transfected with SARS2-S-coding plasmid, even at a two-fold molar ratio, the predominant cleaved S2 band of SARS2-S was almost completely diminished in whole-cell lysate and in cell-surface fraction (FIG. 2A). Thus, F1 strongly inhibited the expression and cell surface translocation of SARS2-S.

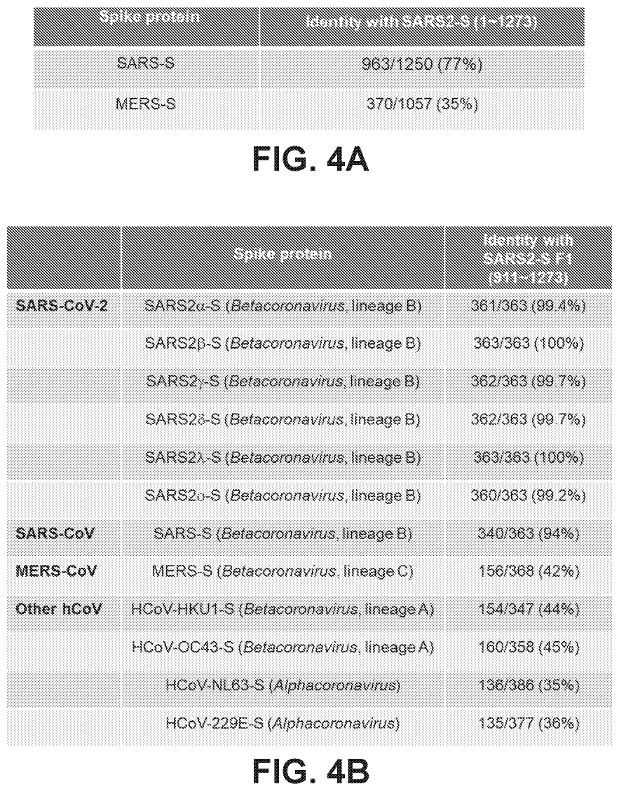

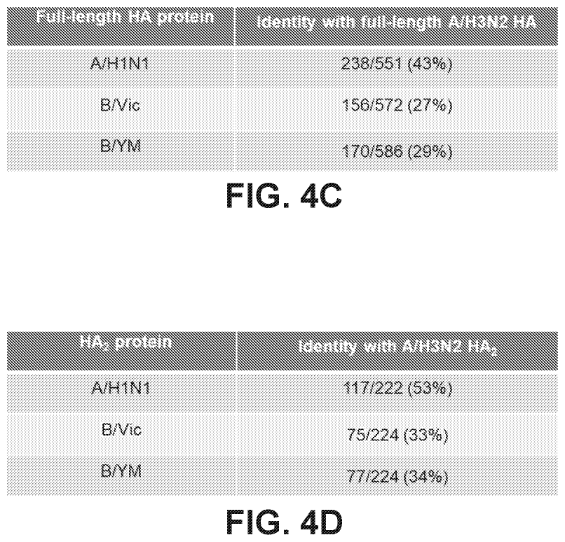

Besides COVID-19 SARS-CoV-2, in the past 20 years have witnessed two other major threats from coronaviruses, the 2002-2003 outbreak from SARS-CoV and the 2012-2014 outbreak from MERS-CoV^{1,2}. Sequence comparison among the spike glycoproteins from these coronaviruses with COVID-19 SARS2-S uncovered a wide range of sequence identity levels (FIG. 3, FIG. 4). The N-terminal S1 regions of the spike proteins, especially the NTB domains, are highly variable, while the C-terminal S2 regions, in particular the regions that are included in the F1 polypeptide, are highly conserved among these proteins. For instance, 2002 SARS-CoV spike protein (SARS-S) shares 77% amino acid sequence identity with full-length COVID-19 SARS2-S (FIG. 3A, FIG. 4A). Comparing only the region included in SARS2-S F1 polypeptide (residues 911-1273), the identical residues increase to 94% (FIG. 3A, FIG. 4B). The MERS spike protein (MERS-S) shares only 35% amino acid sequence identity with full-length SARS2-S and 42% with the SARS2-S F1 polypeptide (FIG. 3B, FIG. 4). Consequently, these spike proteins were used to test the sensitivity of F1-induced inhibition to amino acid mutations.

Even with F1-coding plasmid at a two-fold molar ratio, the cleaved full-length SARS-S and MERS-S bands and the cleaved S2 bands were almost completely diminished in whole-cell lysate and in cell-surface fraction (FIGS. 2B-2C). Thus, despite the lower sequence identity, F1 also strongly interferes with the proper assembly and translocation of SARS-S and MERS-S glycoproteins to cell surface.

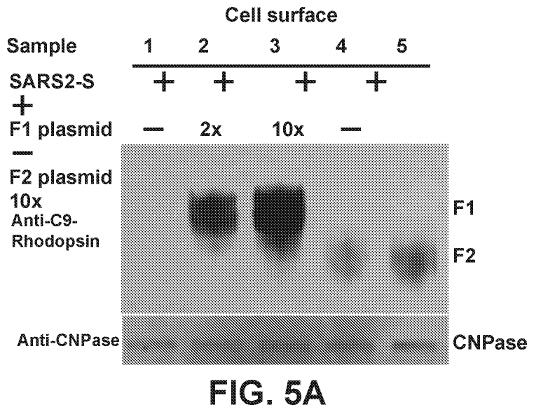

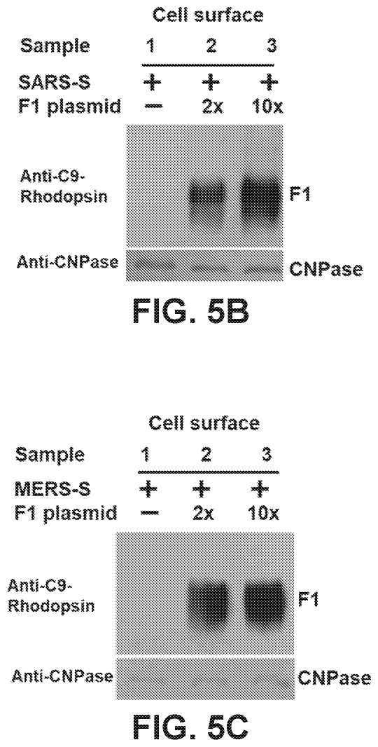

In addition, consistent with the robust inhibitory activity exhibited by F1 (FIG. 2), a high level of F1 was consistently detected in the cell surface fraction (FIG. 5), but only a low level of F2 was detected in the cell surface fraction (FIG. 5A). Without wishing to be bound by theory, F2 may be destabilized by the missing 74 residues at the N-terminus as compared to F1 (FIG. 1A), which is consistent with the non-interference of F2, as shown in FIG. 2A.

### Example 3

### F1 Interacts with Coronavirus Spike Proteins at the Protein Level

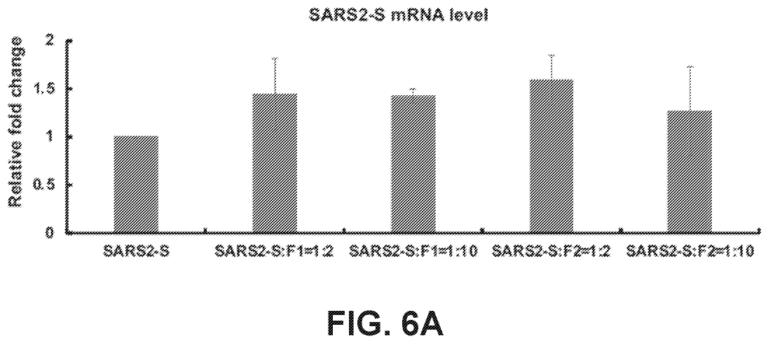

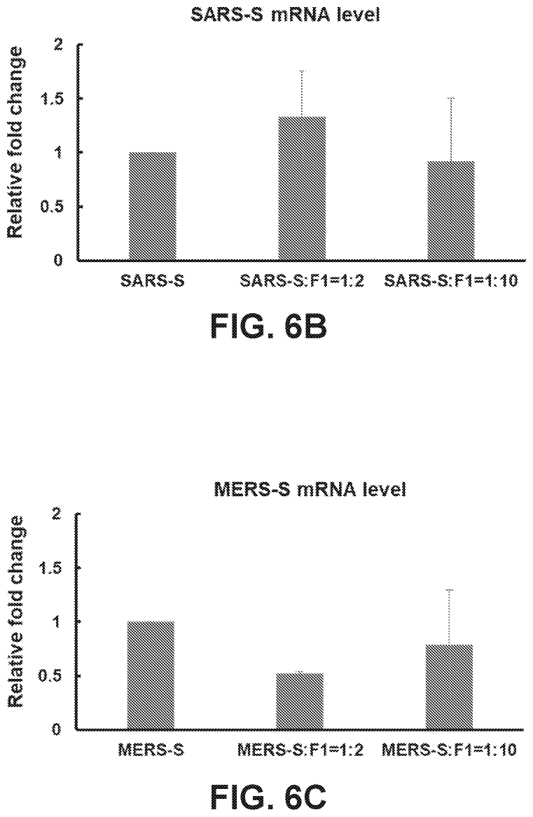

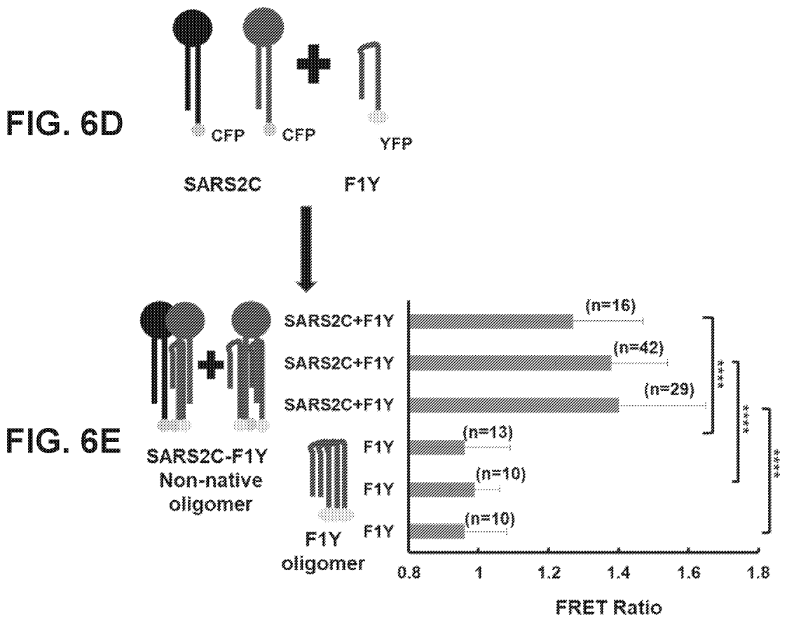

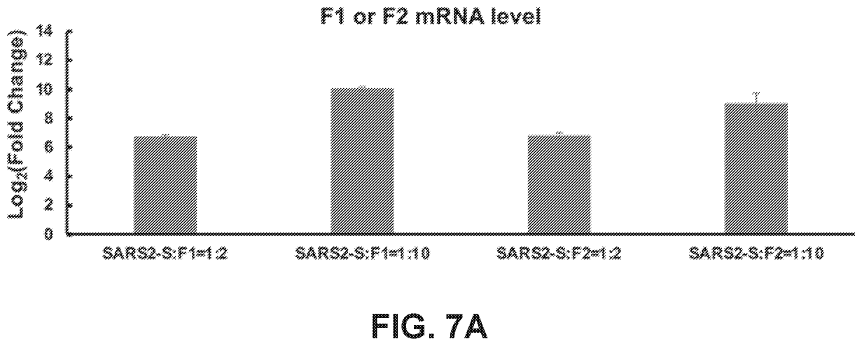

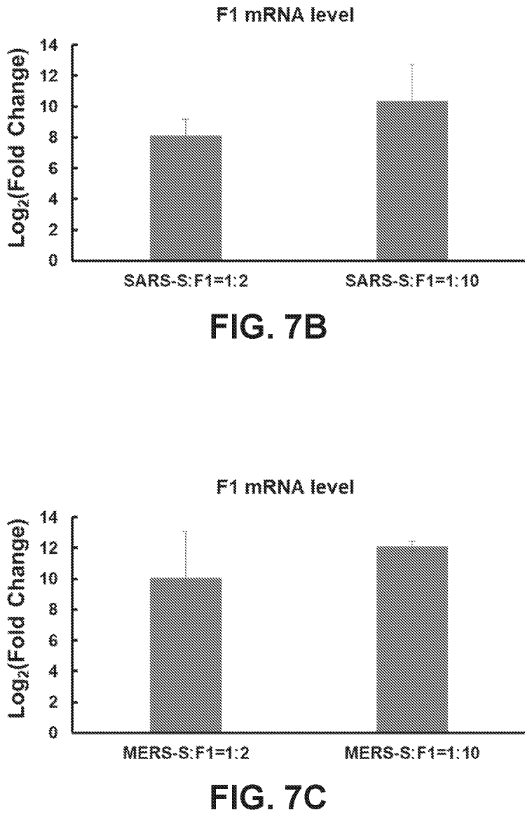

In order to probe the mechanism of F1-mediated inhibition of SARS2-S expression and translocation, the inventors analyzed the mRNA levels of COVID-19 SARS2-S (FIG. 6A) and F1 or F2 (FIG. 7A), 2002 SARS-S (FIG. 6B) and F1 (FIG. 7B), as well as 2012 MERS-S (FIG. 6C) and F1 (FIG. 7C). The presence of F1- or F2-coding plasmids did not significantly change the mRNA levels of coronavirus spike proteins, which were kept at a relatively constant level (between 50%-150%) compared to samples transfected with plasmids coding the respective coronavirus spike proteins only (FIG. 6). The mRNA levels of F1 were on the order of 2^{6.5}-2¹² when normalized against endogenous GAPDH (FIG. 7), agreeing with the high potency of F1-mediated inhibition. The mRNA levels of F2 were comparable with those of F1. Without wishing to be bound by theory, this further supports the notion that non-inhibition of F2 to is due to the instability of the synthesized F2 polypeptide, as suggested by western blot results in FIG. 5A. These data suggest that F1-mediated inhibition of various coronavirus spike glycoproteins was not at the mRNA level.

The inventors next investigated whether F1-mediated inhibition is at the protein level via direct interaction, or competition, with the SARS2-S protein to form non-native oligomeric protein complexes in the cell. SARS2-S and F1 were each tagged with a monomeric green fluorescent protein (GFP) variant at the extreme C-terminus, CFP for SARS2-S (SARS2C) and YFP for F1 (F1Y) (FIG. 6D). SARS2C- and/or F1Y-coding plasmids were used to transiently transfect HEK293T cells plasmids (at a 1:1 molar ratio), and at 48 hours post-transfection, fluorescence resonance energy transfer (FRET) between CFP and YFP was monitored using the three-cube approach.¹⁴ Despite the fact that when F1-harboring plasmid was co-transfected with SARS2-S-carrying plasmid, even at a two-fold molar ratio, the predominant cleaved S2 band was almost completely diminished (FIG. 2A). Although the non-native oligomers formed by SRARS2C and F1Y were expected to be highly unstable, FRET signals between the SARS2C protein and the F1Y polypeptide (at a 1:1 molar ratio in cotransfection) were robustly detected, where the FRET ratio between SARS2C and F1Y was significantly higher than the F1Y-only control (p<0.0001 in an unpaired t-test, FIG. 6E), supporting direct protein-protein interactions and formation of non-native oligomers between SARS2-S protein and F1 polypeptide, as depicted in FIG. 1B.

### Example 4

### F1 Polypeptide Express from a Minicircle Potently Inhibits Expression and Surface Translocation of Coronavirus Spike Glycoproteins

The high potency of F1 in inhibiting the expression and surface translocation of spike glycoproteins from human coronaviruses that caused severe outbreaks or pandemic between 2002 to 2022 suggests that F1 is promising an effective therapeutic agent against different coronavirus lineages over a long time period. Therefore, the inventors sought to identify a convenient way to deliver F1 for therapeutic purposes. Since F1 directly interacts with its target spike protein to form non-native oligomers concomitant with protein synthesis and folding, in some embodiments, the F1-coding gene is delivered to the site of action.

Minicircles are a type of newly-developed DNA carriers for gene therapy²⁶. The main features of minicircles include a clean gene background with minimal viral or bacterial gene elements, little to no risk of genome integration or inflammation, and sustained high-level protein expression, and the small size of the minicircles may greatly facilitate cell entry and/or allow the use of aerosols for drug delivery²⁷. The latter may be a distinct advantage against coronavirus-caused respiratory diseases.

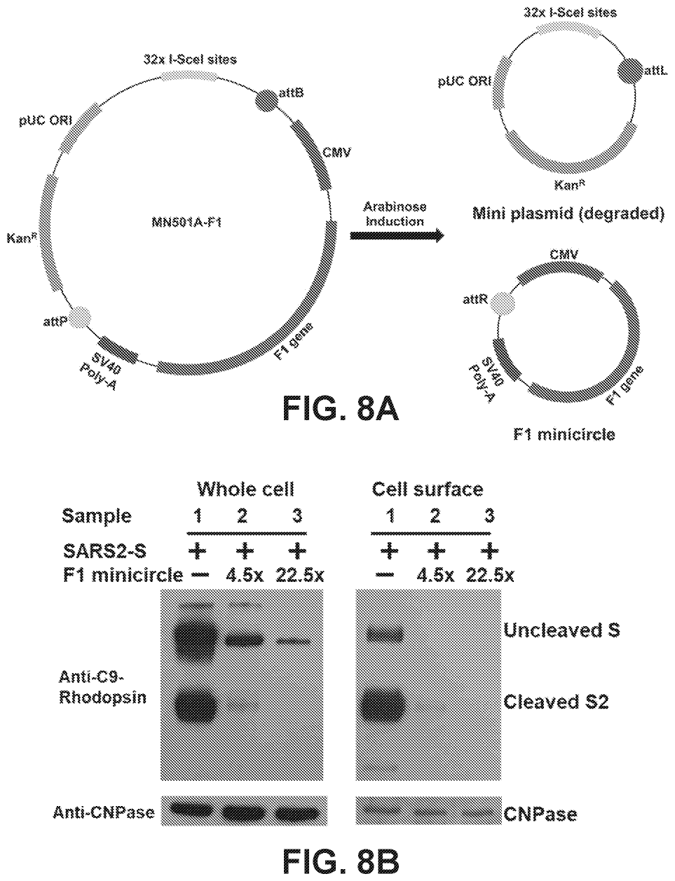

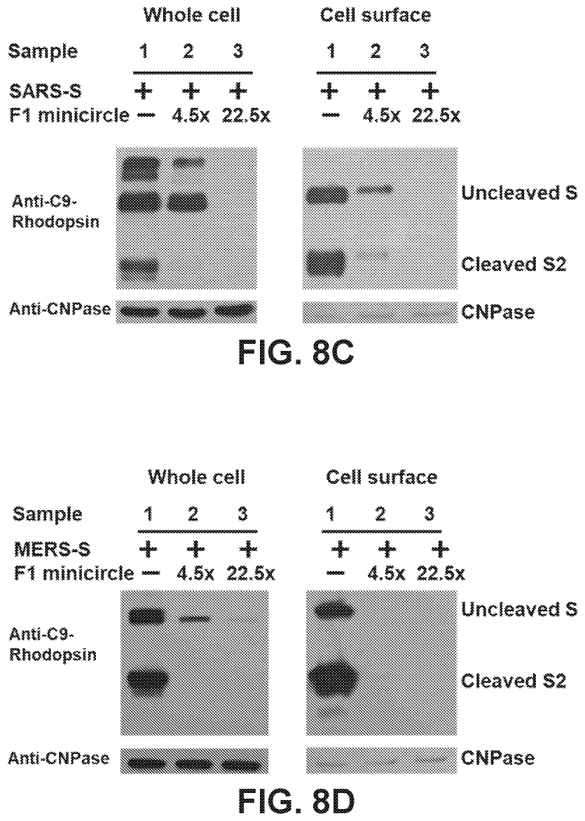

An F1 minicircle was generated by inserting the F1-coding sequence into a modified parental minicircle cloning vector pMC.CMV-MCS-SV40polyA (FIG. 8A) and tested its efficacy in inhibiting expression and surface translocation of COVID-19 SARS2-S (FIG. 8B), 2002 SARS-S (FIG. 8C), and 2012 MERS-S (FIG. 8D) glycoproteins. Compared to controls where no minicircle was used, the presence of F1 minicircles, at a 4.5-fold molar ratio, almost completely abolished cell-surface translocation of all three spike proteins (FIGS. 8B-8D). Importantly, this level of potent interference was achieved under a situation in which pcDNA3.1-based plasmids harboring coronavirus spike protein-coding genes were efficiently replicated in HEK293T cells, while F1 minicircles are not. Therefore, F1 minicircles unexpectedly demonstrated strong inhibition upon proper assembly of these coronavirus spike proteins.

### Example 5

### Minicircle-Expressed F1 Reduces SARS2-S Protein Levels on Intact Pseudoviruses & Impairs Pseudovirus Infectivity

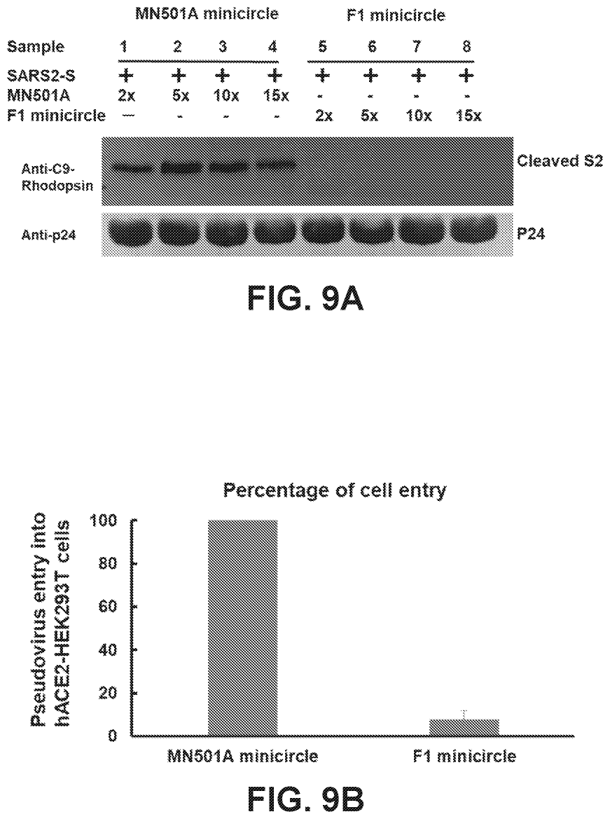

To investigate the consequences of the reduced cell-surface translocation of coronavirus spike proteins expressed from the F1 minicircle, the level of SARS2-S protein on pseudoviruses generated using luciferase-expressing, env-defective HIV-1 genome plasmid pRL4.3-Luc-RE in the presence of different molar ratios of control minicircle made from the empty parental vector (MN501A) or F1 minicircle was compared. In order to make sure that only spike proteins anchored on the pseudovirus envelope were accounted for, a QUICKTITER™ Lentivirus Titer kit was employed to precipitate intact pseudoviruses from cleared supernatant prior to analysis by western blot. Surprisingly, even with only a two-fold molar ratio of F1 minicircle, almost no SARS2-S was detected on the generated intact pseudoviruses (FIG. 9A). Furthermore, these pseudoviruses failed to infect hACE2-expressing HEK293T cells (FIG. 9B).

### Example 6

### Minicircle-Expressed F1 Exhibits Strong Antiviral Activity in Humanized hACE2 Mouse Model

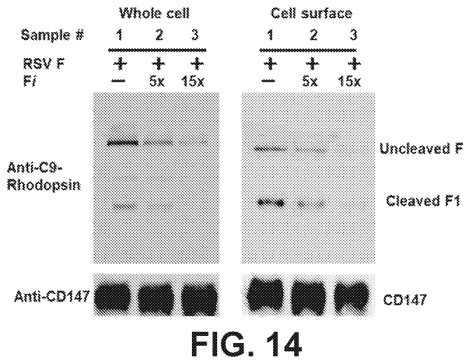

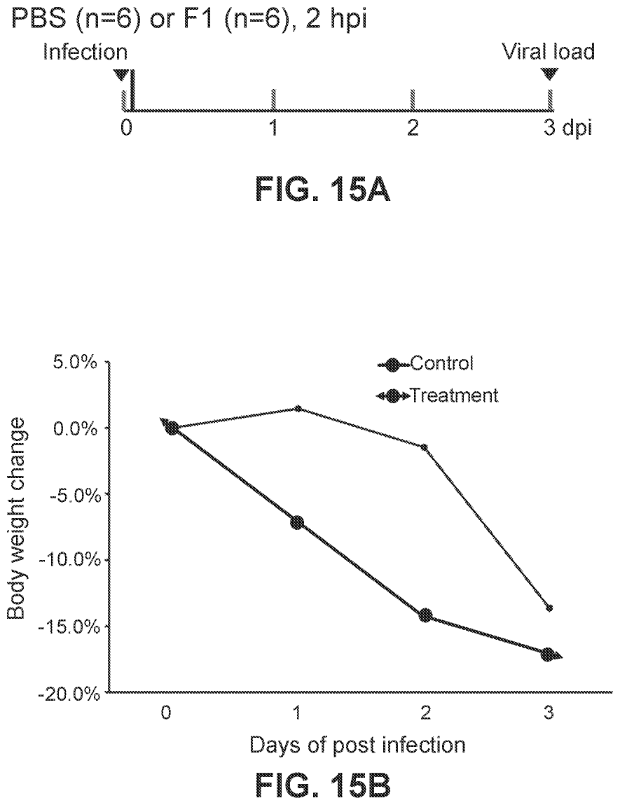

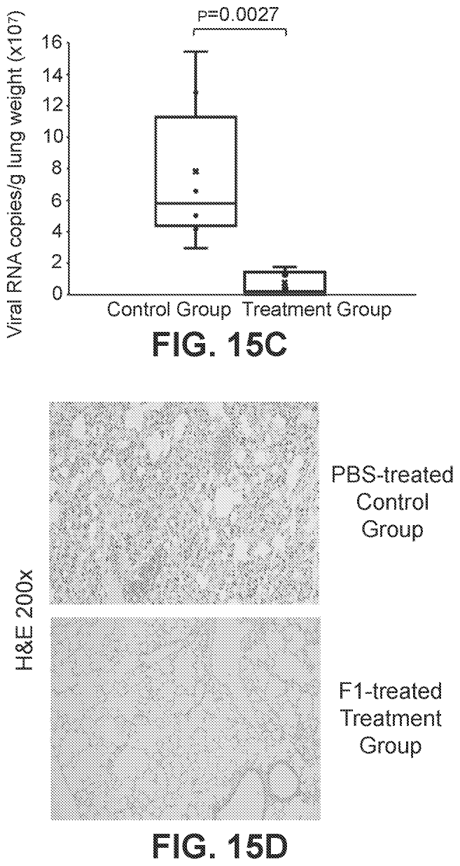

hACE2-IRES-luc mice were used to test the antiviral activity of the F1 polypeptide. Mice were randomly divided into two groups (n=6). On Day 0, mice were inoculated with 10⁴ PFU of SARS-CoV-2 virus. At 2 hours post-inoculatino, mice in the Control Group were treated once with PBS, while mice in the Treatment Group were each treated with 50 μL antiviral F1 suspension (FIG. 15A).

During the course of the experiments, both groups experienced similar changes in body weight (FIG. 15B). On day 3 post-inoculation, average animal body weights in Control and Treatment groups were decreased by 13.7% and 17.1%, respectively (FIG. 15B, Table 1).

TABLE 1
Body weight changes of hACE2-IRES-luc mice

| | | Body Weight (g) | | | |
|---|---|---|---|---|---|
| Groups | Days | Day 0 | Day 1 | Day 2 | Day 3 |
| Control | Mean | 20.53 | 20.82 | 20.20 | 17.73 |
| | BWC* | 0.0% | 1.4% | −1.6% | −13.7% |
| Treatment | Mean | 20.00 | 19.45 | 18.05 | 17.41 |
| | BWC | 0.0% | −7.4% | −14.1% | −17.1% |

*BWC: Percentage of body-weight change.

The viral loads in lungs of SARS-CoV-2 infected mice were significantly lowered (p=0.0027) in the Treatment Group compared to the Control Group (FIG. 15C). Furthermore, the one-time treatment with 50 μL antiviral F1 suspension in the Treatment Group significantly alleviated the severity of interstitial pneumonia caused by SARS-CoV-2 virus in hACE-mice lung tissues (FIG. 15D). These data suggest that, in some embodiments, F1 polypeptide is highly efficacious in preventing the infection and propagation of SARS-CoV-2 virus in vivo.

### Example 7

### GP160/Polypeptide Inhibits Expression and Surface Translocation of HIV Spike Glycoproteins

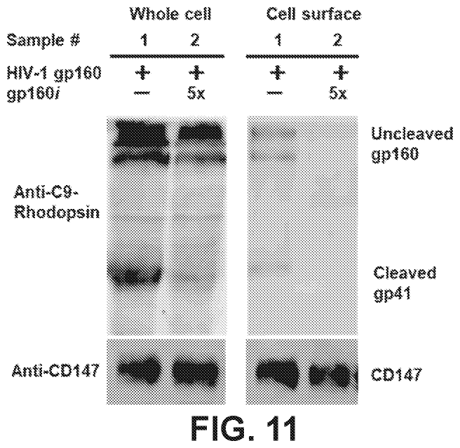

The inventors also assessed the impact of the inhibitory polypeptide gp160i (FIG. 16C; SEQ ID NO:45) on the expression of HIV-1 gp160 glycoprotein and cell surface translocation of the gp160 glycoprotein. Transient transfection of gp160-coding plasmid resulted in a good level of protein detected in HEK293T whole cell lysate, with most of the expressed proteins cleaved (FIG. 11). Cotransfection of the gp160i-coding plasmid with HIV-1 gp160-coding plasmid diminished the cleaved gp41 fraction in whole-cell lysate, especially at a fifteen-fold molar ratio (FIG. 11). Thus, gp160i strongly inhibits expression of HIV-1 gp160 glycoprotein.

### Example 8

### GP/Polypeptide Inhibits Expression and Surface Translocation of Ebola Spike Glycoproteins

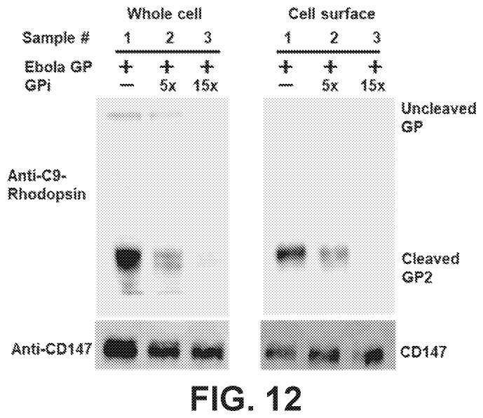

The inventors also assessed the impact of the inhibitory polypeptide GPi (FIG. 16D; SEQ ID NO:46) on the expression of Ebola GP glycoprotein and cell surface translocation of Ebola GP glycoprotein. Transient transfection of Ebola GP-harboring plasmid yielded a good level of protein detected in HEK293T whole cell lysate, with most of the expressed proteins cleaved (FIG. 12). Moreover, only the GP2 fragment of the cleaved Ebola GP protein was detected on cell surfaces that were labeled by biotin and affinity-purified by anti-biotin antibody (FIG. 12), suggesting that only properly cleaved Ebola GP glycoprotein are transported to cell surface. When GPi-coding plasmid was co-transfected with GP-coding plasmid, the cleaved GP2 band was diminished in whole-cell lysate and in the cell-surface fraction, especially at a fifteen-fold molar ratio (FIG. 12). Thus, Ebola GPi strongly inhibits the expression of Ebola GP glycoprotein and its translocation to the cell surface.

### Example 9

### Fi Polypeptide Inhibits Expression and Surface Translocation of RSV Spike Glycoproteins

The inventors also assessed the impact of the inhibitory polypeptide Fi (FIG. 16E; SEQ ID NO:47) on the expression of RSV type A F glycoprotein and cell surface translocation of the F glycoprotein. Transient transfection of RSV F-coding plasmid resulted in a good level of protein detected in HEK293T whole cell lysate, with most of the expressed proteins cleaved (FIG. 14A). Cotransfection of the Fi-coding plasmid with RSV F-coding plasmid diminished the cleaved fraction in whole-cell lysate (FIG. 14A). Thus, Fi strongly inhibits expression of RSV F glycoprotein.

To investigate the consequences of reduced cell surface translocation of RSV glycoproteins when cotransfected with the Fi-coding plasmid, the levels of RSV F glycoprotein on pseudoviruses generated in the presence of different molar ratios of Fi-coding plasmid were measured. Cotransfection of the Fi-coding coding plasmid with F-coding plasmid diminished the RSV F glycoprotein detected on the generated intact pseudoviruses (FIG. 14B).

### Example 10

### HAi Polypeptide Inhibits Expression and Surface Translocation of Influenza Virus Spike Glycoproteins

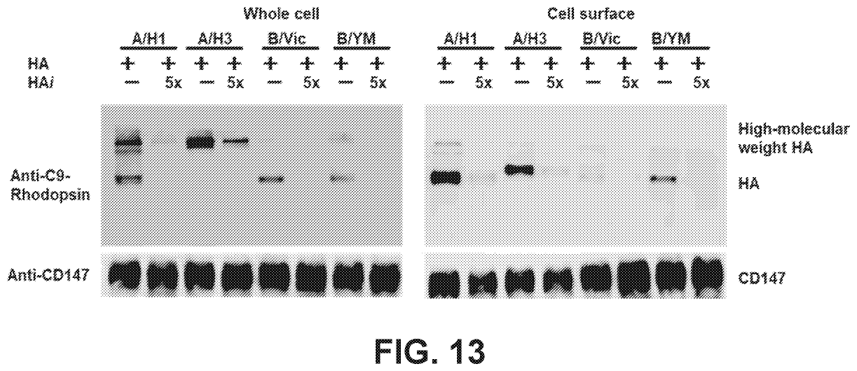

The inventors also assessed the impact of inhibitory polypeptide HAi (FIG. 16F; SEQ ID NO:48) derived from the HA sequence of the 2019-2020 flu vaccine strain A/Hong Kong/45/2019 (H3N2) virus on the expression of influenza HA proteins and their translocation to plasma membrane. Transient transfection of influenza A/H1, A/H3, B/Vic, and B/YM HA-coding plasmid yielded a good level of protein detected in HEK293T whole cell lysate and in the cell-surface fraction (FIG. 13). Cotransfection of the influenza HAi-coding plasmid with influenza A/H1, A/H3, B/Vic, and B/YM HA-coding plasmid diminished the influenza HA fraction in whole cell lysate and cell-surface fraction (FIG. 13). In particular, the HAi polypeptide was able to potently inhibit the expression and translocation of HA proteins of all four flu vaccine strains, A/Hawaii/70/2019 (H1N1), A/Hong Kong/45/2019 (H3N2), B/Washington/02/2019 (B/Victoria lineage (B/Vic)), B/Phuket/3073/2013 (B/Yamagata lineage (B/YM)). Importantly, A/Hong Kong/45/2019 (H3N2) HA from which the HAi polypeptide is derived shares an amino acid sequence identity of 43%, 27%, and 29% with the HA protein from A/Hawaii/70/2019 (H1N1), B/Washington/02/2019 (B/Vic), and B/Phuket/3073/2013 (B/YM), respectively (FIG. 4C, FIG. 4D). Thus, influenza HAi strongly inhibits the expression of influenza HA and its TRANSLOCATION to the cell surface.

These data further demonstrated that the use of a partial sequence is an effective method for targeted reduction of protein expression of important human pathogens, even when the level of sequence identity is very low.

### Example 11

### Exemplary Methods

Plasmids and constructs. The pcDNA3.1 plasmids harboring the genes encoding SARS-CoV spike (GenBank accession number AFR58740.1), SARS-CoV-2 spike (GenBank accession number QHD43416.1), and human ACE2 (GenBank accession number NM_021804) were kind gifts from Dr. Fang Li¹⁹ (Addgene plasmid #145031, 145032 and 145033, respectively). The pcDNA3.1 plasmids harboring the genes encoding MERS-CoV spike (GenBank accession number QBM11748.1), F1, or F2 polypeptides, pEZT-BM plasmids harboring the genes coding HIV-1 gp160 (GenBank accession number NP_057856.1); Zaire EBOV GP (GenBank accession number AAN37507.1); RSV F (GenBank accession number QKN22797.1); HA proteins for A/Hawaii/70/2019 (H1N1), A/Hong Kong/45/2019 (H3N2), B/Washington/02/2019 (B/Vic), and B/Phuket/3073/2013 (B/YM)), gp160i, GPi, Fi and HAi were synthesized by GENSCRIPT® Biotech (Piscataway, NJ, USA). The parental minicircle vector pMC.CMV-MCS-SV40polyA (Cat. #MN501A-1), ZYCY10P3S2T E. coli minicircle producer strain competent cells (Cat. #MN900A-1) and Arabinose Induction Solution (Cat. #MN850A-1) were purchased from System Biosciences (Palo Alto, CA, USA). The luciferase-expressing, env-defective HIV-1 genome plasmid pRL4.3-Luc-R-E- (Cat. No. 3418) was obtained through the NIH AIDS Reagent Program, Division of AIDS, NIAID, NIH, USA. HEK293T cells (Cat. No. CRL-11268) were purchased from American Type Culture Collection (Manassas, VA, USA) and maintained in Dulbecco's Modified Eagle Medium (DMEM) containing 10% fetal bovine serum (GIBCO™) at 37° C. in 5% CO₂ cell culture incubator.

Reagents. C9-rhodopsin antibody (1D4) HRP (Cat. #sc57432 HRP) and 2′,3′-cyclinc nucleotide 3′-phosphodiesterase (CNPase) antibody (Cat. No. A01308), and CD147 antibody (Cat. No. ab108308) were purchased from SANTA CRUZ BIOTECHNOLOGY®, Inc. (Dallas, TX, USA), GENSCRIPT® Biotech (Piscataway, NJ, USA), and ABCAM® (Cambridge, UK), respectively. QUICKTITER™ Lentivirus Titer kit was purchased from Cell Biolabs Inc (San Diego, CA, USA). PIERCE™ Cell Surface Protein Biotinylation and Isolation Kit (Cat. #A44390) was purchased from THERMO SCIENTIFIC®, Waltham, MA, USA. QUICK-RNA™ miniprep Kit (Cat. #R1054) and ZYMOPURE™ II Plasmid Maxiprep Kit (Cat. #D4203) were obtained from Zymo Research, Irvine, CA, USA. ISCRIPT™ Reverse Transcription Supermix (Cat. #1708840) was purchased from BIO-RAD®, Hercules, CA, USA. BIMAKE™ SYBR Green qPCR Master Mix (Cat. #B21203) was obtained from Bimake, Houston, TX, USA. LIPOFECTAMINE™ 3000 was obtained from INVITROGEN®, Carlsbad, CA, USA. The reagent IN VIVO-JETPEI® (Cat. No. 201-10G) was purchased from POLYPLUS-TRANSFECTION® SA, Vectura, France. ONE-GLO™ EX luciferase kit was purchased from PROMEGA®, Madison, WI, USA. The Arabinose Induction Solution (Cat. No. MN850A-1) was purchased from System Biosciences (Palo Alto, CA, USA).

Cell surface biotinylation and protein purification. Cell surface biotinylation and protein purification were performed using PIERCE™Cell surface Protein Biotinylation and Isolation Kit following the manufacturer's instruction. Briefly, cell surface proteins on HEK293T cells were first labeled with Sulfo-NHS-SS-Biotin at 4° C. for 30 minutes, which were then stopped by adding Tris-buffered saline and further washed. After cells were lysed with Lysis Buffer, lysate was cleared by centrifugation. Cleared lysate was incubated with NEUTRAVIDIN™ Agarose to allow binding of biotinylated proteins. After extensive wash, the bound proteins were eluted with Elution Buffer containing 10 mM DTT. The cleared lysate (“Whole cell” fraction) and eluted proteins (“Cell surface” fraction) were run on 10% SDS-PAGE and the spike proteins were detected by C9-rhodopsin antibody 1D4 HRP. Endogenous membrane-anchored protein CNPase detected by anti-CNPase antibody or CD147 detected by anti-CD147 antibody were used as an internal control.

Total RNA isolation and RT-qPCR. Total RNA was purified using QUICK-RNA™ miniprep Kit. Reverse transcription was carried out using ISCRIPT™ Reverse Transcription Supermix. qPCR was performed using BIMAKE™SYBR Green qPCR Master Mix with the following primers:

1060 (SARS-S Forward):
GTTCAAGGACGGCATCTACTT
1061 (SARS-S Reverse):
ACGCTCTGGGACTTGTTATTC
1062 (SARS2-S Forward):
GACAAAGTGCACCCTGAAGA
1063 (SARS2-S Reverse):
GGGCACAGGTTGGTGATATT
1089 (MERS-S Forward):
GAACGCCTCTCTGAACTCTTT
1090 (MERS-S Reverse):
GTCCTCGGTGATGTTGTATGT
1091 (F1 and F2 Forward):
GATTAGAGCCGCCGAGATTAG
1092 (F1 and F2 Reverse):
GGACTGAGGGAAAGACATGAG

Since the synthesized genes of F1 and F2 were optimized for mammalian expression and different from the gene coding for SARS2-S, the qPCR primers 1091 and 1092 were unique to F1 and F2, while 1062 and 1063 were unique to SARS2-S.

Minicircle production. Minicircle parental vector pMC.CMV-MCS-SV40polyA was modified to shorten the size of the final minicircle without affecting its function. F1-coding gene was cloned into the minicircle parental vector pMC.CMV-MCS-SV40polyA (MN501A) to yield MN501A-F1. MN501A or MN501A-F1 was transformed into ZYCY10P3S2T E. coli minicircle producer strain²⁸ competent cells following the manufacturer's instruction. The production of minicircle DNA was induced with the addition of Arabinose Induction Solution. Minicircle DNA was purified by using ZYMOPURE™ II Plasmid Maxiprep Kit per the manufacturer's instruction.

Pseudovirus generation, precipitation and concentration. Pseudovirus generation followed established protocols. HEK293T cells were seeded on 6-well plates the night before. The next day, pcDNA3.1-SARS2-S (0.6 μg) and pRL4.3-Luc-R-E- (0.6 μg) were used to transfect one-well HEK293T cells using LIPOFECTAMINE™ 3000. MN501A minicircle or F1 minicircle at indicated molar ratio was included in the transfection mixture. At 16 hrs post-transfection, the HEK293T cells were fed with fresh medium. At 48 hrs after medium change, the supernatant of each well of the 6-well plates was harvested, and centrifuged at 300 g for 5 min to remove cell debris. Intact pseudoviruses were purified using QUICKTITER™ Lentivirus Titer kit following the manufacturer's instruction. The virus lysate was analyzed by western blot using C9-rhodopsin antibody 1D4 HRP for spike proteins, and FITC-conjugated anti-p24 mAb and HRP-conjugated anti-FITC mAb for p24, which served as an internal control. A portion of pseudovirus-containing supernatant was concentrated by PEG8000 and used for luciferase assay of cell entry.

Luciferase assay of cell entry by recombinant pseudovirus. HEK293T cells were seeded on 100 mm dishes the night before. The next day, HEK293T cells were transfected with g pcDNA3.1-hACE2 using LIPOFECTAMINE™ 3000. At 16 hrs post-transfection, the cells were resuspended in DMEM medium, and plated onto 96-well white plates to which 10 L concentrated pseudovirus was already added to each well. Two hours later, each well was added with equal volume of DMEM containing 20% FBS. The cells were further incubated for 36 hours, then equal volume of ONE-GLO™ EX Luciferase Assay Reagent was added, after incubation for 3 min, the luminescence signals were recorded.

FRET between SARS2C and F1Y. High quality/high resolution automated imaging was performed on a GE Healthcare DeltaVision LIVE epifluorescence image restoration microscope using an Olympus PlanApoN 60X/1.42 NA objective and a 1.9 k×1.9 k pco.EDGE sCMOS_5.5 camera with a 1024×1024 FOV. The filter sets used were: CFP (438/24 excitation, 470/24 emission) and YFP (513/17 excitation, 559/38 emission). Donor and Acceptor control channels were acquired using CFP-CFP and YFP-YFP, respectively. FRET images were acquired using CFP-YFP excitation and emission filter pair. Cells were chosen with similar intensity profiles in donor and acceptor prior to acquisition while under 37° C. and 5% C02 environmental conditions. Z stacks (0.25 μm) covering the whole cell (˜12 μm) were acquired before applying a conservative restorative algorithm for quantitative image deconvolution using SoftWorx v7.0, and saving files as a max pixel intensity projection tiff for each individual channel. The FRET ratio (FR) was determined by using the three-cube approach¹⁴ that is defined as the ratio of YFP emission in the presence of FRET over that in the absence of FRET:

FR=F_{A(D)}/F_{A}=[S_{FRET(DA)}−R_{D1}·S_{CFP(DA)}]/R_{A1}·[S_{YFP(DA)}−R_{D2}·S_{CFP(DA)}],
where R_{D1}=S_{FRET(D)}/S_{CFP(D)}; R_{D2}=S_{YFP(D)}/S_{CFP(D)}; R_{A1}=S_{FRET(A)}/S_{YFP(A)};
and S_{FRET}, S_{CFP} and S_{YFP} with D, A, DA in parenthesis refer to fluorescence signals of the FRET, CFP or YFP channel when only SARS2C (containing CFP as donor (D)), F1Y (containing YFP as acceptor (A)), or both SARS2C and F1Y-containing plasmids (both donor and acceptor (DA)) were used in the transfection. Averages from untransfected cells were subtracted from same-day fluorescence values for each filter cube. According to the definition of FRET ratio, FRET Ratio=1.0 means no FRET, while FRET Ratio>1.0 indicates the presence of FRET between the proteins carrying donor and acceptor proteins.

Antiviral activities in hACE2-mice models. hACE2-IRES-luc mice, female, 8-10 weeks, weighed approximately 17-25 g. A total of 12 mice were housed in a specific pathogen free (SPF) laboratory animal room and had food and water ad libitum. Antiviral F1 suspension was prepared by mixing F1-coding minicircle DNA with IN VIVO-JETPEI® at an N/P ratio of 6 and final DNA concentration of 0.4 μg/L in 5% glucose and incubating at room temperature for 15 minutes prior to use. Mice were randomly divided into two groups (n=6). On Day 0, mice were inoculated with 10⁴ PFU of SARS-CoV-2 (MT627325 strain). At 2 hours post-inoculation, mice in Control Group were treated once with PBS and mice in Treatment Group were treated once with 50 μL antiviral F1 suspension.

Body weight was recorded once a day for Day 0-3 for each group. Percentage of weight change was calculated by the equation:

100%×(Body Weight_{final}−Body Weightᵢₙᵢₜᵢₐₗ)/Body Weightᵢₙᵢₜᵢₐₗ.

On 3 days post-inoculation, mice were euthanized, and the lungs were dissected. Viral genomic RNA of SARS-CoV-2 was extracted by using a QIAAMP® Viral RNA Minikit (QIAGEN®). Reverse transcription was performed by using the HISCRIPT® II Q RT SuperMix (VAZYME®) for qPCR. qPCR was performed to obtain quantification of viral RNA copies by using the APPLIED BIOSYSTEMS™ QUANTSTUDIO™ 5 Real-Time PCR System.

Statistical Analysis. The p-values were calculated from unpaired two-tailed t-test comparing two groups for in vivo antiviral study and FRET experiments.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. Cui J, Li F, Shi Z L. Origin and evolution of pathogenic coronaviruses. Nature reviews Microbiology 2019; 17(3):181-192. DOI: 10.1038/s41579-018-0118-9.
2. de Wit E, van Doremalen N, Falzarano D, Munster V J. SARS and MERS: recent insights into emerging coronaviruses. Nature reviews Microbiology 2016; 14(8):523-34. DOI: 10.1038/nrmicro.2016.81.
3. Rambaut A, Loman N, Pybus O, et al. Preliminary genomic characterisation of an emergent SARS-CoV-2 lineage in the UK defined by a novel set of spike mutations. 2020.
4. Tegally H, Wilkinson E, al. e. Emergence and rapid spread of a new severe acute respiratory syndrome-related coronavirus 2 (SARS-CoV-2) lineage with multiple spike mutations in South Africa. medRxiv 2020; 2020.2012.2021.20248640.
5. Naveca F, Nascimento V, Souza V, et al. Phylogenetic relationship of SARS-CoV-2 sequences from Amazonas with emerging Brazilian variants harboring mutations E484K and N501Y in the Spike protein. virologicalorg 2021.
6. Zhou D, Dejnirattisai W, Supasa P, Liu C, al. e. Evidence of escape of SARS-CoV-2 variant B.1.351 from natural and vaccine induced sera. Cell 2021; In press.
7. Tada T, Dcosta BM, Samanovic-Golden M, et al. Neutralization of viruses with European, South African, and United States SARS-CoV-2 variant spike proteins by convalescent sera and BNT162b2 mRNA vaccine-elicited antibodies. bioRxiv 2021. DOI: 10.1101/2021.02.05.430003.
8. Wang P, Liu L, Iketani S, et al. Increased Resistance of SARS-CoV-2 Variants B.1.351 and B.1.1.7 to Antibody Neutralization. bioRxiv 2021. DOI: 10.1101/2021.01.25.428137.
9. Zhou B, Thi Nhu Thao T, Hoffmann D, et al. SARS-CoV-2 spike D614G change enhances replication and transmission. Nature 2021. DOI: 10.1038/s41586-021-03361-1.
10. Cele S, Gazy I, Jackson L, et al. Escape of SARS-CoV-2 501Y.V2 from neutralization by convalescent plasma. Nature 2021. DOI: 10.1038/s41586-021-03471-w.
11. Dejnirattisai W, Huo J, Zhou D, et al. SARS-CoV-2 Omicron-B.1.1.529 leads to widespread escape from neutralizing antibody responses. Cell 2022; 185(3):467-484 e15. DOI: 10.1016/j.cell.2021.12.046.
12. VanBlargan L A, Errico J M, Halfmann P J, et al. An infectious SARS-CoV-2 B.1.1.529 Omicron virus escapes neutralization by therapeutic monoclonal antibodies. Nat Med 2022. DOI: 10.1038/s41591-021-01678-y.
13. Zhao G, Du L, Ma C, et al. A safe and convenient pseudovirus-based inhibition assay to detect neutralizing antibodies and screen for viral entry inhibitors against the novel human coronavirus MERS-CoV. Virol J 2013; 10:266. DOI: 10.1186/1743-422X-10-266.
14. Erickson M G, Alseikhan B A, Peterson B Z, Yue D T. Preassociation of calmodulin with voltage-gated Ca(2+) channels revealed by FRET in single living cells. Neuron 2001; 31(6):973-85. DOI: 10.1016/s0896-6273(01)00438-x.
15. Kharitonenkov A, Shiyanova T L, Koester A, et al. FGF-21 as a novel metabolic regulator. The Journal of clinical investigation 2005; 115(6):1627-35. DOI: 10.1172/JCI23606.
16. V'Kovski P, Kratzel A, Steiner S, Stalder H, Thiel V. Coronavirus biology and replication: implications for SARS-CoV-2. Nature reviews Microbiology 2021; 19(3):155-170. DOI: 10.1038/s41579-020-00468-6.
17. Wrapp D, Wang N, Corbett K S, et al. Cryo-EM structure of the 2019-nCoV spike in the prefusion conformation. Science 2020; 367(6483):1260-1263. DOI: 10.1126/science.abb2507.
18. Walls A C, Park Y J, Tortorici M A, Wall A, McGuire A T, Veesler D. Structure, Function, and Antigenicity of the SARS-CoV-2 Spike Glycoprotein. Cell 2020; 183(6):1735. DOI: 10.1016/j.cell.2020.11.032.
19. Shang J, Ye G, Shi K, et al. Structural basis of receptor recognition by SARS-CoV-2. Nature 2020; 581(7807):221-224. DOI: 10.1038/s41586-020-2179-y.
20. Cai Y, Zhang J, Xiao T, et al. Distinct conformational states of SARS-CoV-2 spike protein. Science 2020; 369(6511):1586-1592. DOI: 10.1126/science.abd4251.
21. Bangaru S, Ozorowski G, Turner H L, et al. Structural analysis of full-length SARS-CoV-2 spike protein from an advanced vaccine candidate. Science 2020; 370(6520):1089-1094. DOI: 10.1126/science.abe1502.
22. Klein S, Cortese M, Winter S L, et al. SARS-CoV-2 structure and replication characterized by in situ cryo-electron tomography. Nat Commun 2020; 11(1):5885. DOI: 10.1038/s41467-020-19619-7.
23. Stertz S, Reichelt M, Spiegel M, et al. The intracellular sites of early replication and budding of SARS-coronavirus. Virology 2007; 361(2):304-15. DOI: 10.1016/j.virol.2006.11.027.
24. Coutard B, Valle C, de Lamballerie X, Canard B, Seidah N G, Decroly E. The spike glycoprotein of the new coronavirus 2019-nCoV contains a furin-like cleavage site absent in CoV of the same clade. Antiviral Res 2020; 176:104742. DOI: 10.1016/j.antiviral.2020.104742.
25. Johnson B A, Xie X, Bailey A L, et al. Loss of furin cleavage site attenuates SARS-CoV-2 pathogenesis. Nature 2021; 591(7849):293-299. DOI: 10.1038/s41586-021-03237-4.
26. Hardee C L, Arevalo-Soliz L M, Hornstein B D, Zechiedrich L. Advances in Non-Viral DNA Vectors for Gene Therapy. Genes (Basel) 2017; 8(2). DOI: 10.3390/genes8020065.
27. Catanese D J, Jr., Fogg J M, Schrock D E, 2nd, Gilbert B E, Zechiedrich L. Supercoiled Minivector DNA resists shear forces associated with gene therapy delivery. Gene Ther 2012; 19(1):94-100. DOI: 10.1038/gt.2011.77.
28. Kay M A, He C Y, Chen Z Y. A robust system for production of minicircle DNA vectors. Nat Biotechnol 2010; 28(12):1287-9. DOI: 10.1038/nbt.1708.
29. Madeira, F., Park, Y. M., Lee, J., Buso, N., Gur, T., Madhusoodanan, N., Basutkar, P., Tivey, A. R. N., Potter, S. C., Finn, R. D., and Lopez, R. (2019) The EMBL-EBI search and sequence analysis tools APIs in 2019, Nucleic Acids Res 47, W636-W641.

1-415. (canceled)

416. A method for regulating a target protein or biological function thereof in vivo in a subject comprising contacting the target protein or a portion thereof and/or a native interacting partner of the target protein or a portion thereof with a composition comprising an effective amount of a polypeptide having an amino acid sequence corresponding to a sequence of the target protein, wherein the length of the polypeptide is greater than 30 amino acids, and wherein:
the polypeptide and the target protein form a non-native protein complex upon contact of the target protein with the polypeptide, thereby regulating the target protein or biological function thereof in vivo;
the polypeptide and the native interacting partner of the target protein form a non-native protein complex upon contact of the native interacting partner of the target protein with the polypeptide, thereby regulating the target protein or biological function thereof in vivo; and/or
the polypeptide, the target protein, and the native interacting partner of the target protein form a non-native protein complex upon contact of the target protein and the native interacting partner of the target protein with the polypeptide, thereby regulating the target protein or biological function thereof in vivo, optionally, wherein the polypeptide has an amino acid sequence corresponding to an oligomerization domain of the target protein, and:
the polypeptide oligomerizes with the oligomerization domain of the target protein to form a non-native protein complex, thereby regulating the target protein or biological function thereof in vivo;
the polypeptide oligomerizes with the oligomerization domain of the native interacting partner of the target protein to form a non-native protein complex, thereby regulating the target protein or biological function thereof in vivo; and/or
the polypeptide oligomerizes with the oligomerization domain of the target protein and the native interacting partner of the target protein to form a non-native protein complex, thereby regulating the target protein or biological function thereof in vivo.

417. The method of claim 416, wherein the polypeptide having an amino acid sequence corresponding to a sequence of the target protein has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with the sequence of the target protein, or comprises the corresponding sequence of the target protein.

418. The method of claim 416, wherein the target protein is a viral protein, optionally, wherein the target protein is a viral glycoprotein and/or a viral spike protein.

419. The method of claim 416, wherein regulating the target protein or biological function thereof in vivo treats or prevents a disease or condition in the subject, optionally, wherein the disease or condition is a viral infection.

420. The method of claim 416, wherein:
the non-native protein complex is subjected to proteasomal degradation;
formation of the non-native protein complex regulates the target protein or biological function thereof in vivo by inhibiting homo-oligomerization of the target protein;
formation of the non-native protein complex regulates the target protein or biological function thereof in vivo by inhibiting hetero-oligomerization of the target protein; and/or
formation of the non-native protein complex regulates the target protein or biological function thereof in vivo by inhibiting homo-oligomerization and hetero-oligomerization of the target protein.

421. A method for treating or preventing an infection in a subject comprising administering to the subject a therapeutically effective amount of a composition comprising a polypeptide having an amino acid sequence corresponding to a sequence of a target protein and/or a native interacting partner of the target protein, optionally, wherein the sequence of the target protein is an oligomerization domain of the target protein, and having at least 10% identity with SEQ ID NO:2, 4, 6, 8, 10, 12, 14, 16, 18, 20, 25, or 34, wherein the polypeptide contacts the target protein and/or a native interacting partner of the target protein to form a non-native protein complex.

422. The method of claim 421, wherein the non-native protein complex is subjected to proteasomal degradation;
regulating the target protein or biological function thereof in vivo treats or prevents the infection;
formation and translocation of the target protein to cell surfaces of the subject and/or to viral envelopes is inhibited; and/or
the amount of the target protein on cell surfaces of the subject and/or on viral envelopes is reduced.

423. The method of claim 421, wherein:
the infection is a coronavirus infection, and:
the coronavirus comprises SARS-CoV, and the protein comprises a SARS-CoV spike protein;
the coronavirus comprises SARS-CoV-2, and the protein comprises a SARS-CoV-2 spike protein;
the coronavirus comprises MERS-CoV, and the protein comprises a MERS-CoV spike protein;
the coronavirus comprises HCoV-229E, and the protein comprises a HCoV-229E spike protein;
the coronavirus comprises HCoV-NL63, and the protein comprises a HCoV-NL63 spike protein;
the coronavirus comprises HCoV-OC43, and the protein comprises a HCoV-OC43 spike protein; or
the coronavirus comprises HCoV-HKU1, and the protein comprises a HCoV-HKU1 spike protein;
the infection is an HIV infection, and the protein comprises an HIV spike protein, optionally, wherein the HIV spike protein comprises an HIV gp160 spike protein;
the infection is an Ebola infection, and wherein the protein comprises an Ebola glycoprotein, optionally, wherein the Ebola glycoprotein comprises an Ebola GP glycoprotein;
the infection is an influenza infection, and:
the influenza comprises influenza virus A, and the protein comprises an influenza virus A spike protein;
the influenza virus comprises influenza virus A/H1, and the protein comprises an influenza virus A/H1 HA spike protein;
the influenza virus A comprises influenza virus A/H3, and the protein comprises an influenza virus A/H3 HA spike protein;
the influenza comprises influenza virus B, and the protein comprises an influenza virus B spike protein;
the influenza virus B comprises influenza virus B/Victoria, and the protein comprises an influenza virus B/Victoria HA spike protein; or
the influenza virus B comprises influenza virus B/Yamagata, and the protein comprises an influenza virus B/Yamagata HA spike protein; or
the infection is an RSV infection, and the protein comprises an RSV glycoprotein, optionally, wherein the RSV glycoprotein comprises an RSV F glycoprotein.

424. The method of claim 421, wherein:
the infection is a coronavirus infection, and the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:2, SEQ ID NO:4, SEQ ID NO:6, SEQ ID NO:8, SEQ ID NO:10, SEQ ID NO:12, SEQ ID NO:14, or SEQ ID NO:16, or comprises SEQ ID NO:2, SEQ ID NO:4, SEQ ID NO:6, SEQ ID NO:8, SEQ ID NO:10, SEQ ID NO:12, SEQ ID NO:14, or SEQ ID NO:16;
the infection is an HIV infection, and the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:18 or comprises SEQ ID NO:18;
the infection is an Ebola infection, and the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:20, or comprises SEQ ID NO:20;
the infection is an influenza infection, and the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:25, or comprises SEQ ID NO:25;
the infection is an RSV infection, and the polypeptide has at least 10, 15, 20, 25, 30, 35, 40, 45, 50, 55, 60, 65, 70, 75, 80, 85, 86, 87, 88, 89, 90, 91, 92, 93, 94, 95, 96, 97, 98, or 99% identity with SEQ ID NO:34, or comprises SEQ ID NO:34.

425. The method of claim 421, further comprising:
diagnosing the subject with the infection;
diagnosing the subject as having symptoms of the infection; or
diagnosing the subject as being at risk of having the infection.

426. The method of claim 421, wherein the subject is at high risk for having an infection, does not have an infection, has tested negative for an infection, and/or was diagnosed as having an infection, and optionally, wherein the infection is prevented, reduced in severity, and/or delayed in onset.

427. The method of claim 421, wherein the subject is provided an effective amount of a second therapy for the infection, optionally, wherein the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof.

428. The method of claim 421, wherein:
a dose of between 0.1 to 1000 mg/kg or 0.1 to 1000 μg/kg body weight of the subject of the polypeptide is administered to the subject; or
the polypeptide is expressed from a vector encoding the polypeptide, a dose of between 1×10⁸ to 1×10¹⁸ vector copies/kg body weight of the subject is administered to the subject, the vector transduces cells of the subject, and the cells of the subject express the polypeptide.

429. The method of claim 421, wherein the composition further comprises a pharmaceutically acceptable carrier, optionally, wherein the pharmaceutically acceptable carrier comprises liposomes, polymeric micelles, microspheres, or nanoparticles.

430. The method of claim 421, wherein:
a single dose or multiple doses of the composition are administered;
the composition is delivered to the subject once a day, more than once a day, more than once a week, more than once a month, or more than once a year;
the composition is delivered systemically or locally; and/or
the composition is administered to the subject intranasally, intravenously, intraperitoneally, intratracheally, intramuscularly, endoscopically, percutaneously, subcutaneously, regionally, intracranially, by inhalation, by injection, by infusion, or by perfusion.

431. A pharmaceutical composition comprising a vector encoding a polypeptide having an amino acid sequence corresponding to a sequence of a target protein and/or a native interacting partner of the target protein, optionally, wherein the sequence of the target protein is an oligomerization domain of the target protein,
wherein the length of the polypeptide is greater than 30 amino acids, and
wherein the polypeptide has at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with the sequence of the target protein, or comprises the sequence of the target protein.

432. The pharmaceutical composition of claim 431, wherein the vector encodes a polypeptide comprising an amino acid sequence having at least 10, 15, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, or 99% identity with SEQ ID NO:2, 4, 6, 8, 10, 12, 14, 16, 18, 20, 25, or 34, or a sequence comprising SEQ ID NO:2, 4, 6, 8, 10, 12, 14, 16, 18, 20, 25, or 34.

433. The composition of claim 431, further comprising a second therapy for an infection, optionally, wherein the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof.

434. The composition of claim 431, wherein the composition further comprises a pharmaceutically acceptable carrier, optionally, wherein the pharmaceutically acceptable carrier comprises liposomes, polymeric micelles, microspheres, or nanoparticles.
